(19) United States
(12) Patent Application Publication
(10) Pub. No.: US 2024/0115711 A1
(43) Pub. Date: Apr. 11, 2024

(54) Novel Bifunctional Molecules For Targeted Protein Degradation

(71) Applicant: Amphista Therapeutics Limited, Cambridge (GB)

(72) Inventors: Andrea TESTA, Glasgow (GB); Callum MACGREGOR, Glasgow (GB); David MCGARRY, Airdrie (GB); Gregor MEIER, Glasgow (GB); Ian CHURCHER, Huntingdon (GB); Michael MATHIESON, Glasgow (GB)

(21) Appl. No.: 18/266,294

(22) PCT Filed: Dec. 16, 2021
(86) PCT No.: PCT/GB2021/053332

§ 371 Date: Jun. 9, 2023

(30) Foreign Application Priority Data
Dec. 18, 2020 (GB) 2020186.9
Feb. 22, 2021 (GB) 2102494.8

(51) Int. Cl. A61K 47/54; A61P 35/00; G01N 33/50

(52) U.S. Cl. CPC A61K 47/545; A61P 35/00; G01N 33/5011; G01N 2500/10

(57) ABSTRACT

The present disclosure relates to a novel class of bifunctional molecules that are useful in a targeted or selective degradation of a protein.

### FIELD

The present disclosure relates to a novel class of bifunctional molecules that are useful in a targeted or selective degradation of a protein.

### BACKGROUND

Targeted Protein Degradation (TPD) is a therapeutic modality, which relies on the use of synthetic molecules to repurpose cellular degradation machinery to induce degradation of specific disease-causing proteins. TPD approaches offer a number of advantages over other drug modalities (e.g. small molecule inhibitors, antibodies & protein-based agents, antisense oligonucleotides & related knockdown approaches) including: potentiated pharmacology due to catalytic protein removal from within cells; ability to inhibit multiple functions of a specific drug target including e.g. scaffolding function through target knockdown; opportunity for systemic dosing with good biodistribution; potent in vivo efficacy due to catalytic potency and long duration of action limited only by de novo protein resynthesis; and facile chemical synthesis and formulation using application of small molecule processes.

The majority of physiologic post-translational regulation of protein levels as well as removal of damaged, misfolded, or excess proteins is mediated by the ubiquitin-proteasome system (UPS). The UPS can be repurposed to degrade specific proteins using bifunctional chemical molecules as therapeutic agents, which act by inducing the proximity of desired substrates with UPS proteins to initiate a cascade of events which ultimately lead to degradation, and removal from the cell, of the desired targets by the proteasome.

Proteolysis targeting chimeras (PROTACs) constitute one such class of bifunctional degraders, which induce proximity of target proteins to the UPS by recruitment of specific ubiquitin E3 ligases. PROTACs are composed of two ligands joined by a linker—one ligand to engage a desired target protein and another ligand to recruit a ubiquitin E3 ligase.

The E3 ligases used most frequently in PROTACs are von Hippel-Lindau (VHL) and Cereblon (CRBN). PROTACs recruiting VHL are typically based on hydroxyproline-containing ligands, whereas PROTACs recruiting CRBN are typically characterised by the presence of a glutarimide moiety, such as thalidomide, pomalidomide and lenalidomide or close analogues to act as the warhead. Other ligases including mdm2 and the IAP family have also shown utility in PROTAC design.

However, these approaches suffer from a range of limitations, which restrict their utility to treat a wide range of diseases. For example, limitations of current PROTAC approaches include: inability to efficiently degrade some targets; poor activity of PROTACs in many specific cells due to low and variable expression of E3 ligases and other proteins required for efficient degradation; chemical properties which make it more difficult to prepare degraders with suitable drug-like properties including good drug metabolism & pharmacokinetic profiles; and high susceptibility to induced resistance mechanisms in tumours.

Because of these limitations, there remains a need to identify novel degrading mechanisms and warheads able to deliver new bifunctional degrader molecules, which show efficient degradation across a range of targets and cellular systems and/or with improved profiles suitable for drug development.

Further bifunctional degrader molecules have been described in WO 2019/238886, WO 2019/238817 and WO 2019/238816.

### SUMMARY

The present disclosure is based on the identification of a novel class of bifunctional molecules that are useful in a targeted and/or selective degradation of a protein, e.g. a “target protein”. In particular, the present disclosure provides bifunctional molecules, which facilitate proteasomal degradation of selected target protein(s) using a novel class of warhead.

The bifunctional molecules described herein comprise a general structure of:

TBL-L-Z

wherein TBL is a target protein binding ligand and L is a linker. The moiety “Z” (sometimes referred to herein as a “warhead”) modulates, facilitates and/or promotes proteasomal degradation of the target protein and may, in some cases, be referred to as a modulator, facilitator and/or promoter of proteasomal degradation. For example, in use, the TBL moiety of the bifunctional molecule binds to a target protein. The moiety Z (which is joined to the TBL moiety via the linker) then modulates, facilitates and/or promotes the degradation of this target protein, e.g. by acting to bring the target protein into proximity with a proteasome and/or by otherwise causing the target protein to be marked for proteasomal degradation within a cell.

The bifunctional molecules described in the present disclosure have been shown to be effective degraders against a wide range of targets. Without being bound by theory, it is hypothesised that the Z moiety of the bifunctional molecules described herein does not bind to the particular E3 ligases typically relied on in the classical PROTAC approaches discussed above (such as CRBN and VHL). Accordingly, the bifunctional molecules described herein are believed to modulate, facilitate and/or promote proteasomal degradation via an alternative mechanism. Thus, the present class of bifunctional molecules may be useful against a wider range of diseases (including those that are resistant to many PROTAC degraders).

The bifunctional molecules described herein may provide degraders with one or more properties that will facilitate, enhance and/or promote their use in vivo (e.g. one or more drug-like properties). In particular, bifunctional molecules comprising the warhead Z may offer improvements in levels of bioavailability (e.g. oral bioavailability) over many classical PROTAC degraders. Additionally, or alternatively, bifunctional molecules comprising the warhead Z may provide improved levels of CNS (central nervous system) penetration (in contrast to many other degrader molecules currently known in the art).

Furthermore, the present disclosure is based on the finding that a series of N-alkylated compounds can provide particularly effective modulators, facilitators and/or promoters of proteasomal degradation, e.g. in bifunctional molecules intended for use in targeted and/or selective protein degradation. In particular, it has been found that this N-alkylated series of compounds can provide significant improvements in the protein degrader activity of the bifunctional molecule.

According to a first aspect of the disclosure, there is provided a bifunctional molecule comprising the general formula:

TBL-L-Z

wherein TBL is a target protein binding ligand;
L is a linker; and
Z comprises a structure according to formula (I):

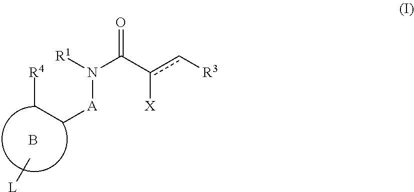

wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR²R^{2′};
B is selected from aryl, heteroaryl, substituted aryl and substituted heteroaryl;
R² and R^{2′} are each independently selected from H and C₁ to C₆ alkyl, optionally wherein the C₁ to C₆ alkyl is substituted with one or more heteroatoms selected from N, O or S, or wherein R² and R^{2′} together form a 3-, 4-, 5- or 6-membered carbocyclic or heterocyclic ring;
R³ is selected from C₁ to C₆ alkyl, aryl, heteroaryl, substituted aryl, substituted heteroaryl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
X is selected from H and an electron-withdrawing group;
R⁴ is H, C₁ to C₆ alkyl, optionally wherein the C₁ to C₆ alkyl is substituted with one or more heteroatoms selected from N, O or S;
or wherein R¹ and R⁴ together form a 5-, 6-, or 7-membered heterocyclic ring;
or wherein when A is CR²R^{2′}:
R¹ and R² together form a 5-, 6-, or 7-membered heterocyclic ring; or
R² and R⁴ together form a 5-, 6-, or 7-membered heterocyclic or carbocyclic ring;
wherein [symbol] is a single bond or double bond; and
L shows the point of attachment of the linker.

On ring B, groups R⁴ and A may be held at adjacent positions on the aryl, heteroaryl, substituted aryl or substituted heteroaryl ring. In other words, the R⁴ and A groups may be in a 1,2 substitution pattern with one another, or may be separated by 3 bonds. For the avoidance of doubt, where B is a heteroaryl or substituted heteroaryl, a heteroatom contained within ring B may be directly bonded to A or R⁴.

As shown in formula (I) above, the linker is appended to moiety Z via ring B. The linker may be attached to moiety Z by way of a covalent bond between an atom on the linker and an atom contained in the ring system of the optionally substituted aryl or heteroaryl group of ring B. This linker may be attached to ring B at any position on the optionally substituted aromatic or heteroaromatic ring (provided it has the correct valency and/or is chemically suitable). For example, the linker may replace a hydrogen atom at any position on the aromatic or heteroaromatic ring.

In other examples, Z may comprise a structure as shown in formula (I) above, wherein:
A, B, X and R⁴ are as defined above; and wherein
R¹ is selected from optionally substituted C₁ to C₆ alkyl, optionally substituted C₁ to C₆ haloalkyl, optionally substituted benzyl, optionally substituted carbocyclyl, and optionally substituted heterocyclyl;
R² and R^{2′} are each independently selected from H and optionally substituted C₁ to C₆ alkyl, or wherein R² and R^{2′} together form a 3-, 4-, 5- or 6-membered optionally substituted carbocyclic or heterocyclic ring; and
R³ is selected from optionally substituted C₁ to C₆ alkyl, optionally substituted aryl, optionally substituted heteroaryl, optionally substituted carbocyclyl and optionally substituted heterocyclyl.

In those cases where R¹ and R⁴ together form a 5-, 6-, or 7-membered heterocyclic ring, Z may be represented by formula (Ia):

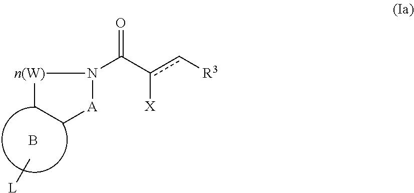

wherein A, B, R³, X and L are as defined for formula (I); and
n is 1, 2 or 3;
W is selected from CR^{W1}R^{W2}, O, NR^{W3}, and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

In those cases, where R¹ and R² together form a 5-, 6-, or 7-membered heterocyclic ring, Z may be represented as formula (Ib):

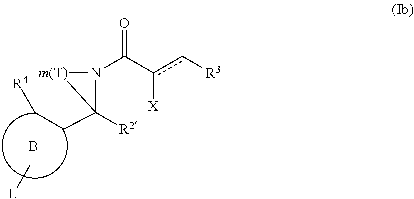

Wherein B, R^{2′}, R³, R⁴, X and L are as defined for formula (I);
m is 3, 4 or 5;
each T is independently selected from CR^{T1}R^{T2}, O, NR^{T3}, and S; and
R^{T1}, R^{T2} and R^{T3} are each independently selected from H and C₁ to C₆ alkyl.

In those cases where R² and R⁴ together form a 5-, 6-, or 7-membered heterocyclic or carbocyclic ring, Z may be represented as formula (Ic):

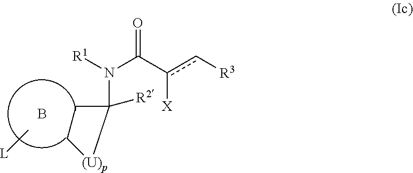

Wherein B, R¹, R^{2′}, R³, X and L are as defined for formula (I);
p is 2, 3 or 4; and
each U is independently selected from CR^{U1}R^{U2}, O, NR^{U3}, and S; and
R^{U1}, R^{U2} and R^{U3} are each independently selected from H and C₁ to C₆ alkyl.

As used herein, “C₁-C₆ alkyl” may be selected from straight or branched chain hydrocarbyl groups containing from 1 to 6 carbon atoms. Representative examples are methyl, ethyl, n-propyl, iso-propyl, n-butyl, sec-butyl, iso-butyl, tert-butyl, n-pentyl, isopentyl, neopentyl, n-hexyl, isohexyl, neohexyl, etc. When a C₁-C₆ alkyl group is substituted, any hydrogen atom(s), CH₃, CH₂ or CH group(s) may be replaced with the substituent(s), providing valencies are satisfied. Where the C₁-C₆ alkyl comprises a divalent hydrocarbon radical (containing from 1 to 6 carbon atoms), this moiety may sometimes be referred to herein as a C₁-C₆ alkylene.

“Benzyl” as used herein refers to a —CH₂Ph group. As used herein, a “substituted benzyl” refers to a benzyl group as defined herein which comprises one or more substituents on the aromatic ring. When a benzyl group is substituted, any hydrogen atom(s) may be replaced with the substituent(s), providing valencies are satisfied.

As used herein, the term “aryl” refers to a mono- or polycyclic aromatic hydrocarbon system having 6 to 14 carbon atoms. Representative examples of suitable “aryl” groups include, but are not limited to, phenyl, biphenyl, naphthyl, 1-naphthyl, 2-naphthyl and anthracenyl. As used herein, “substituted aryl” refers to an aryl group as defined herein which comprises one or more substituents on the aromatic ring. When an aryl group is substituted, any hydrogen atom(s) may be replaced with the substituent(s), providing valencies are satisfied.

As used herein, “heteroaryl” may be a single or fused ring system having one or more aromatic rings containing 1 or more O, N and/or S heteroatoms. Representative examples of heteroaryl groups may include, but are not limited to, pyrrolyl, furanyl, thiophenyl, pyrazolyl, imidazolyl, oxazolyl, isoxazolyl, thiazolyl, pyridinyl, pyrimidinyl, pyridazinyl, pyrazinyl, indolyl, benzofuranyl, benzothiazolyl, benzimidazolyl, indazolyl, benzoxazolyl, benzisoxazolyl etc. As used herein, “substituted heteroaryl” refers to a heteroaryl group as defined herein which comprises one or more substituents on the heteroaromatic ring.

As used herein, a “carbocyclic ring” is a ring containing 3 to 10 carbon atoms, in some cases 3 to 8 carbon atoms. The ring may be aliphatic. Thus, as used herein, references to “carbocyclyl” and “substituted carbocyclyl” groups may refer to aliphatic carbocyclyl groups and aliphatic substituted carbocyclyl groups. The ring may be saturated or unsaturated, e.g. in some cases the ring may contain one or more double or triple bonds. Representative examples of carbocyclyl groups include cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, cyclooctynl etc. As used herein, “substituted carbocyclyl” refers to a carbocyclyl group as defined herein which comprises one or more substituents on the carbocyclic ring. When a carbocyclyl group is substituted, any hydrogen atom(s) may be replaced with the substituent(s), providing valencies are satisfied.

As used herein, a “heterocyclic ring” may comprise at least 1 heteroatom selected from O, N and S. The heterocyclic ring may be a ring comprising 3 to 10 atoms, in some cases 3 to 8 atoms. The ring may be aliphatic. Thus, as used herein, references to “heterocyclyl” and “substituted heterocyclyl” groups may refer to aliphatic heterocyclyl groups and aliphatic substituted heterocyclyl groups. The ring may be saturated or unsaturated, e.g. in some cases the ring may contain one or more double or triple bonds. Any N heteroatom present in the heterocyclic group may be C₁ to C₆ alkyl-substituted. Representative examples of heterocyclyl groups include, but are not limited to, pyrrolidinyl, tetrahydrofuranyl, dioxolanyl, dithiolanyl, thiazolidinyl, isothiazolidinyl, oxazolidinyl, isoxazolidinyl, pyrazolidinyl, imidazolidinyl, piperidinyl, piperazinyl, N-alkylpiperazinyl, morpholinyl, dioxanyl, oxazolidinyl, tetrahydropyranyl etc. As used herein, “substituted heterocyclyl” refers to a heterocyclyl group as defined herein which comprises one or more substituents on the heterocyclic ring.

As used herein, the term “optionally substituted” means that the moiety may comprise one or more substituents.

As used herein, a “substituent” may include, but is not limited to, hydroxyl, thiol, carboxyl, cyano (CN), nitro (NO₂), halo, haloalkyl (e.g. a C₁ to C₆ haloalkyl), an alkyl group (e.g. C₁ to C₁₀ or C₁ to C₆), aryl (e.g. phenyl and substituted phenyl for example benzyl or benzoyl), alkoxy group (e.g. C₁ to C₆ alkoxy) or aryloxy (e.g. phenoxy and substituted phenoxy), thioether (e.g. C₁ to C₆ alkyl or aryl thioether), keto (e.g. C₁ to C₆ keto), ester (e.g. C₁ to C₆ alkyl or aryl ester, which may be present as an oxyester or carbonylester on the substituted moiety), thioester (e.g. C₁ to C₆ alkyl or aryl thioester), alkylene ester (such that attachment is on the alkylene group, rather than at the ester function which is optionally substituted with a C₁ to C₆ alkyl or aryl group), amine (including a five- or six-membered cyclic alkylene amine, further including a C₁ to C₆ alkyl amine or a C₁ to C₆ dialkyl amine which alkyl groups may be substituted with one or two hydroxyl groups), amido (e.g. which may be substituted with one or two C₁ to C₆ alkyl groups (including a carboxamide which is optionally substituted with one or two C₁ to C₆ alkyl groups), alkanol (e.g. C₁ to C₆ alkyl or aryl alkanol), or carboxylic acid (e.g. C₁ to C₆ alkyl or aryl carboxylic acid), sulfoxide, sulfone, sulfonamide, and urethane (such as —O—C(O)—NR₂ or —N(R)—C(O)—O—R, wherein each R in this context is independently selected from C₁ to C₆ alkyl or aryl).

In some examples, and unless the context indicates otherwise, a “substituent” may include, but is not limited to, halo, C₁ to C₆ alkyl, NH₂, NH(C₁ to C₆ alkyl), N(C₁ to C₆ alkyl)₂, OH, O(C₁ to C₆ alkyl), NO₂, CN, C₁-C₆ haloalkyl, CONH₂, CONH(C₁ to C₆ alkyl), CON(C₁ to C₆ alkyl)₂, C(O)OC₁ to C₆ alkyl, CO(C₁ to C₆ alkyl), S(C₁ to C₆alkyl), S(O)(OC₁ to C₆ alkyl) and SO(C₁ to C₆ alkyl).

As used herein, a “halo” group may be F, Cl, Br, or I, typically F.

As used herein, “haloalkyl” may be an alkyl group in which one or more hydrogen atoms thereon have been replaced with a halogen atom. By way of a representative example, a C₁-C₆ haloalkyl may be a fluoroalkyl, such as trifluoromethyl (—CF₃) or 1,1-difluoroethyl (—CH₂CHF₂).

As used herein, an electron withdrawing group may refer to any group which draws electron density away from neighbouring atoms and towards itself. Typically, the electron withdrawing group draws electron density away from neighbouring atoms and towards itself more strongly than a hydrogen substituent. Representative examples of suitable electron withdrawing groups include, but are not limited to, —CN, halo, —NO₂, —CONH₂, —CONH(C₁ to C₆ alkyl), —CON(C₁ to C₆ alkyl)₂, —SO₂(C₁ to C₆ alkyl), —CO₂(C₁ to C₆ alkyl), —CO(C₁ to C₆ alky) and C₁ to C₆ haloalkyl.

With respect to the various structures for Z defined by the formulae herein, R¹ may be C₁ to C₆ alkyl, such as C₁ to C₄ alkyl. For example, R¹ may be selected from the group consisting of methyl, ethyl, n-propyl, iso-propyl.

As stated above for formula (I), A is either absent or is CR²R^{2′}. In some cases, where A is CR²R^{2′}, R² and R^{2′} are each independently selected from H and C₁ to C₆ alkyl, such as methyl, ethyl, n-propyl, iso-propyl and n-butyl. In some examples, one of R² and R^{2′} is a hydrogen and the other is C₁ to C₆ alkyl. For example, R² may be methyl, ethyl or n-propyl and R^{2′} may be H.

As stated above, R³ is selected from C₁ to C₆ alkyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group. For example, R³ may be selected from C₁ to C₆ alkyl, aryl, heteroaryl, substituted aryl and substituted heteroaryl. By way of further example, R³ may be selected from aryl, heteroaryl, substituted aryl, substituted heteroaryl and C₁-C₆ alkyl substituted with a heterocyclic group. Representative examples of suitable R³ groups include, but are not limited to, phenyl, thiazolyl, benzothiazolyl, pyridinyl, tert-butyl, pyrazolyl, imidazolyl, oxazolyl, N—C₁ to C₆ alkylenemorpholine, imidazo(1,2-a)pyridinyl, thiophenyl and 4,5,6,7-tetrahydro-1,3-benzothiazolyl, such as phenyl, thiazolyl, benzothiazolyl, pyridinyl and tert-butyl. In each case, these R³ groups may be substituted, such as substituted phenyl, substituted thiazolyl, substituted benzothiazolyl, substituted pyridinyl, substituted tert-butyl, substituted pyrazolyl, substituted imidazolyl, substituted oxazolyl, substituted N—C₁ to C₆ alkylenemorpholine, substituted imidazo(1,2-a)pyridinyl, substituted thiophenyl and substituted 4,5,6,7-tetrahydro-1,3-benzothiazolyl. Where R³ is a substituted aryl or heteroaryl group, there may be one or more substituents on the aromatic ring e.g. it may be mono-, di- or tri-substituted. Where R³ is optionally substituted pyrazolyl or imidazolyl, a nitrogen atom of the pyrazolyl or imidazolyl ring may be substituted with C₁ to C₆ alkyl, such as methyl.

Examples of suitable R³ groups are shown below:

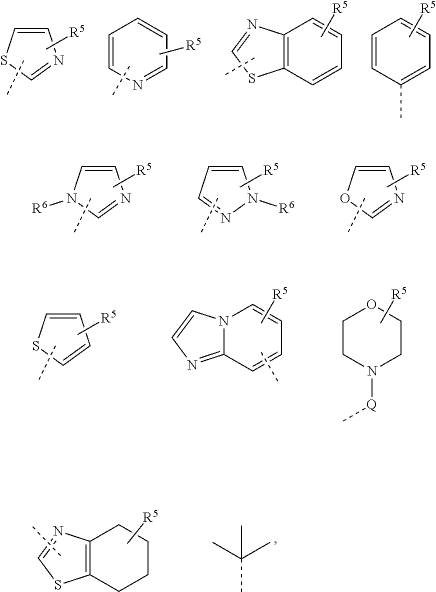

wherein the dotted line on the structures indicates the position that each of the respective R³ groups may be joined to the structure shown in formulae (I) to (Ic). Where the dotted line is not shown connected directly to an atom, the R³ group may be connected to the structure shown in formulae (I) to (Ic) by a covalent bond to an atom at any position on the aromatic ring (provided that it has the correct valency and/or is chemically suitable). For example, a hydrogen at any position on the R³ group may be replaced with a bond to the structure shown in formula (I).

R⁵ may be any substituent as described herein or may be absent. In some examples, R⁵ may be selected from halo (e.g. F, Cl, Br, I), CF₃, —CH₂F, —CHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe. As stated above, there may be one or more substituents on the aromatic ring. Where two or more substituents are present, each substituent may be independently selected from the R⁵ groups noted above.

R⁶ may be C₁ to C₆ alkyl, such as methyl.

Q may be C₁ to C₆ alkylene such as dimethylmethylene (—C(CH₃)₂—) or dimethylethylene (—C(CH₃)₂CH₂—).

By way of further example, a suitable R³ group may be selected from the following:

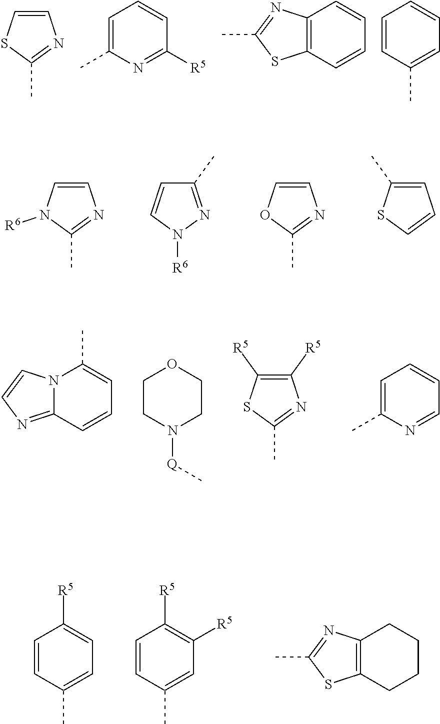

wherein the dotted line on the structures indicates the position that each of the respective R³ groups may be joined to the structure shown in formulae (I) to (Ic).

As stated above, X may be selected from H or an electron-withdrawing group. By way of example only, the electron-withdrawing group may be selected from the group consisting of —CN, halo, —CF₃, —NO₂, —CONH₂, —CONH(C₁ to C₆ alkyl), —CON(C₁ to C₆ alkyl)₂, —SO₂(C₁ to C₆ alkyl), —CO₂(C₁ to C₆ alkyl), —CO(C₁ to C₆ alkyl) and C₁ to C₆ haloalkyl. In some examples, X is —H, —F, —CF₃, —SO₂Me or —CN. In particular, for any of the formulae (I) to (IV) as described herein, X may be —CN.

In certain examples, Z comprises a structure according to formula (II):

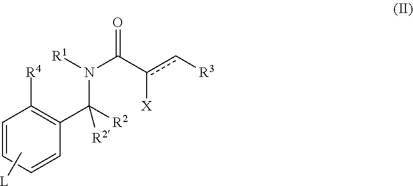

wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclyl or heterocyclyl group;
R² and R^{2′} are each independently selected from H and C₁ to C₆ alkyl;
R³ is selected from C₁ to C₆ alkyl, aryl, heteroaryl, substituted aryl, substituted heteroaryl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclyl or heterocyclyl group;
X is selected from H or an electron-withdrawing group;
R⁴ is H, C₁-C₆ alkyl, optionally wherein the C₁-C₆ alkyl is substituted with one or more heteroatoms selected from N, O or S;
or wherein R¹ and R⁴ together form a 5-, 6-, or 7-membered heterocyclic ring;
or wherein R¹ and R² together form a 5-, 6-, or 7-membered heterocyclic ring;
or wherein R² and R⁴ together form a 5-, 6-, or 7-membered heterocyclic or carbocyclic ring;
wherein [symbol] is a single bond or double bond; and
L shows the position of attachment of the linker.

As shown in formula (II) above, the linker is appended to moiety Z via the aromatic ring. In particular, the linker is attached to moiety Z by way of a covalent bond between an atom on the linker and a carbon atom of the aryl ring system. The linker may be attached to the aromatic ring at any position (provided it has the correct valency and/or is chemically suitable). For example, the linker may replace a hydrogen atom at any position on the aromatic ring.

A representative example of a compound according to formula (II) includes, but is not limited to:

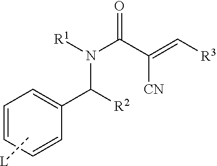

Wherein R³ and L are as defined for formulae (I) and (II) herein;
R¹ is selected from C₁ to C₆ alkyl; and
R² is selected from C₁ to C₆ alkyl.

In some cases, R¹ is methyl and R² is n-propyl.

In certain examples, when R¹ and R⁴ together form a 5-, 6-, or 7-membered heterocyclic ring, Z may be represented as formula (IIaa):

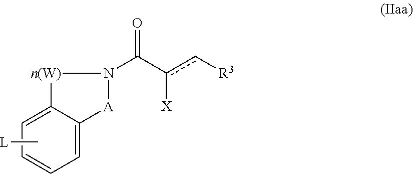

Wherein A, R³, X and L are as defined for formulae (I) and (II) herein;
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and wherein when n is 2 or 3, each W is independently selected from CR^{W1}R^{W2}, O, NR^{W3}, and S.

In some cases, each W is CR^{W1}R^{W2} and/or X is CN.

Representative examples of compounds according to formula (IIaa) include, but are not limited to:

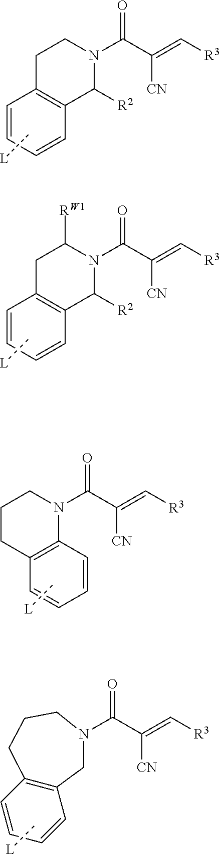

Wherein R and L are as defined herein for formula (I) above;
R² may be selected from H or C₁-C₆ alkyl (such as methyl or ethyl); and
R^{W1} may be selected from C₁-C₆ alkyl (such as methyl or ethyl).

By way of further example, when R¹ and R⁴ together form a 5-, 6-, or 7-membered heterocyclic ring, Z may be represented as formula (IIa):

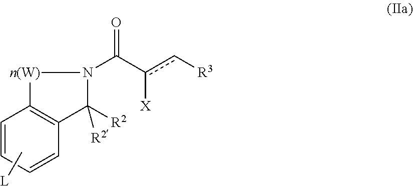

Wherein R², R^{2′}, R³, X and L are as defined for formula (II);
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and wherein when n is 2 or 3, each W is independently selected from CR^{W1}R^{W2}, O, NR^{W3}, and S.

In some cases, each W is CH₂ and/or X is CN.

When R¹ and R² together form a 5-, 6-, or 7-membered heterocyclic ring, Z may be represented as formula (IIb):

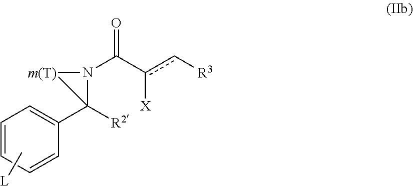

Wherein R^{2′}, R³, X and L are as defined for formula (II);
m is 3, 4 or 5;
each T is independently selected from CR^{T1}R^{T2}, O, NR^{T3} and S; and
R^{T1}, R^{T2} and R^{T3} are each independently selected from H and C₁ to C₆ alkyl.

For example, in some cases, each T is CH₂ and/or X is CN.

When R² and R⁴ together form a 5-, 6-, or 7-membered heterocyclic or carbocyclic ring, Z may be represented as formula (IIc):

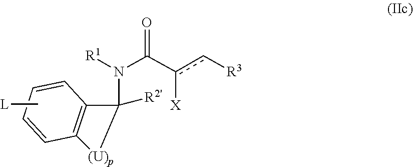

Wherein R¹, R^{2′}, R³, X and L are as defined for formula (II);
p is 2, 3 or 4; and
each U is independently selected from CR^{U1}R^{U2}, O, NR^{U3} and S; and
R^{U1}, R^{U2} and R^{U3} are each independently selected from H and C₁ to C₆ alkyl.

For example, in some cases, each T is CH₂ and/or X is CN.

Representative examples of Z are shown below:

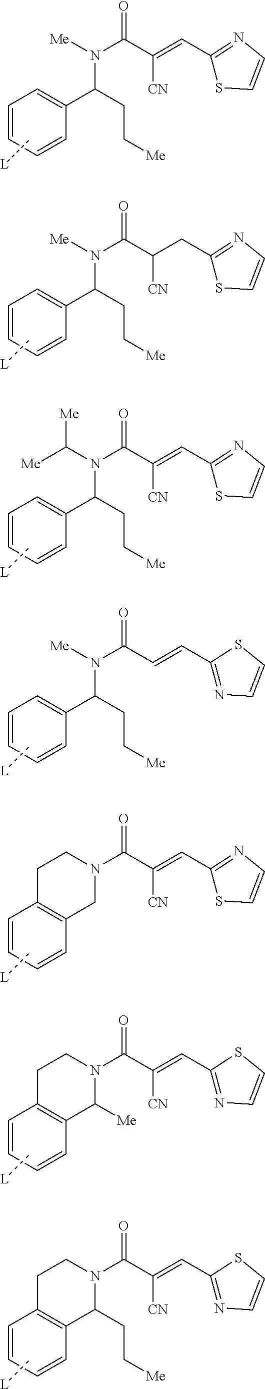

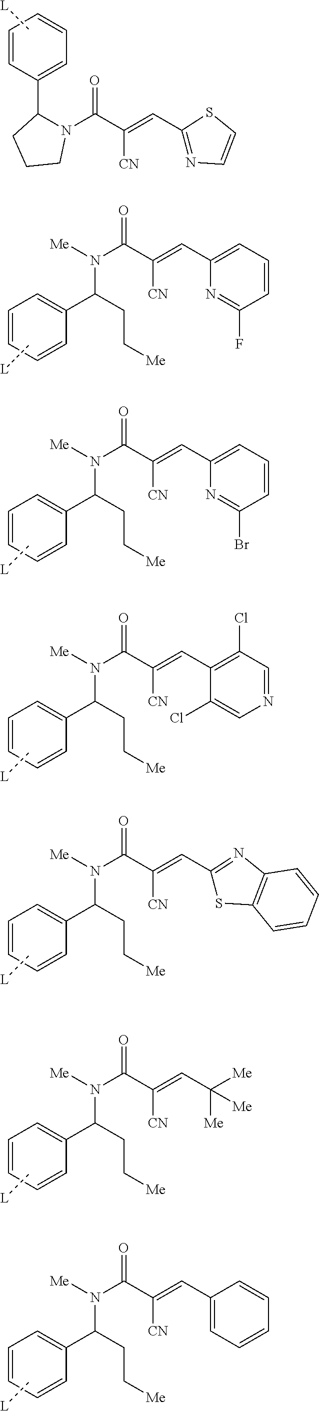

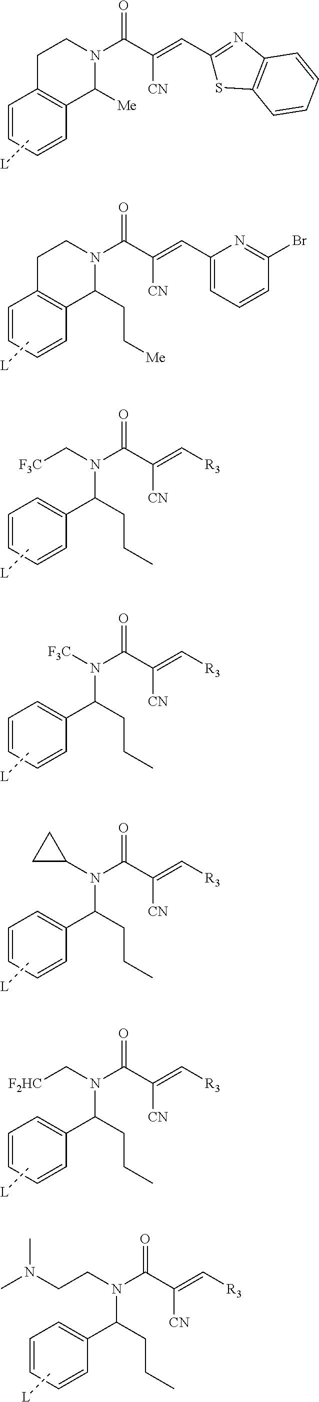

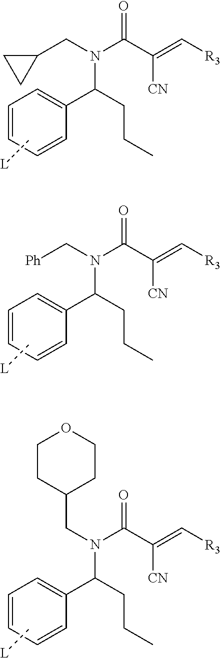

Wherein R³ in the structures shown above is any of those defined above in respect of formula (I). In some examples, R³ may be phenyl, thiazolyl, benzothiazolyl, pyridinyl, tert-butyl, pyrazolyl, imidazolyl, oxazolyl, N—C₁ to C₆ alkylenemorpholine, imidazo(1,2-a)pyridinyl, thiophenyl and 4,5,6,7-tetrahydro-1,3-benzothiazole, such as phenyl, thiazole, benzothiazole, pyridinyl, substituted pyridinyl or tert-butyl.

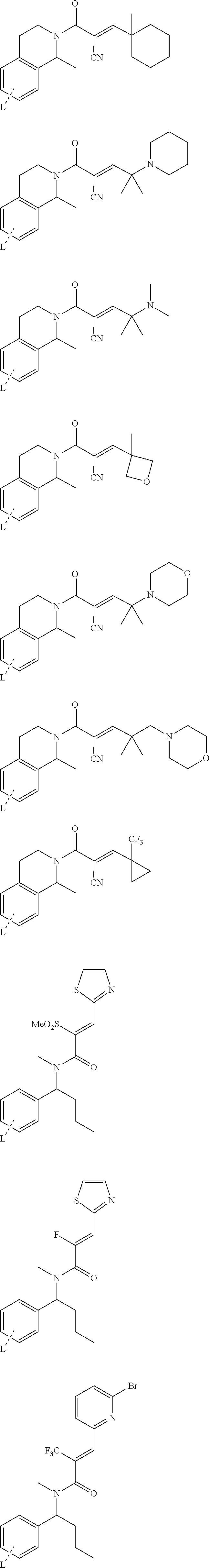

Particular examples of Z include:

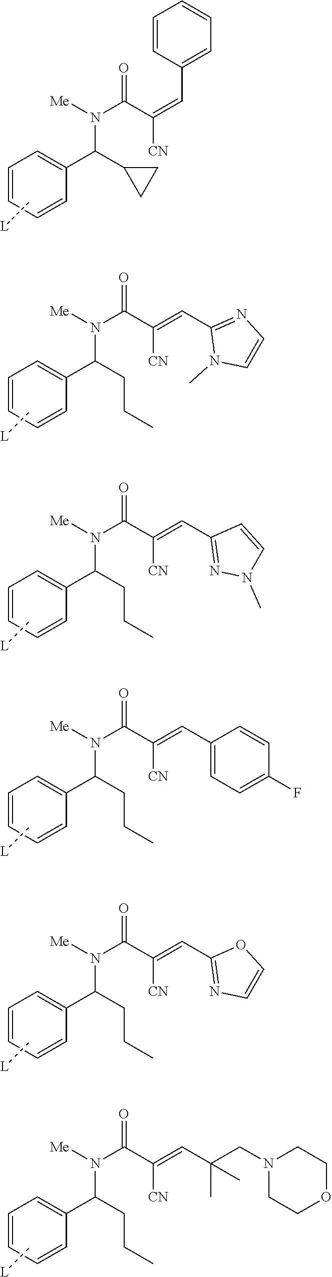

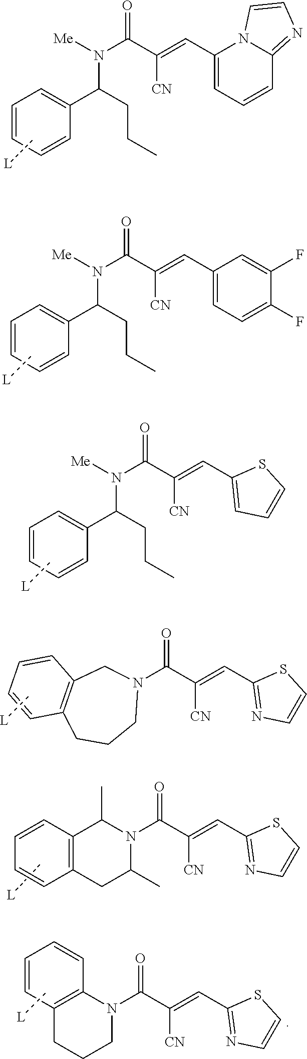

The dotted line on the structures above indicates that the linker may be joined to the Z moiety at any position on the aromatic ring (provided that it has the correct valency and/or is chemically suitable). For example, the linker may replace a hydrogen atom at any position on the aromatic ring. By way of further example, in cases where B is a phenyl ring, the linker may be attached in a para-substitution pattern with the pendant amide group as illustrated in formula IId below.

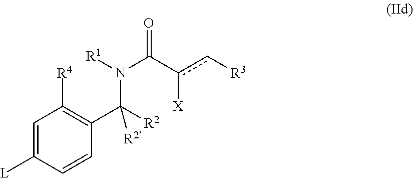

Alternatively it is noted, that whilst the formulae (I) to (IId) indicate that the linker is joined to the Z moiety via ring B (which may in some cases be an aromatic ring), the present disclosure also extends to examples wherein the linker is attached at any other position in the Z moiety (provided that it has the correct valency and/or is chemically suitable). For example, the linker may replace a hydrogen atom at any position in the Z moiety. Thus, in some examples, Z may be represented as shown in formulae (III):

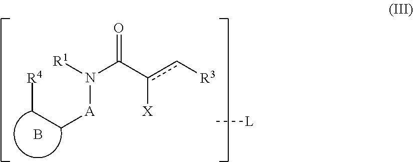

wherein R¹, A, R³, R⁴, X, B and L are as defined for formula (I) (or any of formulae (Ia) to (IId)).

The dotted line shown through the square brackets on formula (III) indicates that the linker may be joined via a covalent bond to any atom on the Z moiety provided that it has the correct valency, is chemically suitable and/or provided that the attachment of the linker at this alternative position does not disrupt the function of the Z moiety in promoting and/or facilitating proteasomal degradation.

It will be appreciated that the bifunctional molecules of the present disclosure may exist in different stereoisomeric forms. The present disclosure includes within its scope the use of all stereoisomeric forms, or the use of a mixture of stereoisomers of the bifunctional molecules, By way of example, where the bifunctional molecule comprises one or more chiral centres, the present disclosure encompasses each individual enantiomer of the bifunctional molecule as well as mixtures of enantiomers including racemic mixtures of such enantiomers. By way of further example, where the bifunctional molecule comprises two or more chiral centres, the present disclosure encompasses each individual diastereomer of the bifunctional molecule, as well as mixtures of the various diastereomers.

In many cases, a double bond is present in Z (i.e. where [symbol] is a double bond in any one of formulae I to III). The stereochemistry of this double bond may be either E or Z. The designation of this moiety as either E or Z may depend on the identity of the X group.

In some examples where [symbol] is a double bond, Z may comprise a mixture of E and Z stereoisomers. Thus, the present disclosure includes within its scope the use of each individual E and Z stereoisomers of any of the disclosed Z moieties (e.g. in a substantially stereopure form), as well as the use of mixtures of these E and Z isomers.

According to a further aspect of the disclosure, there is also provided compounds comprising a general structure of:

L-Z

wherein moiety Z is as defined in any one of formula (I) to (III); and L is a linker as defined herein.

Such compounds may be useful in a synthesis of the described bifunctional molecules, e.g. via a modular approach, wherein each of moieties TBL, Z and L are provided as separate building blocks. In some examples, L and Z may be joined to provide the compounds L-Z as described above (which may then be further reacted to join to an appropriate TBL moiety).

Intermediate Z

According to a further aspect, there is provided a compound comprising the Z moiety according to formula (IV):

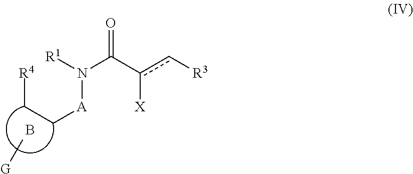

wherein A, B, X, R¹, R³ and R⁴ are as defined above for any of formulae (I) to (IId).

As shown in formula (IV), G is appended to moiety Z via ring B. G is attached to moiety Z by way of a covalent bond with an atom contained in the ring system of the optionally substituted aryl or heteroaryl group of ring B. G may be attached to ring B at any position on the optionally substituted aromatic or heteroaromatic ring (provided it has the correct valency and/or is chemically suitable). For example, G may replace a hydrogen atom at any position on the optionally substituted aromatic or heteroaromatic ring.

The group G in formula (IV) is configured to enable attachment of the Z moiety to another chemical structure (such as a linker moiety or a linker-target protein binding ligand moiety) via formation of a new covalent bond. Following the formation of this new covalent bond, the group G may form part of the linker as defined herein.

In some examples, G may comprise a functional group that is able to facilitate the formation of a new covalent bond between Z and another moiety, e.g. via formation of an amide, ester, thioester, keto, urethane, amine, or ether linkage, or via formation of a new carbon-carbon bond or new carbon-nitrogen bond.

By way of example only, G may be represented as shown below:

X^{G}-R^{G}

wherein R^{G} is absent or is a C₁ to C₆ alkyl, optionally substituted with one or more heteroatoms selected from N, O and S;
X^{G} is a group that is selected from —CO₂H, —(CO)—N-hydroxysuccinimide and —(CO)-pentafluorphenol esters, —CHO, —COR^{G1}, —OH, —NH₂, —NHR^{G2}, halo (e.g. iodo and bromo), —OTs (tosylate), OMs (mesylate), —OTf (triflate), alkynyl, azide, dienyl, aminoxy, tetrazinyl, (E)-cyclooctenyl, cyclooctynyl, norbornyl, boronic acid, boronate ester, alkylboranes or an organometallic group (e.g. organotin, zinc or other suitable reagent); and
R^{G1} and R^{G2} are each independently selected from C₁ to 06 alkyl.

G is linked to ring B shown in formula (IV) by way of the R^{G} group. In those cases where R^{G} is absent, the group X^{G} is directly attached to ring B.

Representative examples of suitable G moieties are shown below:

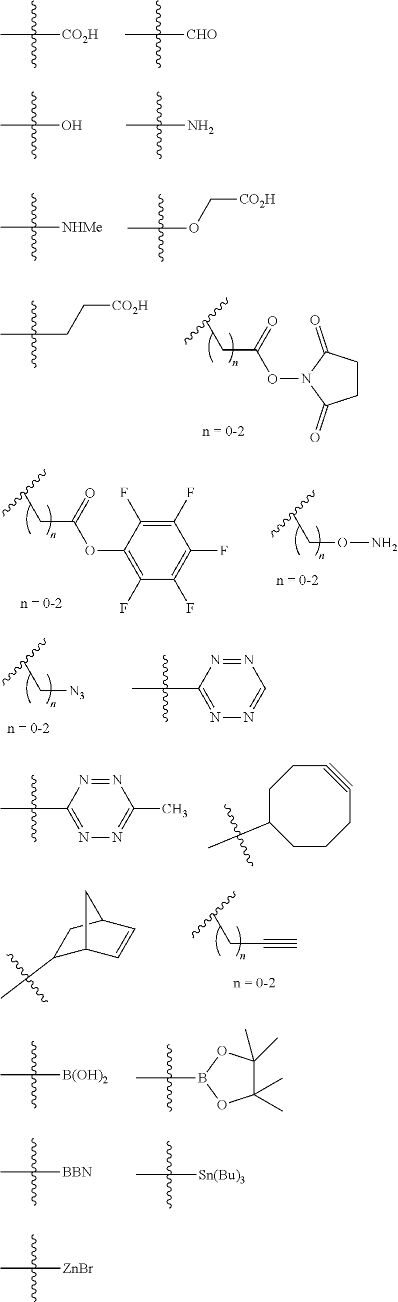

It is noted that the disclosure further extends to any of the structures for Z shown in formulae (I) to (III) or other representative examples of Z, wherein the group L on these structures has been replaced with the group G as defined above in respect of formula (IV).

Linker (L)

As described herein, the TBL is linked or coupled to moiety Z via a linker L. The linker may be a chemical linker (e.g. a chemical linker moiety) and, for example, may be a covalent linker, by which is meant that the linker is coupled to Z and/or TBL by a covalent bond.

The linker acts to tether the target protein binding ligand and Z moieties to one another whilst also allowing both of these portions to bind to their respect targets and/or perform their intended function. In particular, the linker may act to tether the target protein binding ligand to Z whilst also mitigating the possibility of the Z moiety disrupting, interfering with and/or inhibiting the binding of the target protein binding ligand to the target protein. Additionally or alternatively, the linker may act to tether Z to the target protein binding ligand whilst also mitigating the possibility of the target protein binding ligand disrupting, interfering with and/or inhibiting the cellular interactions of Z (e.g. its function in modulating, facilitating and/or promoting the proteasomal degradation of the target protein).

In other words, the linker may function to facilitate targeted protein degradation by allowing each end of the bifunctional molecule to be available for binding (or another type of cellular interaction) with various components of the cellular environment. For example, the linker may be configured to allow the target protein binding ligand to bind to the target protein without interference, disruption and/or inhibition from the Z moiety of the bifunctional molecule. Additionally or alternatively, the linker may be configured to allow the Z moiety to interact with the various components in the cellular environment to modulate, facilitate and/or promote the proteasomal degradation of the target protein without interference, disruption and/or inhibition from the target protein binding ligand of the bifunctional molecule.

In many cases, a broad range of linkers will be tolerated. The selection of linker may depend upon the protein being targeted for degradation (the target protein) and/or the particular target protein binding ligand.

The linker may be selected to provide a particular length and/or flexibility, e.g. such that the target protein binding ligand and the Z moiety are held within a particular distance and/or geometry. As will be appreciated by one of skill in the art, the length and/or flexibility of the linker may be varied dependent upon the structure and/or nature of the target protein binding ligand.

By way of example only, the linker may comprise any number of atoms between 1 and 200, between 1 and 100, between 1 and 50, between 1 and 30 or between 1 and 10.

The degree of flexibility of the linker may depend upon the number of rotatable bonds present in the linker. A rotatable bond is defined as a single non-ring bond, bound to a nonterminal heavy atom. As described herein, an amide (C—N) bond is not considered rotatable because of the high rotational energy barrier. In some cases, the linkers may comprise one or more moieties selected from rings, double bonds and amides to reduce the flexibility of the linker. In other cases, the linker may comprise a greater number and/or proportion of single bonds (e.g. may predominantly comprise single non-ring bonds) to increase the flexibility of the linker. It may also be appreciated that the length of the linker may affect the degree of flexibility. For example, a shorter linker comprising fewer bonds may also reduce the flexibility of a linker.

The structure of the linker (L) may be represented as follows:

(Lₓ)_{q}

wherein each Lₓ represents a subunit of L; and
q is an integer greater than or equal to 1.

For example, q may be any integer between 1 and 30, between 1 and 20 or between 1 and 5.

By way of example, in the case where q is 1, the linker comprises only one Lₓ subunit and may be represented as L₁. In the case where q is 2, the linker comprises two Lₓ subunits that are covalently linked to one another and which may be represented as L₁-L₂. In another example, where q is 3, the linker comprises three Lₓ subunits that are covalently linked to one another and may be represented as L₁-L₂-L₃. For even higher integer values of q, L may comprise the following subunits L₁, L₂, L₃, L₄ . . . up to L_{q}.

Each of Lₓ may be independently selected from CR^{L1}R^{L2}, O, C═O, S, S═O, SO₂, NR^{L3}, SONR^{L4}, SONR^{L5}C═O, CONR^{L6}, NR^{L7}CO, C(R^{L8})C(R^{L9}), C≡C, aryl, substituted aryl, heteroaryl, substituted heteroaryl, carbocyclyl, substituted carbocyclyl, heterocyclyl and substituted heterocyclyl groups.

Each of R^{L1}, R^{L2}, R^{L3}, R^{L4}, R^{L5}, R^{L6}, R^{L7}, R^{L8}, and R^{L9} may be independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —S(O)OC₁ to C₆ alkyl, —C(O)OC₁ to C₆ alkyl, and —CO(C₁ to C₆ alkyl). In some examples, each of R^{L1}R^{L2}, R^{L3}, R^{L4}, R^{L5}, R^{L6}, R^{L7}, R^{L8}, and R^{L9} may be independently selected from H and C₁ to C₆ alkyl.

The terms aryl, substituted aryl, heteroaryl, substituted heteroaryl, carbocyclyl and substituted carbocyclyl, heterocyclyl and substituted heterocyclyl groups are defined above.

The terminal Lₓ subunits may link or couple the linker moiety to the TBL and Z moieties of the bifunctional molecule. For example, if the terminal Lₓ subunits are designated as L₁ and L_{q}, L₁ may link the linker to the TBL moiety and L_{q} may link the linker to the Z moiety. In those cases where q is 1, the one Lₓ subunit (e.g. L₁) provides the link between the TBL and Z moieties of the bifunctional molecule.

The TBL and Z moieties may be covalently linked to L through any group which is appropriate and stable to the chemistry of the linker. By way of example only, the linker may be covalently bonded to the TBL moiety via a carbon-carbon bond, keto, amino, amide, ester or ether linkage. Similarly, the linker may be covalently bonded to the Z moiety via a carbon-carbon bond, carbon-nitrogen bond, keto, amino, amide, ester or ether linkage.

In some cases, each terminal Lₓ subunit (e.g. L₁ and L_{q}) is independently selected from O, C═O, CR^{L1}R^{L2}, NR^{L3}, CONR^{L6}, NR^{L7}CO, aryl, substituted aryl, heteroaryl, substituted heteroaryl, carbocyclyl, substituted carbocyclyl, heterocyclyl and substituted heterocyclyl groups.

In some examples, at least one of Lₓ comprises a ring structure and is, for example, selected from a heterocyclyl, heteroaryl, carbocylyl or aryl group.

In alternative examples, the linker may be or comprise an alkyl linker comprising, a repeating subunit of —CH₂—; where the number of repeats is from 1 to 50, for example, 1-50, 1-40, 1-30, 1-20, 1-19, 1-18, 1-17, 1-16, 1-15, 1-14, 1-13, 1-12, 1-11, 1-10, 1-9. 1-8, 1-7, 1-6, 1-5, 1-4, 1-3 and 1-2.

In other examples, the linker may be or comprise a polyalkylene glycol. By way of example only, the linker may be or comprise a polyethylene glycol (PEG) comprising repeating subunits of ethylene glycol (C₂H₄O), for example, having from about 1-50 ethylene glycol subunits, for example where the number of repeats is from 1 to 100, for example, 1-50, 1-40, 1-30, 1-20, 1-19 1-18, 1-17, 1-16, 1-15, 1-14, 1-13, 1-12 or 1-5 repeats.

In any of the examples described herein, the linker is or comprises one or more of:

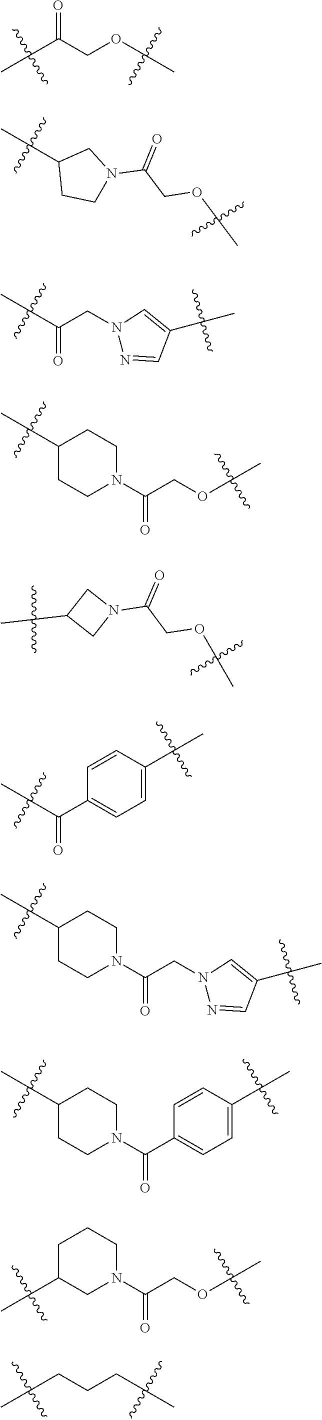

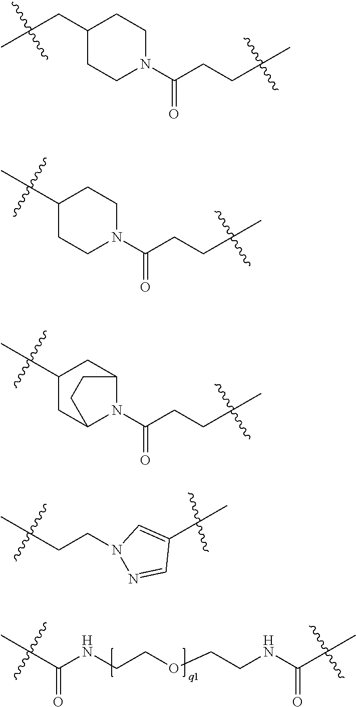

wherein q1 is any integer between 1 and 20, or between 1 and 10 (e.g. between 1 and 5).

Alternatively, in any of the examples described herein, the linker is or comprises one or more of:

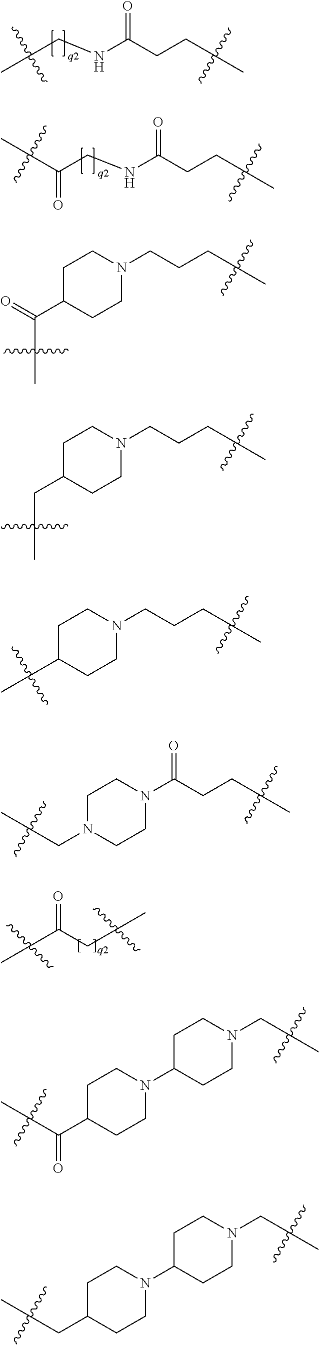

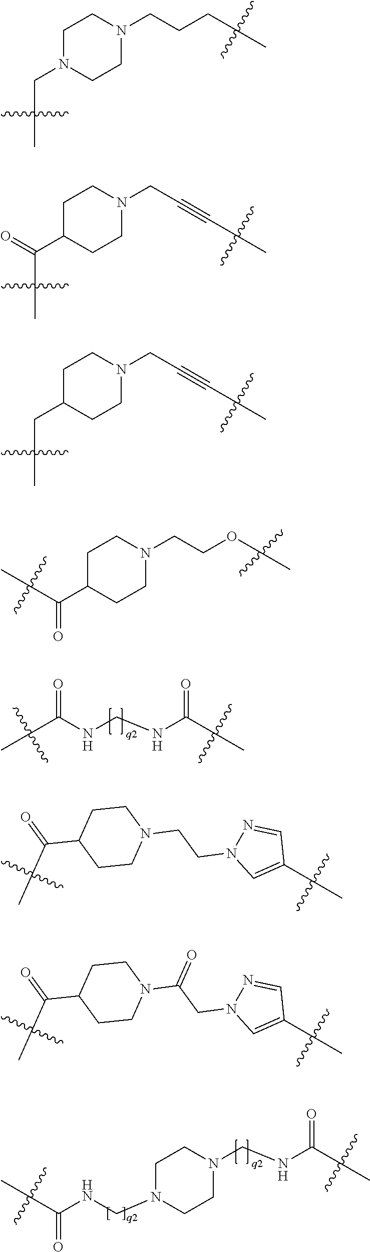

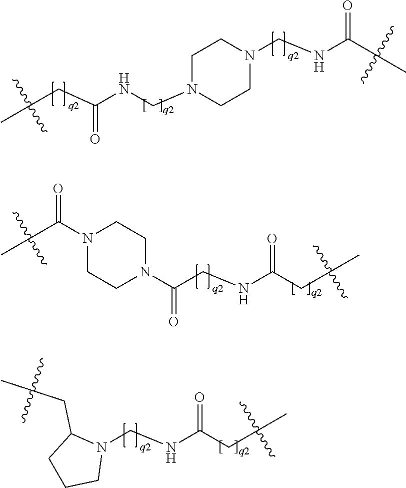

wherein q2 is any integer between 1 and 20, or between 1 and 10 (e.g. 3, 4, 6 or 10).

Thus, in some cases, the structures shown above represent the entire linker. In other examples, the linker of the bifunctional molecule may comprise a plurality of the structures shown above.

In these structures, the wavy lines are shown over the bond(s) that forms the link with the TBL and Z moieties respectively.

In some examples, the bond(s) that forms the link with the TBL and/or Z moieties is (are) attached to a ring structure. On many of the structures described herein, this bond is shown as being attached at a particular position on the ring structure. However, the disclosure also encompasses joining or coupling to the TBL and Z moieties at any chemically suitable position on these ring structures.

The present disclosure encompasses the use of any of the linkers disclosed herein in combination with any of the Z moieties and TBL moieties described herein.

Target Protein

As used herein, a “target protein” may be any polypeptide or protein that the skilled practitioner wishes to selectively degrade in a cell or a mammal, e.g., a human subject.

In other words, a “target protein” may be a protein or polypeptide that is selected by the skilled practitioner for increased proteolysis in a cell. The term “selected target protein” may be any polypeptide or protein which has been selected to be targeted for protein degradation and/or increased proteolysis.

According to the disclosure, degradation of a target protein may occur when the target protein is subjected to and/or contacted with a bifunctional molecule as described herein, e.g. when the target protein is subjected to and/or contacted with any one of the bifunctional molecules in a cell.

Selective degradation and/or increased proteolysis of the target protein will reduce protein levels and so can reduce the effects of the target protein in the cell. The control of protein levels afforded by the bifunctional molecules described herein may provide treatment of a disease state or condition, which is modulated through the target protein by lowering the level of that protein in the cells of a subject.

Target proteins that may be subject to increased proteolysis and/or selective degradation when contacted to the bifunctional molecules of this disclosure (and the associated methods of using such molecules) include any proteins and polypeptides. Target proteins include proteins and polypeptides having a biological function or activity such as structural, regulatory, hormonal, enzymatic, genetic, immunological, contractile, storage, transportation, and signal transduction functions and activities.

By way of example, target proteins may include structural proteins, receptors, enzymes, cell surface proteins, proteins pertinent to the integrated function of a cell, including proteins involved in catalytic activity, epigenetic regulation, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioural proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, and translation regulator activity.

Target proteins may include proteins from eukaryotes and prokaryotes, including humans, other animals, including domesticated animals, microbes, viruses, fungi and parasites, among numerous other targets for drug therapy.

In some examples, target proteins may include, but are not limited to: (i) kinases (such as serine/threonine kinases and receptor tyrosine kinases); (ii) bromodomain-containing proteins (such as BET family proteins); (iii) epigenetic proteins (including histone or DNA methyl transferases, acetyl transferases, deacetylases and demethylases); (iv) transcription factors (including STAT3 and myc); (v) GTPases (including KRAS, NRAS, and HRAS); (vi) phosphatases; (vii) ubiquitin E3 ligases; (viii) nuclear receptors (including androgen receptor (AR) and estrogen receptor (ER)); (ix) aggregation-prone proteins (including Beta-amyloid, tau, Htt, alpha-synuclein and polyQ-expanded proteins); and (x) apoptotic & anti-apoptotic factors (including Bcl2, Bcl-xl and Mcl-1), and (xi) polymerases (including PARP) among numerous others.

A target protein may also be selected from targets for human therapeutic drugs. These include proteins which may be used to restore function in numerous diseases, e.g. polygenic diseases, including for example, target proteins selected from B7.1 and B7, TNFR1, TNFR2, NADPH oxidase, BclI/Bax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAK STAT, RXR and similar, HIV 1 protease, HIV 1 integrase, influenza, neuraminidase, hepatitis B reverse transcriptase, sodium channel, multi drug resistance (MDR), protein P-glycoprotein (and MRP), serine/threonine kinases, tyrosine kinases, CD23, CD124, tyrosine kinase p56 Ick, CD4, CD5, IL-2 receptor, IL-1 receptor, TNF-alphaR, ICAM1, Cat+ channels, VCAM, VLA-4 integrin, selectins, CD40/CD40L, neurokinins and receptors, inosine monophosphate dehydrogenase, p38 MAP Kinase, Ras/Raf/MEK/ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-1), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

Target proteins may also be haloalkane dehalogenase enzymes. By way of example, bifunctional molecules according to the disclosure which contain chloroalkane peptide binding moieties (C1-C12 often about C2-C10 alkyl halo groups) may be used to inhibit and/or degrade haloalkane dehalogenase enzymes which are used in fusion proteins or related diagnostic proteins as described in PCT/US2012/063401 filed Dec. 6, 2011 and published as WO 2012/078559 on Jun. 14, 2012, the contents of which is incorporated by reference herein.

Target Protein Binding Ligand (TBL)

As used herein, a “target protein binding ligand” refers to a ligand or moiety, which binds to a target protein, e.g. a selected target protein. By way of example, a target protein binding ligand may be any moiety, which selectively and/or specifically binds a target protein. A bifunctional molecule according to this disclosure may comprise a target protein binding ligand, which binds to the target protein with sufficient binding affinity such that the target protein is more susceptible to degradation or proteolysis than if unbound by the bifunctional molecule.

A target protein binding ligand may comprise or be derived from a small molecule (or analogue or fragment thereof) already known to act as a modulator, promoter and/or inhibitor of protein function (e.g. any small molecule known to bind to the target protein). By way of example, the target protein binding ligand may comprise or be derived from a small molecule that is known to inhibit activity of a given target protein.

Non-limiting examples of small molecules that can be comprised in the target protein binding ligand moiety of the bifunctional molecules described herein include: (i) binders to kinases (including serine/threonine kinases e.g. RAF, receptor tyrosine kinases and other classes), (ii) compounds binding to bromodomain-containing proteins (including BET family and others), (iii) epigenetic modulator compounds (including binders to histone or DNA methyl transferases, acetyl transferases, deacetylases & demethylases and others e.g. histone deacetylase (HDAC)), (iv) binders to transcription factors including STAT3, myc and others, (v) binders to GTPases (including KRAS, NRAS, HRAS and others), (vi) binders of phosphatases, (vii) binders of ubiquitin E3 ligases (e.g. MDM2), (viii) immunosuppressive and immunomodulatory compounds, (ix) modulators of nuclear receptors (including androgen receptor (AR), estrogen receptor (ER), thyroid hormone receptor (TR) and others), (x) binders to aggregation-prone proteins (including Beta-amyloid, tau, Htt, alpha-synuclein, polyQ-expanded proteins and others), (xi) binders to apoptotic & anti-apoptotic factors (including Bcl2, Bcl-xl, Mcl-1 and others), and (xii) binders to polymerases (including PARP and others) among numerous others.

Other non-limiting examples of small molecules that can be comprised in the target protein binding ligand moiety of the bifunctional molecules described herein include: (i) Hsp90 inhibitors, (ii) human lysine methyltransferase inhibitors, (iii) angiogenesis inhibitors, (iv) compounds targeting the aryl hydrocarbon receptor (AHR), (v) compounds targeting FKBP, (vi) compounds targeting HIV protease, (vii) compounds targeting HIV integrase, (viii) compounds targeting HCV protease, (ix) compounds targeting acyl-protein thioesterase-1 and -2 (APT1 and APT2) among numerous others.

In some instances, the target protein binding ligand is derived from a BET inhibitor (e.g. the BET inhibitor IBET276). In such examples, the target protein binding ligand may comprise the following structure:

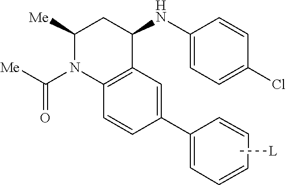

wherein L shows the position of attachment of the linker and the dotted line on the structure above indicates that the linker may be joined to the target protein binding ligand via any position on the aromatic ring (e.g. in some examples, L may be present at the 4-position on this aromatic ring). However, the present disclosure also encompasses joining or coupling to the linker at any chemically suitable position on this target protein binding ligand.

Alternatively, the target protein binding ligand may be derived from a BRD9 inhibitor, for example the target protein binding ligand may comprise the following structure:

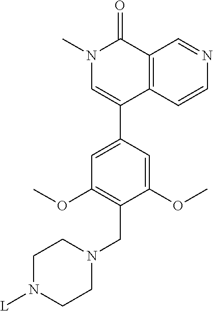

wherein L shows the position of attachment of the linker.

In other examples, the target protein binding ligand is derived from a kinase inhibitor. In such examples, the target protein binding ligand may comprise the following structure:

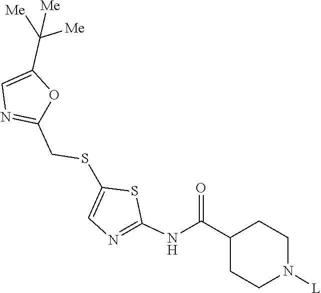

wherein L shows the position of attachment of the linker. However, again, the present disclosure also encompasses joining or coupling to the linker at any chemically suitable position on this target protein binding ligand.

The target protein binding ligand may be derived from a kinase inhibitor, such as a CDK9 inhibitor, and may comprise the following structure:

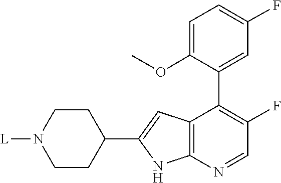

where L shows the position of attachment of the linker.

Alternatively, the target protein binding ligand may be derived from a kinase inhibitor such as a mutant EGFR inhibitor, and may have the following structure:

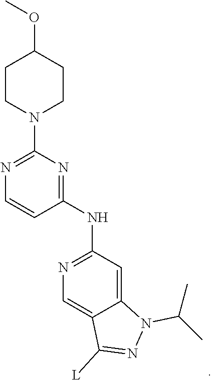

where L shows the position of attachment of the linker.

In some instances, the target protein binding ligand may be derived from a GTPase inhibitor, such as a KRAS G12C inhibitor. For example, the target protein binding ligand may have the following structure:

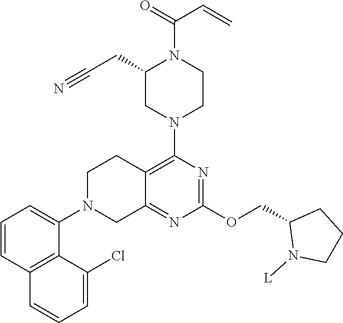

where L shows the position of attachment of the linker.

In other instances, the target protein binding ligand may be derived from a polymerase inhibitor, such as a PARP1 inhibitor. For example, the target protein binding ligand may have the following structure:

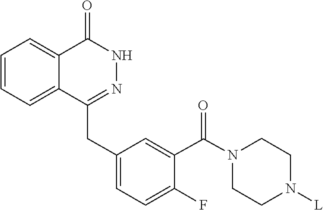

where L shows the position of attachment of the linker.

Representative examples of possible target protein binding ligand moieties for each of the various classes of target protein binding ligands are described below.

I. Kinase and Phosphatase Inhibitors:

Examples of kinase inhibitors may be found in Jones et al. Small-Molecule Kinase Downregulators (2017, Cell Chem. Biol., 25: 30-35). Further kinase inhibitors that may be used according to some examples of the disclosure include, but are not limited to:
1. Erlotinib Derivative Tyrosine Kinase Inhibitor:

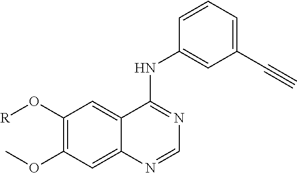

where R is a linker attached, for example, via an ether group;
2. The kinase inhibitor sunitinib (derivatized):

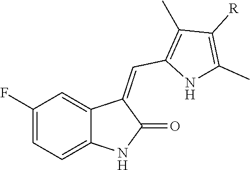

(derivatized where R is a linker attached, for example, to the pyrrole moiety);
3. Kinase Inhibitor sorafenib (derivatized):

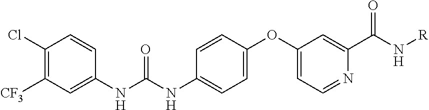

(derivatized where R is a linker attached, for example, to the amide moiety);
4. The kinase inhibitor dasatinib (derivatized):

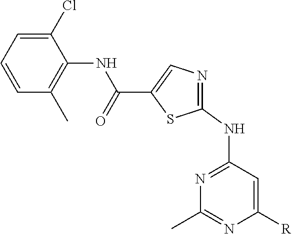

(derivatized where R is a linker attached, for example, to the pyrimidine);
5. The kinase inhibitor lapatinib (derivatized):

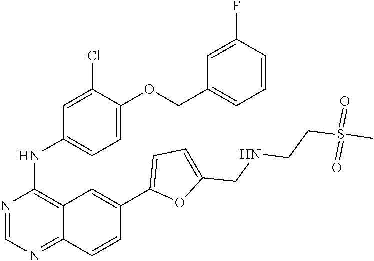 [SMILES: CS(=O)(=O)CCNCc1ccc(-c2ccc3ncnc(Nc4ccc(OCc5cccc(F)c5)c(Cl)c4)c3c2)o1]

(derivatized where a linker is attached, for example, via the terminal methyl of the sulfonyl methyl group);
6. The kinase inhibitor U09-CX-5279 (derivatized):

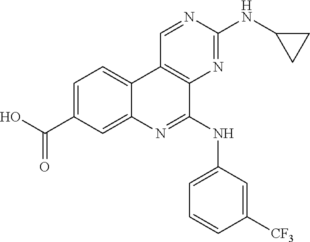 [SMILES: O=C(O)c1ccc2c(c1)nc(Nc1cccc(C(F)(F)F)c1)c1nc(NC3CC3)ncc12]

derivatized where a linker is attached, for example, via the amine (aniline), carboxylic acid or amine alpha to cyclopropyl group, or cyclopropyl group;
7. The kinase inhibitors identified in Millan, et al., Design and Synthesis of Inhaled P38 Inhibitors for the Treatment of Chronic Obstructive Pulmonary Disease, (2011, J. Med. Chem. 54:7797), including the kinase inhibitors Y1W and Y1X (Derivatized) having the structures:

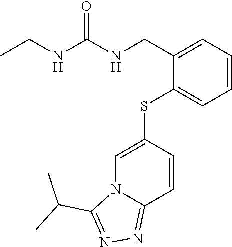 [SMILES: CCNC(=O)NCc1ccccc1Sc1ccc2nnc(C(C)C)n2c1]

YIX

### (I-ethyl-3-(2-{[3-(1-methylethyl)[I,2,4]triazolo[4,3-a]pyridine-6-yl]sulfanyl}benzyl)urea

derivatized where a linker is attached, for example, via the iso-propyl group;

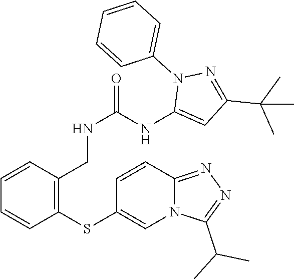 [SMILES: CC(C)c1nnc2ccc(Sc3ccccc3CNC(=O)Nc3cc(C(C)(C)C)nn3-c3ccccc3)cn12]

Y1W

### 1-(3-tert-butyl-1-phenyl-1H-pyrazol-5-yl)-3-(2-{[3-(1-methylethyl)[1,2,4]triazolo[4,3-a]pyridin-6-yl]sulfanyl}benzyl)urea

derivatized where a linker is attached, for example, preferably via either the iso-propyl group or the tert-butyl group;
8. The kinase inhibitors identified in Schenkel, et al., Discovery of Potent and Highly Selective Thienopyridine Janus Kinase 2 Inhibitors (2011, J. Med. Chem., 54(24):8440-8450), including the compounds 6TP and OTP (Derivatized) having the structures:

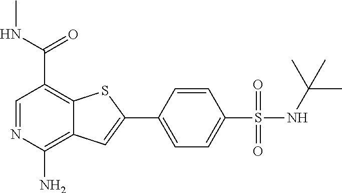 [SMILES: CNC(=O)c1cnc(N)c2cc(-c3ccc(S(=O)(=O)NC(C)(C)C)cc3)sc12]

6TP

### 4-amino-2-[4-(tert-butylsulfamoyl)phenyl]-N-methylthieno[3,2-c]pyridine-7-carboxamide

Thienopyridine 19

derivatized where a linker is attached, for example, via the terminal methyl group bound to amide moiety;

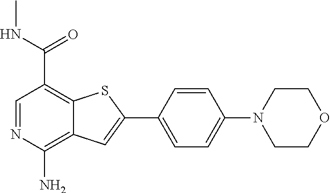 [SMILES: CNC(=O)c1cnc(N)c2cc(-c3ccc(N4CCOCC4)cc3)sc12]

OTP

### 4-amino-N-methyl-2-[4-(morpholin-4-yl)phenyl]thieno[3,2-c]pyridine-7-carboxamide

Thienopyridine 8

derivatized where a linker is attached, for example, via the terminal methyl group bound to the amide moiety;
9. The kinase inhibitors identified in Van Eis, et al., “2,6-Naphthyridines as potent and selective inhibitors of the novel protein kinase C isozymes”, (2011 Dec., Biorg. Med. Chem. Lett., 15, 21(24):7367-72), including the kinase inhibitor 07U having the structure:

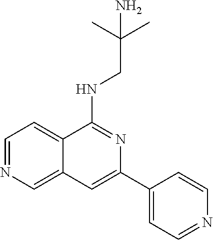 [SMILES: CC(C)(N)CNc1nc(-c2ccncc2)cc2cnccc12]

07U

### 2-methyl-N-1-[3-(pyridin-4-yl)-2,6-naphthyridin-1-yl]propane-1,2-diamine

derivatized where a linker is attached, for example, via the secondary amine or terminal amino group;
10. The kinase inhibitors identified in Lountos, et al., “Structural Characterization of Inhibitor Complexes with Checkpoint Kinase 2 (Chk2), a Drug Target for Cancer Therapy”, (2011, J. Struct. Biol., 176:292), including the kinase inhibitor YCF having the structure:

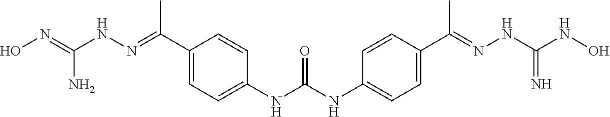 [SMILES: C/C(=N\NC(=N)NO)c1ccc(NC(=O)Nc2ccc(/C(C)=N/N/C(N)=N/O)cc2)cc1]

derivatized where a linker is attached, for example, via either of the terminal hydroxyl groups;
11. The kinase inhibitors identified in Lountos, et al., “Structural Characterization of Inhibitor Complexes with Checkpoint Kinase 2 (Chk2), a Drug Target for Cancer Therapy”, (2011, J. Struct. Biol. 176292), including the kinase inhibitors XK9 and NXP (derivatized) having the structures:

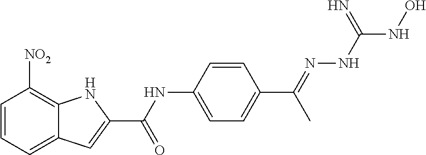 [SMILES: C/C(=N\NC(=N)NO)c1ccc(NC(=O)c2cc3cccc([N+](=O)[O-])c3[nH]2)cc1]

XK9

### N-{4-[(1E)-N—(N-hydroxycarbamimidoyl)ethanehydrazonoyl]phenyl}-7-nitro-1H-indole-2-carboxamide

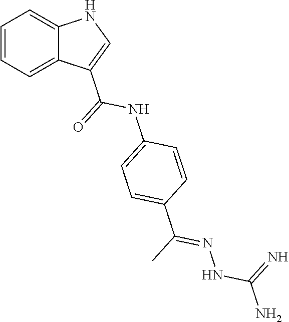 [SMILES: C/C(=N\NC(=N)N)c1ccc(NC(=O)c2c[nH]c3ccccc23)cc1]

NXP

### N-{4-[(1E)-N-carbamimidoylethanehydrazonoyl]phenyl}-1H-indole-3-carboxamide

derivatized where a linker is attached, for example, via the terminal hydroxyl group (XK9) or the hydrazone group (NXP);
12. The kinase inhibitor afatinib (derivatized) (N-[4-[(3-chloro-4-fluorophenyl)amino]-7-[[(3S)-tetrahydro-3-furanyl]oxy]-6-quinazolinyl]-4(dimethylamino)-2-butenamide) (Derivatized where a linker is attached, for example, via the aliphatic amine group);
13. The kinase inhibitor fostamatinib (derivatized) ([6-({5-fluoro-2-[(3,4,5-trimethoxyphenyl)amino]pyrimidin-4-yl}amino)-2,2-dimethyl-3-oxo-2,3-dihydro-4H-pyrido[3,2-b]-I,4-oxazin-4-yl]methyl disodium phosphate hexahydrate) (Derivatized where a linker is attached, for example, via a methoxy group);
14. The kinase inhibitor gefitinib (derivatized) (N-(3-chloro-4-fluoro-phenyl)-7-methoxy-6-(3-morpholin-4-ylpropoxy)quinazolin-4-amine):

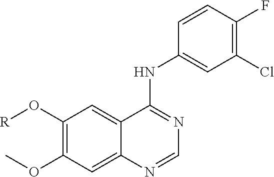

(derivatized where a linker is attached, for example, via a methoxy or ether group);
15. The kinase inhibitor lenvatinib (derivatized) (4-[3-chloro-4-(cyclopropylcarbamoylamino)phenoxy]-7-methoxy-quinoline-6-carboxamide) (derivatized where a linker is attached, for example, via the cyclopropyl group);
16. The kinase inhibitor vandetanib (derivatized) (N-(4-bromo-2-fluorophenyl)-6-methoxy-7-[(I-methylpiperidin-4-yl)methoxy]quinazolin-4-amine) (derivatized where a linker is attached, for example, via the methoxy or hydroxyl group);
17. The kinase inhibitor vemurafenib (derivatized) (propane-1-sulfonic acid {3-[5-(4-chlorophenyl)-1H-pyrrolo[2,3-b]pyridine-3-carbonyl]-2,4-difluoro-phenyl}-amide) (derivatized where a linker is attached, for example, via the sulfonyl propyl group);
18. The kinase inhibitor Gleevec (also known as Imatinib) (derivatized):

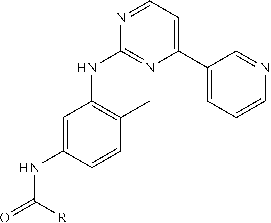

(derivatized where R is a linker attached, for example, via the amide group or via the aniline amine group);
19. The kinase inhibitor pazopanib (derivatized) (VEGFR3 inhibitor):

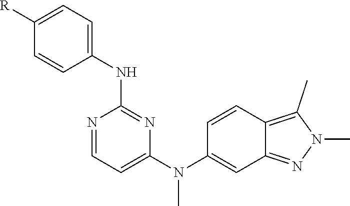

(derivatized where R is a linker attached, for example, to the phenyl moiety or via the aniline amine group);
20. The kinase inhibitor AT-9283 (Derivatized) Aurora Kinase Inhibitor

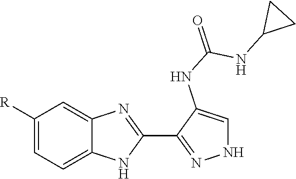

(where R is a linker attached, for example, to the phenyl moiety);
21. The kinase inhibitor TAE684 (derivatized) ALK inhibitor

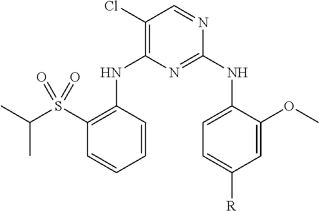

(where R is a linker attached, for example, to the phenyl moiety);
22. The kinase inhibitor nilotanib (derivatized) AbN inhibitor:

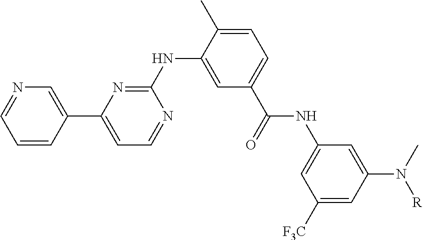

(derivatized where R is a linker attached, for example, to the phenyl moiety or the aniline amine group);
23. Kinase Inhibitor NVP-BSK805 (derivatized) JAK2 Inhibitor

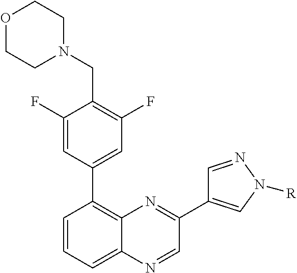

(derivatized where R is a linker attached, for example, to the phenyl moiety or the diazole group);
24. Kinase Inhibitor crizotinib Derivatized Alk Inhibitor

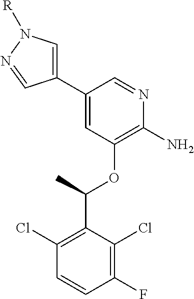

(derivatized where R is a linker attached, for example, to the phenyl moiety or the diazole group);
25. Kinase Inhibitor JNJ FMS (derivatized) Inhibitor

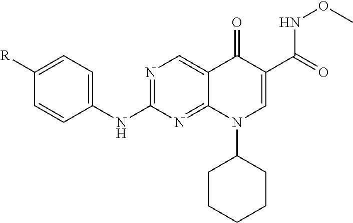

(derivatized where R is a linker attached, for example, to the phenyl moiety);
26. The kinase inhibitor foretinib (derivatized) Met Inhibitor

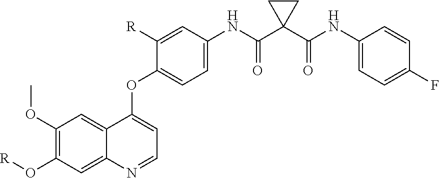

(derivatized where R is a linker attached, for example, to the phenyl moiety or a hydroxyl or ether group on the quinoline moiety);
27. The allosteric Protein Tyrosine Phosphatase Inhibitor PTPIB (derivatized):

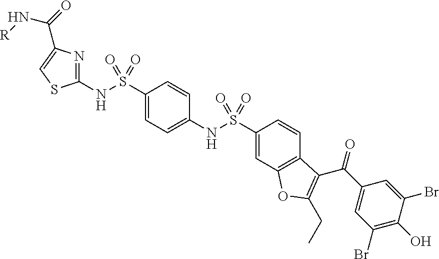

derivatized where a linker is attached, for example, at R, as indicated;
28. The inhibitor of SHP-2 Domain of Tyrosine Phosphatase (derivatized):

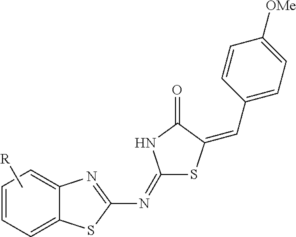

derivatized where a linker is attached, for example, at R;29. The inhibitor (derivatized) of BRAF (BRAFV600E)/MEK:

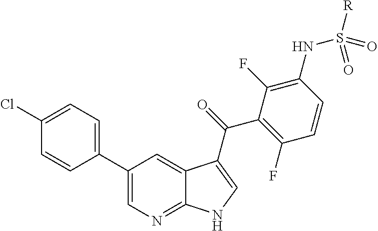

derivatized where a linker group is attached, for example, at R;
30. Inhibitor (derivatized) of Tyrosine Kinase ABL

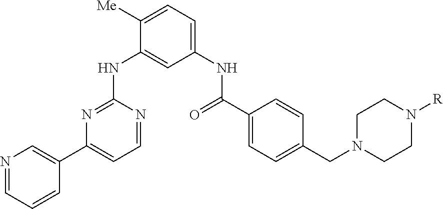

derivatized where a linker is attached, for example, at R;
31. The kinase inhibitor OSI-027 (derivatized) mTORCI/2 inhibitor

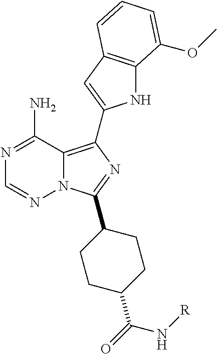

derivatized where a linker is attached, for example, at R;
32. The kinase inhibitor OSI-930 (derivatized) c-Kit/KDR inhibitor

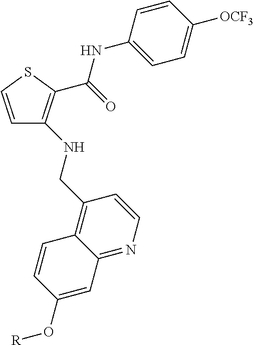

derivatized where a linker is attached, for example, at R; and
33. The kinase inhibitor OSI-906 (derivatized) IGFIR/IR inhibitor

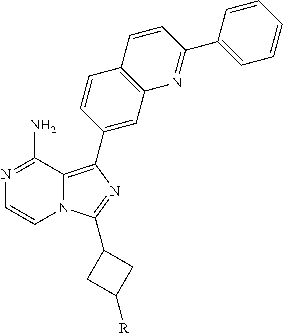

derivatized where a linker is attached, for example, at R;
(derivatized where “R” designates a site for attachment of a linker on the piperazine moiety).

II. Compounds Targeting Human BET Bromodomain-Containing Proteins:

Compounds targeting Human BET Bromodomain-containing proteins include, but are not limited to the compounds associated with the targets as described below, where “R” designates a site for linker attachment, for example:

JQI, Filippakopoulos et al. Selective inhibition of BET bromodomains. Nature (2010):

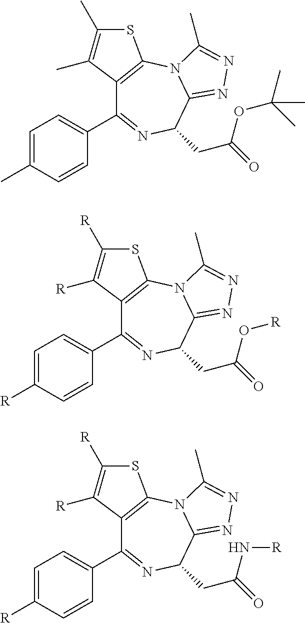

2. I-BET, Nicodeme et al. Supression of Inflammation by a Synthetic Histone Mimic. Nature (2010). Chung et al. Discovery and Characterization of Small Molecule Inhibitors of the BET Family Bromodomains. J. Med Chem. (2011):

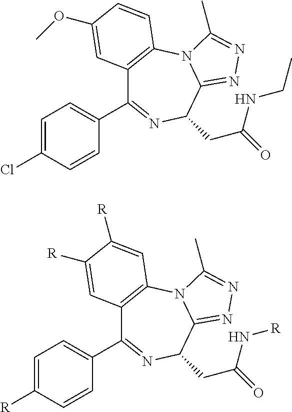

3. Compounds described in Hewings et al. 3,5-Dimethylisoxazoles Act as Acetyl-lysine Bromodomain Ligands. (2011, J. Med. Chem. 54:6761-6770).

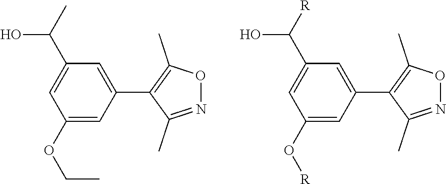

4. I-BET151, Dawson et al. Inhibition of BET Recruitment to Chromatin as an Effective Treatment for MLL-fusion Leukemia. Nature (2011):

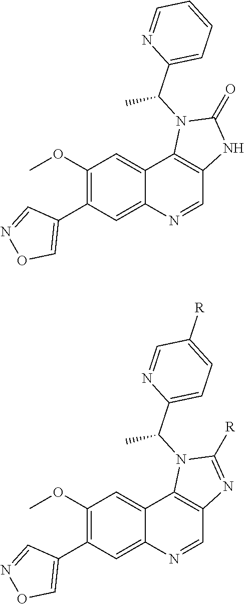

(Where R, in each instance, designates a site for attachment of a linker.)

III. Heat Shock Protein 90 (HSP90) Inhibitors:

HSP90 inhibitors useful according to the present disclosure include but are not limited to:
1. The HSP90 inhibitors identified in Vallee, et al., “Tricyclic Series of Heat Shock Protein 90 (HSP90) Inhibitors Part I: Discovery of Tricyclic Imidazo[4,5-C]Pyridines as Potent Inhibitors of the HSP90 Molecular Chaperone (2011, J. Med. Chem., 54:7206), including YKB (N-[4-(3H-imidazo[4,5-C]Pyridin-2-yl)-9H-Fluoren-9-yl]-succinamide):

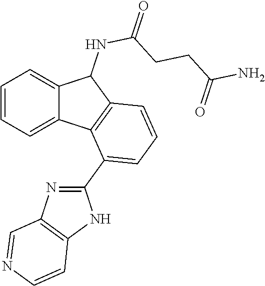 [SMILES: NC(=O)CCC(=O)NC1c2ccccc2-c2c(-c3nc4cnccc4[nH]3)cccc21]

derivatized where a linker is attached, for example, via the terminal amide group;
2. The HSP90 inhibitor p54 (modified) (8-[(2,4-dimethylphenyl)sulfanyl]-3]pent-4-yn-1-yl-3H-purin-6-amine):

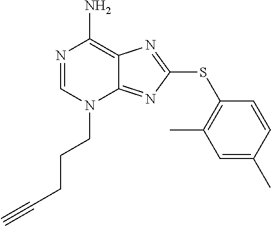 [SMILES: C#CCCCn1cnc(N)c2nc(Sc3ccc(C)cc3C)nc1-2]

where a linker is attached, for example, via the terminal acetylene group;
3. The HSP90 inhibitors (modified) identified in Brough, et al., “4,5-Diarylisoxazole HSP90 Chaperone Inhibitors: Potential Therapeutic Agents for the Treatment of Cancer”, (2008, J. Med. Chem., 51:196), including the compound 2GJ (5-[2,4-dihydroxy-5-(1-methylethyl)phenyl]-n-ethyl-4-[4-(morpholin-4-ylmethyl)phenyl]isoxazole-3-carboxamide) having the structure:

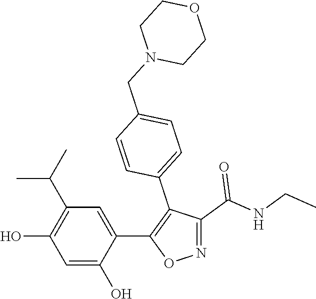 [SMILES: CCNC(=O)c1noc(-c2cc(C(C)C)c(O)cc2O)c1-c1ccc(CN2CCOCC2)cc1]

derivatized, where a linker is attached, for example, via the amide group (at the amine or at the alkyl group on the amine);
4. The HSP90 inhibitors (modified) identified in Wright, et al., Structure—Activity Relationships in Purine-Based Inhibitor Binding to HSP90 Isoforms, (2004 Jun., Chem Biol. 11(6):775-85), including the HSP90 inhibitor PU3 having the structure:

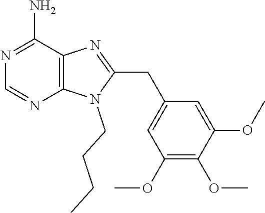 [SMILES: CCCCn1c(Cc2cc(OC)c(OC)c(OC)c2)nc2c(N)ncnc21]

where a linker group is attached, for example, via the butyl group; and
5. The HSP90 inhibitor geldanamycin ((4E,6Z,8S,9S,10E,12S,13R,14S,16R)-13-hydroxy-8,14,19-trimethoxy-4,10,12,16-tetramethyl-3,20,22-trioxo-2-azabicyclo[6.3. I](derivatized) or any its derivatives (e.g. 17-alkylamino-17-desmethoxygeldanamycin (“17-AAG”) or 17-(2-dimethylaminoethyl)amino-17-desmethoxygeldanamycin (“17-DMAG”)) (derivatized, where a is attached, for example, via the amide group).

IV. HDM2/MDM2 Inhibitors:

HDM2/MDM2 inhibitors of the invention include, but are not limited to:
1. The HDM2/MDM2 inhibitors identified in Vassilev, et al., In vivo activation of the p53 pathway by small-molecule antagonists of MDM2, (2004, Science, 303844-848), and Schneekloth, et al., Targeted intracellular protein degradation induced by a small molecule: En route to chemical proteomics, (2008, Biorg. Med. Chem. Lett., 18:5904-5908), including (or additionally) the compounds nutlin-3, nutlin-2, and nutlin-1 (derivatized) as described below, as well as all derivatives and analogs thereof:

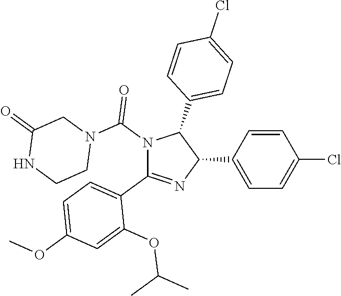 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker is attached, for example, at the methoxy group or as a hydroxyl group);

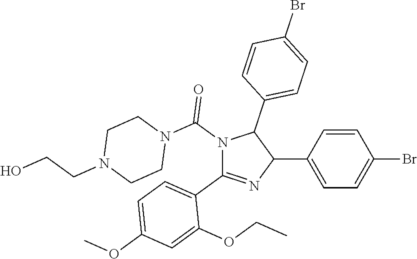 [SMILES: CCOc1cc(OC)ccc1C1=NC(c2ccc(Br)cc2)C(c2ccc(Br)cc2)N1C(=O)N1CCN(CCO)CC1]

(derivatized where a linker is attached, for example, at the methoxy group or hydroxyl group);

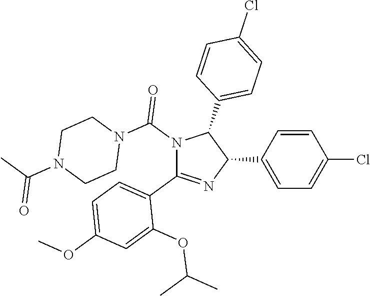 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker is attached, for example, via the methoxy group or as a hydroxyl group); and
2. Trans-4-Iodo-4′-Boranyl-Chalcone

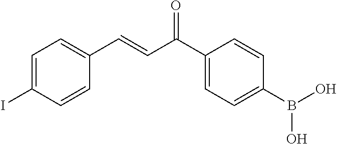 [SMILES: O=C(/C=C/c1ccc(I)cc1)c1ccc(B(O)O)cc1]

(derivatized where a linker is attached, for example, via a hydroxy group).

V. HDAC Inhibitors:

HDAC Inhibitors (derivatized) useful in some examples of the disclosure include, but are not limited to:
1. Finnin, M. S. et al. Structures of Histone Deacetylase Homologue Bound to the TSA and SAHA Inhibitors. (1999, Nature, 40:188-193).

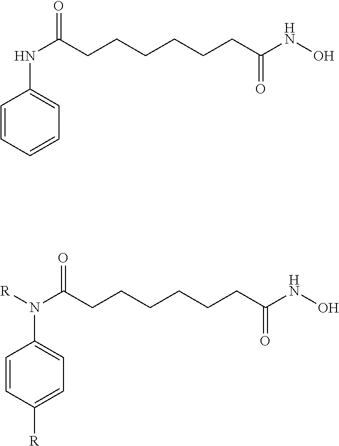

(Derivatized where “R” designates a site for attachment, for example, of a linker; and
2. Compounds as defined by formula (I) of PCT WO0222577 (the entire contents of which are incorporated herein by reference) (“DEACETYLASE INHIBITORS”) (Derivatized where a linker is attached, for example, via the hydroxyl group);

VI. Human Lysine Methyltransferase Inhibitors:

Human Lysine Methyltransferase inhibitors useful in some examples of the disclosure include, but are not limited to:
1. Chang et al. Structural Basis for G9a-Like protein Lysine Methyltransferase Inhibition by BIX-1294 (2009, Nat. Struct. Biol., 16(3):312).

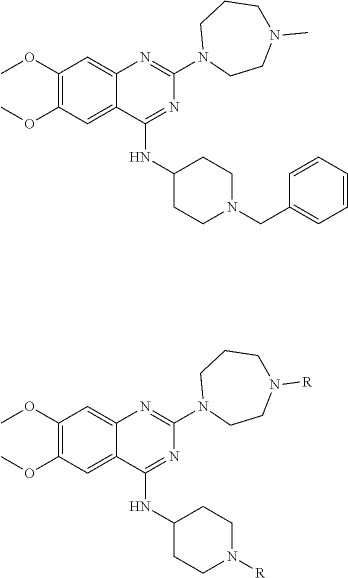

(Derivatized where “R” designates a site for attachment, for example, of a linker;
2. Liu, F. et al Discovery of a 2,4-Diamino-7-aminoalkoxyquinazoline as a Potent and Selective Inhibitor of Histone Methyltransferase G9a. (2009, J. Med. Chem., 52(24):7950).

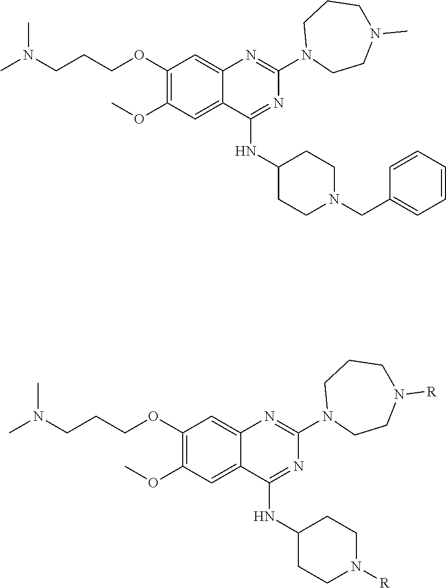

(Derivatized where “R” designates a potential site for attachment of a linker);
3. Azacitidine (derivatized) (4-amino-1-D-ribofuranosyl-1,3,5-triazin-2(1H)-one) (Derivatized where a linker is attached, for example, via the hydroxy or amino groups); and
4. Decitabine (derivatized) (4-amino-1-(2-deoxy-b-D-erythro-pentofuranosyl)-1, 3, 5-triazin-2(1H)-one) (Derivatized where a linker is attached, for example, via either of the hydroxy groups or at the amino group).

VII. Angiogenesis Inhibitors:

Angiogenesis inhibitors useful in some aspects of the disclosure include, but are not limited to:
1. GA-1 (derivatized) and derivatives and analogs thereof, having the structure(s) and binding to linkers as described in Sakamoto, et al., Development of Protacs to target cancer-promoting proteins for ubiquitination and degradation, (2003 Dec., Mol. Cell Proteomics, 2(12):1350-1358);
2. Estradiol (derivatized), which may be bound to a linker as is generally described in Rodriguez-Gonzalez, et al., Targeting steroid hormone receptors for ubiquitination and degradation in breast and prostate cancer, (2008, Oncogene 27:7201-7211);
3. Estradiol, testosterone (derivatized) and related derivatives, including but not limited to DHT and derivatives and analogs thereof, having the structure(s) and binding to a linker as generally described in Sakamoto, et al., Development of Protacs to target cancer-promoting proteins for ubiquitination and degradation, (2003 Dec., Mol. Cell Proteomics, 2(12):1350-1358); and
4. Ovalicin, fumagillin (derivatized), and derivatives and analogs thereof, having the structure(s) and binding to a linker as is generally described in Sakamoto, et al., Protacs: chimeric molecules that target proteins to the Skp1—Cullin-F box complex for ubiquitination and degradation (2001 Jul., Proc. Natl. Acad. Sci. USA, 98(15):8554-8559) and U.S. Pat. No. 7,208,157, the entire contents of which are incorporated herein by reference.

VIII. Immunosuppressive Compounds:

Immunosuppressive compounds useful in some examples of the disclosure include, but are not limited to:
1. AP21998 (derivatized), having the structure(s) and binding to a linker as is generally described in Schneekloth, et al., Chemical Genetic Control of Protein Levels: Selective in Vivo Targeted Degradation (2004, J. Am. Chem. Soc., 126:3748-3754);
2. Glucocorticoids (e.g., hydrocortisone, prednisone, prednisolone, and methylprednisolone) (Derivatized where a linker is bound, e.g. to any of the hydroxyls) and beclometasone dipropionate (Derivatized where a linker is bound, e.g. to a proprionate);
3. Methotrexate (Derivatized where a linker can be bound, e.g. to either of the terminal hydroxyls);
4. Ciclosporin (Derivatized where a linker can be bound, e.g. at a of the butyl groups);
5. Tacrolimus (FK-506) and rapamycin (Derivatized where a linker group can be bound, e.g. at one of the methoxy groups); and
6. Actinomycins (Derivatized where a linker can be bound, e.g. at one of the isopropyl groups).

IX. Compounds Targeting the Aryl Hydrocarbon Receptor (AHR):

Compounds targeting the aryl hydrocarbon receptor (AHR) according to some examples of the disclosure include, but are not limited to:
1. Apigenin (Derivatized in a way which binds to a linker as is generally illustrated in Lee, et al., Targeted Degradation of the Aryl Hydrocarbon Receptor by the PROTAC Approach: A Useful Chemical Genetic Tool, ChemBioChem Volume 8, Issue 17, pages 2058-2062, Nov. 23, 2007); and
2. SRI and LGC006 (derivatized such that a linker is bound), as described in Boitano, et al., Aryl Hydrocarbon Receptor Antagonists Promote the Expansion of Human Hematopoietic Stem Cells (2010 Sep., Science, 329(5997):1345-1348).

X. Compounds Targeting RAF Receptor (Kinase):

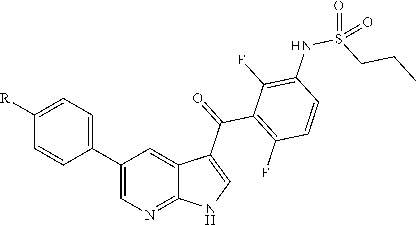

PLX4032

(Derivatized where “R” designates a site for linker attachment).

XI. Compounds Targeting FKBP:

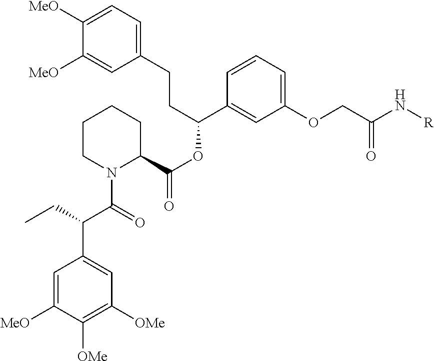

(Derivatized where “R” designates a site for linker attachment).

XII. Compounds Targeting Androgen Receptor (AR)
1. RU59063 Ligand (derivatized) at Androgen Receptor

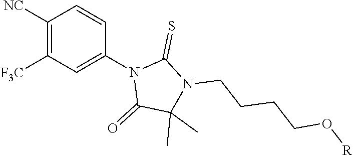

(Derivatized where “R” designates a site for linker attachment).
2. SARM Ligand (derivatized) of Androgen Receptor

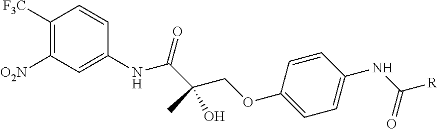

(Derivatized where “R” designates a site for linker attachment).
3. Androgen Receptor Ligand DHT (derivatized)

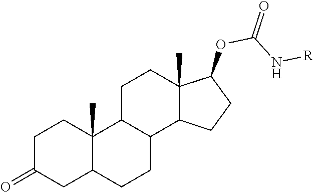

(Derivatized where “R” designates a site for linker attachment).
4. MDV3100 Ligand (derivatized)

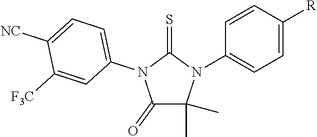

5. ARN-509 Ligand (derivatized)

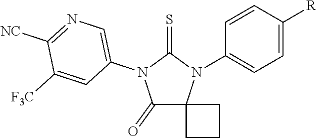

6. Hexahydrobenzisoxazoles

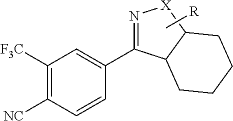

7. Tetramethylcyclobutanes

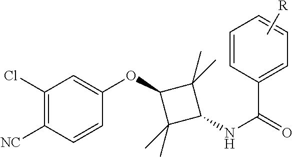

XIII. Compounds Targeting Estrogen Receptor (ER) ICI-182780
1. Estrogen Receptor Ligand

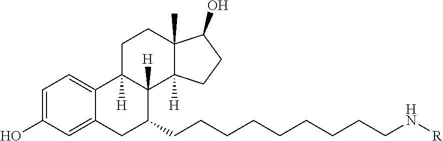

(Derivatized where “R” designates a site for linker attachment).

XIV. Compounds Targeting Thyroid Hormone Receptor (TR)
1. Thyroid Hormone Receptor Ligand (derivatized)

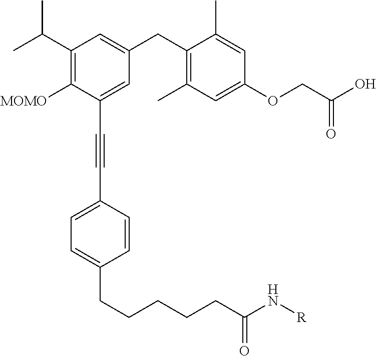

(Derivatized where “R” designates a site for linker attachment and MOMO indicates a methoxymethoxy group).

XV. Compounds Targeting HIV Protease
1. Inhibitor of HIV Protease (derivatized)

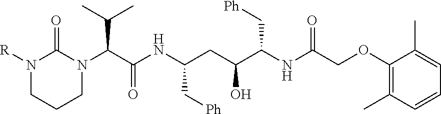

(Derivatized where “R” designates a site for linker attachment). See, 2010, J. Med. Chem., 53:521-538.
2. Inhibitor of HIV Protease

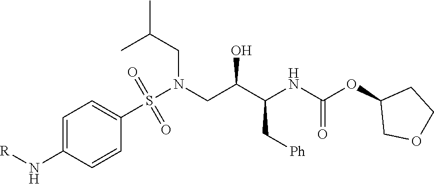

(Derivatized where “R” designates a potential site for linker attachment). See, 2010, J. Med. Chem., 53:521-538.

XVI. Compounds Targeting HIV Integrase
1. Inhibitor of HIV Integrase (derivatized)

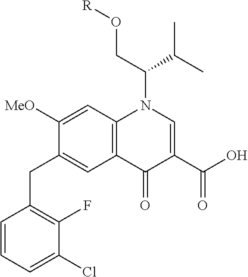

(Derivatized where “R” designates a site for linker attachment). See, 2010, J. Med. Chem., 53:6466.
2. Inhibitor of HIV Integrase (derivatized)

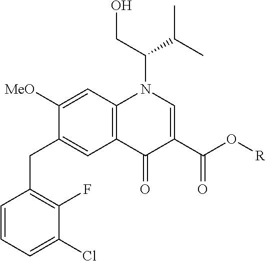

3. Inhibitor of HIV integrase Isentress (derivatized)

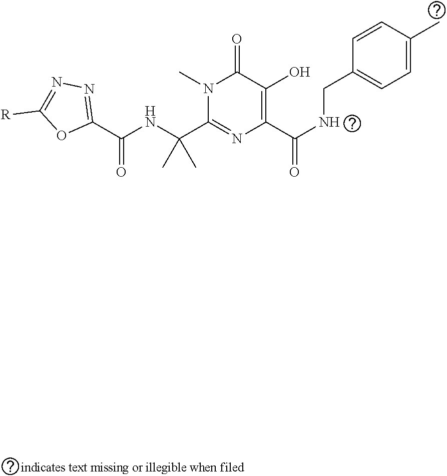

(Derivatized where “R” designates a site for linker attachment). See, 2010, J. Med. Chem., 53:6466.

XVII. Compounds Targeting HCV Protease
1. Inhibitors of HCV Protease (derivatized)

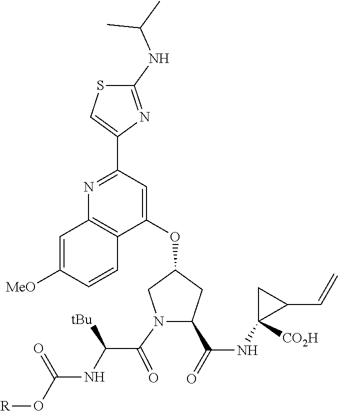

(Derivatized where “R” designates a site for linker attachment).

XVIII. Compounds Targeting Acyl-protein Thioesterase-1 and -2 (APTI and APT2)
1. Inhibitor of APTI and APT2 (derivatized)

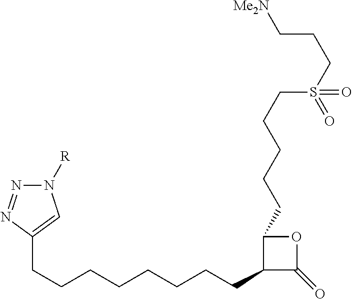

(Derivatized where “R” designates a site for linker attachment). See 2011, Angew. Chem. Int. Ed., 50:9838-9842.

Degradation Activity

Degradation may be determined by measuring the amount of a target protein in the presence of a bifunctional molecule as described herein and/or comparing this to the amount of the target protein observed in the absence of the bifunctional molecule. For example, the amount of target protein in a cell that has been contacted and/or treated with a bifunctional molecule as described herein may be determined. This amount may be compared to the amount of target protein in a cell that has not been contacted and/or treated with the bifunctional molecule. If the amount of target protein is decreased in the cell contacted and/or treated with the bifunctional molecule, the bifunctional molecule may be considered as facilitating and/or promoting the degradation and/or proteolysis of the target protein.

The amount of the target protein can be determined using methods known in the art, for example, by performing immunoblotting assays, Western blot analysis and/or ELISA with cells that have been contacted and/or treated with a bifunctional molecule.

Selective degradation and/or increased proteolysis may be considered to have occurred if at least a 10% decrease in the amount of a target protein is observed, for example, 20%, 30%, 40%, 50%, 60%, 70%, 80%, 90% or 100% following administration of the bifunctional molecule to the cell.

For example, selective degradation and/or increased proteolysis may be considered to have occurred if at least a 10% decrease in the amount of a target protein is observed, (e.g. at least 20%, 30%, 40%, 50%, 60%, 70%, 80%, 90% or 100% decrease) within 4 hours or more (e.g. 4 hours, 8 hours, 12 hours, 24 hours, 30 hours, 36 hours, 42 hours, 48 hours, 54 hours, 60 hours, 66 hours and 72 hours) following administration of the bifunctional molecule to the cell. The bifunctional molecule may be administered at any concentration, e.g. a concentration between 0.01 nM to 10 μM, such as 0.01 nM, 0.1 nM, 1 nM, 10 nM, 100 nM, 1 μM, and 10 μM. In some instances, an increase of at least 30%, 40%, 50%, 60%, 70%, 80%, 90%, or approximately 100% in the degradation of the target protein is observed following administration of the bifunctional molecule at a concentration of approximately 100 nM (e.g. following an incubation period of approximately 8 hours).

One measure of degrader activity of the bifunctional molecules is the DC₅₀ value. As used herein, DC₅₀ is the concentration required to reach 50% of the maximal degradation of the target protein. The bifunctional molecules described herein may comprise a DC₅₀ of less than or equal to 10000 nM, less than or equal to 1000 nM, less than or equal to 500 nM, less than or equal to 100 nM or less than or equal to 75 nM. In some cases, the bifunctional molecules comprise a DC₅₀ less than or equal to 50 nM, less than or equal to 25 nM, or less than or equal to 10 nM.

Another measure of the degrader activity of the bifunctional molecules is the Dₘₐₓ value. As used herein, Dₘₐₓ represents the maximal percentage of target protein degradation.

The bifunctional molecules described herein may comprise a Dₘₐₓ of at least 50%, at least 60%, at least 70%, at least 80%, at least 90%, at least 95% or about 100%.

Yet another measure of the efficacy of the described bifunctional molecules may be their effect on cell viability and/or their IC₅₀ value. For example, an anti-proliferative effect of a bifunctional molecule as described herein may be assessed in a cell viability assay to provide an IC₅₀ value. As used herein, the IC₅₀ value represents the concentration at which 50% cell viability was observed in the cell viability assay (following administration of a bifunctional molecule as described herein). In terms of cell viability, the bifunctional molecules described herein may comprise an IC₅₀ of less than 1000 nM, less than 500 nM, less than 100 nM, less than 50 nM, less than 25 nM, less than 20 nM, or less than 10 nM. In some cases, the bifunctional molecules described herein may comprise an IC50 value of less than 5 nM.

Bioavailability

The bifunctional molecules described herein may provide degraders with improved levels of bioavailability, such as improved levels of oral bioavailability.

As used herein, bioavailability is a fraction or proportion of an administered active agent (e.g. a bifunctional molecule as described herein) that reaches the systemic circulation in a subject. As used herein, oral bioavailability is a fraction or proportion of an orally administered active agent that reaches the systemic circulation in a subject.

Oral bioavailability is calculated by comparing the area under the curve (AUC) for an intravenous administration of a particular active agent to the AUC for an oral administration of that active agent. The AUC value is the definite integral of a curve that shows the variation of active agent concentration in the blood plasma as a function of time. As used herein, AUC_{0-INF} is the area under the curve from time zero which has been extrapolated to infinity and represents the total active agent exposure over time

Oral bioavailability (F) may be calculated using the following formula:

$$F = 100. \frac{{AUC}_{po} . {D}_{iv}}{{AUC}_{iv} . {D}_{po}}$$

Wherein:
Dᵢᵥ=dose administered intravenously;
Dₚₒ=dose administered orally;
AUCᵢᵥ=Area under the curve from time zero to infinity following intravenous administration; and
AUCₚₒ=Area under the curve from time zero to infinity following oral administration.

The bifunctional molecules described herein may have an oral bioavailability of at least about 1%, at least about 2%, at least about 3%, at least about 4%, at least about 5%, at least about 6%, or at least about 7%. In some cases, the oral bioavailability of a bifunctional molecule as described herein may be approximately 7%.

CNS (Central Nervous System) Penetration

The bifunctional molecules described herein may provide degraders which can cross the blood-brain barrier and/or which show CNS penetration.

A level of CNS penetration and/or a degree to which an active agent is able to cross the blood brain barrier in a subject may be determined by comparing the concentration of an active agent in the blood plasma to the concentration of that active agent in the brain following administration of the active agent to a subject. The degree of CNS penetration may be expressed as a ratio of the concentration of the active agent in the brain to the concentration of the active agent in the blood plasma (Cb:Cp).

The bifunctional molecules as described herein may have a Cb:Cp ratio of at least about 0.01:1, at least about 0.05:1, at least about 0.1:1, at least about 0.2:1, at least about 0.3:1, at least about 0.4:1, at least about 0.5:1 or at least about 0.6:1.

Pharmaceutical Compositions

The present disclosure provides a pharmaceutical composition comprising the bifunctional molecules described herein. In such compositions, the bifunctional molecule may be suitably formulated such that it can be introduced into the environment of the cell by a means that allows for a sufficient portion of the molecule to enter the cell to induce degradation of the target protein.

Accordingly, there is provided a pharmaceutical composition comprising a bifunctional molecule as described herein together with a pharmaceutically acceptable carrier.

Pharmaceutically acceptable carriers are well known to those skilled in the art and include, but are not limited to, phosphate buffer solutions and/or saline. Pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

In addition to the aforementioned carrier ingredients the pharmaceutical compositions described above may alternatively or additionally include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutical compositions may be present in any formulation typical for the administration of a pharmaceutical compound to a subject. Representative examples of typical formulations include, but are not limited to, capsules, granules, tablets, powders, lozenges, suppositories, pessaries, nasal sprays, gels, creams, ointments, sterile aqueous preparations, sterile solutions, aerosols, implants etc.

A pharmaceutical composition is formulated to be compatible with its intended route of administration. Examples of routes of administration include parenteral, e.g., intravenous, intradermal, subcutaneous, oral, transdermal, topical, transmucosal, vaginal and rectal administration.

The pharmaceutical compositions may include those suitable for oral, parenteral (including subcutaneous, intradermal, intramuscular and intravenous), topical (including dermal, buccal and sublingual), rectal, nasal and pulmonary administration e.g., by inhalation. The composition may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical compositions suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. The bifunctional molecules may also be formulated as dispersible granules, which may for example be suspended in water before administration, or sprinkled on food. The granules may be packaged, e.g., in a sachet. Compositions suitable for oral administration wherein the carrier is a liquid may be presented as a solution or a suspension in an aqueous or non-aqueous liquid, or as an oil-in-water liquid emulsion. Compositions for oral administration include controlled release dosage forms, e.g., tablets wherein an active compound is formulated in an appropriate release-controlling matrix, or is coated with a suitable release-controlling film.

Pharmaceutical compositions suitable for parenteral administration include sterile solutions or suspensions of an active compound in aqueous or oleaginous vehicles. Injectable preparations may be adapted for bolus injection or continuous infusion. Such preparations are conveniently presented in unit dose or multi-dose containers, which are sealed after introduction of the formulation until required for use. Alternatively, the bifunctional molecule may be in powder form, which is constituted with a suitable vehicle, such as sterile, pyrogen-free water, before use.

The pharmaceutical composition may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins.

Pharmaceutical compositions suitable for topical formulation may be provided for example as gels, creams or ointments.

The bifunctional molecules described herein may be present in the pharmaceutical compositions as a pharmaceutically and/or physiologically acceptable salt, solvate or derivative.

Representative examples of pharmaceutically and/or physiologically acceptable salts of the bifunctional molecules of the disclosure may include, but are not limited to, acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Pharmaceutically and/or physiologically functional derivatives of compounds of the present invention are derivatives, which may be converted in the body into the parent compound. Such pharmaceutically and/or physiologically functional derivatives may also be referred to as “pro-drugs” or “bioprecursors”. Pharmaceutically and/or physiologically functional derivatives of compounds of the present disclosure may include hydrolysable esters or amides, particularly esters, in vivo.

It may be convenient or desirable to prepare, purify, and/or handle a corresponding pharmaceutically and/or physiologically acceptable solvate of the bifunctional molecules described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

Uses of Moiety Z

As described herein, the moiety Z may form part of a bifunctional molecule intended for use in a method of targeted protein degradation, wherein the moiety Z acts to modulate, facilitate and/or promote proteasomal degradation of the target protein.

As such, according to a further aspect of the disclosure, there is provided a use of the moiety Z or a compound comprising moiety Z (e.g. as defined in any one of formula (I) to (III)) in a method of targeted protein degradation (e.g. an in vitro or in vivo method of targeted protein degradation). For example, moiety Z may find particular application as a promoter or facilitator of targeted protein degradation.

There is also provided a use of moiety Z or a compound comprising moiety Z (e.g. as defined in any one of formula (I) to (III)) in the manufacture of a bifunctional molecule suitable for targeted protein degradation.

Therapeutic Methods and Uses

The bifunctional molecules of the present disclosure may modulate, facilitate and/or promote proteasomal degradation of a target protein. As such, there is provided a method of selectively degrading and/or increasing proteolysis of a target protein in a cell, the method comprising contacting and/or treating the cell with a bifunctional molecule as described herein. The method may be carried out in vivo or in vitro.

In particular, there is provided a method of selectively degrading and/or increasing proteolysis of a target protein in a subject in need thereof, the method comprising administering to the subject a therapeutically effective amount of a bifunctional molecule of the present disclosure.

As such, the bifunctional molecules of the present disclosure may find application in medicine and/or therapy. Specifically, the bifunctional molecules of the present disclosure may find use in the treatment and/or prevention of any disease or condition, which is modulated through the target protein. For example, the bifunctional molecules of the present disclosure may be useful in the treatment of any disease, which is modulated through the target protein by lowering the level of that protein in the cell, e.g. cell of a subject.

There is further provided the use of the bifunctional molecules as described herein in the manufacture of a medicament for the treatment and/or prevention of any disease or condition, which is modulated through the target protein. Additionally, there is provided the use of a moiety Z (e.g as defined in any one of formulae (I) to (III) in the manufacture of a medicament for the treatment and/or prevention of any disease or condition, which is modulated through the target protein.

Diseases and/or conditions that may be treated and/or prevented by the molecules of the disclosure include any disease, which is associated with and/or is caused by an abnormal level of protein activity.

Such diseases and conditions include those whose pathology is related at least in part to an abnormal (e.g. elevated) level of a protein and/or the overexpression of a protein. For example, the bifunctional molecules may find use in the treatment and/or prevention of diseases where an elevated level of a protein is observed in a subject suffering from the disease. In other examples, the diseases and/or conditions may be those whose pathology is related at least in part to inappropriate protein expression (e.g., expression at the wrong time and/or in the wrong cell), excessive protein expression or expression of a mutant protein. In one example, a mutant protein disease is caused when a mutant protein interferes with the normal biological activity of a cell, tissue, or organ.

Accordingly, there is provided a method of treating and/or preventing a disease or condition, which is associated with and/or is caused by an abnormal level of protein activity, which comprises administering a therapeutically effective amount of a bifunctional compound as described herein.

Representative examples of the diseases and/or conditions that may be treated and/or prevented by the use of the described bifunctional compounds include (but are not limited to) cancer, asthma, multiple sclerosis, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKDI) or 4 (PKD2) Prader-Willi syndrome, Sickle-cell disease, Tay-Sachs disease, and Turner syndrome.

Further examples include, Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, and Vasculitis.

Yet further examples include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis, Alstrom syndrome, Alexander disease, Amelogenesis imperfecta, ALA dehydratase deficiency, Anderson-Fabry disease, androgen insensitivity syndrome, Anemia, Angiokeratoma Corporis Diffusum, Angiomatosis retinae (von Hippel-Lindau disease), Apert syndrome, Arachnodactyly (Marfan syndrome), Stickler syndrome, Arthrochalasis multiplex congenital (Ehlers-Danlos syndrome #arthrochalasia type), ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria, Erythroid 5-aminolevulinate synthetase deficiency, Erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (S ADD AN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita), SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease Tomaculous neuropathy (hereditary neuropathy with liability to pressure palsies) Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Weissenbacher-Zweymuller syndrome, Wolf-Hirschhorn syndrome, Wolff Periodic disease, Weissenbacher-Zweymuller syndrome and Xeroderma pigmentosum.

Representative examples of cancers that may be treated and/or prevented using the described bifunctional molecules include but, are not limited to squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Further examples include, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

As used herein, the term “patient” or “subject” is used to describe an animal, such as a mammal (e.g. a human or a domesticated animal), to whom treatment, including prophylactic treatment, with the compositions according to the present disclosure is provided. For treatment of those infections, conditions or disease states which are specific to a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

Assays

The disclosure also encompasses a method of identifying suitable target protein binding ligands and linkers for use in the bifunctional molecules described herein, e.g. a bifunctional molecule that is able to effectively modulate, facilitate and/or promote proteolysis of a target protein. This method may assist in identifying suitable linkers for a particular target protein binding partner such that the level of degradation is further optimised.

The method may comprise:
a. providing a bifunctional molecule comprising:
   (i) a first ligand comprising a structure according to Z (as defined in any of formulae (I) to (III);
   (ii) a second ligand that binds to a target protein (a target protein binding ligand); and
   (iii) a linker that covalently attaches the first and second ligands;
b. contacting a cell with the bifunctional molecule; and
c. detecting degradation of the target protein in the cell.

This method may further comprise the steps of:
d. detecting degradation of the target protein in the cell in the absence of the bifunctional molecule; and
e. comparing the level of degradation of the target protein in the cell contacted with the bifunctional molecule to the level of degradation of the target protein in the absence of the bifunctional molecule;
   wherein an increased level of degradation of the target protein in the cell contacted with the bifunctional molecule indicates that the bifunctional molecule has facilitated and/or promoted the degradation of the target protein.

In such methods, a step of detecting degradation of the target protein may comprise detecting changes in levels of a target protein in a cell. For example, a reduction in the level of the target protein indicates degradation of the target protein. An increased reduction in the level of the target protein in the cell contacted with the bifunctional molecule (compared to any reduction in the levels of target protein observed in the cell in the absence of the bifunctional molecule) indicates that the bifunctional molecule has facilitated and/or promoted the degradation of the target protein.

The method may further comprise providing a plurality of linkers, each one being used to covalently attach the first and second ligands together to form a plurality of bifunctional molecules. The level of degradation provided by each one of the plurality of bifunctional molecules may be detected and compared. Those bifunctional molecules showing higher levels of target protein degradation indicate preferred and/or optimal linkers for use with the selected target protein binding partner.

The method may be carried out in vivo or in vitro.

Compound Library

The disclosure also provides a library of bifunctional molecules, the library comprising a plurality of bifunctional molecules, the plurality of bifunctional molecules comprising a plurality of Z moieties covalently linked to a selected target protein binding partner.

As such, the target protein binding partner may be pre-selected and the Z moiety may not be determined in advance. The library may be used to determine the activity of a candidate Z moiety of a bifunctional molecule in modulating, promoting and/or facilitating selective protein degradation of a target protein.

The disclosure also includes a library of bifunctional molecules, the library comprising a plurality of bifunctional molecules, the plurality of bifunctional molecules comprising a plurality of target protein binding ligands and a selected Z moiety. As such, the Z moiety of the bifunctional molecule may be pre-selected and the target protein may not be determined in advance. The library may be used to determine the activity of a putative target protein binding ligand and its value as a binder of a target protein to facilitate target protein degradation.

Methods of Manufacture

According to a further aspect of the disclosure, there is provided a method of making a bifunctional molecule as described herein.

The method of making the bifunctional molecule may comprise the steps of:
(a) providing a first ligand or moiety comprising a structure according to Z (as defined in any of formulae (I) to (IV);
(b) providing a second ligand or moiety that binds to a target protein (e.g. a target protein binding ligand as defined herein); and
(c) linking (e.g. covalently linking) the first and second ligands or moieties using a linker as defined herein.

In other examples, the method of making the bifunctional molecule may comprise the steps of:
(a) providing a target protein binding ligand (as defined herein);
(b) linking (e.g. covalently linking) a linker (as defined herein) to the target protein binding ligand to provide a target protein binding ligand-linker conjugate (TBL-L);
(c) further reacting the linker moiety of the conjugate to add and/or form a structure according to Z (as defined in any of formulae (I) to (III)) thereon to provide the bifunctional molecule having the general formula TBL-L-Z.

### DETAILED DESCRIPTION

The present invention will now be described in detail with reference to the following figures which show:

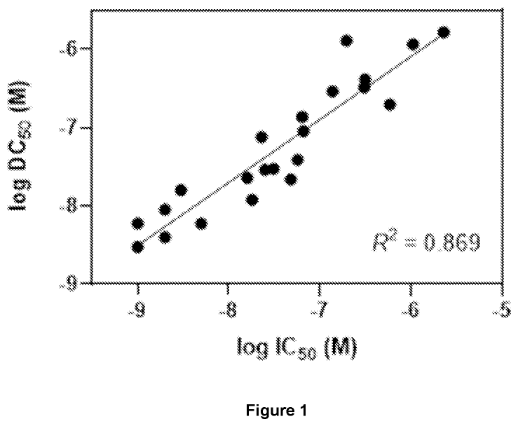

FIG. 1 shows plot of correlation between the IC₅₀ and DC₅₀ values for a number of bifunctional molecules that are useful in targeted protein degradation.

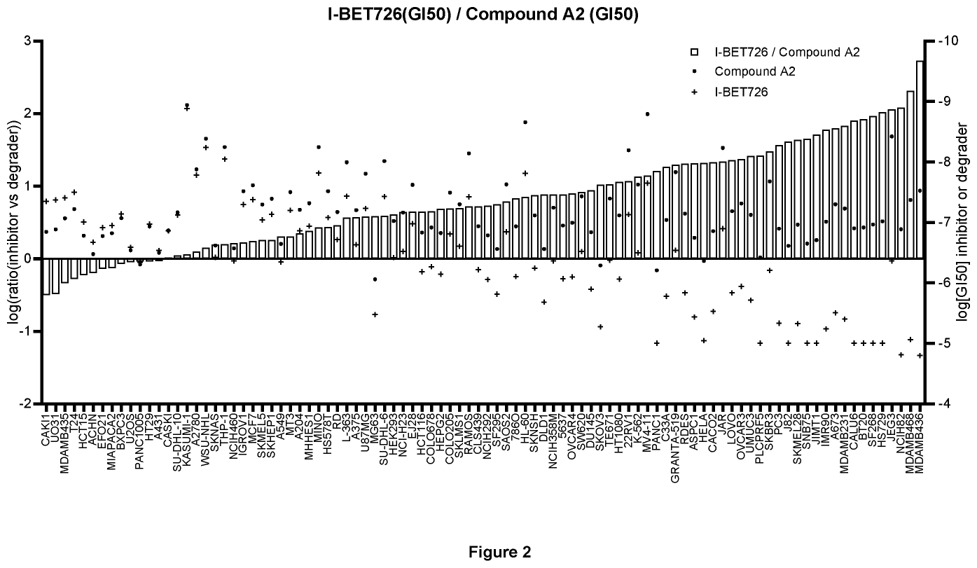

FIG. 2 shows log ratio of the GI₅₀ determined for I-BET726 versus the GI₅₀ determined for compound A2 plotted for each cell line tested (bars, left y axis). Values>0 indicate cell lines where BET-degradation by A2 shows greater efficacy than the inhibitor I-BET726 due to catalytic activity, whereas values<0 indicate cell lines where BET degrader A2 is less efficacious than BET-inhibition with I-BET726 suggestive of weaker protein degradation.

### PART A—SYNTHETIC METHODS

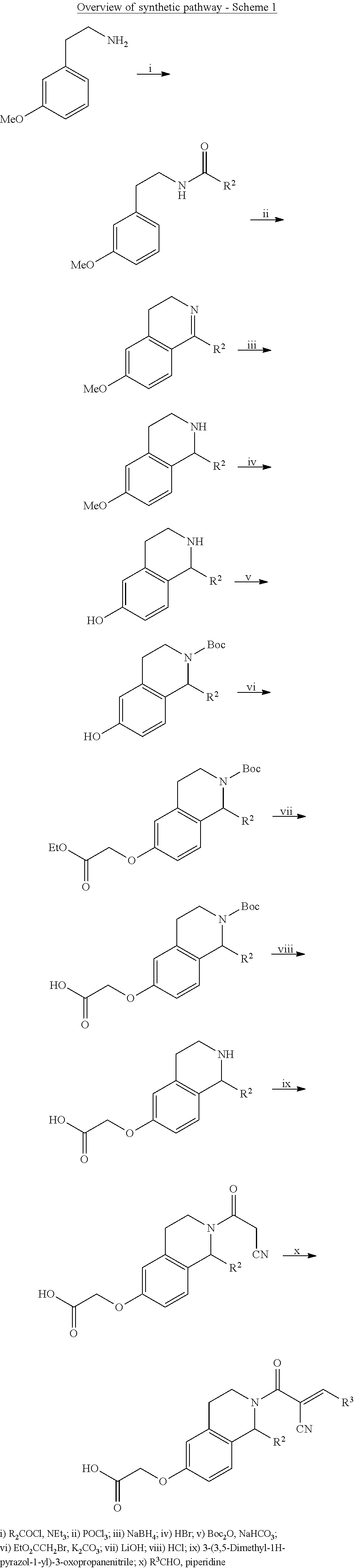

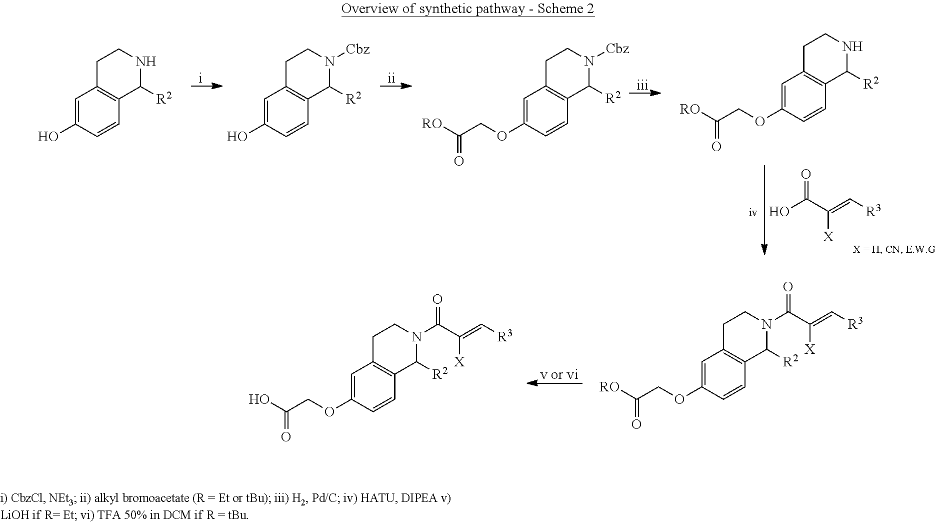

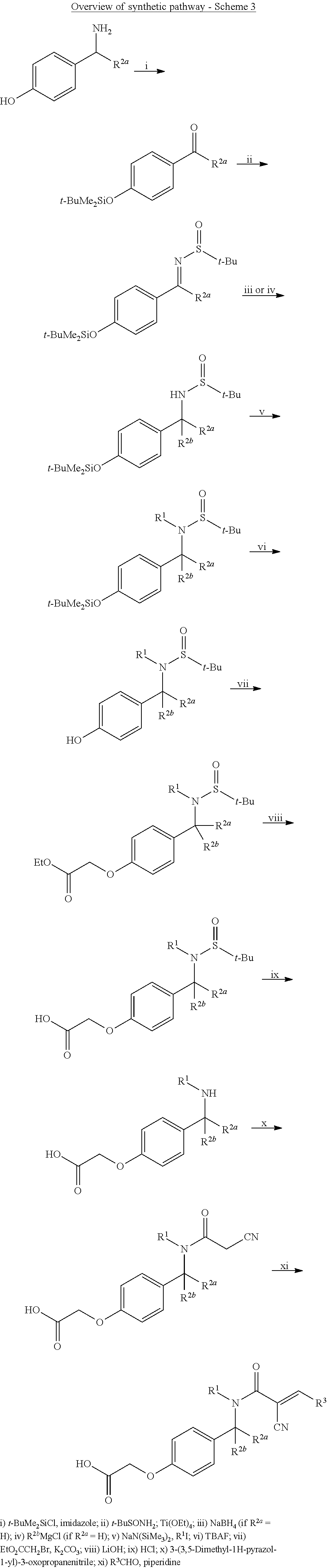

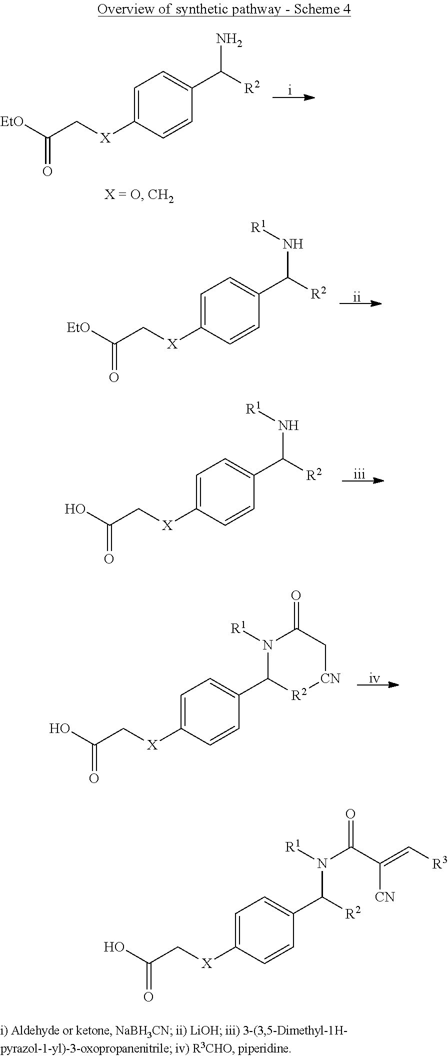

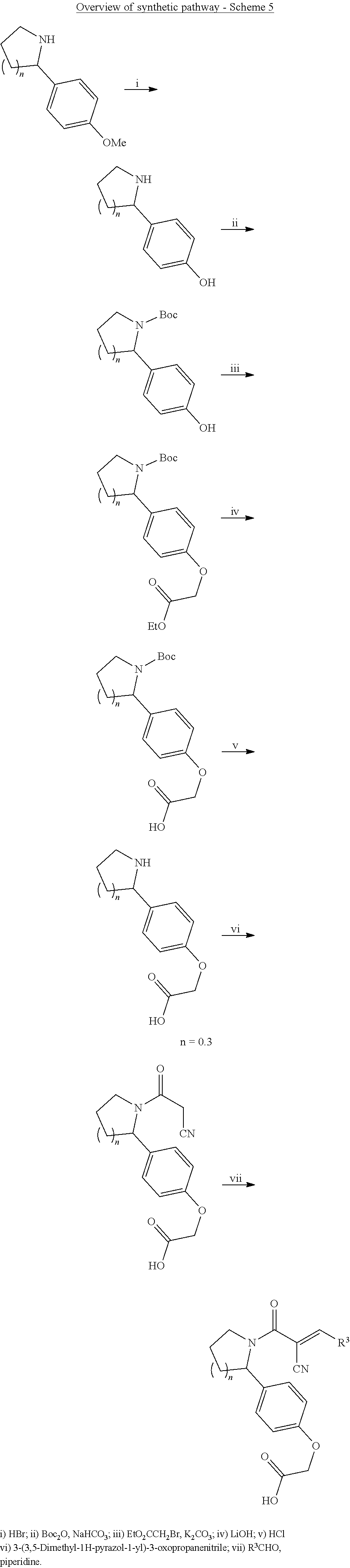

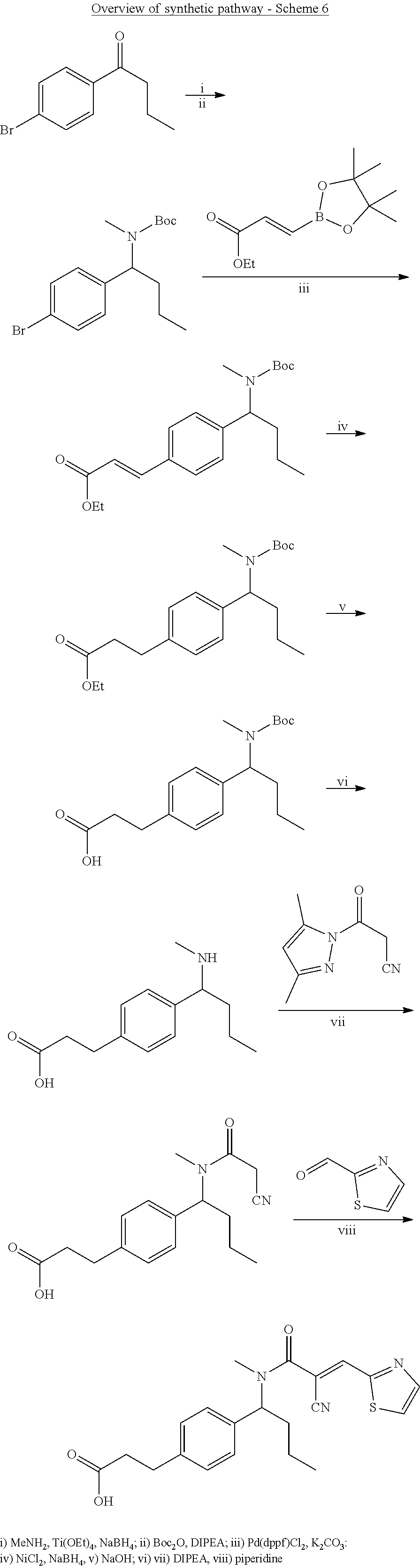

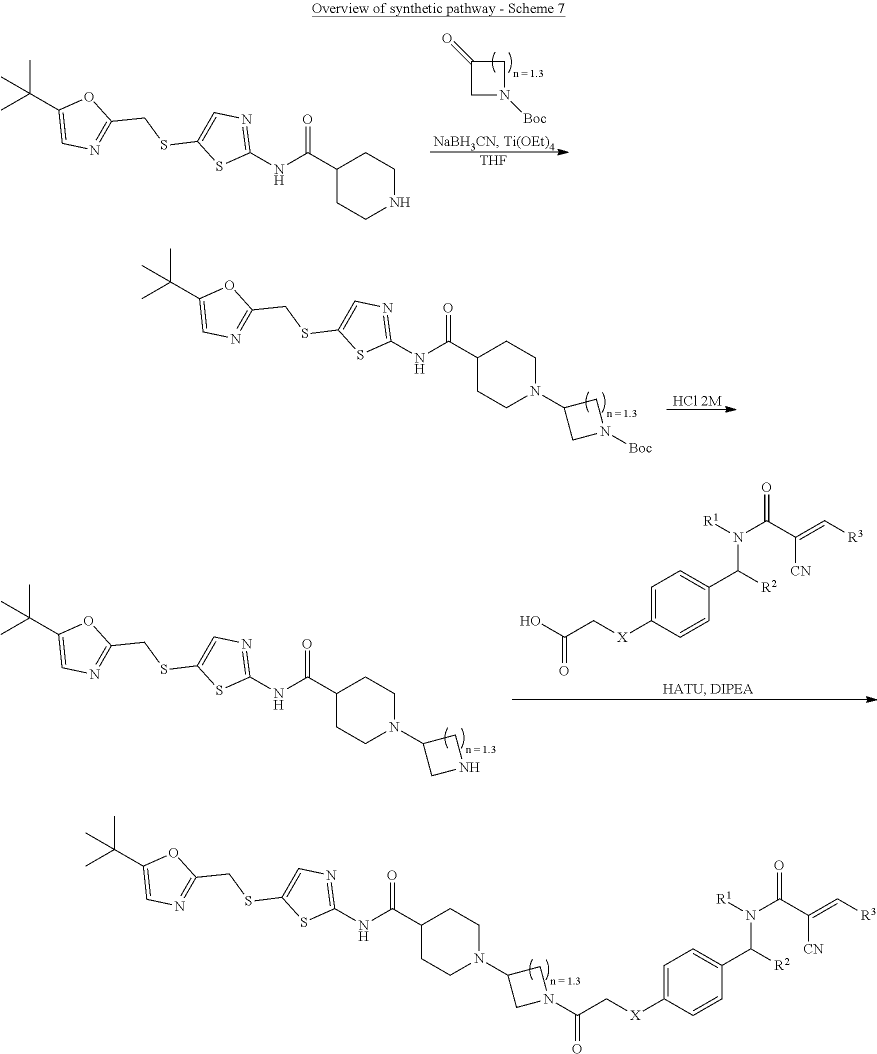

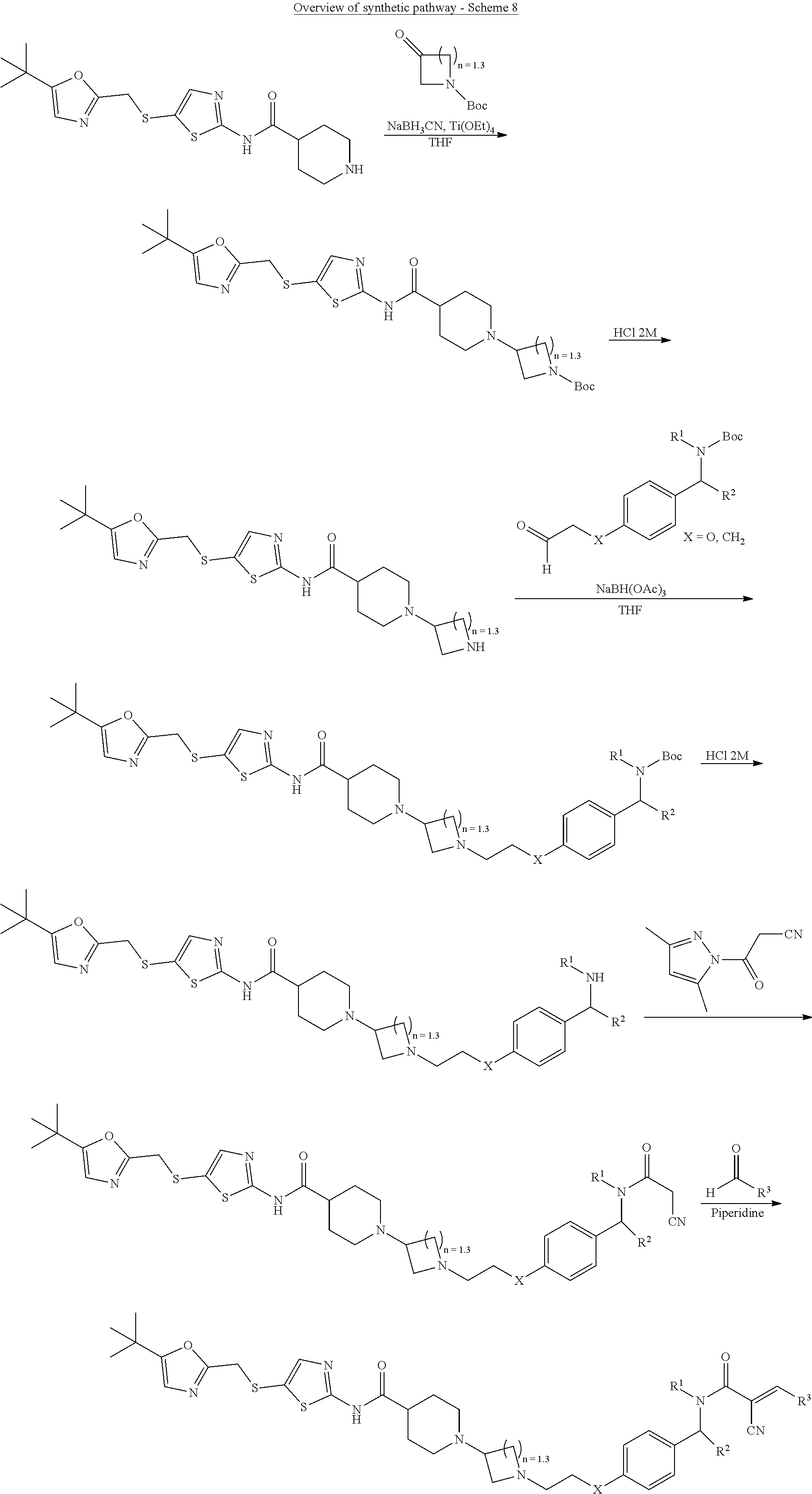

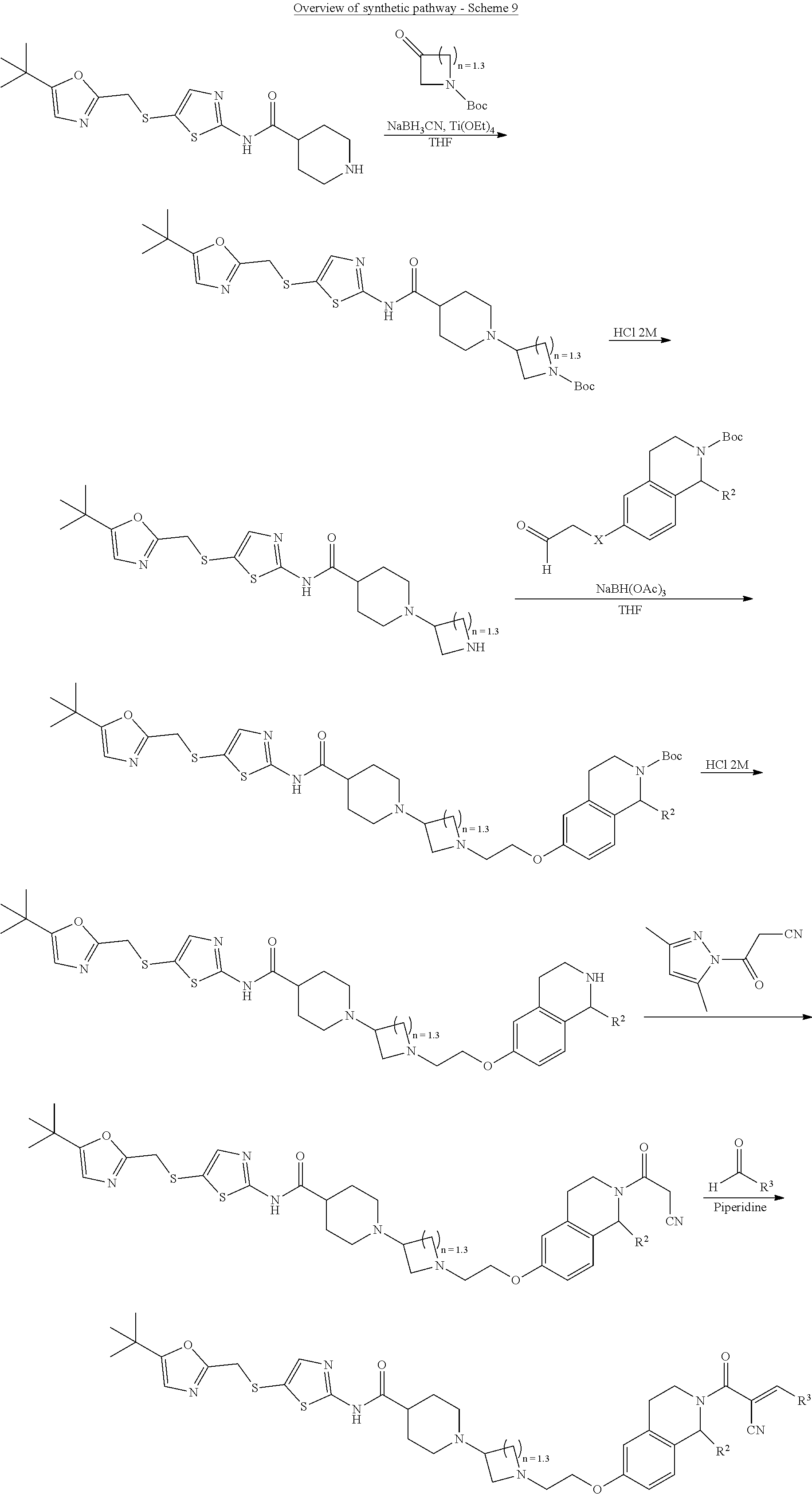

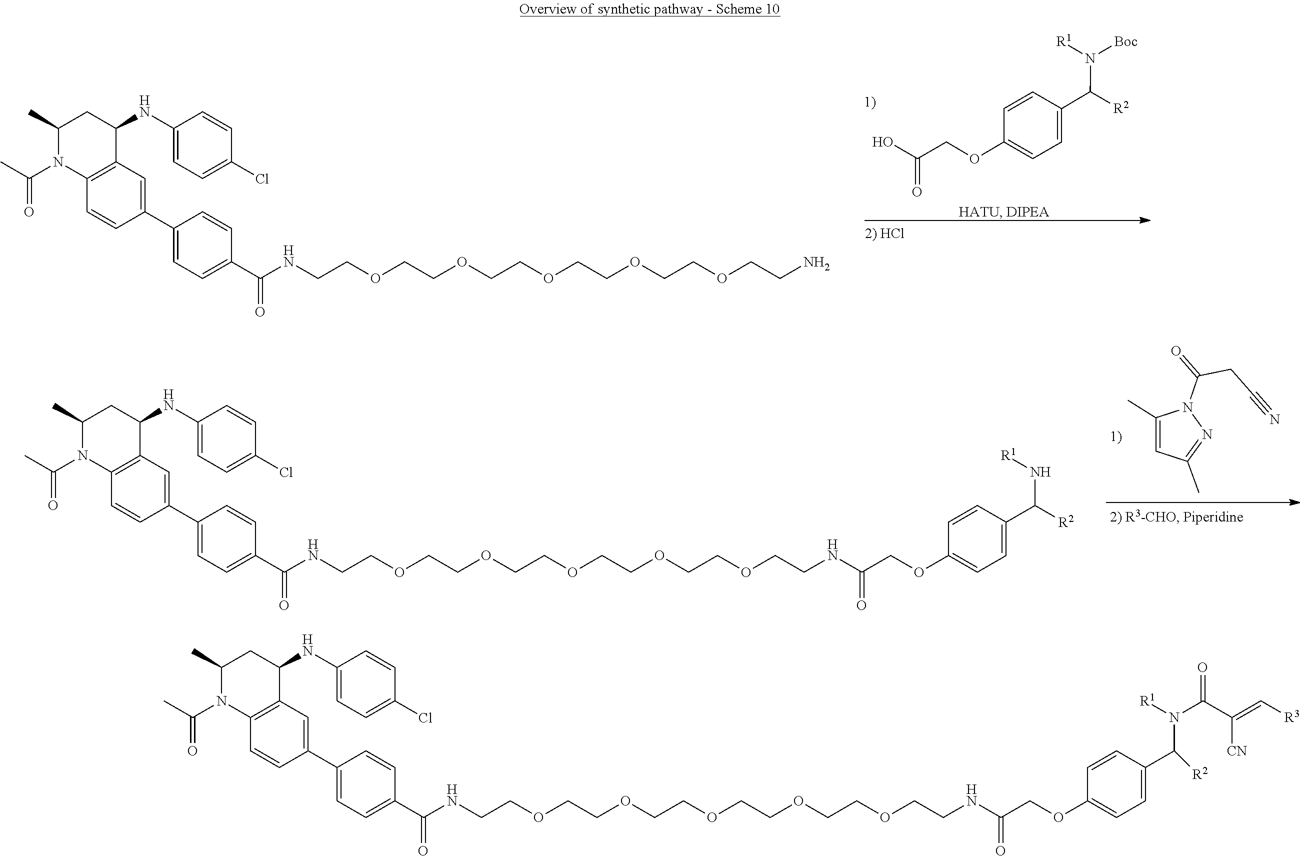

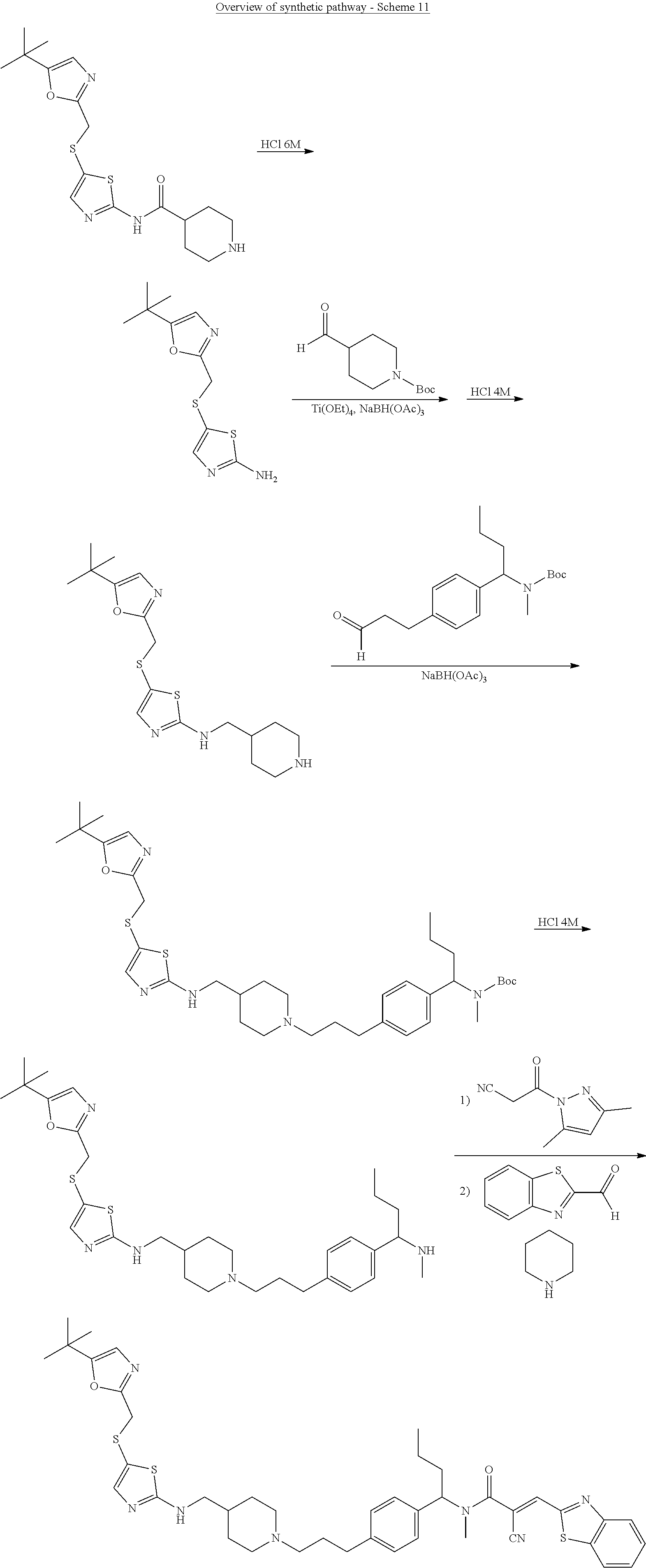

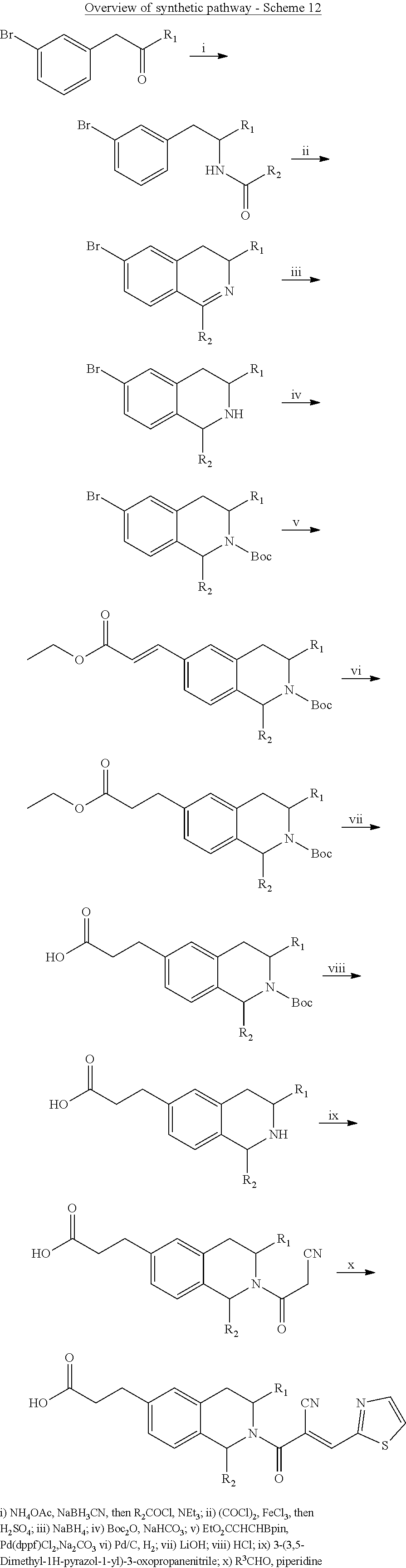

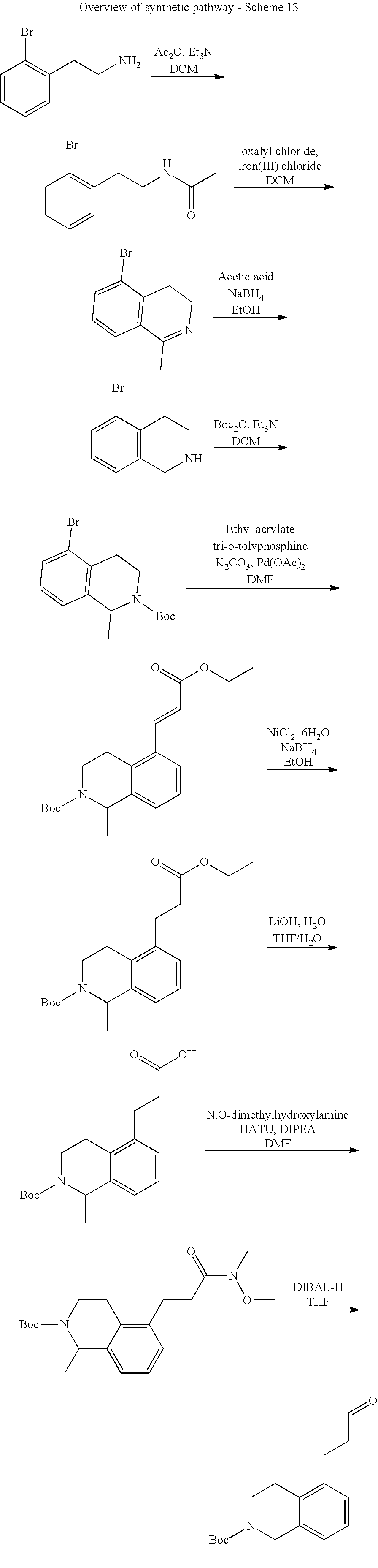

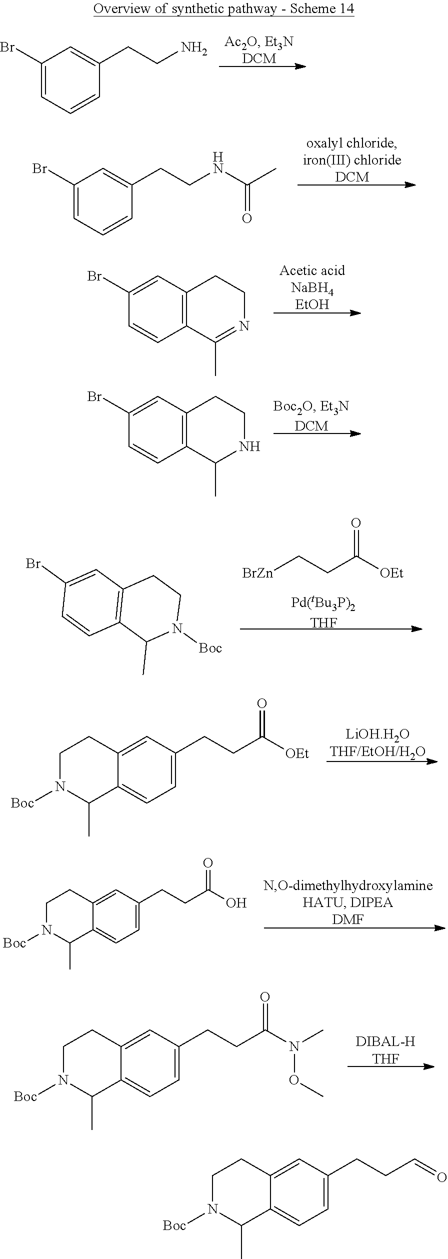

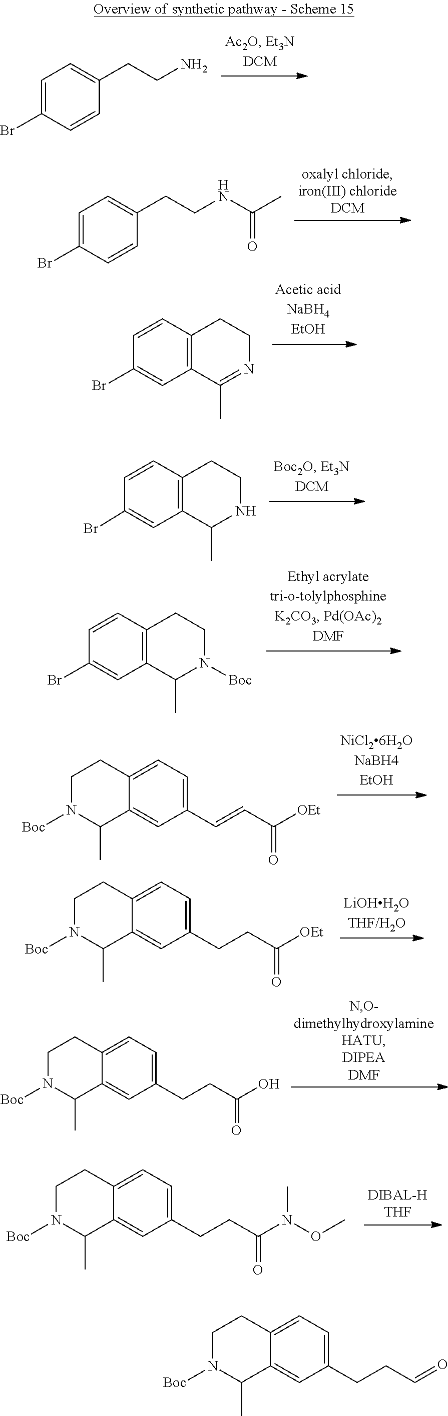

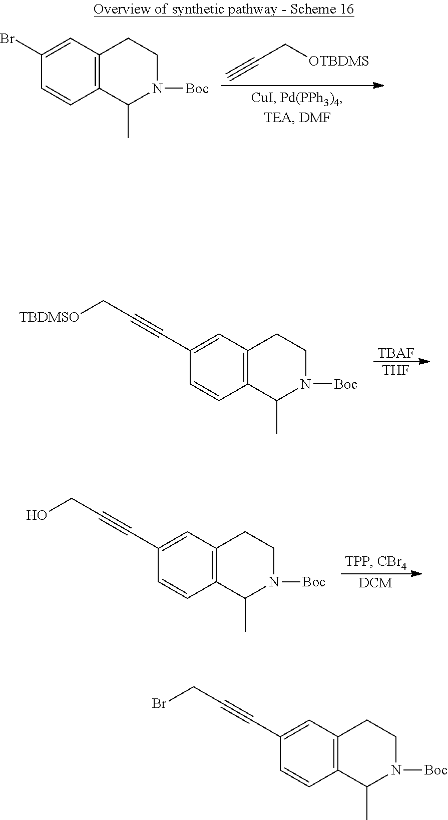

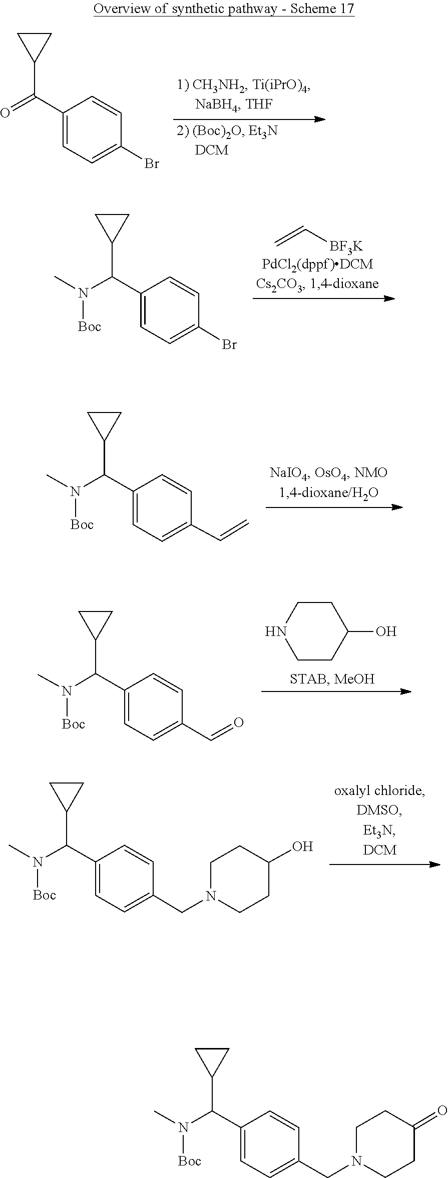

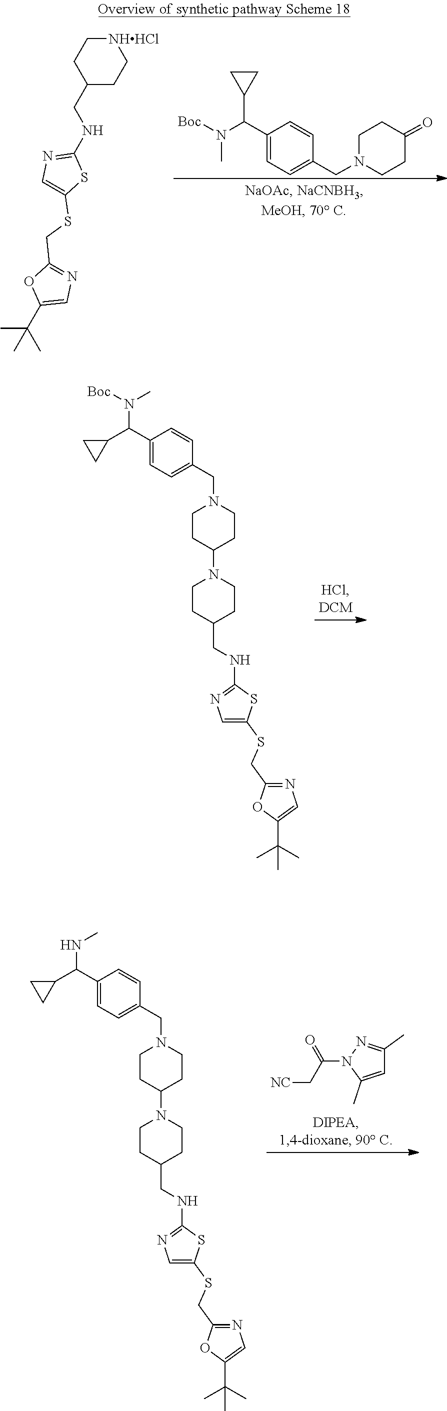

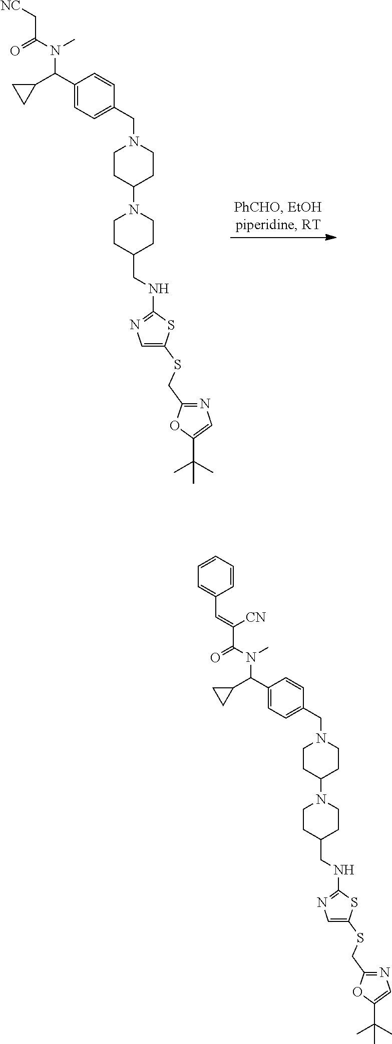 [SMILES: CN(C(=O)CC#N)C(c1ccc(CN2CCC(N3CCC(CNc4ncc(SCc5ncc(C(C)(C)C)o5)s4)CC3)CC2)cc1)C1CC1 ; CN(C(=O)/C(C#N)=C/c1ccccc1)C(c1ccc(CN2CCC(N3CCC(CNc4ncc(SCc5ncc(C(C)(C)C)o5)s4)CC3)CC2)cc1)C1CC1]

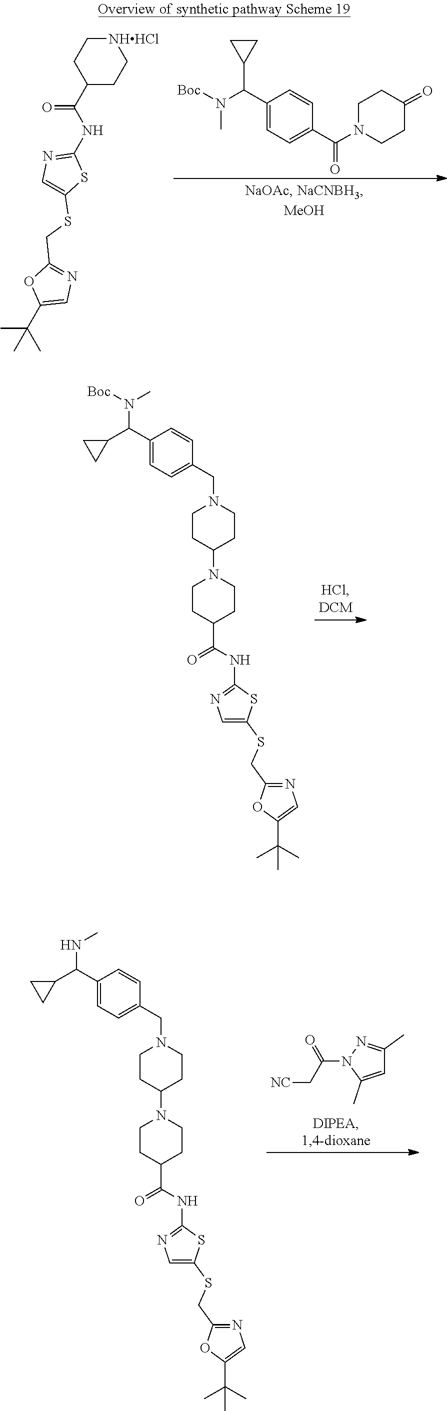

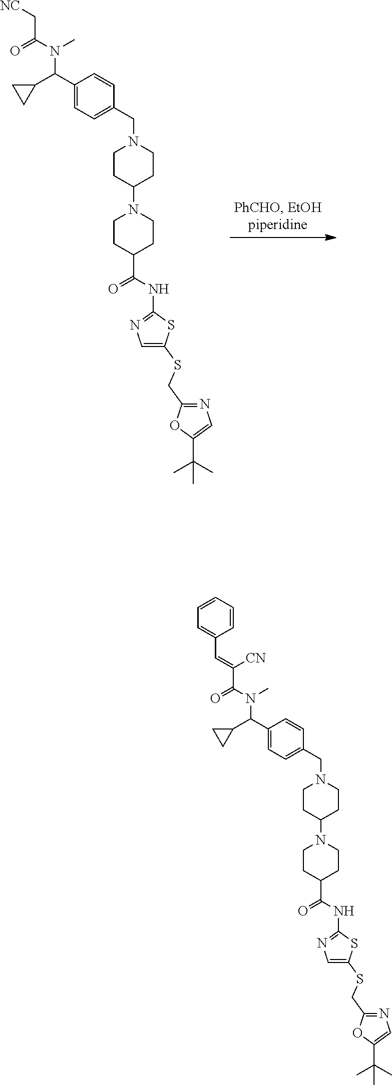 [SMILES: CN(C(=O)CC#N)C(c1ccc(CN2CCC(N3CCC(C(=O)Nc4ncc(SCc5ncc(C(C)(C)C)o5)s4)CC3)CC2)cc1)C1CC1 ; CN(C(=O)/C(C#N)=C/c1ccccc1)C(c1ccc(CN2CCC(N3CCC(C(=O)Nc4ncc(SCc5ncc(C(C)(C)C)o5)s4)CC3)CC2)cc1)C1CC1]

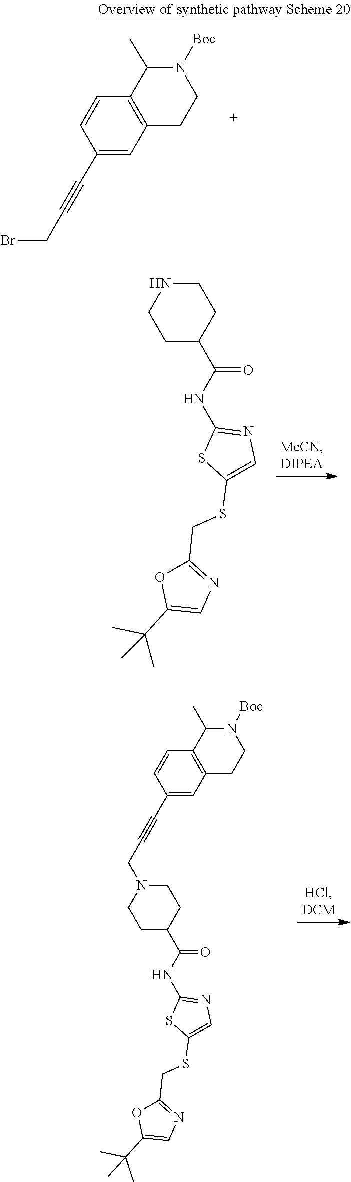 [SMILES: CC1c2ccc(C#CCBr)cc2CCN1C(=O)OC(C)(C)C ; CC(C)(C)c1cnc(CSc2cnc(NC(=O)C3CCNCC3)s2)o1 ; CC1c2ccc(C#CCN3CCC(C(=O)Nc4ncc(SCc5ncc(C(C)(C)C)o5)s4)CC3)cc2CCN1C(=O)OC(C)(C)C]

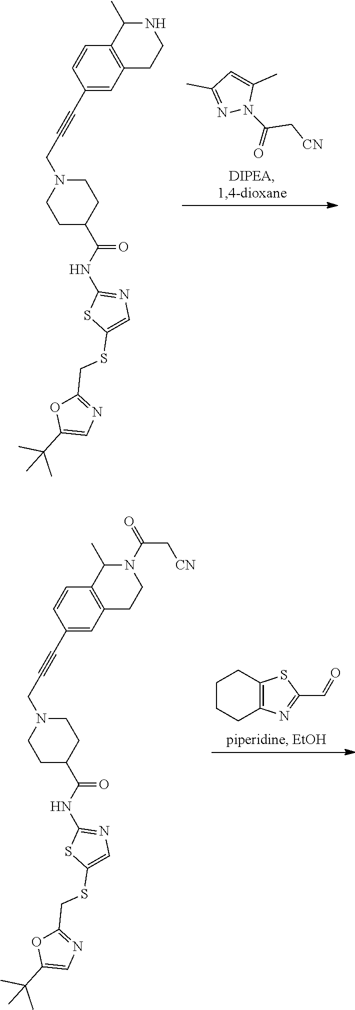 [SMILES: CC1NCCc2cc(C#CCN3CCC(C(=O)Nc4ncc(SCc5ncc(C(C)(C)C)o5)s4)CC3)ccc21 ; Cc1cc(C)n(C(=O)CC#N)n1 ; CC1c2ccc(C#CCN3CCC(C(=O)Nc4ncc(SCc5ncc(C(C)(C)C)o5)s4)CC3)cc2CCN1C(=O)CC#N ; O=Cc1nc2c(s1)CCCC2]

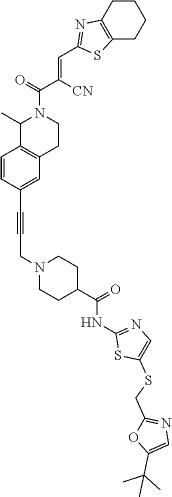 [SMILES: CC1c2ccc(C#CCN3CCC(C(=O)Nc4ncc(SCc5ncc(C(C)(C)C)o5)s4)CC3)cc2CCN1C(=O)/C(C#N)=C/c1nc2c(s1)CCCC2]

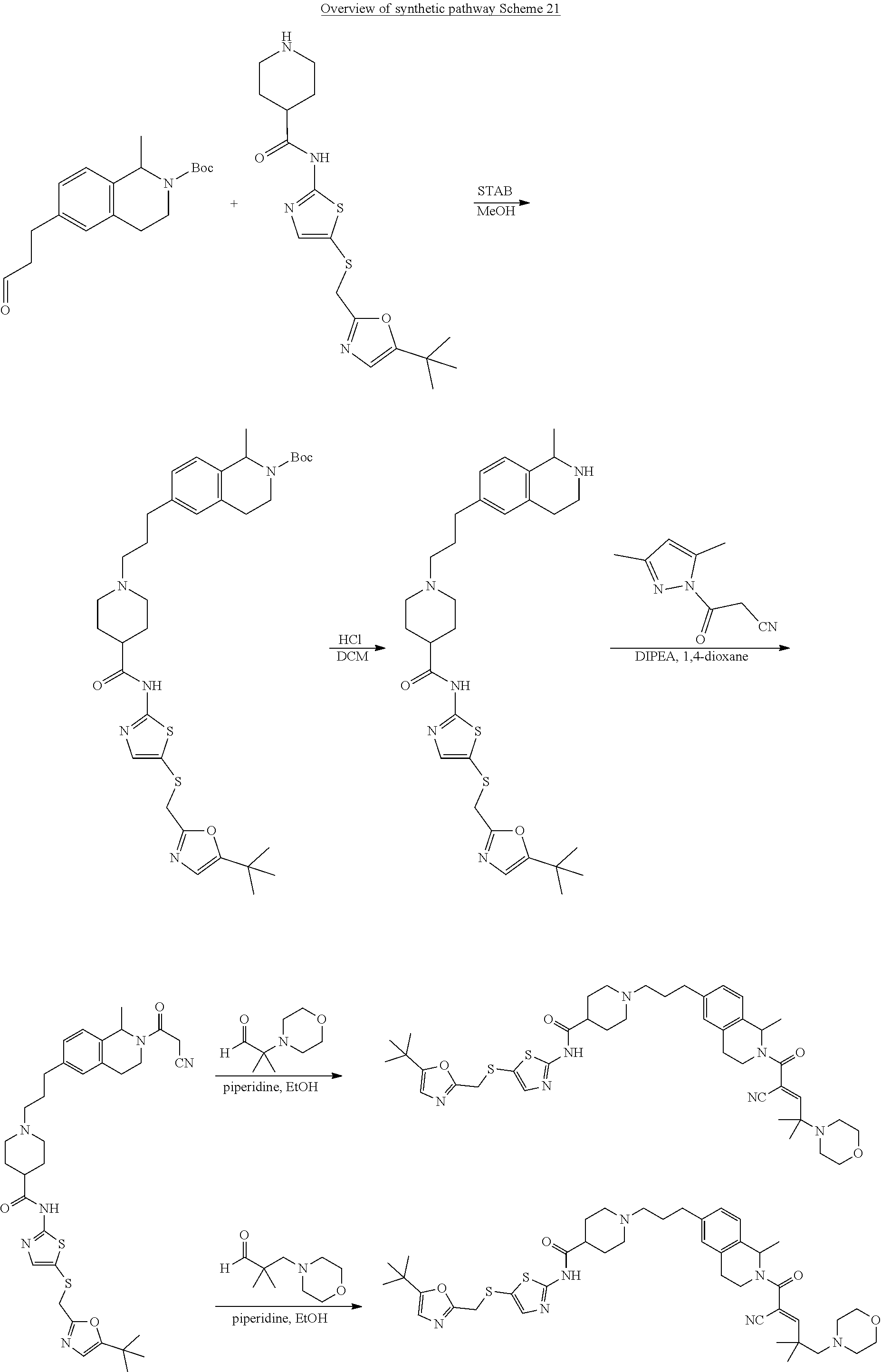

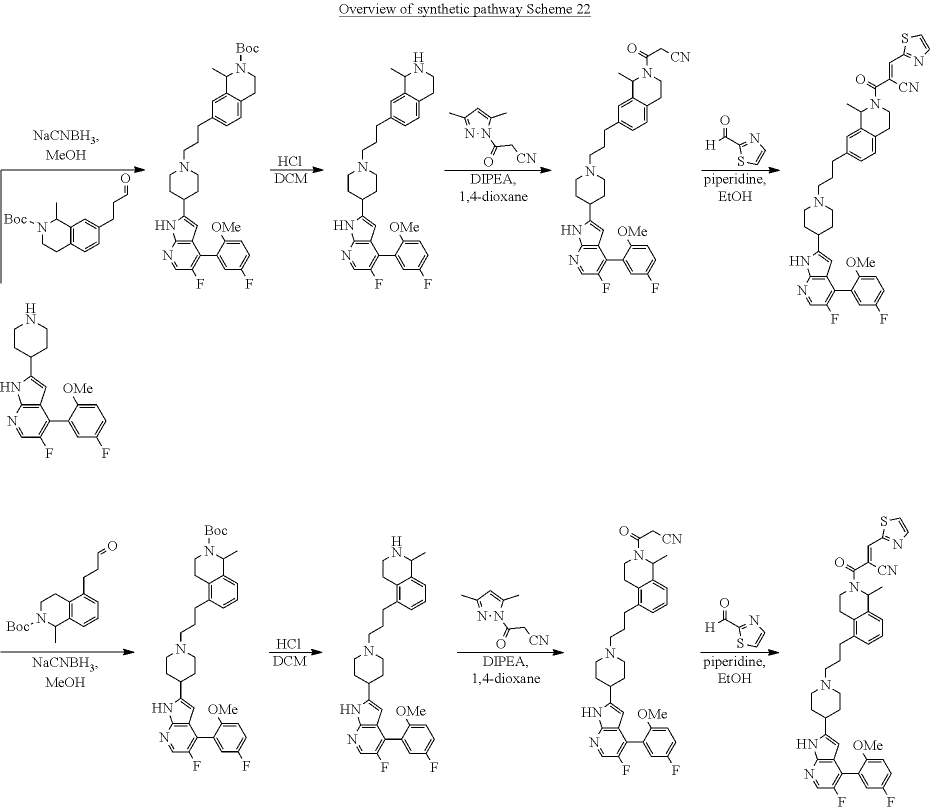

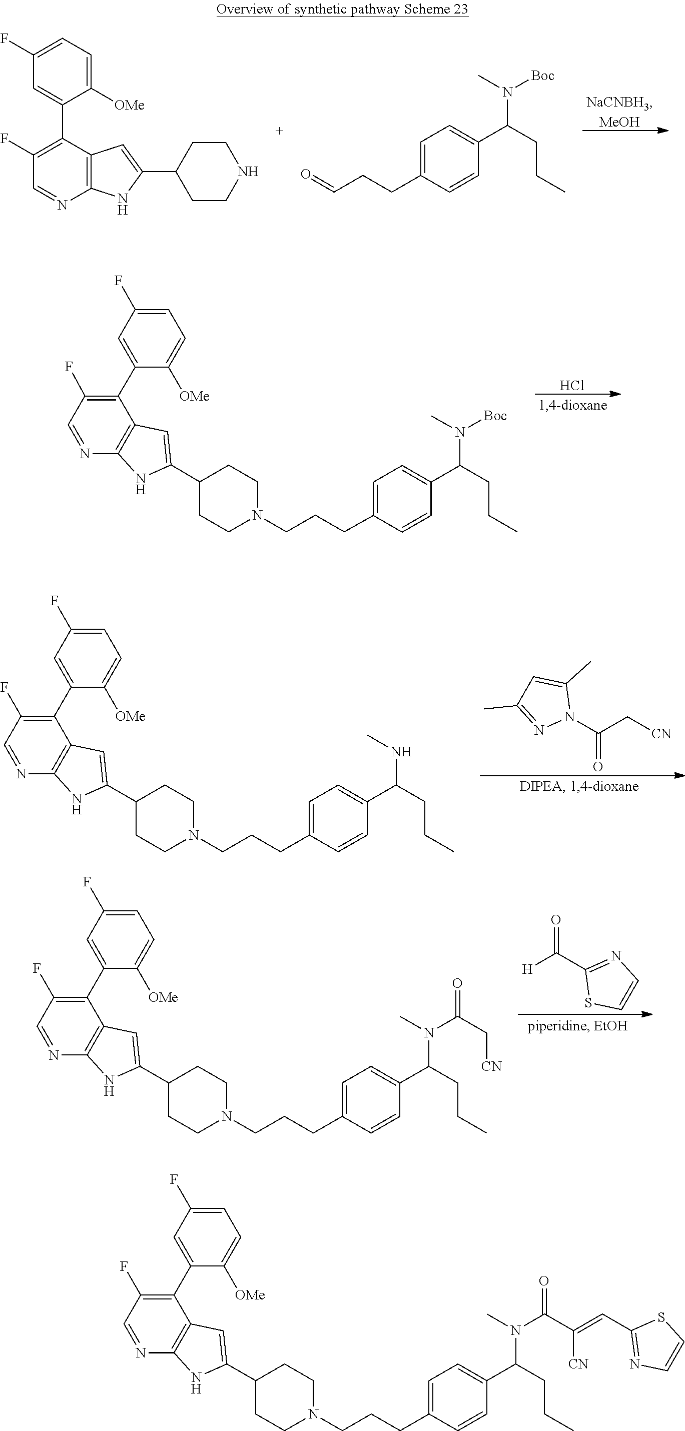

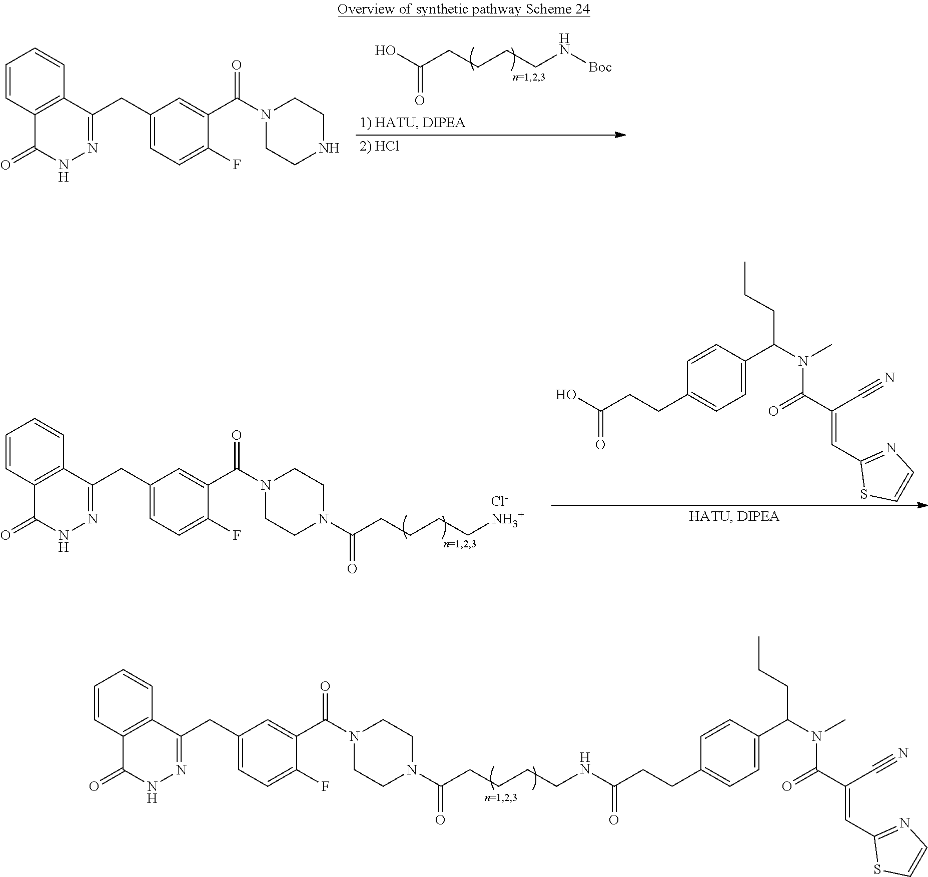

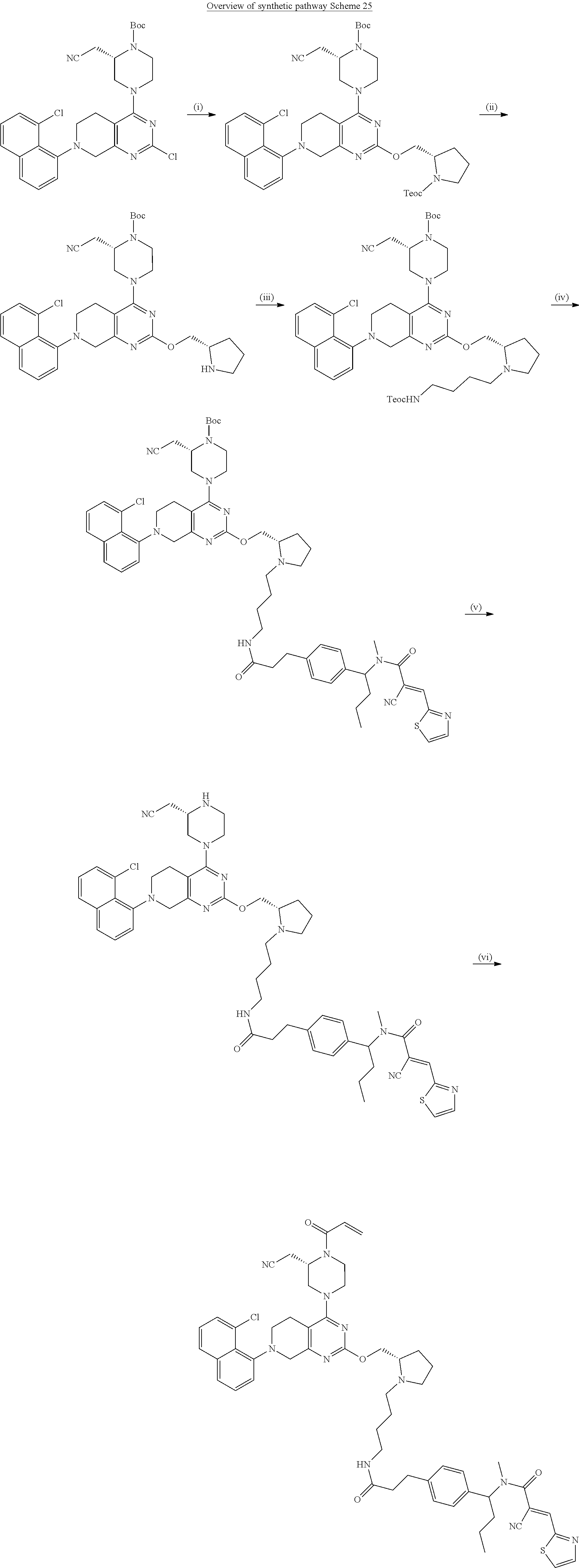

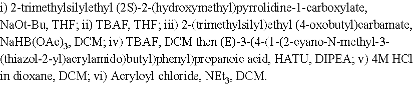

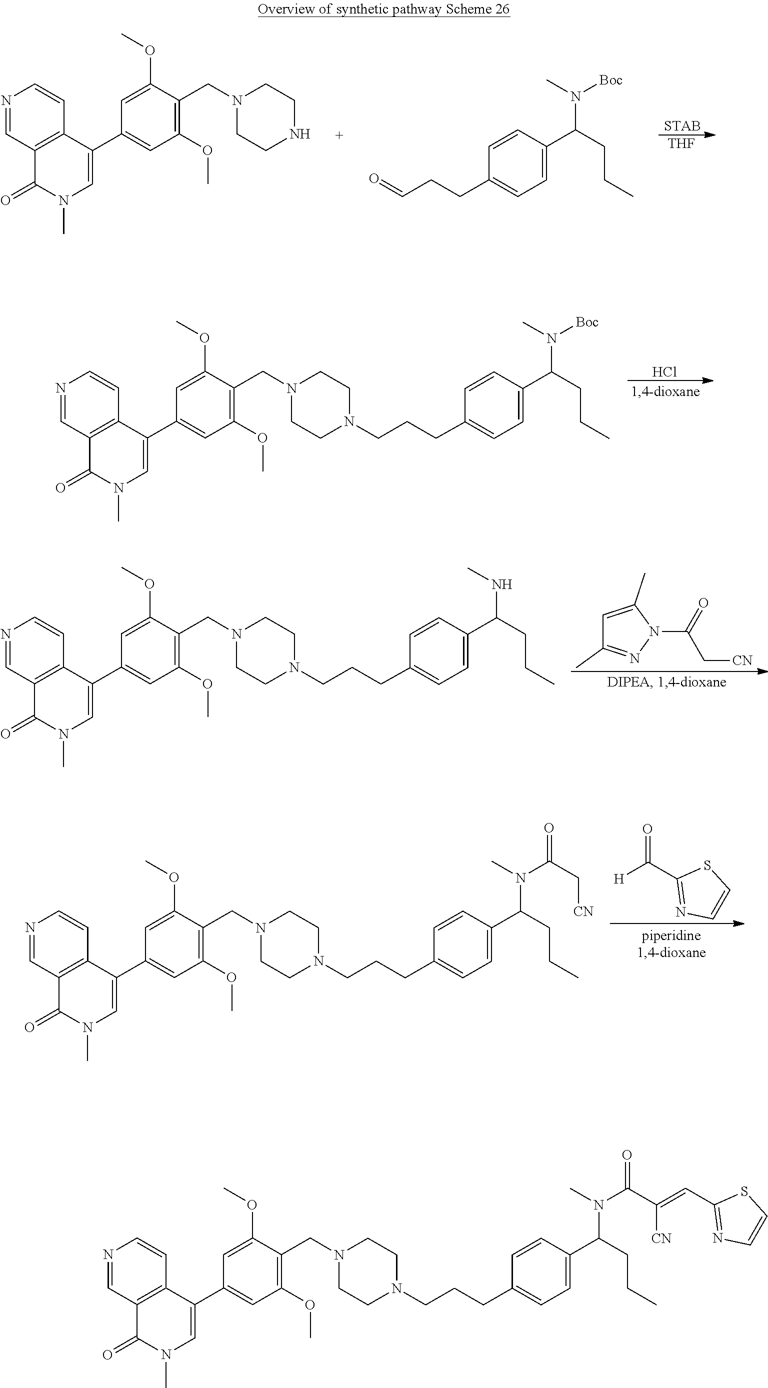

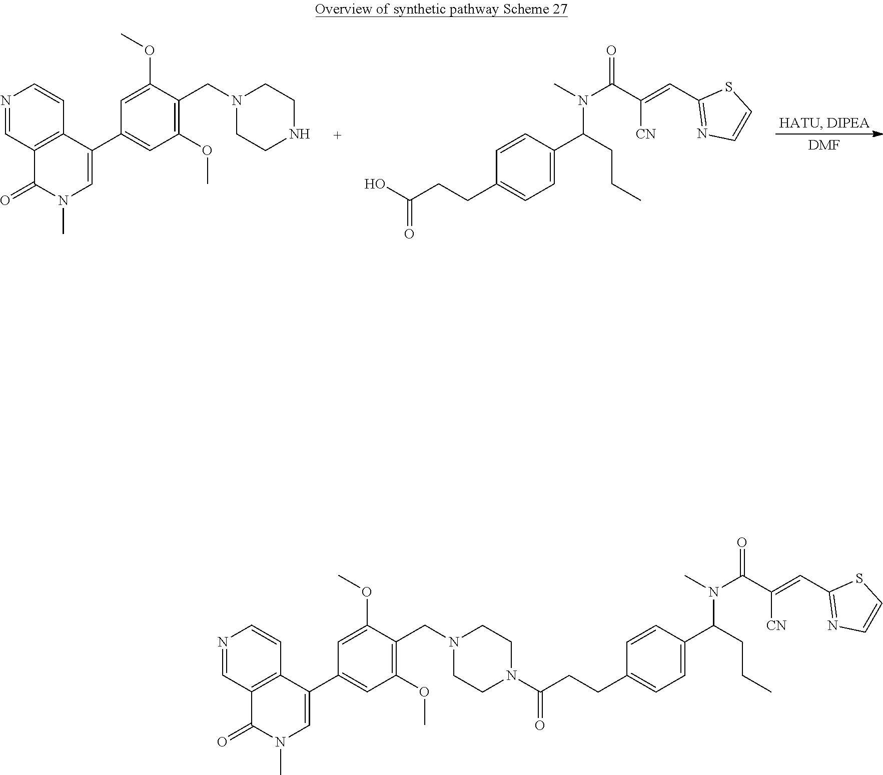

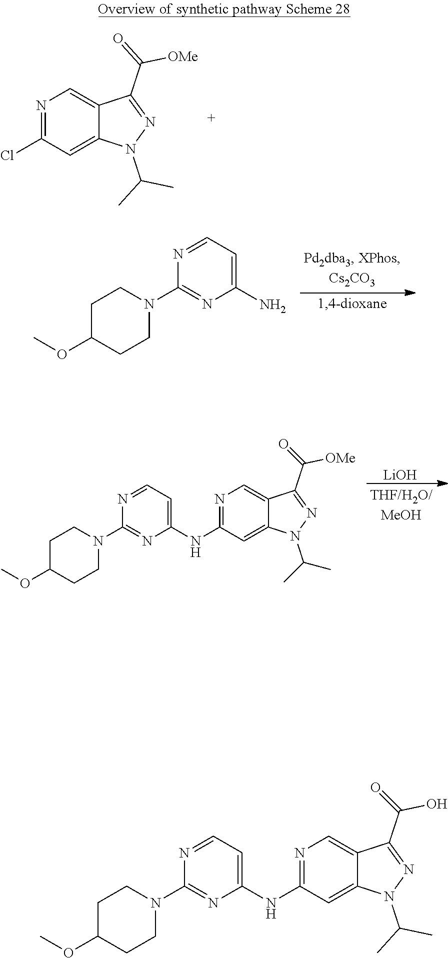

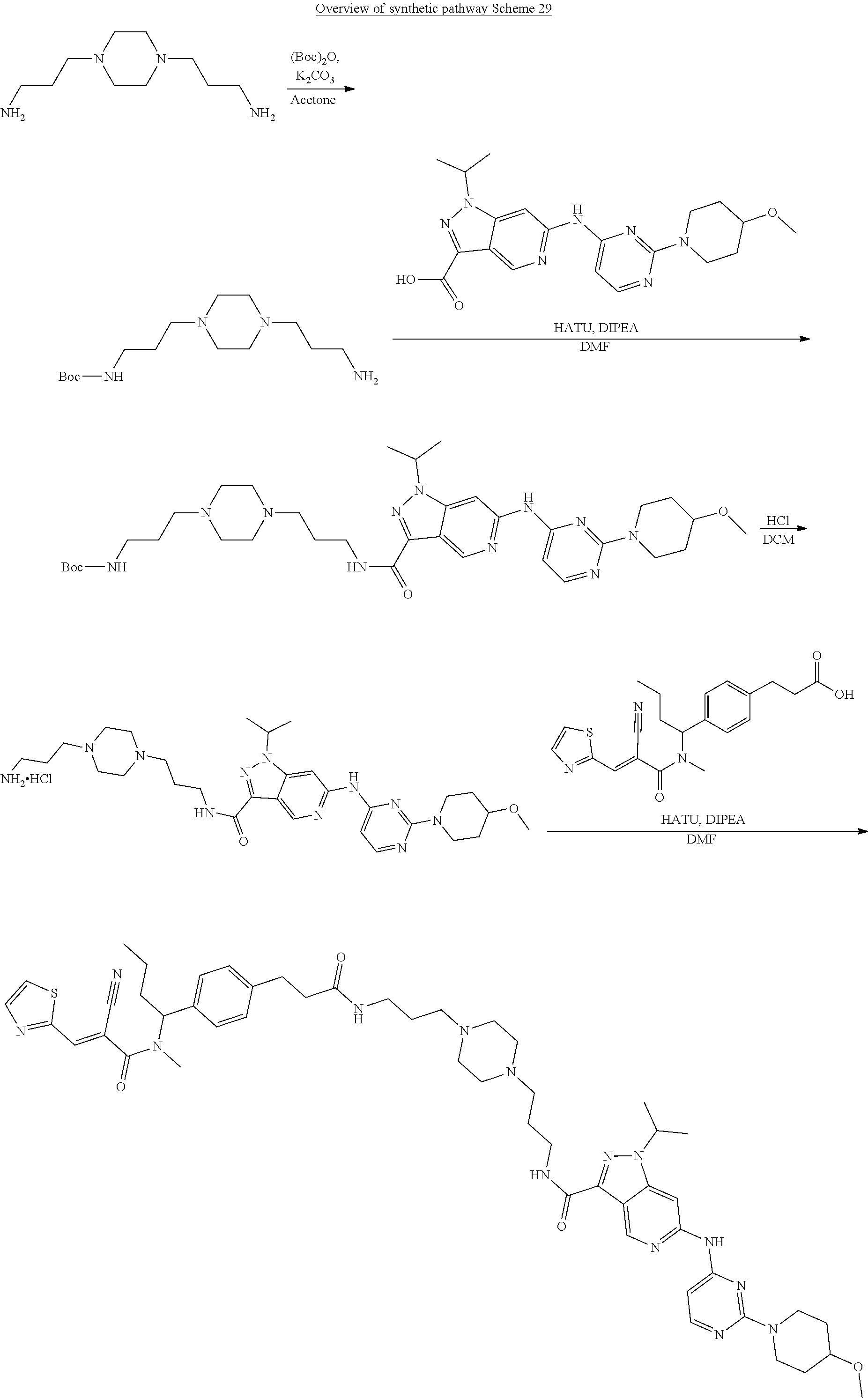

N-Acylation—General Procedure 1

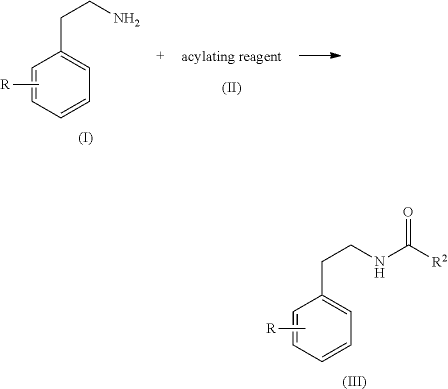

A solution of phenethylamine (1) (1.0 equiv.) in CH₂Cl₂ (0.2 M) was treated with Et₃N (2.0 equiv.) and acylating agent (II) (e.g. acetic anhydride, or acyl chloride (1.0 equiv.)) at 0° C. After full conversion of the starting material was observed the reaction was quenched by addition of water. The mixture was extracted with CH₂Cl₂ and the combined organic layers were washed with water and brine, dried over MgSO₄ and concentrated under reduced pressure. Purification by flash chromatography yielded the corresponding N-acylated product (III).

Bischler-Napieralski Cyclisation—General Procedure 2

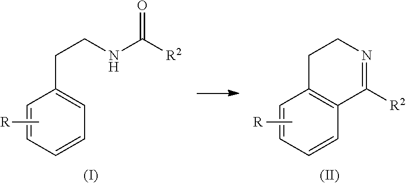

A solution of N-Acylphenethylamine (I) (1.0 equiv.) in toluene (0.1 M) was treated with POCl₃ (5.0 equiv.) and heated to 120° C. for 5 hours. The resulting pale yellow solution was cooled to rt and poured onto ice. The biphasic mixture was adjusted to pH˜10 with NaOH (4 M in water). The mixture was extracted with EtOAc and the combined organic phases were washed with brine, dried over MgSO₄ and concentrated under reduced pressure. Purification by flash chromatography yielded the corresponding cyclised imine (II).

Imine Reduction—General Procedure 3

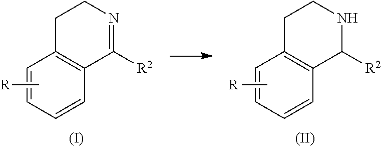

A solution of dihydroisoquinoline (1) (1.0 equiv.) in MeOH (0.1 M) (or EtOH (0.1 M) and acetic acid (0.5 equiv.) was treated with sodium borohydride (2.0 equiv.) at 0° C. and stirred for 2 hours. LC-MS showed complete conversion. The reaction was quenched by addition of HCl (1 M) and then adjusted to pH˜8 with NaOH and extracted with DCM. The combined organic layers were washed with water and brine, dried over MgSO₄ and concentrated under reduced pressure to yield the corresponding tetrahydroisoquinoline.

Demethylation—General Procedure 4

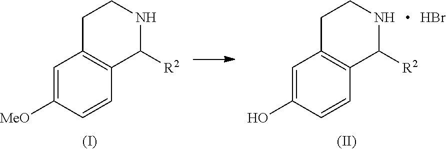

A suspension of phenol methyl ether (1) (1.0 equiv.) in 48% aqueous HBr (10 equiv.) was heated to 100° C. for 6 h. The volatiles were evaporated under reduced pressure and the residue was dried until a solid was obtained. Trituration from EtOAc yielded the corresponding phenol (II).

Boc Protection—General Procedure 5

A solution of amine (1.0 equiv.) in THF or DCM (0.1 M) was treated with di-tert-butyl dicarbonate (1.1 equiv.) and saturated aqueous solution of sodium hydrogen carbonate or triethylamine (5.0 equiv.) and the reaction mixture was stirred at rt for 3 h. The reaction mixture was diluted with water and extracted with EtOAc. The combined organic layers were washed with water and brine, dried over MgSO₄ and concentrated under reduced pressure. Purification by flash chromatography yielded the corresponding Boc-carbamate.

Phenol Alkylation—General Procedure 6

A solution of phenol (1.0 equiv.) in DMF (0.2 M) was treated with K₂CO₃ (3.0 equiv.) and alkyl halide (2.0 equiv.) and the mixture was stirred for 2 h. The mixture was diluted with EtOAc and washed with water, LiCl (5%) and brine. Purification by flash chromatography yielded the corresponding phenol ether (II).

Ester Hydrolysis—General Procedure 7

A solution of ester (1.0 equiv.) in THF (0.2 M) was treated with lithium hydroxide monohydrate (3.0 equiv.) dissolved in water and the mixture was stirred for 4 h. The mixture was adjusted to pH˜3 by addition of 5% KHSO₄ and extracted with EtOAc. The combined organic layers were washed with water and brine, dried over MgSO₄ and concentrated under reduced pressure to yield the corresponding carboxylic acid.

Boc Deprotection—General Procedure 8

A solution of Boc protected amine (1.0 equiv.) in CH₂Cl₂ (0.05 M) was treated with HCl (4 M in dioxane, 50 equiv.) and the mixture was stirred for 2 h. The volatiles were evaporated under reduced pressure to yield the corresponding amine hydrochloride.

Amine Cyano Acetylation—General Procedure 9

A suspension of amine (1.0 equiv.) in 1,4-dioxane (0.05 M) was treated with Et₃N or DIPEA (4.0 equiv.) and 3-(3,5-dimethyl-1H-pyrazol-1-yl)-3-oxopropanenitrile (1.1 equiv.) and the mixture was heated to 90° C. for 2 h. The volatiles were evaporated under reduced pressure. Purification by flash chromatography yielded the corresponding cyanoacetamide.

Knoevenagel Condensation—General Procedure 10

A solution of cyanoacetamide (1.0 equiv.) in THF or EtOH (0.1 M) was treated with aldehyde (2.5 equiv.) and piperidine (0.5 equiv.) and the mixture was heated to reflux for 72 h (if in THF) or at room temperature for 16 h (if in EtOH). The volatiles were evaporated under reduced pressure. Purification by flash chromatography or reverse phase preparative HPLC yielded the corresponding cyanoacrylamide.

Cbz Protection—General Procedure 11

A solution of amine (1.0 equiv.) in THF (0.1 M) was treated with benzyl chloroformate (1.1 equiv.) and saturated aqueous solution of sodium hydrogen carbonate (5.0 equiv.) and the reaction mixture was stirred at rt for 3 h. The reaction mixture was diluted with water and extracted with EtOAc. The combined organic layers were washed with water and brine, dried over MgSO₄ and concentrated under reduced pressure. Purification by flash chromatography yielded the corresponding Cbz-carbamate.

Cbz Cleavage—General Procedure 12

A solution of Cbz-carbamate (1.0 equiv.) in EtOAc (0.1 M) was treated with Pd/C (10%, 0.05 equiv.) and the suspension was stirred for 2 h under balloon pressure hydrogen atmosphere. The solids were removed by filtration over celite and the filtrate was concentrated under reduced pressure to yield the corresponding amine.

Amide Coupling with Substituted Acrylic Acid and tBu Ester Deprotection—General Procedure 13

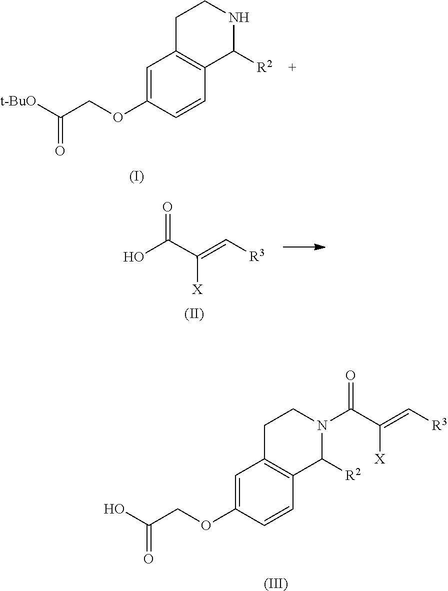

A solution of amine (I) (1.0 equiv.) in DMF (0.1 M) was treated with a solution of acrylic acid (II) (1.0 equiv.) HATU (1.1 equiv.) and DIPEA (2.5 equiv.) in DMF (0.1 M) and the reaction mixture was stirred at rt for 15 min. The reaction was quenched with water and extracted with EtOAc. The combined organic layers were washed with LiCl (5%), water and brine, dried over MgSO₄ and concentrated under reduced pressure. Purification by flash chromatography yielded the corresponding amide. The resulting amide (1.0 equiv.) in CH₂Cl₂ (0.05 M) was treated with TFA (50 equiv.) and the reaction mixture was stirred at rt for 1 h. The volatiles were evaporated and repeated concentration from CH₂Cl₂ solution followed by prolonged drying yielded the corresponding carboxylic acid (III).

Doebner-Knoevenagel—General Procedure 14

A solution of aldehyde (1.0 equiv.) and malonic acid (6.0 equiv.) in pyridine (1.5 M) was treated with piperidine (0.1 equiv.) and the reaction mixture was heated to 100° C. for 14 h. The volatiles were evaporated, and the residue was treated with HCl (1 M). The precipitate was collected by filtration to yield the corresponding acrylic acid.

Fluoroacrylate Ester Synthesis—General Procedure 15

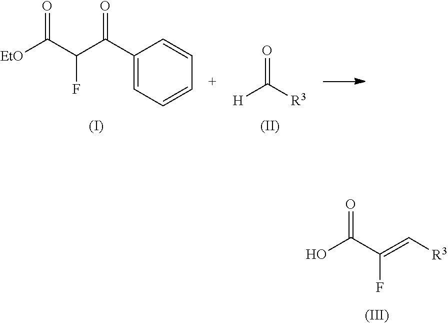

A solution of aldehyde (II) (1.0 equiv.) and 2-fluoro-3-oxo-3-phenylpropionate (I) (1.2 equiv.) in MeCN (0.3 M) was treated with Cs₂CO₃ (2.0 equiv.) and the reaction mixture was heated to 40° C. for 14 h. The reaction mixture was cooled to rt, diluted with EtOAc and filtered over celite. Purification by flash chromatography yielded the corresponding fluoroacrylate ester. The ester was then treated with sodium hydroxide (2 M, 2 equiv.) in THF and left to react until complete deprotection. The mixture was treated with KHSO₄ (10%) to pH=3. The precipitate was filtered and dried to obtain fluoroacrylate (III).

Trifluoromethyl Acrylic Acid Synthesis—General Procedure 16

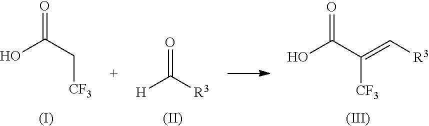

A solution of aldehyde (II) (1.5 equiv.) and 3,3,3-trifluoropropionic acid (I) (1.0 equiv.) in THF (0.2 M) was cooled to 0° C. and treated with TiCl₄ (1.0 M in CH₂Cl₂, 2.0 equiv.) and the mixture was stirred for 30 min. The resulting solution was treated with Et₃N (4.0 equiv.), warmed to rt and stirred for 72 h. The reaction was quenched by addition of water and extracted with CH₂Cl₂. The combined organic layers were washed with water and brine, dried over MgSO₄ and concentrated under reduced pressure. Purification by flash chromatography yielded the corresponding trifluoromethyl acrylic acid (III).

Malonamic Amide Knoevenagel Condensation—General Procedure 17

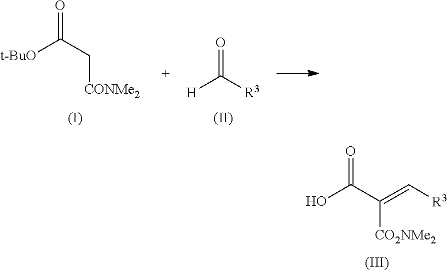

A solution of N,N-dimethylmalonamic acid tert-butyl ester (1) (1.0 equiv.) in THF (0.2 M) was treated with aldehyde (II) (2.5 equiv.) and piperidine (0.5 equiv.) and the mixture was heated to 66° C. for 72 h. The volatiles were evaporated under reduced pressure. Purification by flash chromatography yielded the corresponding amido-acrylamide tert-butyl ester. The ester was then treated with a 50% solution of TFA in DCM and left to react until complete deprotection of the tert-butyl ester was observed by HPLC. Volatiles were removed under reduced pressure to obtain malonamic amide (III).

Sulfone Knoevenagel Condensation—General Procedure 18

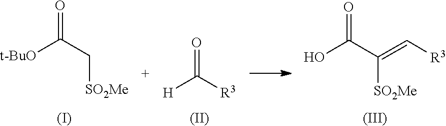

A solution of tert-butyl methylsulfonyl acetate (1) (1.0 equiv.) in THF (0.2 M) was treated with aldehyde (II) (2.5 equiv.) and piperidine (0.5 equiv.) and the mixture was heated to 66° C. for 72 h. The volatiles were evaporated under reduced pressure. Purification by flash chromatography yielded the corresponding sulfonacrylate ester. The ester was then treated with a 50% solution of TFA in DCM and left to react until complete deprotection of the tertbutyl ester was observed by HPLC. Volatiles were removed under reduced pressure and the acid was crystallised from ethyl acetate to obtain sulfonacrylate (III).

TBS Protection—General Procedure 19

A solution of alcohol (1.0 equiv.) in CH₂Cl₂ was treated with TBSCI (1.3 equiv.) and imidazole (1.0 equiv.). The colourless suspension was stirred at rt for 4 h. The reaction mixture was quenched by addition of NH₄Cl (sat. aq.) and extracted with CH₂Cl₂. The combined organic layers were washed with water and brine, dried over MgSO₄ and concentrated under reduced pressure. Purification by flash chromatography yielded the corresponding silylether.

Sulfinimide Formation—General Procedure 20

A solution of aldehyde or ketone (1.0 equiv.) in THF was treated with titanium (IV) ethoxide (2.0 equiv.) followed by 2-methylpropane-2-sulfinamide (1.3 equiv.) and the mixture was heated to 66° C. for 16 h. The reaction was quenched by addition of NH₄Cl (sat. aq.), diluted with EtOAc and the precipitated solids were removed by filtration. The biphasic mixture was extracted with EtOAc, and the combined organic layers were washed with water and brine, dried over MgSO₄ and concentrated under reduced pressure. Purification by flash chromatography yielded the corresponding sulfinimide.

Sulfinimine Reduction—General Procedure 21

A solution of sulfinimide (1.0 equiv.) in THF (0.1 M) was cooled to 0° C. and treated with NaBH₄ (2.0 equiv.) and the mixture was stirred for 2 h and then warmed to rt. The reaction was quenched by addition of NH₄Cl (sat. aq.) and extracted with EtOAc. The combined organic layers were washed with water and brine, dried over MgSO₄ and concentrated under reduced pressure. Purification by flash chromatography yielded the corresponding sulfinamide.

Grignard Addition to Sulfinimine—General Procedure 22

A solution of sulfinimide (1.0 equiv.) in CH₂Cl₂ was cooled to −78° C. and treated with organomagnesium halide (II) (2.0 equiv.) and the mixture was stirred for 2 h and then warmed to rt. The reaction was quenched by addition of NH₄Cl (sat. aq.) and extracted with EtOAc. The combined organic layers were washed with water and brine, dried over MgSO₄ and concentrated under reduced pressure. Purification by flash chromatography yielded the corresponding sulfinamide.

Sulfinamide Alkylation—General Procedure 23

A solution of sulfinamide (1.0 equiv.) in THF was cooled to 0° C. and treated with LiHMDS (2.0 equiv.) and the mixture was stirred for 15 min. The resulting solution was treated with organohalide, and warmed to rt. The reaction was quenched by addition of water and extracted with EtOAc. The combined organic layers were washed with water and brine, dried over MgSO₄ and concentrated under reduced pressure. Purification by flash chromatography yielded the corresponding N-alkylated sulfinamide.

Silylether Cleavage—General Procedure 24

A solution of silyl ether (1.0 equiv.) in CH₂Cl₂ was treated with TBAF (1 M in THF, 1.2 equiv.) and the mixture was stirred for 12 h. The reaction was quenched by addition of NH₄Cl (sat. aq.) and extracted with CH₂Cl₂. The combined organic layers were washed with water and brine, dried over MgSO₄ and concentrated under reduced pressure. Purification by flash chromatography yielded the corresponding alcohol.

Sulfinamide Deprotection—General Procedure 25

To a solution of sulfinamide (1.0 equiv.) in diethyl ether (0.2 M) was added HCl (4 M in dioxane, 3.0 equiv.). The reaction was monitored by TLC. Once no starting material was observed, the suspension was filtered, washed with diethyl ether and the isolated amine hydrochloride salt was used without further purification.

Reductive Amination—General Procedure 26

A solution of amine (1.0 equiv.) in MeOH (or THF or DCM) (0.1 M) was treated with ketone or aldehyde (2.0 equiv.) and sodium cyanoborohydride (or sodium triacetoxyborohydride, or polymer-bound cyanoborohydride) (4.0 equiv.) and the mixture was stirred for 12 h. The reaction was quenched by addition of water and extracted with EtOAc. The combined organic layers were washed with water and brine, dried over MgSO₄ and concentrated under reduced pressure. Purification by flash chromatography yielded the corresponding amine.

Suzuki Coupling—General Procedure 27

A solution of aryl halide (1.0 equiv.), boronic acid or ester (1.5 equiv.), K₂CO₃ (2.5 equiv.) and Pd(dppf)Cl₂ (0.1 equiv.) in 1,4-dioxane/water was deoxygenated by sparging with N₂ and consequently heated to 100° C. for 1 h. The reaction was cooled to rt, diluted with EtOAc and the solids were removed by filtration. The residue was concentrated under reduced pressure. Purification by flash chromatography yielded the corresponding coupling product.

Alkene Hydrogenation—General Procedure 28

A solution of alkene (1.0 equiv.) in MeOH was treated with Pd/C (10%, 0.05 equiv.) and the suspension was stirred for 2 h under hydrogen atmosphere. The solids were removed by filtration over celite and the filtrate was concentrated under reduced pressure to yield the corresponding hydrogenation product.

Ester Reduction to Aldehyde—General Procedure 29

A solution of ester (1.0 equiv.) in CH₂Cl₂ (0.1 M) was cooled to −78° C. and treated with DIBAL-H (1 M in heptane, 1.2 equiv.) and the resulting solution was stirred for 2 h. The reaction was quenched by addition of MeOH at −78° C., and warmed to rt. The reaction was diluted with CH₂Cl₂ and treated with Rochelle's salt (10% aq.) and stirred until a clear biphasic mixture was obtained. The mixture was extracted with CH₂Cl₂, the combined organic layers were washed with water and brine, dried over MgSO₄ and concentrated under reduced pressure. Purification by flash chromatography yielded the corresponding aldehyde.

Amine Alkylation—General Procedure 30

To a stirred solution of amine (1 equiv.), DIPEA (3.0 equiv.) and potassium iodide (0.3 equiv.) in DMF was added alkyl-Br (1 equiv.) at RT. The reaction mixture was stirred at RT for 5 h then diluted in water and extracted with EtOAc. The combined organic layers were dried (Na₂SO₄) and concentrated in vacuo. The crude product was purified by flash chromatography to give the desired product.

Amide Coupling—General Procedure 31

To a stirred solution of carboxylic acid (1.0 equiv.) in DMF was added DIPEA (2.5 equiv.) and HATU (1.5 equiv.). The reaction mixture was stirred for 5 min, then relevant amine (1.5 equiv.) was added and the reaction mixture was stirred for 12 h at RT. The reaction was quenched with ice cold water and extracted with EtOAc. The combined organic layers were concentrated in vacuo to afford the crude product. Where stated, the crude product was purified by flash chromatography/reverse phase preparative HPLC to give the desired compound.

Reduction of Weinreb Amide to Aldehyde Using DIBALH—General Procedure 32

To a stirred solution of Weinreb amide (1.0 equiv.) in THF at 0° C. was added DIBALH (3.0 equiv.) dropwise. The reaction was stirred for 4 h at RT then was quenched with HCl (1.5 N) and water, then filtered through celite. The filtrate was extracted with EtOAc, and the combined organic layers were dried (Na₂SO₄), filtered and concentrated in vacuo to give the desired compound

Tandem Boc/tBu Deprotection—General Procedure 33:

A solution of N-Boc,CO₂t-Bu-amino acid (1) (1.0 equiv.) in DCM (0.1 M) was treated with HCl (4 M in dioxane, 50 equiv.) and the mixture was stirred at rt for 48 h. The volatiles were evaporated under reduced pressure to yield the corresponding amino acid.

Reductive Amination with Ammonium Acetate/Acylation: General Procedure 34:

A solution of ketone (1.0 equiv.) in methanol (0.15 M) at rt was treated with ammonium acetate (10.0 equiv.) followed by sodium cyanoborohydride (1.50 equiv.) and stirred for 12 h. The reaction was quenched with water and the mixture was extracted with DCM. The combined organic extracts were washed with brine, dried over MgSO₄ and concentrated under reduced pressure. The crude residue was redissolved in DCM (0.15 M), cooled to 0° C., treated with triethylamine (2.00 equiv.) and acyl chloride (1.50 equiv.) and stirred for 2 h. The reaction was quenched with water and extracted with DCM. The combined organic extracts were washed with brine, dried over MgSO₄ and concentrated under reduced pressure. Purification by flash chromatography yielded the N-acylated product.

FeCl₃-Mediated Bischler-Napieralski Cyclisation: General Procedure 35:

To a stirred solution of N-acylphenethylamine (1.00 equiv.) in DCM (0.3 M) at 0° C. was added drop wise oxalyl chloride (12.0 equiv.). The resulting reaction mixture was warmed to rt and stirred for 2 h. The reaction mixture was then cooled to −78° C., and iron(III) chloride (6.00 equiv.) was added portion wise. The mixture was warmed to rt slowly and stirred for 16 h. The reaction was quenched with HCl (1 M) and extracted with DCM. The combined extracts were concentrated under reduced pressure. The residue was redissolved in H₂SO₄:MeOH (1:10, 0.1 M) and stirred at 80° C. for 16 h. The mixture was concentrated under reduced pressure and redissolved in water. The pH was adjusted to ˜7 with 25% aq. ammonia and the mixture was extracted with DCM. The combined extracts were washed with brine, dried over MgSO₄ and concentrated under reduced pressure. Purification by flash chromatography yielded the cyclised imine.

Preparative HPLC conditions: a Xbridge C18 (19×150 mm) 5 μm silica_column was used. When not specified otherwise, a 5-95% gradient of acetonitrile in 10 mM ammonium acetate was used, with a flow rate of 15 ml/min.

### PREPARATIVE EXAMPLES

### Example 1: N-(3-methoxyphenethyl)acetamide

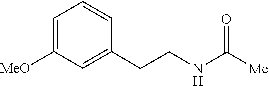 [SMILES: COc1cccc(CCNC(C)=O)c1]

Prepared following general procedure 1. Obtained 1.46 g, 79.4% yield.

¹H NMR (400 MHz, CDCl₃) δ 7.23 (t, J=7.8 Hz, 1H), 6.82-6.76 (m, 2H), 6.74 (t, J=2.0 Hz, 1H), 5.48 (s, 1H), 3.80 (s, 3H), 3.52 (td, J=6.9, 5.8 Hz, 2H), 2.79 (t, J=6.9 Hz, 2H), 1.94 (s, 3H).

### Example 2: N-(3-methoxyphenethyl)butyramide

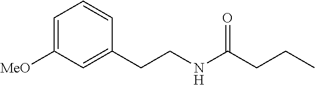 [SMILES: CCCC(=O)NCCc1cccc(OC)c1]

Prepared following general procedure 1. Obtained 870 mg, 65.2% yield.

¹H NMR (400 MHz, CDCl₃) δ 7.23 (t, J=7.9 Hz, 1H), 6.86-6.72 (m, 3H), 5.40 (s, 1H), 3.80 (s, 3H), 3.53 (td, J=6.9, 5.8 Hz, 2H), 2.79 (t, J=6.9 Hz, 2H), 2.10 (dd, J=8.2, 6.8 Hz, 2H), 1.62 (dt, J=14.6, 7.3 Hz, 2H), 0.92 (t, J=7.4 Hz, 3H).

### Example 3: 6-methoxy-1-methyl-3,4-dihydroisoquinoline

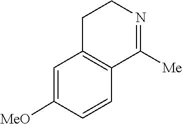 [SMILES: COc1ccc2c(c1)CCN=C2C]

Prepared following general procedure 2. Obtained 446 mg, 76.6% yield.

¹H NMR (400 MHz, CDCl₃) δ 7.47 (d, J=8.6 Hz, 1H), 6.81 (dd, J=8.6, 2.7 Hz, 1H), 6.72 (d, J=2.4 Hz, 1H), 3.85 (s, 3H), 3.67 (tq, J=7.5, 1.5 Hz, 2H), 2.74 (dd, J=8.5, 6.5 Hz, 2H), 2.43 (s, 3H).

### Example 4: 6-methoxy-1-propyl-3,4-dihydroisoquinoline

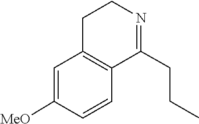

Prepared following general procedure 2. Obtained 553 mg, 69.2% yield.

¹H NMR (400 MHz, CDCl3) δ 7.51 (d, J=8.6 Hz, 1H), 6.83 (dd, J=8.6, 2.7 Hz, 1H), 6.75 (d, J=2.6 Hz, 1H), 3.86 (s, 3H), 3.70 (t, J=7.5 Hz, 2H), 2.89-2.67 (m, 4H), 1.72 (h, J=7.4 Hz, 2H), 1.01 (t, J=7.4 Hz, 3H).

### Example 5: 6-methoxy-1-methyl-1,2,3,4-tetrahydroisoquinoline

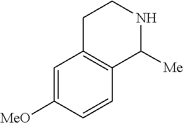 [SMILES: COc1ccc2c(c1)CCNC2C]

Prepared following general procedure 3. Obtained 417 mg, 92.8% yield

¹H NMR (400 MHz, CDCl₃) δ 7.06 (dd, J=8.6, 0.8 Hz, 1H), 6.74 (dd, J=8.6, 2.8 Hz, 1H), 6.62 (d, J=2.8 Hz, 1H), 4.10 (qd, J=6.4, 0.8 Hz, 1H), 3.78 (s, 3H), 3.28 (dt, J=12.4, 5.1 Hz, 1H), 3.04 (ddd, J=12.5, 8.9, 4.7 Hz, 1H), 2.96-2.84 (m, 1H), 2.74 (dt, J=16.4, 4.7 Hz, 1H), 2.40 (s, 1H), 1.47 (d, J=6.7 Hz, 3H).

### Example 6: 6-methoxy-1-propyl-1,2,3,4-tetrahydroisoquinoline

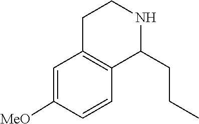

Prepared following general procedure 3. Obtained 452 mg, 80.9% yield.

¹H NMR (400 MHz, CDCl₃) δ 7.05 (d, J=8.6 Hz, 1H), 6.72 (dd, J=8.5, 2.8 Hz, 1H), 6.61 (d, J=2.7 Hz, 1H), 3.94 (dd, J=9.2, 3.8 Hz, 1H), 3.82-3.79 (m, 1H), 3.78 (s, 3H), 3.24 (dt, J=12.4, 5.4 Hz, 1H), 2.98 (ddd, J=12.5, 7.8, 5.0 Hz, 1H), 2.87-2.78 (m, 1H), 2.72 (dt, J=16.3, 5.2 Hz, 1H), 1.87-1.62 (m, 2H), 1.58-1.36 (m, 2H), 0.97 (t, J=7.3 Hz, 3H).

### Example 7: 1-methyl-1,2,3,4-tetrahydroisoquinolin-6-ol hydrobromide

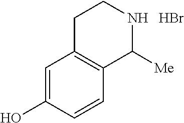 [SMILES: CC1NCCc2cc(O)ccc21 ; Br]

Prepared following general procedure 4. Obtained 492 mg, 85.7% yield.

¹H NMR (400 MHz, DMSO) δ 9.48 (s, 1H), 9.05 (s, 1H), 8.70 (s, 1H), 7.09 (d, J=8.5 Hz, 1H), 6.68 (dd, J=8.5, 2.6 Hz, 1H), 6.58 (d, J=2.5 Hz, 1H), 4.44 (s, 1H), 3.41 (s, 2H), 2.92 (qt, J=11.3, 6.2 Hz, 2H), 1.52 (d, J=6.8 Hz, 3H).

### Example 8: 1-propyl-1,2,3,4-tetrahydroisoquinolin-6-ol hydrobromide

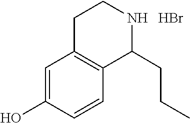

Prepared following general procedure 4. Obtained 485 mg, 80.9% yield.

¹H NMR (400 MHz, DMSO) δ 9.49 (s, 1H), 9.05 (s, 1H), 8.55 (s, 1H), 7.07 (d, J=8.6 Hz, 1H), 6.67 (dd, J=8.5, 2.6 Hz, 1H), 6.A (d, J=2.6 Hz, 1H), 4.35 (s, 1H), 3.43-3.36 (m, 1H), 3.25 (dd, J=12.7, 6.5 Hz, 1H), 2.91 (dtt, J=17.6, 12.6, 6.6 Hz, 2H), 1.95-1.70 (m, 2H), 1.42 (ddt, J=13.5, 9.4, 6.7 Hz, 2H), 0.94 (t, J=7.3 Hz, 3H).

### Example 9: tert-butyl 6-hydroxy-1-methyl-3,4-dihydroisoquinoline-2(1H)-carboxylate

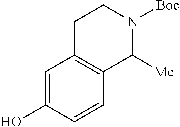 [SMILES: CC1c2ccc(O)cc2CCN1C(=O)OC(C)(C)C]

Prepared following general procedure 5. Obtained 485 mg, 80.9% yield.

¹H NMR (400 MHz, DMSO) δ 9.20 (s, 1H), 6.97 (d, J=8.4 Hz, 1H), 6.58 (dd, J=8.3, 2.6 Hz, 1H), 6.50 (d, J=2.5 Hz, 1H), 4.94 (s, 1H), 3.98-3.77 (m, 1H), 3.23-3.00 (m, 1H), 2.73-2.56 (m, 2H), 1.42 (s, 9H), 1.30 (d, J=6.7 Hz, 3H).

### Example 10: tert-butyl 6-hydroxy-1-propyl-3,4-dihydroisoquinoline-2(1H)-carboxylate

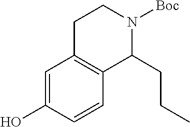 [SMILES: CCCC1c2ccc(O)cc2CCN1C(=O)OC(C)(C)C]

Prepared following general procedure 5. Obtained 216 mg, 80.7% yield.

¹H NMR (400 MHz, DMSO) δ 9.19 (s, 1H), 6.92 (d, J=8.4 Hz, 1H), 6.56 (dd, J=8.3, 2.6 Hz, 1H), 6.49 (d, J=2.5 Hz, 1H), 4.87 (d, J=27.0 Hz, 1H), 3.95-3.75 (m, 1H), 3.24-3.01 (m, 2H), 2.74-2.57 (m, 1H), 1.67 (s, 1H), 1.60-1.47 (m, 1H), 1.40 (s, 9H), 1.34-1.24 (m, 2H), 0.89 (t, J=9.3 Hz, 3H).

### Example 11: tert-butyl 6-(2-ethoxy-2-oxoethoxy)-1-methyl-3,4-dihydroisoquinoline-2(1H)-carboxylate

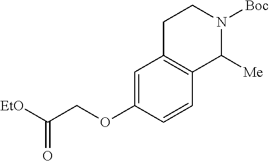 [SMILES: CCOC(=O)COc1ccc2c(c1)CCN(C(=O)OC(C)(C)C)C2C]

Prepared following general procedure 6. Obtained 229 mg, 79.9% yield.

¹H NMR (400 MHz, CDCl3) δ 7.03 (d, J=8.6 Hz, 1H), 6.76 (dd, J=8.4, 2.6 Hz, 1H), 6.64 (d, J=2.7 Hz, 1H), 5.25-5.01 (m, 1H), 4.59 (s, 2H), 4.27 (q, J=7.1 Hz, 2H), 4.20-3.96 (m, 1H), 3.30-3.06 (m, 1H), 2.92-2.80 (m, 1H), 2.68 (dt, J=16.1, 3.6 Hz, 1H), 1.48 (s, 9H), 1.40 (d, J=6.7 Hz, 3H), 1.30 (t, J=7.1 Hz, 3H).

### Example 12: tert-butyl 6-(2-ethoxy-2-oxoethoxy)-1-propyl-3,4-dihydroisoquinoline-2(1H)-carboxylate

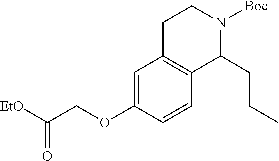 [SMILES: CCCC1c2ccc(OCC(=O)OCC)cc2CCN1C(=O)OC(C)(C)C]

Prepared following general procedure 6. Obtained 244 mg, 87.2% yield.

¹H NMR (400 MHz, CDCl₃) δ 7.01 (d, J=8.4 Hz, 1H), 6.74 (d, J=8.4 Hz, 1H), 6.64 (d, J=2.7 Hz, 1H), 5.15-4.92 (m, 1H), 4.58 (s, 2H), 4.32-4.18 (m, 2H), 3.97-3.83 (m, 1H), 3.20-3.07 (m, 1H), 2.96-2.79 (m, 1H), 2.71-2.62 (m, 1H), 1.84-1.70 (m, 1H), 1.65-1.59 (m, 1H), 1.45 (s, 9H), 1.45-1.35 (m, 2H), 1.32-1.28 (m, 3H), 0.95 (t, J=7.4 Hz, 3H).

### Example 13: benzyl 6-(2-(tert-butoxy)-2-oxoethoxy)-3,4-dihydroisoquinoline-2(1H)-carboxylate

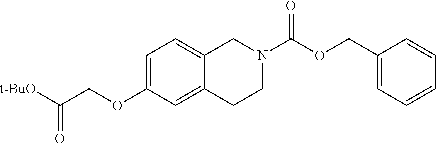 [SMILES: CC(C)(C)OC(=O)COc1ccc2c(c1)CCN(C(=O)OCc1ccccc1)C2]

Prepared following general procedure 6. Obtained 330 mg, 84.0% yield. ¹H NMR (400 MHz, DMSO) δ 7.41-7.27 (m, 5H), 7.10 (d, J=8.4 Hz, 1H), 6.77-6.67 (m, 2H), 5.12 (s, 2H), 4.60 (s, 2H), 4.56-4.43 (m, 2H), 3.60 (s, 2H), 2.76 (t, J=6.0 Hz, 2H), 1.42 (s, 9H).

### Example 14: 2-((2-(tert-butoxycarbonyl)-1-methyl-1,2,3,4-tetrahydroisoquinolin-6-yl)oxy)acetic acid

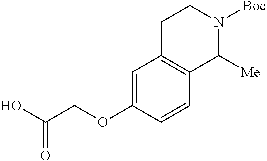 [SMILES: CC1c2ccc(OCC(=O)O)cc2CCN1C(=O)OC(C)(C)C]

Prepared following general procedure 7. Obtained 51.3 mg, 97.0% yield.

¹H NMR (400 MHz, DMSO) δ 12.95 (s, 1H), 7.11 (d, J=8.6 Hz, 1H), 6.73 (dd, J=8.5, 2.8 Hz, 1H), 6.66 (d, J=2.7 Hz, 1H), 5.00 (s, 1H), 4.61 (s, 2H), 3.92 (s, 2H), 3.12 (s, 2H), 1.42 (s, 9H), 1.32 (d, J=6.6 Hz, 3H).

### Example 15: 2-((2-(tert-butoxycarbonyl)-1-propyl-1,2,3,4-tetrahydroisoquinolin-6-yl)oxy)acetic acid

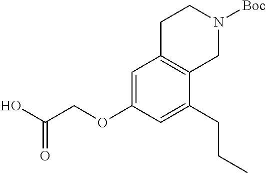 [SMILES: CCCc1cc(OCC(=O)O)cc2c1CN(C(=O)OC(C)(C)C)CC2]

Prepared following general procedure 7. Obtained 48.8 mg, 86% yield.

¹H NMR (400 MHz, DMSO) δ 12.95 (s, 1H), 7.06 (d, J=8.5 Hz, 1H), 6.72 (dd, J=8.5, 2.8 Hz, 1H), 6.66 (d, J=2.7 Hz, 1H), 4.99-4.86 (m, 1H), 4.62 (s, 2H), 3.99-3.78 (m, 1H), 3.25-3.05 (m, 1H), 2.75-2.64 (m, 2H), 1.75-1.51 (m, 2H), 1.41 (s, 9H), 1.34-1.23 (m, 2H), 0.97-0.85 (m, 3H).

### Example 16: benzyl 6-hydroxy-3,4-dihydroisoquinoline-2(1H)-carboxylate

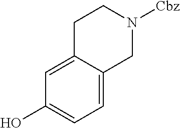 [SMILES: O=C(OCc1ccccc1)N1CCc2cc(O)ccc2C1]

Prepared following general procedure 11. Obtained 558 mg, 74% yield. ¹H NMR (400 MHz, DMSO) δ 9.22 (s, 1H), 7.40-7.27 (m, 5H), 6.96 (d, J=8.3 Hz, 1H), 6.58 (dd, J=8.2, 2.5 Hz, 1H), 6.55 (d, J=2.5 Hz, 1H), 5.11 (s, 2H), 4.45 (d, J=16.0 Hz, 2H), 3.57 (s, 2H), 2.70 (t, J=6.0 Hz, 2H).

### Example 17: tert-butyl 2-((1,2,3,4-tetrahydroisoquinolin-6-yl)oxy)acetate

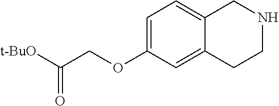 [SMILES: CC(C)(C)OC(=O)COc1ccc2c(c1)CCNC2]

Prepared following general procedure 12. Obtained 233 mg, 92.5% yield. ¹H NMR (400 MHz, DMSO) δ 6.90 (d, J=8.4 Hz, 1H), 6.63 (dd, J=8.4, 2.8 Hz, 1H), 6.56 (d, J=2.7 Hz, 1H), 4.56 (s, 2H), 3.74 (s, 2H), 2.89 (t, J=5.9 Hz, 2H), 2.62 (t, J=5.9 Hz, 2H), 1.42 (s, 9H).

### Example 18: (E)-2-((2-(2-cyano-3-(thiazol-2-yl)acryloyl)-1,2,3,4-tetrahydroisoquinolin-6-yl)oxy)acetic acid

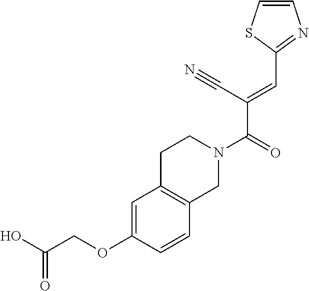 [SMILES: N#C/C(=C\c1nccs1)C(=O)N1CCc2cc(OCC(=O)O)ccc2C1]

Prepared following general procedure 13. Obtained 2.2 mg, 95.7% yield. m/z=370.0 (M+H)⁺.

### Example 19: (E)-3-(thiazol-2-yl)acrylic acid

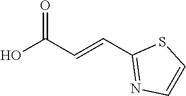

Prepared following general procedure 14. Obtained 350 mg, 25.5% yield.

¹H NMR (400 MHz, DMSO) δ 12.79 (s, 1H), 8.00 (d, J=3.2 Hz, 1H), 7.93 (dd, J=3.2, 0.6 Hz, 1H), 7.68 (dd, J=15.8, 0.6 Hz, 1H), 6.66 (d, J=15.8 Hz, 1H).

### Example 20: (Z)-3-(6-bromopyridin-2-yl)-2-fluoroacrylic acid

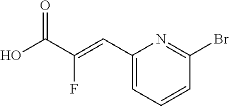 [SMILES: O=C(O)/C(F)=C/c1cccc(Br)n1]

Prepared following general procedure 15. Obtained 190 mg, 77% yield. m/z=246.0 (M+H)⁺. ¹H NMR (400 MHz, DMSO) δ 14.07 (s, 1H), 7.90-7.80 (m, 2H), 7.66 (dd, J=7.0, 1.7 Hz, 1H), 6.93 (d, J_{H}-F=34.1 Hz, 1H).

### Example 20a: (Z)-2-fluoro-3-(thiazol-2-yl)acrylic acid

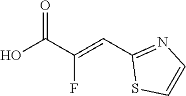 [SMILES: O=C(O)/C(F)=C/c1nccs1]

Prepared following general procedure 15. Obtained 88 mg, 56% yield. m/z=174.0 (M+H)⁺.

### Example 21: (Z)-3-(6-bromopyridin-2-yl)-2-(trifluoromethyl)acrylic acid

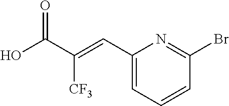 [SMILES: O=C(O)/C(=C/c1cccc(Br)n1)C(F)(F)F]

Prepared following general procedure 16. Obtained 64.8 mg, 27.5% yield.

¹H NMR (400 MHz, DMSO) δ 13.82 (s, 1H), 7.86 (t, J=7.8 Hz, 1H), 7.73-7.65 (m, 2H), 7.46 (s, 1H).

### Example 22: (E)-2-(dimethylcarbamoyl)-3-(thiazol-2-yl)acrylic acid

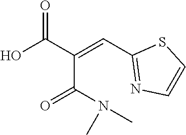

Prepared following general procedure 17. Obtained 14.8 mg, 24.5% yield. m/z=227.2 (M+H)⁺

### Example 23: (Z)-2-(methylsulfonyl)-3-(thiazol-2-yl)acrylic acid

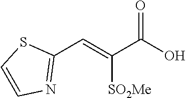 [SMILES: CS(=O)(=O)/C(=C\c1nccs1)C(=O)O]

Prepared following general procedure 18. Obtained 88 mg, 42% yield.

¹H NMR (400 MHz, DMSO) δ 13.41 (s, 1H), 8.16 (dd, J=3.1, 0.6 Hz, 1H), 8.14 (d, J=3.1 Hz, 1H), 7.84 (d, J=0.7 Hz, 1H), 3.31 (s, 3H).

### Example 24: 6-((tert-butyldimethylsilyl)oxy)-3,4-dihydronaphthalen-1(2H)-one

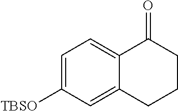 [SMILES: CC(C)(C)[Si](C)(C)Oc1ccc2c(c1)CCCC2=O]

Prepared following general procedure 19. Obtained 1.35 g, 79.2% yield. m/z=277.2 (M+H)⁺.

### Additional Examples

| Example | | | m/z |
|---|---|---|---|
| number | Structure | Preparation | (M + H)⁺ |
| 25 | 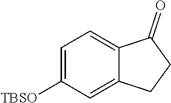 [SMILES: CC(C)(C)[Si](C)(C)Oc1ccc2c(c1)CCC2=O] | Prepared following general procedure 19 | 263.2 |
| 25a | 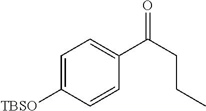 [SMILES: CCCC(=O)c1ccc(O[Si](C)(C)C(C)(C)C)cc1] | Prepared following general procedure 19 | 279.2 |

### Example 26: (E)-N-(6-((tert-butyldimethylsilyl)oxy)-3,4-dihydronaphthalen-1(2H)-ylidene)-2-methylpropane-2-sulfinamide

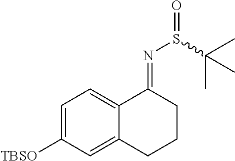 [SMILES: CC(C)(C)S(=O)/N=C1\CCCc2cc(O[Si](C)(C)C(C)(C)C)ccc21]

Prepared following general procedure 20. Obtained 1.12 g, 60.4% yield.

¹H NMR (400 MHz, CDCl₃) δ 8.10 (d, J=8.7 Hz, 1H), 6.71 (dd, J=8.8, 2.5 Hz, 1H), 6.61 (d, J=2.3 Hz, 1H), 3.23 (ddd, J=17.5, 9.1, 4.8 Hz, 1H), 3.00 (ddd, J=17.5, 7.4, 4.5 Hz, 1H), 2.88-2.72 (m, 2H), 2.08-1.88 (m, 2H), 1.31 (s, 9H), 0.98 (s, 9H), 0.23 (s, 6H).

### Additional Examples

| Example | | | m/z |
|---|---|---|---|
| number | Structure | Preparation | (M + H)⁺ |
| 26a | 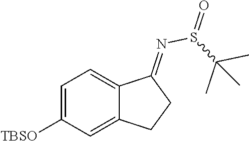 [SMILES: CC(C)(C)S(=O)/N=C1\CCc2cc(O[Si](C)(C)C(C)(C)C)ccc21] | Prepared following general procedure 20 | 366.2 |
| 26b | 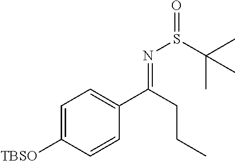 [SMILES: CCC/C(=N\S(=O)C(C)(C)C)c1ccc(O[Si](C)(C)C(C)(C)C)cc1] | Prepared following general procedure 20 | 382.2 |
| 26c | 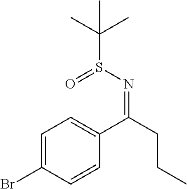 [SMILES: CCC/C(=N/S(=O)C(C)(C)C)c1ccc(Br)cc1] | Prepared following general procedure 20 | 330.0 |

### Example 27: N-(6-((tert-butyldimethylsilyl)oxy)-1,2,3,4-tetrahydronaphthalen-1-yl)-2-methylpropane-2-sulfinamide

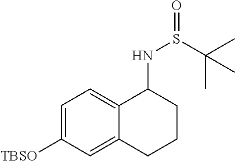 [SMILES: CC(C)(C)S(=O)NC1CCCc2cc(O[Si](C)(C)C(C)(C)C)ccc21]

Prepared following general procedure 21. Obtained 1.02 g, 90.6% yield. m/z=382.3 (M+H)⁺

### Additional Examples

| Example | | | m/z |
|---|---|---|---|
| number | Structure | Preparation | (M + H)⁺ |
| 27a | 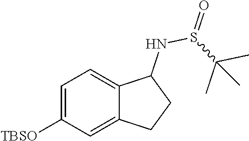 [SMILES: CC(C)(C)S(=O)NC1CCc2cc(O[Si](C)(C)C(C)(C)C)ccc21] | Prepared following general procedure 21 | 368.2 |
| 27b | 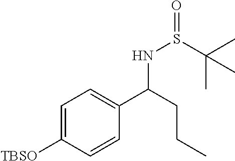 [SMILES: CCCC(NS(=O)C(C)(C)C)c1ccc(O[Si](C)(C)C(C)(C)C)cc1] | Prepared following general procedure 21 | 384.2 |
| 27c | 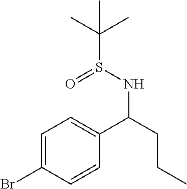 [SMILES: CCCC(NS(=O)C(C)(C)C)c1ccc(Br)cc1] | Prepared following general procedure 21 | 332.1 |

### Example 28: N-(1-(4-((tert-butyldimethylsilyl)oxy)phenyl)heptyl)-2-methylpropane-2-sulfinamide

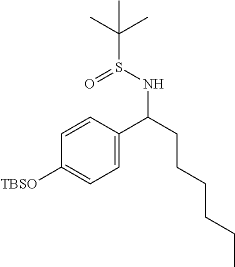 [SMILES: CCCCCCC(NS(=O)C(C)(C)C)c1ccc(O[Si](C)(C)C(C)(C)C)cc1]

Prepared following general procedure 22. Obtained 663 mg, 86.7% yield.

¹H NMR (400 MHz, CDCl₃) δ 7.18-7.09 (m, 2H), 6.84-6.74 (m, 2H), 4.28 (ddd, J=8.5, 6.2, 2.6 Hz, 1H), 3.32 (d, J=2.8 Hz, 1H), 2.05-1.69 (m, 2H), 1.28-1.18 (m, 8H), 1.16 (s, 9H), 0.98 (s, 9H), 0.88-0.78 (m, 3H), 0.19 (s, 6H).

### Example 29: N-benzyl-N-(1-(4-((tert-butyldimethylsilyl)oxy)phenyl)butyl)-2-methylpropane-2-sulfinamide

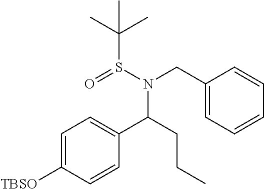 [SMILES: CCCC(c1ccc(O[Si](C)(C)C(C)(C)C)cc1)N(Cc1ccccc1)S(=O)C(C)(C)C]

Prepared following general procedure 23. Obtained 594 mg, 80.2% yield.

¹H NMR (400 MHz, CDCl₃) δ 7.42-7.27 (m, 5H), 7.13-7.05 (m, 2H), 6.81-6.72 (m, 2H), 4.53 (d, J=16.4 Hz, 1H), 3.96-3.87 (m, 2H), 2.29-2.14 (m, 1H), 2.15-2.02 (m, 1H), 1.22-1.01 (m, 2H), 0.98 (s, 9H), 0.96 (s, 9H), 0.83 (t, J=7.3 Hz, 3H), 0.18 (s, 6H).

### Additional Examples

| Example | | | m/z |
|---|---|---|---|
| number | Structure | Preparation | (M + H)⁺ |
| 29a | 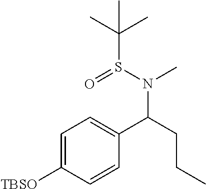 [SMILES: CCCC(c1ccc(O[Si](C)(C)C(C)(C)C)cc1)N(C)S(=O)C(C)(C)C] | Prepared following general procedure 23 | 398.3 |
| 29b | 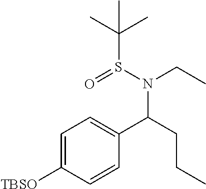 [SMILES: CCCC(c1ccc(O[Si](C)(C)C(C)(C)C)cc1)N(CC)S(=O)C(C)(C)C] | Prepared following general procedure 23 | 412.3 |
| 29c | 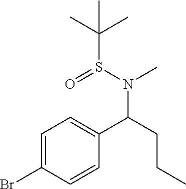 [SMILES: CCCC(c1ccc(Br)cc1)N(C)S(=O)C(C)(C)C] | Prepared following general procedure 23 | 347.3 |

### Example 30: N-ethyl-N-(1-(4-hydroxyphenyl)butyl)-2-methyl propane-2-sulfinamide

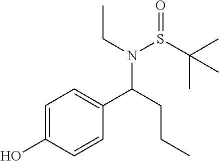 [SMILES: CCCC(c1ccc(O)cc1)N(CC)S(=O)C(C)(C)C]

Prepared following general procedure 24. Obtained 213 mg, 79.5% yield.

¹H NMR (400 MHz, CDCl₃) δ 7.19 (d, J=8.5 Hz, 2H), 6.77 (d, J=8.4 Hz, 2H), 5.98 (s, 1H), 4.21 (dd, J=9.6, 5.6 Hz, 1H), 3.27 (dq, J=14.8, 7.4 Hz, 1H), 2.61 (dq, J=14.0, 7.0 Hz, 1H), 2.15-1.95 (m, 2H), 1.35-1.25 (m, 2H), 1.19 (t, J=7.2 Hz, 3H), 1.07 (d, J=0.9 Hz, 9H), 0.92 (t, J=7.4 Hz, 3H).

### Additional Examples

| Example | | | m/z |
|---|---|---|---|
| number | Structure | Preparation | (M + H)⁺ |
| 30a | 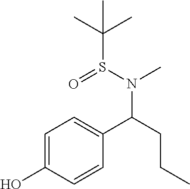 [SMILES: CCCC(c1ccc(O)cc1)N(C)S(=O)C(C)(C)C] | Prepared following general procedure 24 | 284.2 |
| 30b | 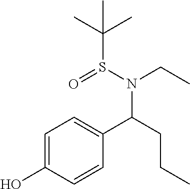 [SMILES: CCCC(c1ccc(O)cc1)N(CC)S(=O)C(C)(C)C] | Prepared following general procedure 24 | 298.2 |

### Example 31: 1-(4-bromophenyl)-N-methylbutan-1-amine

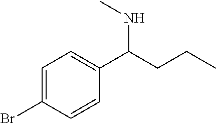

Prepared following general procedure 25. Obtained 112 mg, quantitative yield. m/z=211.0 corresponding to the 1-(4-bromophenyl)butan-1-ylium cation.

### Example 32

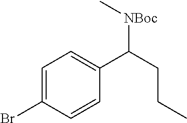 [SMILES: CCCC(c1ccc(Br)cc1)N(C)C(=O)OC(C)(C)C]

To a stirred solution of 1-(4-bromophenyl)butan-1-one (50 g, 220 mmol) in methyl amine (2M in THF, 330 ml, 660 mmol) was added Titanium ethoxide (60.5 ml, 286 mmol) at RT under nitrogen atmosphere. The colour of the reaction mixture was turned from colorless to turbid. The reaction mixture was stirred for 16 h at RT. Thereafter, reaction mixture was turned to pale yellow colour when NaBH₄ (8.33 g, 220 mmol) was added to it portionwise manner at 0° C. The resultant reaction mixture was stirred for 4 h at RT. The progress of the reaction was monitored by UPLC. The reaction mixture was diluted with excess of MTBE and quenched with saturated sodium bicarbonate solution. The solid formed of TiO₂ was filtered and the filtrate was concentrated to afford crude 1-(4-bromophenyl)-N-methylbutan-1-amine (52 g, 204 mmol, 93% yield) as pale yellow liquid. This crude used as such without further purification. To a stirred solution of 1-(4-bromophenyl)-N-methylbutan-1-amine (52 g, 215 mmol) in DCM (250 ml) was added N-ethyl-N-isopropylpropan-2-amine (74.8 ml, 429 mmol) at RT. Then after 10 min, di-tert-butyl dicarbonate (74.0 ml, 322 mmol) was added slowly dropwise at RT to the reaction mixture, the reaction mixture turned from colorless to white turbid solution and the resultant reaction mixtured was stirred for 16 h at RT under nitrogen atmosphere. The progress of the reaction was monitored by UPLC. The reaction mixture was concentrated under reduced pressure and the obtained residue was diluted with aqueous sodium bicarbonate solution and extracted with ethyl acetate. The organic layer was washed with aqueous sodium bicarbonate solution, brine solution, dried over sodium sulfate and concentrated under reduced pressure. The obtained crude was purified by silica gel column chromatography (100-200 mesh, 0-4% ethyl acetate in n-hexane) to afford tert-butyl (1-(4-bromophenyl)butyl)(methyl)carbamate (57 g, 165 mmol, 77% yield) as colorless oil.

¹H-NMR (400 MHz, CDCl3): δ 7.46 (d, J=8.40 Hz, 2H), 7.18 (d, J=8.00 Hz, 2H), 5.32 (m, 1H), 2.56 (s, 3H), 1.84 (d, J=7.20 Hz, 2H), 1.53 (s, 9H), 1.37 (q, J=7.60 Hz, 2H), 1.01 (t, J=7.60 Hz, 3H).

### Example 33: ethyl 2-(4-(1-(isopropylamino)butyl)phenoxy)acetate

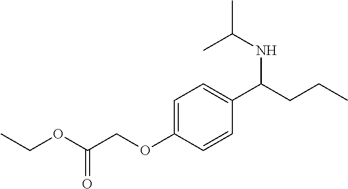 [SMILES: CCCC(NC(C)C)c1ccc(OCC(=O)OCC)cc1]

Prepared following general procedure 26. Obtained 115 mg, 75.2% yield.

¹H NMR (400 MHz, CDCl₃) δ 7.42 (d, J=8.2 Hz, 2H), 6.93 (d, J=8.7 Hz, 2H), 4.62 (s, 2H), 4.29 (q, J=7.1 Hz, 2H), 3.95 (d, J=10.3 Hz, 1H), 2.87-2.75 (m, 1H), 2.07 (d, J=51.2 Hz, 2H), 1.31 (t, J=7.1 Hz, 6H), 1.24 (d, J=6.3 Hz, 3H), 1.20-1.00 (m, 2H), 0.85 (t, J=7.3 Hz, 3H).

### Example 34: 2-(4-(1-(2-cyano-N-methylacetamido)butyl)phenoxy)acetic acid

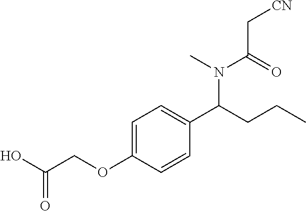 [SMILES: CCCC(c1ccc(OCC(=O)O)cc1)N(C)C(=O)CC#N]

Prepared following general procedure 9. Obtained 230 mg, 81.1% yield. m/z=609.7 (2M+H)⁺

### Additional Example

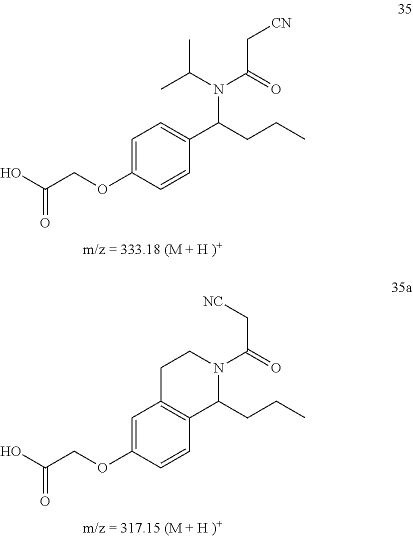 [SMILES: CCCC(c1ccc(OCC(=O)O)cc1)N(C(=O)CC#N)C(C)C ; CCCC1c2ccc(OCC(=O)O)cc2CCN1C(=O)CC#N]

### Example 36: (E)-2-(4-(1-(2-cyano-N-isopropyl-3-(thiazol-2-yl)acrylamido)butyl)phenoxy)acetic acid

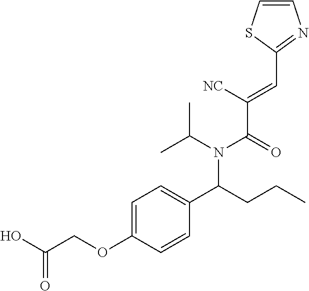 [SMILES: CCCC(c1ccc(OCC(=O)O)cc1)N(C(=O)/C(C#N)=C/c1nccs1)C(C)C]

Prepared following general procedure 10. Obtained 34.4 mg, 81.0% yield. m/z=428.4 (M+H)⁺.

### Additional Example

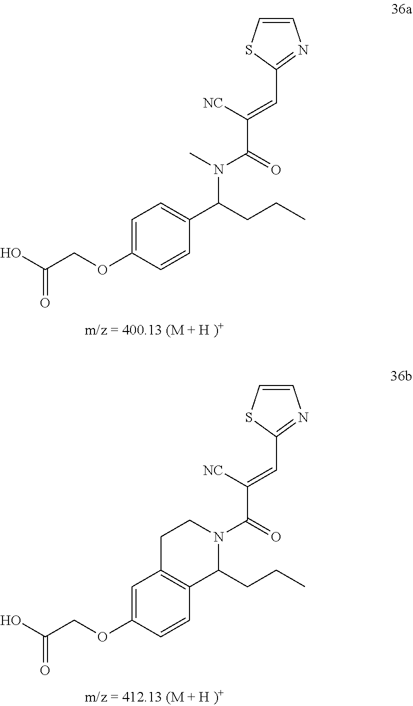 [SMILES: CCCC(c1ccc(OCC(=O)O)cc1)N(C)C(=O)/C(C#N)=C/c1nccs1 ; CCCC1c2ccc(OCC(=O)O)cc2CCN1C(=O)/C(C#N)=C/c1nccs1]

### Example 37: ethyl (E)-3-(4-(1-((tert-butoxycarbonyl)(methyl)amino)butyl)phenyl)acrylate

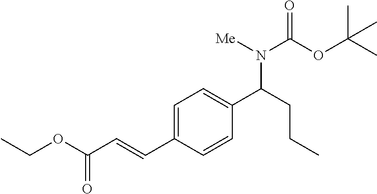 [SMILES: CCCC(c1ccc(/C=C/C(=O)OCC)cc1)N(C)C(=O)OC(C)(C)C]

Prepared following general procedure 27. Obtained 115 mg, 75.2% yield.

¹H NMR (400 MHz, CDCl₃) δ 7.65 (d, J=16.0 Hz, 1H), 7.51-7.43 (m, 2H), 7.29 (dd, J=9.7, 4.9 Hz, 2H), 6.41 (d, J=16.0 Hz, 1H), 5.29 (d, J=56.7 Hz, 1H), 4.25 (q, J=7.1 Hz, 2H), 2.55 (s, 3H), 1.84 (dt, J=13.0, 6.3 Hz, 2H), 1.47 (s, 9H), 1.43-1.33 (m, 2H), 1.32 (t, J=7.1 Hz, 3H), 0.98 (t, J=7.4 Hz, 3H).

### Example 38: ethyl 2-(4-(4-(1-((tert-butoxycarbonyl)(methyl)amino)butyl)phenyl)-1H-pyrazol-1-yl)acetate

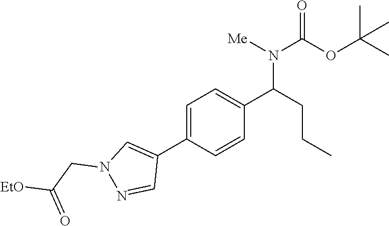 [SMILES: CCCC(c1ccc(-c2cnn(CC(=O)OCC)c2)cc1)N(C)C(=O)OC(C)(C)C]

Prepared following general procedure 27. Obtained 56 mg, 76% yield.

¹H NMR (400 MHz, CDCl₃) δ 7.83 (s, 1H), 7.74 (s, 1H), 7.49-7.44 (m, 2H), 7.33-7.28 (m, 2H), 5.48-5.19 (m, 1H), 4.95 (s, 2H), 4.28 (q, 2H, J=7.2 Hz), 2.59 (s, 3H), 1.90 (m, 2H), 1.52 (s, 9H), 1.44-1.35 (m, 2H), 1.32 (t, 3H, J=7.2 Hz), 1.02 (t, 3H, J=7.3 Hz).

### Example 39: methyl 4′-(1-((tert-butoxycarbonyl)(methyl)amino)butyl)-[1,1′-biphenyl]-4-carboxylate

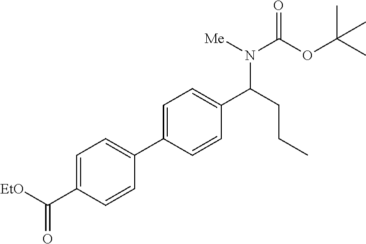 [SMILES: CCCC(c1ccc(-c2ccc(C(=O)OCC)cc2)cc1)N(C)C(=O)OC(C)(C)C]

Prepared following general procedure 27. Obtained 66 mg, 87% yield.

¹H NMR (400 MHz, CDCl₃) δ 8.12 (m, 2H), 7.67 (m, 2H), 7.61 (m, 2H), 7.40 (m, 2H), 5.50-5.19 (m, 1H), 3.96 (s, 3H), 2.62 (s, 3H), 1.92 (m, 2H), 1.53 (s, 9H), 1.47-1.33 (m, 2H), 1.02 (t, 3H, J=7.3 Hz).

### Example 40: ethyl 3-(4-(1-((tert-butoxycarbonyl)(methyl)amino)butyl)phenyl)propanoate

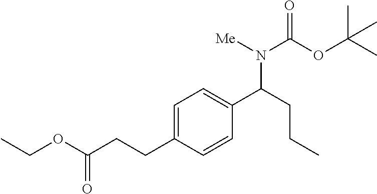 [SMILES: CCCC(c1ccc(CCC(=O)OCC)cc1)N(C)C(=O)OC(C)(C)C]

Prepared following general procedure 28. Obtained 4.81 g, 73.2% yield.

¹H NMR (400 MHz, CDCl₃) δ 7.20 (d, J=8.0 Hz, 2H), 7.15 (d, J=8.2 Hz, 2H), 5.46-5.13 (m, 1H), 4.12 (q, J=7.2 Hz, 2H), 2.93 (t, J=7.8 Hz, 2H), 2.61 (t, J=7.8 Hz, 2H), 2.53 (s, 3H), 1.83 (q, J=7.7, 6.8 Hz, 2H), 1.48 (s, 9H), 1.38-1.30 (m, 2H), 1.23 (t, J=7.1 Hz, 3H), 0.98 (t, J=7.4 Hz, 3H).

### Example 41: 3-(4-(1-((tert-butoxycarbonyl)(methyl)amino)butyl)phenyl)propanoic acid

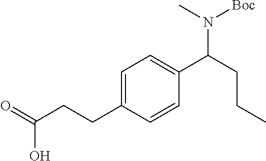 [SMILES: CCCC(c1ccc(CCC(=O)O)cc1)N(C)C(=O)OC(C)(C)C]

To a stirred solution of ethyl 3-(4-(1-((tert-butoxycarbonyl)(methyl)amino) butyl)phenyl)propanoate (17 g, 46.8 mmol) in THF (200 ml) and EtOH (100 ml), sodium hydroxide (6.55 g, 164 mmol) (dissolved in Water (25 ml)) was added dropwise at 0° C. The reaction mixture was stirred at 40° C. for 5 h. The status of the reaction was monitored by UPLC. The reaction mixture was concentrated under reduced pressure and the residue was diluted with cold water. The aqueous portion was washed with n-hexane. Then the layers were separated and aqueous portion was acidified with aqueous potassium bisulphate. The aqueous portion was extracted with DCM and organic layer was concentrated under reduced pressure to 3-(4-(1-((tert-butoxycarbonyl)(methyl)amino)butyl)phenyl)propanoic acid (15 g, 40.2 mmol, 86% yield) as colorless oil. The obtained crude was taken to next step without further purification.

¹H NMR (400 MHz, DMSO-d₆) δ (ppm)=12.08 (s, 1H), 7.40-7.04 (m, 4H), 5.32-4.91 (m, 1H), 3.27 (s, 3H), 2.85-2.76 (m, 2H), 2.56-2.52 (m, 2H), 1.81 (d, J=6.1 Hz, 2H), 1.42 (s, 9H), 1.33-1.20 (m, 2H), 0.95 (t, J=7.3 Hz, 3H). m/z: 334.2 [M+H]⁺

### Example 42: ethyl 3-(4-(1-((tert-butoxycarbonyl)(methyl)amino)butyl)phenyl)propanoate

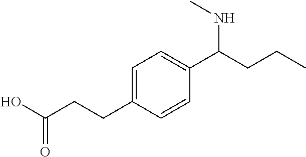 [SMILES: CCCC(NC)c1ccc(CCC(=O)O)cc1]

Prepared following general procedure 8. Obtained 4.5 g, 100.0% yield.

¹H-NMR (400 MHz, CDCl₃): δ 12.15 (s, 1H), 7.42 (d, J=8.0 Hz, 2H), 7.3 (d, J=8.0 Hz, 2H), 4.08-0.08 (m, 1H), 3.59-3.40 (m, 3H), 2.85 (t, J=7.6 Hz, 2H), 2.30 (m, 2H), 2.61 (t, J=8.0 Hz, 2H), 1.84-1.98 (m, 2H), 1.12-1.05 (m, 2H), 0.83 (t, J=7.2 Hz, 3H). m/z: 236.3 [M+H]⁺

### Example 43: 3-(4-(1-(2-cyano-N-methylacetamido)butyl)phenyl)propanoic acid

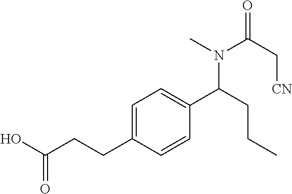 [SMILES: CCCC(c1ccc(CCC(=O)O)cc1)N(C)C(=O)CC#N]

Prepared following general procedure 9. Obtained 3.4 g, 60.9% yield.

¹H-NMR (400 MHz, DMSO-d₆): δ 12.15 (s, 1H), 7.27-7.20 (m, 4H), 5.59-5.57 (m, 1H), 3.87 (s, 2H), 2.82-2.78 (m, 2H), 2.68 (s, 3H), 2.51-2.50 (m, 2H), 1.84 (q, J=8.0 Hz, 2H), 1.30-1.27 (m, 2H), 0.90 (t, J=7.2 Hz, 3H): m/z: 301.2 [M+H]⁺

### Example 44: (E)-3-(4-(1-(2-cyano-N-methyl-3-(thiazol-2-yl)acrylamido)butyl)phenyl)propanoic acid

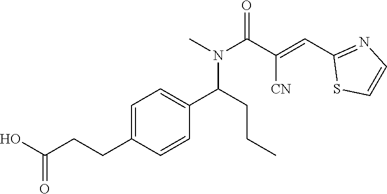 [SMILES: CCCC(c1ccc(CCC(=O)O)cc1)N(C)C(=O)/C(C#N)=C/c1nccs1]

Prepared following general procedure 10. Obtained 4.2 g, 89% yield.

¹H-NMR (400 MHz, CDCl₃): δ 11.75 (s, 1H), 8.14 (dd, J=19.6, 3.2 Hz, 1H), 8.01-7.83 (m, 1H), 7.35-7.25 (m, 4H), 5.68-5.45 (m, 1H), 3.04-2.82 (m, 5H), 2.75 (s, 1H), 2.57-2.51 (m, 2H), 2.05-1.99 (m, 2H), 1.38-1.36 (m, 2H), 0.98 (t, J=7.2 Hz, 3H). m/z: 398.1 [M+H]

### Example 45: 4-(pyrrolidin-2-yl)phenol hydrobromide

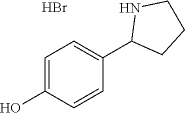 [SMILES: Oc1ccc(C2CCCN2)cc1 ; Br]

Prepared following general procedure 4 from commercially available 2-(4-methoxyphenyl)pyrrolidine. Obtained 184 mg, 66.8% yield. m/z=164.3 ((M+H)⁺

### Example 46: 4-(piperidin-2-yl)phenol hydrobromide

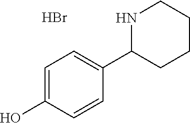 [SMILES: Oc1ccc(C2CCCCN2)cc1 ; Br]

Prepared following general procedure 4 from commercially available 2-(4-methoxyphenyl)piperidine. Obtained 38.4 mg, 28.5% yield. m/z=178.3 ((M+H)⁺

### Example 47: tert-butyl 2-(4-hydroxyphenyl)pyrrolidine-1-carboxylate

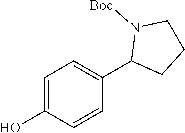 [SMILES: CC(C)(C)OC(=O)N1CCCC1c1ccc(O)cc1]

Prepared following general procedure 5 from 4-(pyrrolidin-2-yl)phenol hydrobromide. Obtained 142 mg, 71.5% yield.

¹H NMR (400 MHz, DMSO-d₆) δ 9.18 (s, 1H), 6.94 (d, J=8.0 Hz, 2H), 6.68 (d, J=8.3 Hz, 2H), 4.75-4.56 (m, 1H), 3.51-3.38 (m, 2H), 2.26-2.12 (m, 1H), 1.87-1.72 (m, 2H), 1.71-1.61 (m, 1H), 1.43-1.06 (m, 9H).

### Example 48: tert-butyl 2-(4-hydroxyphenyl)piperidine-1-carboxylate

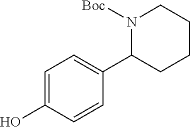 [SMILES: CC(C)(C)OC(=O)N1CCCCC1c1ccc(O)cc1]

Prepared following general procedure 5 from 4-(piperidin-2-yl)phenol hydrobromide. Obtained 34.6 mg, 83.9%.

m/z=222.2 ((M-t-Bu)+H)⁺

### Example 49: tert-butyl 2-(4-(2-ethoxy-2-oxoethoxy)phenyl)pyrrolidine-1-carboxylate

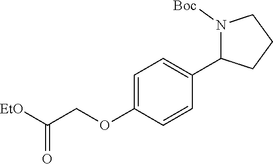 [SMILES: CCOC(=O)COc1ccc(C2CCCN2C(=O)OC(C)(C)C)cc1]

Prepared following general procedure 6. Obtained 155 mg, 82.3% yield.

¹H NMR (400 MHz, CDCl₃) δ 7.13-7.04 (m, 2H), 6.88-6.80 (m, 2H), 4.87-4.72 (m, 1H), 4.59 (s, 2H), 4.27 (q, J=7.1 Hz, 2H), 3.66-3.50 (m, 2H), 2.32-2.20 (m, 1H), 1.95-1.73 (m, 3H), 1.41-1.08 (m, 12H).

### Example 50: tert-butyl 2-(4-(2-ethoxy-2-oxoethoxy)phenyl)piperidine-1-carboxylate

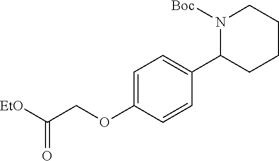 [SMILES: CCOC(=O)COc1ccc(C2CCCCN2C(=O)OC(C)(C)C)cc1]

Prepared following general procedure 6. Obtained 34.7 mg, 76.8% yield.

m/z=308.3 ((M-t-Bu)+H)⁺

### Example 51: 2-(4-(1-(tert-butoxycarbonyl)pyrrolidin-2-yl)phenoxy)acetic acid

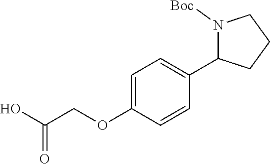 [SMILES: CC(C)(C)OC(=O)N1CCCC1c1ccc(OCC(=O)O)cc1]

Prepared following general procedure 7. Obtained 71.3 mg, 95% yield. m/z=266.2 ((M-t-Bu)+H)⁺

### Example 52: 2-(4-(1-(tert-butoxycarbonyl)piperidin-2-yl)phenoxy)acetic acid

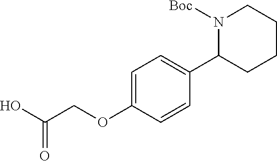 [SMILES: CC(C)(C)OC(=O)N1CCCCC1c1ccc(OCC(=O)O)cc1]

Prepared following general procedure 7. Obtained 31.0 mg, 96.5% yield. m/z=280.2 ((M-t-Bu)+H)⁺

### Example 53: tert-butyl 6-hydroxy-3,4-dihydroquinoline-1(2H)-carboxylate

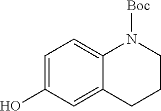 [SMILES: CC(C)(C)OC(=O)N1CCCc2cc(O)ccc21]

Prepared following general procedure 5 from commercially available 1,2,3,4-tetrahydroquinolin-6-ol. Obtained 662 mg, 79.2% yield.

¹H NMR (400 MHz, DMSO-d₆) δ 9.06 (s, 1H), 7.29 (d, J=8.8 Hz, 1H), 6.51 (dd, J=8.8, 2.5 Hz 1H), 6.47 (d, J=2.5 Hz, 1H), 3.59-3.51 (m, 2H), 2.62 (t, J=6.6 Hz, 2H), 1.83-1.72 (m, 2H), 1.43 (s, 9H), m/z=194.2 ((M-tBu)+H)⁺.

### Example 54: tert-butyl 6-(2-(tert-butoxy)-2-oxoethoxy)-3,4-dihydroquinoline-1(2H)-carboxylate

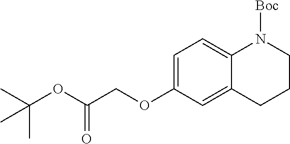 [SMILES: CC(C)(C)OC(=O)COc1ccc2c(c1)CCCN2C(=O)OC(C)(C)C]

Prepared following general procedure 6. Obtained 782 mg, 81.3% yield.

¹H NMR (400 MHz, CDCl₃) δ 7.54 (d, J=9.0 Hz, 1H), 6.69 (dd, J=9.0, 3.0 Hz, 1H), 6.61 (d, J=3.0 Hz, 1H), 4.46 (s, 2H), 3.71-3.63 (m, 2H), 2.72 (t, J=6.6 Hz, 2H), 1.95-1.84 (m, 2H), 1.50 (s, 9H), 1.49 (s, 9H), m/z=252.2 ((M−2tBu)+H)⁺.

### Example 55: 2-((1,2,3,4-tetrahydroquinolin-6-yl)oxy)acetic acid

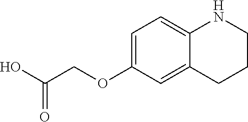 [SMILES: O=C(O)COc1ccc2c(c1)CCCN2]

Prepared following general procedure 33. Obtained 489 mg, 95.0% yield. m/z=208.2 (M+H)⁺.

### Example 56: 2-((1-(2-cyanoacetyl)-1,2,3,4-tetrahydroquinolin-6-yl)oxy)acetic acid

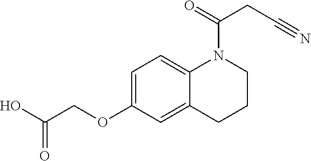 [SMILES: N#CCC(=O)N1CCCc2cc(OCC(=O)O)ccc21]

Prepared following general procedure 9. Obtained 174 mg, 68.2% yield. m/z=275.2 (M+H)⁺.

### Example 57: (E)-2-((1-(2-cyano-3-(thiazol-2-yl)acryloyl)-1,2,3,4-tetrahydroquinolin-6-yl)oxy)acetic acid

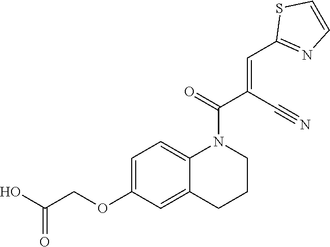 [SMILES: N#C/C(=C\c1nccs1)C(=O)N1CCCc2cc(OCC(=O)O)ccc21]

Prepared following general procedure 9. Obtained 73 mg, 47.1% yield

¹H NMR (400 MHz, DMSO-d₆) δ 12.96 (s, 1H), 8.21-8.16 (m, 2H), 8.11 (s, 1H), 7.16 (d, J=8.8 Hz, 1H), 6.86 (d, J=2.9 Hz, 1H), 6.72 (dd, J=8.8, 2.9 Hz, 1H), 4.65 (s, 2H), 3.76 (t, J=6.6 Hz, 2H), 2.72 (t, J=6.6 Hz, 2H), 1.94 (p, J=6.6 Hz, 2H); m/z=370.3 (M+H)⁺.

### Example 58: 4-((2S,4R)-1-acetyl-4-((4-chlorophenyl)amino)-2-methyl-1,2,3,4-tetrahydroquinolin-6-yl)-N-(17-amino-3,6,9,12,15-pentaoxaheptadecyl)benzamide-hydrochloride salt

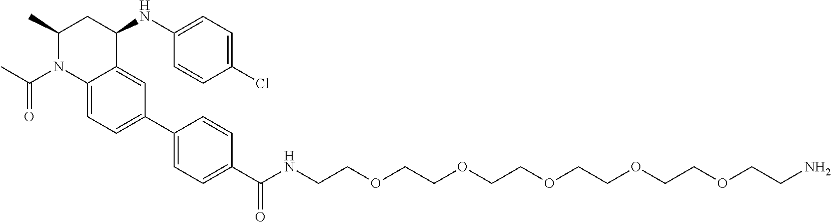 [SMILES: CC(=O)N1c2ccc(-c3ccc(C(=O)NCCOCCOCCOCCOCCOCCN)cc3)cc2[C@H](Nc2ccc(Cl)cc2)C[C@@H]1C]

To a solution of tert-butyl (17-amino-3,6,9,12,15-pentaoxaheptadecyl)carbamate (38.5 mg, 1.1 equiv., 101 μmol) in DMF (1 mL), iBET726 (40.0 mg, 1.0 equiv., 92.0 μmol), HATU (35.0 mg, 1.0 equiv., 92.0 μmol) and N-ethyl-N-isopropylpropan-2-amine (35.7 mg, 48.1 μL, 3.0 equiv., 276 μmol) in DMF (1 mL) were added. The mixture was stirred at rt for 15 min. The crude was diluted in EtOAc and washed with water and brine. Purification by flash chromatography (DCM/MeOH (0-15%)) yielded the desired product as a yellow oil, 74 mg (quantitative yield).

The oil was dissolved in DCM (1 mL) and treated with HCl (1.49 g, 1.00 mL, 4 M, 40 equiv., 4.00 mmol) and the resulting heterogenous mixture was stirred at rt for 1 h. The reaction mixture was directly evaporated and dried at high vacuum to deliver the desired product as HCl salt (70 mg, 98%) as a colourless solid, m/z=697.7 (M+H)⁺.

### Example A1: 4-((2S,4R)-1-acetyl-4-((4-chlorophenyl)amino)-2-methyl-1,2,3,4-tetrahydroquinolin-6-yl)-N-(1-(4-(1-((E)-2-cyano-3-(thiazol-2-yl)acrylamido)butyl)phenoxy)-2-oxo-6,9,12,15,18-pentaoxa-3-azaicosan-20-yl)benzamide

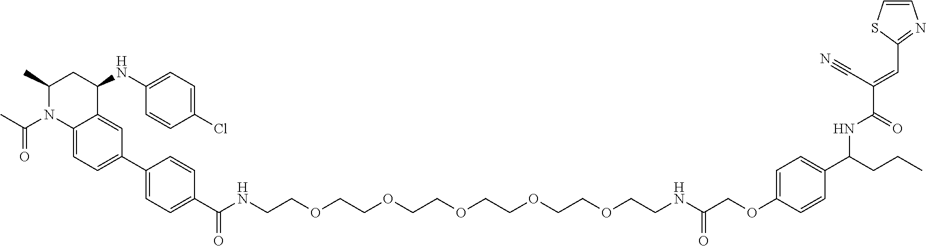 [SMILES: CCCC(NC(=O)/C(C#N)=C/c1nccs1)c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1]

A solution of preparative compound 58 (5.00 mg, 1.0 equiv., 7.17 μmol) in DMF (0.3 mL) was treated with a solution of 2-(4-(1-(2-cyano-3-(thiazol-2-yl)acrylamido)butyl)phenoxy)acetic acid (2.76 mg, 1.0 equiv., 7.17 μmol) in DMF (0.3 mL), HATU (5.45 mg, 2.0 equiv., 14.3 μmol) and DIPEA (2.78 mg, 3.75 μL, 3.0 equiv., 21.5 μmol) in DMF (0.3 mL). The mixture was stirred at rt for 15 min. The crude mixture was diluted to 1 mL with MeOH and directly purified using preparative HPLC (using a gradient from 5% to 95% of acetonitrile in water containing 0.1% of formic acid over 10 minutes) to deliver the product A1 (5.0 mg, 4.7 μmol, 65%) as a pale yellow wax; m/z=1064.4 (M+H)⁺.

### Example A2: 4-((2S,4R)-1-acetyl-4-((4-chlorophenyl)amino)-2-methyl-1,2,3,4-tetrahydroquinolin-6-yl)-N-(1-(4-(1-((E)-2-cyano-N-methyl-3-(thiazol-2-yl)acrylamido)butyl)phenoxy)-2-oxo-6,9,12,15,18-pentaoxa-3-azaicosan-20-yl)benzamide

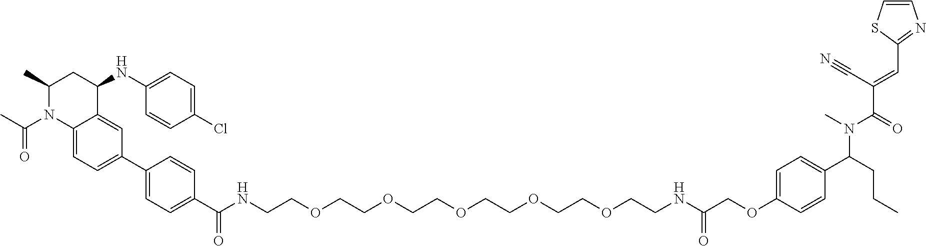 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)/C(C#N)=C/c1nccs1]

In a test tube preparative compound 58 (8.0 mg, 1.1 equiv., 11 μmol) was dissolved in 100 μL of DMF. 15 μL of DIPEA was added and the tube was mixed. (E)-2-(4-(1-(2-cyano-N-methyl-3-(thiazol-2-yl)acrylamido)butyl)phenoxy)acetic acid (4.0 mg, 1.0 equiv., 10 μmol) was dissolved in DMF (0.3 mL). 15 μL of DIPEA was added, followed by HATU (4.6 mg, 1.2 equiv., 12 μmol). This was mixed and added to the tube containing the amine solution. The tube was shaken and left for around 1 hr. LCMS showed consumption of the starting materials and formation of the desired product. 0.5 mL of MeOH was added and the product was purified on preparative HPLC HPLC (using a gradient from 20% to 95% of acetonitrile in water containing 0.1% of formic acid over 10 minutes) to give the purified product, 2.7 mg (26% yield), m/z=1079.0 (M+H)⁺.

### Additional Examples

| Example | | m/z |
|---|---|---|
| number | Structure/Preparation | (M + H)⁺ |
| A3 | 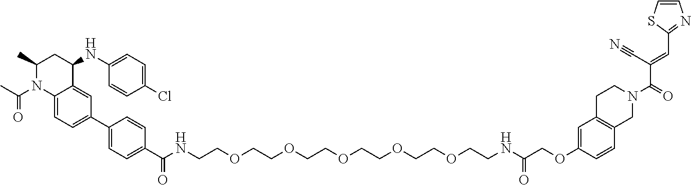 [SMILES: CC(=O)N1c2ccc(-c3ccc(C(=O)NCCOCCOCCOCCOCCOCCNC(=O)COc4ccc5c(c4)CCN(C(=O)/C(C#N)=C/c4nccs4)C5)cc3)cc2[C@H](Nc2ccc(Cl)cc2)C[C@@H]1C] Prepared as described for example A2 | 1048.4 |
| A5 | 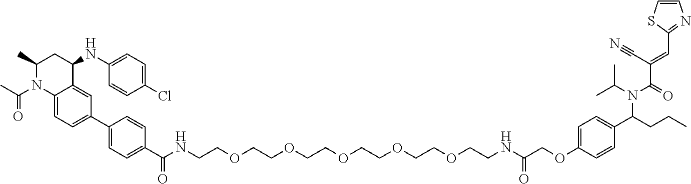 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C(=O)/C(C#N)=C/c1nccs1)C(C)C] Prepared as described for example A2 | 1106.5 |
| A6 | 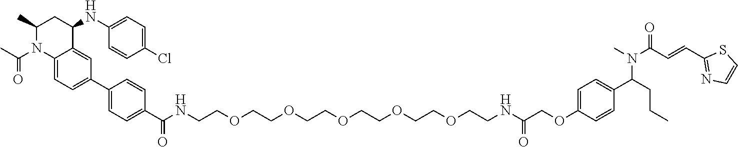 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)/C=C/c1nccs1] Prepared as described for example A2 | 1053.5 |
| A40 | 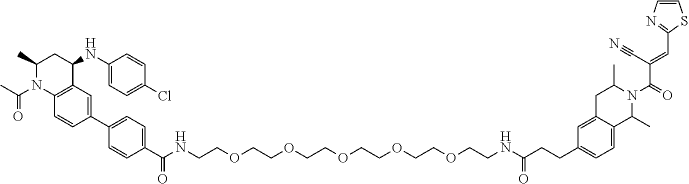 [SMILES: CC(=O)N1c2ccc(-c3ccc(C(=O)NCCOCCOCCOCCOCCOCCNC(=O)CCc4ccc5c(c4)CC(C)N(C(=O)/C(C#N)=C/c4nccs4)C5C)cc3)cc2[C@H](Nc2ccc(Cl)cc2)C[C@@H]1C] Prepared as described for example A2 | 1075.2 |
| A41 | 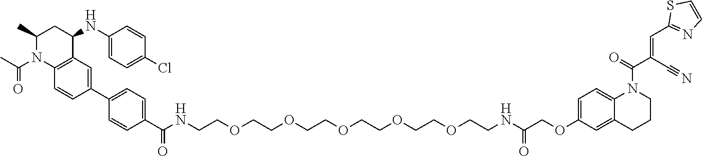 [SMILES: CC(=O)N1c2ccc(-c3ccc(C(=O)NCCOCCOCCOCCOCCOCCNC(=O)COc4ccc5c(c4)CCCN5C(=O)/C(C#N)=C/c4nccs4)cc3)cc2[C@H](Nc2ccc(Cl)cc2)C[C@@H]1C] Prepared as described for example A2 | 1049.1 |

### Example A4: 4-((2S,4R)-1-acetyl-4-((4-chlorophenyl)amino)-2-methyl-1,2,3,4-tetrahydroquinolin-6-yl)-N-(1-(4-(1-(2-cyano-N-methyl-3-(thiazol-2-yl)propanamido)butyl)phenoxy)-2-oxo-6,9,12,15,18-pentaoxa-3-azaicosan-20-yl)benzamide

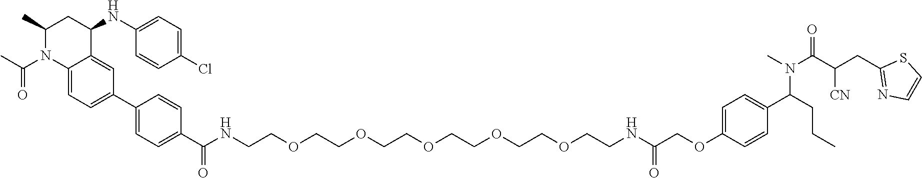 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)C(C#N)Cc1nccs1]

A solution preparative compound A2 (13.7 mg, 1.0 equiv., 12.7 μmol) in THF (1 mL) was treated sodium triacetoxyborohydride (13.5 mg, 5.0 equiv., 63.5 μmol) and stirred at 50° C. overnight. The reaction was quenched by addition of NH₄Cl and extracted with CH₂Cl₂. The combined organic layers were washed with water and brine, dried over MgSO₄ and concentrated under reduced pressure. Purification by preparative HPLC (using a gradient from 5% to 95% of acetonitrile in water containing 0.1% of formic acid over 10 minutes) yielded the desired product as a colourless solid, m/z=1080.5 (M+H)⁺.

### Example 59: tert-butyl 2-(4-((1-(4-((2S,4R)-1-acetyl-4-((4-chlorophenyl)amino)-2-methyl-1,2,3,4-tetrahydroquinolin-6-yl)phenyl)-1,21-dioxo-5,8,11,14,17-pentaoxa-2,20-diazadocosan-22-yl)oxy)phenyl)pyrrolidine-1-carboxylate

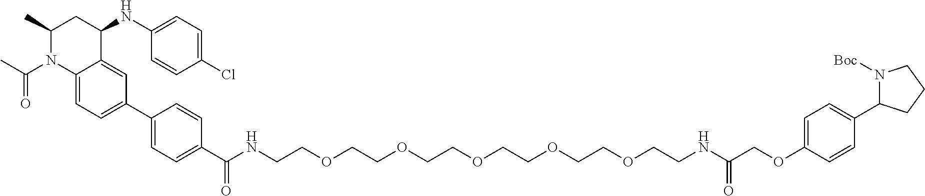 [SMILES: CC(=O)N1c2ccc(-c3ccc(C(=O)NCCOCCOCCOCCOCCOCCNC(=O)COc4ccc(C5CCCN5C(=O)OC(C)(C)C)cc4)cc3)cc2[C@H](Nc2ccc(Cl)cc2)C[C@@H]1C]

A solution of 4-((2S,4R)-1-acetyl-4-((4-chlorophenyl)amino)-2-methyl-1,2,3,4-tetrahydroquinolin-6-yl)-N-(17-amino-3,6,9,12,15-pentaoxaheptadecyl)benzamide, HCl (20.5 mg, 280 μL, 0.1 M, 1.0 equiv., 28.0 μmol) in DMF (1 mL) was treated sequentially with a solution of 2-(4-(1-(tert-butoxycarbonyl)pyrrolidin-2-yl)phenoxy)acetic acid (9.00 mg, 280 μL, 0.1 M, 1.0 equiv., 28.0 μmol), 2-(3H-[1,2,3]triazolo[4,5-b]pyridin-3-yl)-1,1,3,3-tetramethylisouronium hexafluorophosphate(V) (10.6 mg, 1.0 equiv., 28.0 μmol) and N-ethyl-N-isopropylpropan-2-amine (10.9 mg, 14.6 μL, 3.0 equiv., 84.0 μmol). The mixture was stirred at rt for 15 min. LC-MS showed complete conversion to a new product with the expected mass. The reaction was diluted with CH₂Cl₂ (5 mL) and quenched with H₂O (2 mL). The phases were separated, and the organic layer was washed with H₂O (2 mL) and NaHCO₃ (2 mL). Purification by flash chromatography (CH₂Cl₂/MeOH) yielded the desired product (27.3 mg, 27.3 μmol, 97.4%) as a yellow oil. m/z=1000.5 (M+H)⁺.

### Additional Examples

| Example | | m/z |
|---|---|---|
| number | Structure/Preparation | (M + H)⁺ |
| 60 | 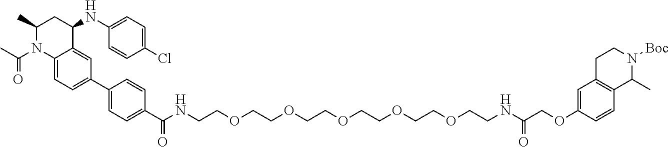 [SMILES: CC(=O)N1c2ccc(-c3ccc(C(=O)NCCOCCOCCOCCOCCOCCNC(=O)COc4ccc5c(c4)CCN(C(=O)OC(C)(C)C)C5C)cc3)cc2[C@H](Nc2ccc(Cl)cc2)C[C@@H]1C] Prepared as described for example 59 | 1000.4 |
| 60a | 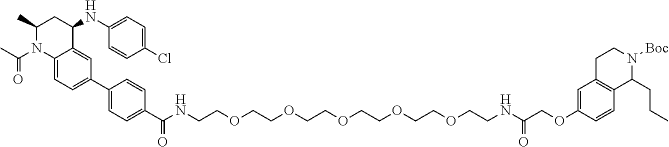 [SMILES: CCCC1c2ccc(OCC(=O)NCCOCCOCCOCCOCCOCCNC(=O)c3ccc(-c4ccc5c(c4)[C@H](Nc4ccc(Cl)cc4)C[C@H](C)N5C(C)=O)cc3)cc2CCN1C(=O)OC(C)(C)C] Prepared as described for example 59 | 1028.5 |
| 60b | 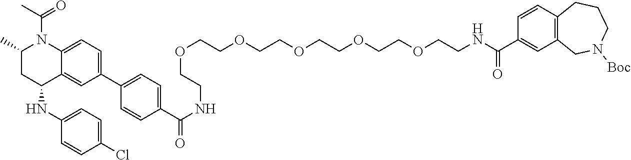 [SMILES: CC(=O)N1c2ccc(-c3ccc(C(=O)NCCOCCOCCOCCOCCOCCNC(=O)c4ccc5c(c4)CN(C(=O)OC(C)(C)C)CCC5)cc3)cc2[C@H](Nc2ccc(Cl)cc2)C[C@@H]1C] Prepared as described for example 59 using commercially available 2-(tert-butoxycarbonyl)-2,3,4,5-tetrahydro-1H- benzo[c]azepine-8-carboxylic acid | [M − H]⁺ 969.4 |
| 60c | 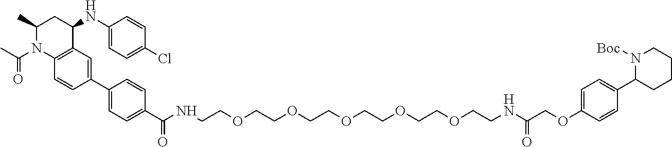 [SMILES: CC(=O)N1c2ccc(-c3ccc(C(=O)NCCOCCOCCOCCOCCOCCNC(=O)COc4ccc(C5CCCCN5C(=O)OC(C)(C)C)cc4)cc3)cc2[C@H](Nc2ccc(Cl)cc2)C[C@@H]1C] Prepared as described for example 59 | 1015.1 |

### Example 61: 4-((2S,4R)-1-acetyl-4-((4-chlorophenyl)amino)-2-methyl-1,2,3,4-tetrahydroquinolin-6-yl)-N-(1-(4-(1-(2-cyanoacetyl)pyrrolidin-2-yl)phenoxy)-2-oxo-6,9,12,15,18-pentaoxa-3-azaicosan-20-yl)benzamide

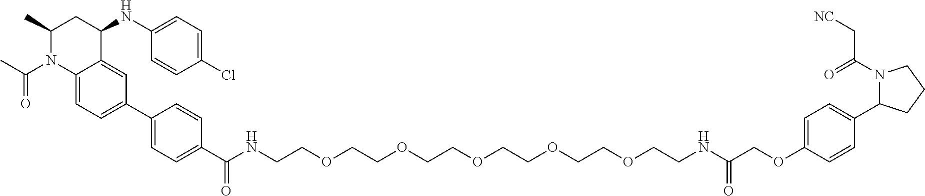 [SMILES: CC(=O)N1c2ccc(-c3ccc(C(=O)NCCOCCOCCOCCOCCOCCNC(=O)COc4ccc(C5CCCN5C(=O)CC#N)cc4)cc3)cc2[C@H](Nc2ccc(Cl)cc2)C[C@@H]1C]

To a solution of tert-butyl 2-(4-((1-(4-((2S,4R)-1-acetyl-4-((4-chlorophenyl)amino)-2-methyl-1,2,3,4-tetrahydroquinolin-6-yl)phenyl)-1,21-dioxo-5,8,11,14,17-pentaoxa-2,20-diazadocosan-22-yl)oxy)phenyl)pyrrolidine-1-carboxylate (27 mg, 1.0 equiv., 27 μmol), in DCM (2 mL), HCl (4M in dioxane, 340 μL, 50 equiv., 1.38 mmol) was added. The mixture was stirred for one hour at r.t., then evaporated to dryness. The crude was dissolved in dioxane (2 mL) and treated with 3-(3,5-dimethyl-1H-pyrazol-1-yl)-3-oxopropanenitrile (4.9 mg, 1.1 equiv., 30 μmol) and triethylamine (11.4 μL, 3.0 equiv., 82 μmol) at 90° C. for 2 hours. Complete conversion to a new product was observed. Volatiles were evaporated under reduced pressure and the crude was purified by flash chromatography (CH₂Cl₂/MeOH) to obtain the desired product (16.3 mg, 61.7%), m/z=968.1 (M+H)⁺.

### Additional Examples

| Example | | m/z |
|---|---|---|
| number | Structure/Preparation | (M + H)⁺ |
| 62 | 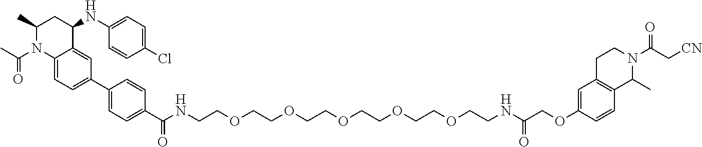 [SMILES: CC(=O)N1c2ccc(-c3ccc(C(=O)NCCOCCOCCOCCOCCOCCNC(=O)COc4ccc5c(c4)CCN(C(=O)CC#N)C5C)cc3)cc2[C@H](Nc2ccc(Cl)cc2)C[C@@H]1C] Prepared as described for example 61 | 967.5 |
| 63 | 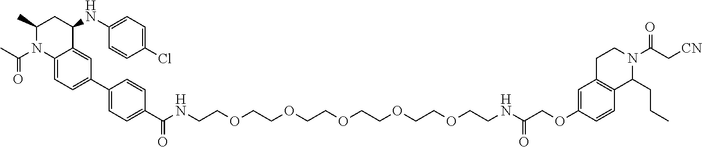 [SMILES: CCCC1c2ccc(OCC(=O)NCCOCCOCCOCCOCCOCCNC(=O)c3ccc(-c4ccc5c(c4)[C@H](Nc4ccc(Cl)cc4)C[C@H](C)N5C(C)=O)cc3)cc2CCN1C(=O)CC#N] Prepared as described for example 61 | 995.5 |
| 64 | 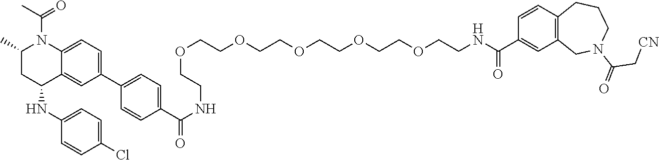 [SMILES: CC(=O)N1c2ccc(-c3ccc(C(=O)NCCOCCOCCOCCOCCOCCNC(=O)c4ccc5c(c4)CN(C(=O)CC#N)CCC5)cc3)cc2[C@H](Nc2ccc(Cl)cc2)C[C@@H]1C] Prepared as described for example 61 | 937.2 |
| 65 | 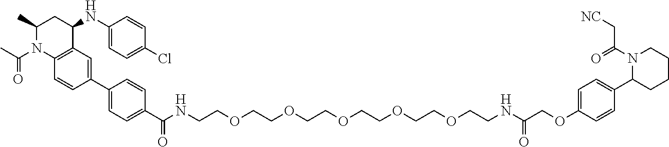 [SMILES: CC(=O)N1c2ccc(-c3ccc(C(=O)NCCOCCOCCOCCOCCOCCNC(=O)COc4ccc(C5CCCCN5C(=O)CC#N)cc4)cc3)cc2[C@H](Nc2ccc(Cl)cc2)C[C@@H]1C] Prepared as described for example 61 | 982.1 |

### Example A7: 4-((2S,4R)-1-acetyl-4-((4-chlorophenyl)amino)-2-methyl-1,2,3,4-tetrahydroquinolin-6-yl)-N-(1-(4-(1-((E)-2-cyano-3-(thiazol-2-yl)acryloyl)pyrrolidin-2-yl)phenoxy)-2-oxo-6,9,12,15,18-pentaoxa-3-azaicosan-20-yl)benzamide

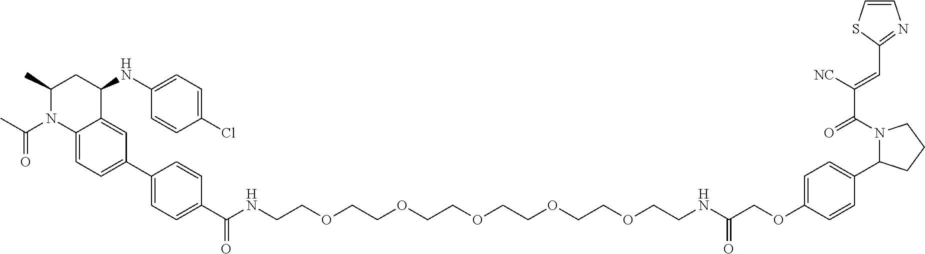 [SMILES: CC(=O)N1c2ccc(-c3ccc(C(=O)NCCOCCOCCOCCOCCOCCNC(=O)COc4ccc(C5CCCN5C(=O)/C(C#N)=C/c5nccs5)cc4)cc3)cc2[C@H](Nc2ccc(Cl)cc2)C[C@@H]1C]

A solution of 4-((2S,4R)-1-acetyl-4-((4-chlorophenyl)amino)-2-methyl-1,2,3,4-tetrahydroquinolin-6-yl)-N-(1-(4-(1-(2-cyanoacetyl)pyrrolidin-2-yl)phenoxy)-2-oxo-6,9,12,15,18-pentaoxa-3-azaicosan-20-yl)benzamide (16.3 mg, 1.0 equiv., 16.8 μmol) in dioxane (2 mL) was treated with thiazole-2-carbaldehyde (3.7 μL, 2.5 equiv., 42.1 μmol) and piperidine (1.66 μL, 1.0 equiv., 16.8 μmol) and the mixture was heated to 66° C. for 72 h. The volatiles were evaporated under reduced pressure. Purification by reverse phase HPLC (using a gradient from 5% to 95% of acetonitrile in water containing 0.1% of formic acid over 10 minutes) gave the desired product (8 mg, 45% yield). m/z=1063.7 (M+H)⁺.

### Additional Examples

| Example | | m/z |
|---|---|---|
| number | Structure/Preparation | (M + H)⁺ |
| A8 | 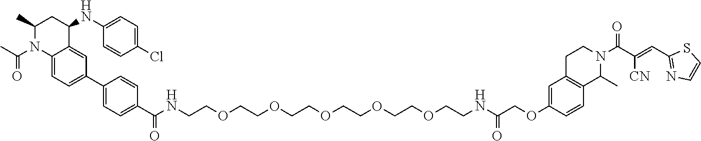 [SMILES: CC(=O)N1c2ccc(-c3ccc(C(=O)NCCOCCOCCOCCOCCOCCNC(=O)COc4ccc5c(c4)CCN(C(=O)/C(C#N)=C/c4nccs4)C5C)cc3)cc2[C@H](Nc2ccc(Cl)cc2)C[C@@H]1C] Prepared as described for example A7 | 1062.2 |
| A9 | 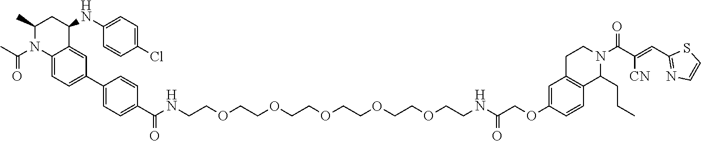 [SMILES: CCCC1c2ccc(OCC(=O)NCCOCCOCCOCCOCCOCCNC(=O)c3ccc(-c4ccc5c(c4)[C@H](Nc4ccc(Cl)cc4)C[C@H](C)N5C(C)=O)cc3)cc2CCN1C(=O)/C(C#N)=C/c1nccs1] Prepared as described for example A7 | 1090.5 |
| A42 | 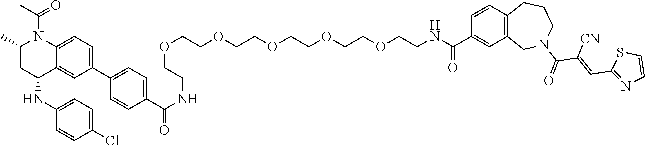 [SMILES: CC(=O)N1c2ccc(-c3ccc(C(=O)NCCOCCOCCOCCOCCOCCNC(=O)c4ccc5c(c4)CN(C(=O)/C(C#N)=C/c4nccs4)CCC5)cc3)cc2[C@H](Nc2ccc(Cl)cc2)C[C@@H]1C] Prepared as described for example A7 | 1032.3 |
| A43 | 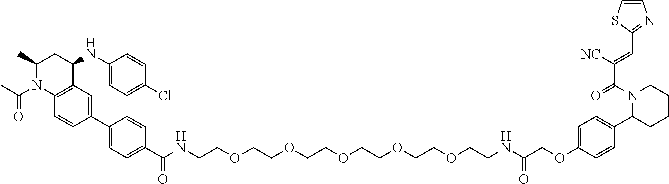 [SMILES: CC(=O)N1c2ccc(-c3ccc(C(=O)NCCOCCOCCOCCOCCOCCNC(=O)COc4ccc(C5CCCCN5C(=O)/C(C#N)=C/c5nccs5)cc4)cc3)cc2[C@H](Nc2ccc(Cl)cc2)C[C@@H]1C] Prepared as described for example A7 | 1077.2 |

### Example 66: tert-butyl 4-((5-(((5-(tert-butyl)oxazol-2-yl)methyl) thio)thiazol-2-yl)carbamoyl)-[1,4′-bipiperidine]-1′-carboxylate

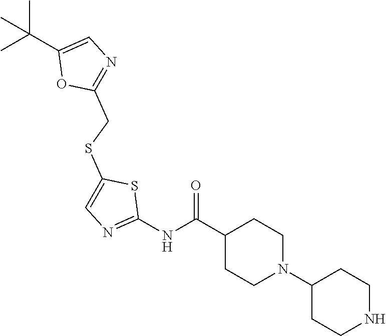 [SMILES: CC(C)(C)c1cnc(CSc2cnc(NC(=O)C3CCN(C4CCNCC4)CC3)s2)o1]

SNS-032 (60 mg, 1.0 equiv., 0.16 mmol) and tert-butyl 4-oxopiperidine-1-carboxylate (47 mg, 1.5 equiv., 0.24 mmol) were suspended in DCM (1.5 mL). Tetraethoxytitanium (72 mg, 66 μL, 2.0 equiv., 0.32 mmol) was added and all suspended solids went into solution. The reaction was stirred at room temperature overnight. Sodium cyanoborohydride (20 mg, 2.0 equiv., 0.32 mmol) was added and the reaction was stirred at room temperature for 2 hours. The reaction was quenched by addition of sat. NaHCO₃ solution, filtered over celite and extracted three times with EtOAc. The organic layer was reduced in vacuo and dry loaded onto silica. The reaction was purified by flash chromatography (12 g column, 0 to 20% MeOH in DCM) to give tert-butyl 4-((5-(((5-(tert-butyl)oxazol-2-yl)methyl)thio)thiazol-2-yl)carbamoyl)-[1,4′-bipiperidine]-1′-carboxylate (73 mg, 0.13 mmol, 82%) as a yellow solid. The Boc protected intermediate was then dissolved in DCM (2 ml) and treated with HCl (4 M in dioxane, 2 ml) for 1 hour. The reaction mixture was evaporated to dryness to obtain the desired product as hydrochloride salt in quantitative yield, m/z=464.2 (M+H)⁺.

### Additional Examples

| Example | | | m/z |
|---|---|---|---|
| number | Structure | Preparation | (M + H)⁺ |
| 67 | 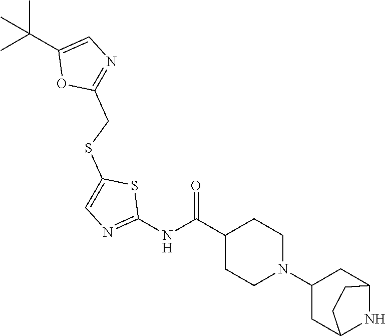 [SMILES: CC(C)(C)c1cnc(CSc2cnc(NC(=O)C3CCN(C4CC5CCC(C4)N5)CC3)s2)o1] | Prepared as described for example 66 | 490.2 |
| 68 | 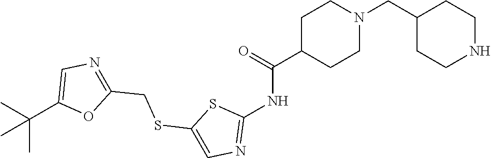 [SMILES: CC(C)(C)c1cnc(CSc2cnc(NC(=O)C3CCN(CC4CCNCC4)CC3)s2)o1] | Prepared as described for example 66 | 478.2 |
| 69 | 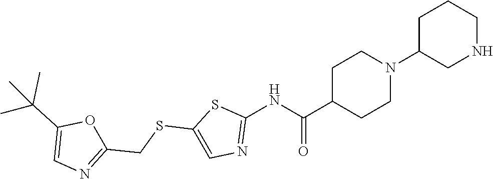 [SMILES: CC(C)(C)c1cnc(CSc2cnc(NC(=O)C3CCN(C4CCCNC4)CC3)s2)o1] | Prepared as described for example 66 | 464.2 |
| 70 | 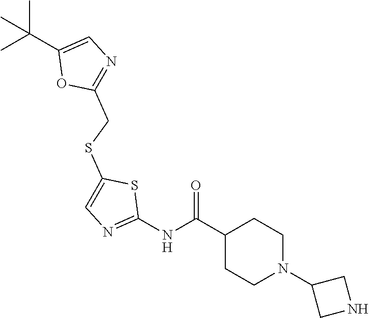 [SMILES: CC(C)(C)c1cnc(CSc2cnc(NC(=O)C3CCN(C4CNC4)CC3)s2)o1] | Prepared as described for example 66 | 436.2 |
| 71 | 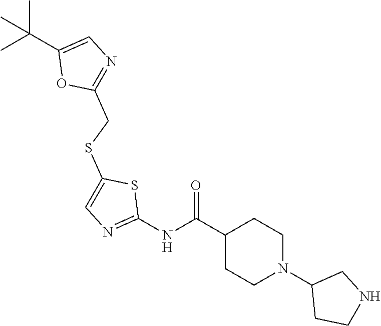 [SMILES: CC(C)(C)c1cnc(CSc2cnc(NC(=O)C3CCN(C4CCNC4)CC3)s2)o1] | Prepared as described for example 66 | 450.2 |

### Example A38: (E)-N-(5-(((5-(tert-butyl)oxazol-2-yl)methyl)thio)thiazol-2-yl)-1-(2-((2-(2-cyano-3-(thiazol-2-yl)acryloyl)-1-propyl-1,2,3,4-tetrahydroisoquinolin-6-yl)oxy)acetyl)piperidine-4-carboxamide

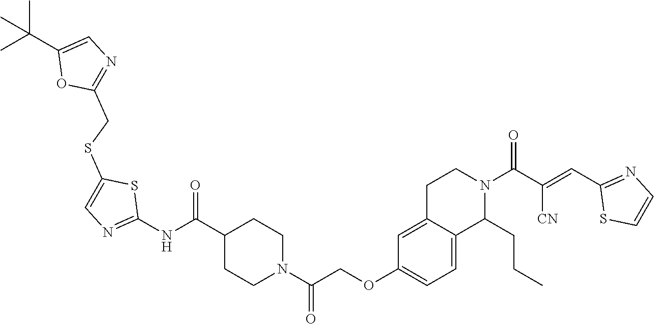 [SMILES: CCCC1c2ccc(OCC(=O)N3CCC(C(=O)Nc4ncc(SCc5ncc(C(C)(C)C)o5)s4)CC3)cc2CCN1C(=O)/C(C#N)=C/c1nccs1]

A solution of SNS032 (1.0 equiv.) in DMF (0.1 M) was treated with a solution of 36b (1.0 equiv.) HATU (1.1 equiv.) and DIPEA (2.5 equiv.) in DMF (0.1 M) and the reaction mixture was stirred at rt for 15 min. The reaction was quenched with water and extracted with EtOAc. The combined organic layers were washed with LiCl (5%), water and brine, dried over MgSO₄ and concentrated under reduced pressure. Purification by preparative HPLC (using a gradient from 5% to 95% of acetonitrile in water containing 0.1% of formic acid over 10 minutes) yielded the desired product A38, m/z=774.3 (M+H)⁺.

This reaction protocol is exemplified in relation to a THIQ analogue but is also applicable to the synthesis of N-alkylated analogues.

### Additional Examples

| Exam- | | | |
|---|---|---|---|
| ple | | | m/z |
| number | Structure | Preparation | (M + H)⁺ |
| A31 | 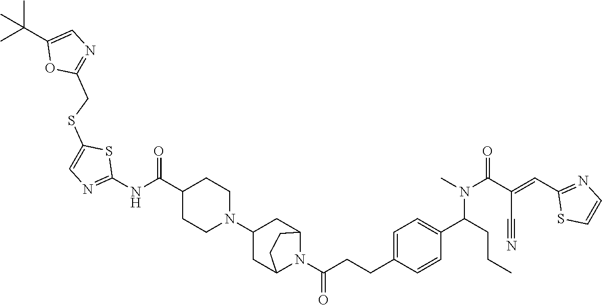 [SMILES: CCCC(c1ccc(CCC(=O)N2C3CCC2CC(N2CCC(C(=O)Nc4ncc(SCc5ncc(C(C)(C)C)o5)s4)CC2)C3)cc1)N(C)C(=O)/C(C#N)=C/c1nccs1] | Prepared as described for example A38 | 869.4 |
| A28 | 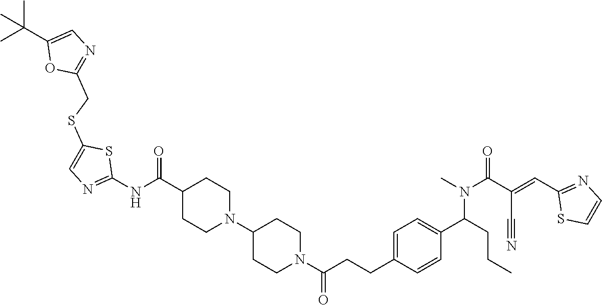 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)/C(C#N)=C/c1nccs1] | Prepared as described for example A38 | 843.4 |
| A27 | 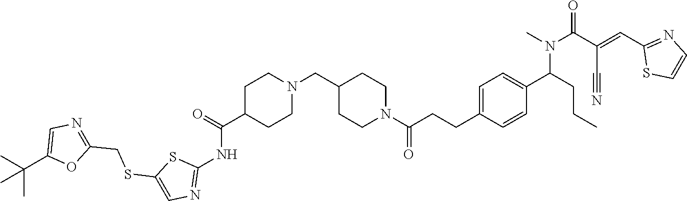 [SMILES: CCCC(c1ccc(CCC(=O)N2CCC(CN3CCC(C(=O)Nc4ncc(SCc5ncc(C(C)(C)C)o5)s4)CC3)CC2)cc1)N(C)C(=O)/C(C#N)=C/c1nccs1] | Prepared as described for example A38 | 857.4 |
| A20 | 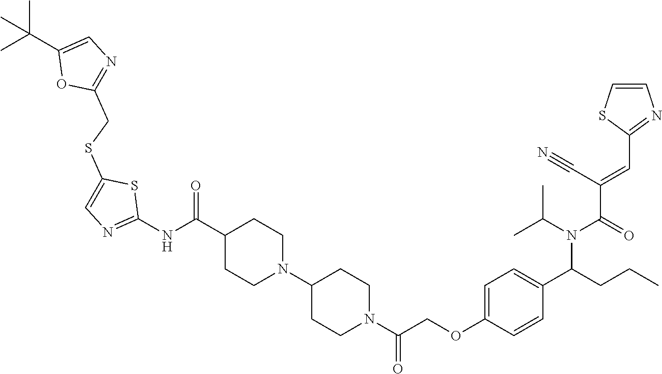 [SMILES: CCCC(c1ccc(OCC(=O)N2CCC(N3CCC(C(=O)Nc4ncc(SCc5ncc(C(C)(C)C)o5)s4)CC3)CC2)cc1)N(C(=O)/C(C#N)=C/c1nccs1)C(C)C] | Prepared as described for example A38 | 873.4 |
| A19 | 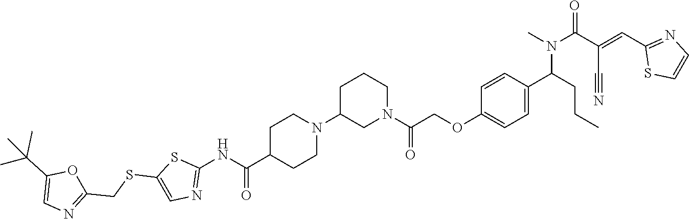 [SMILES: CCCC(c1ccc(OCC(=O)N2CCCC(N3CCC(C(=O)Nc4ncc(SCc5ncc(C(C)(C)C)o5)s4)CC3)C2)cc1)N(C)C(=O)/C(C#N)=C/c1nccs1] | Prepared as described for example A38 | 845.3 |
| A13 | 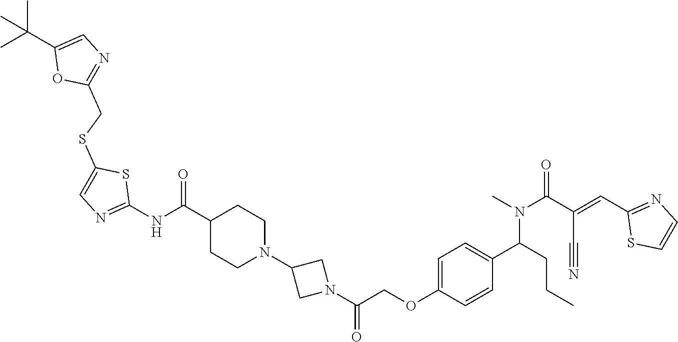 [SMILES: CCCC(c1ccc(OCC(=O)N2CC(N3CCC(C(=O)Nc4ncc(SCc5ncc(C(C)(C)C)o5)s4)CC3)C2)cc1)N(C)C(=O)/C(C#N)=C/c1nccs1] | Prepared as described for example A38 | 817.3 |
| A12 | 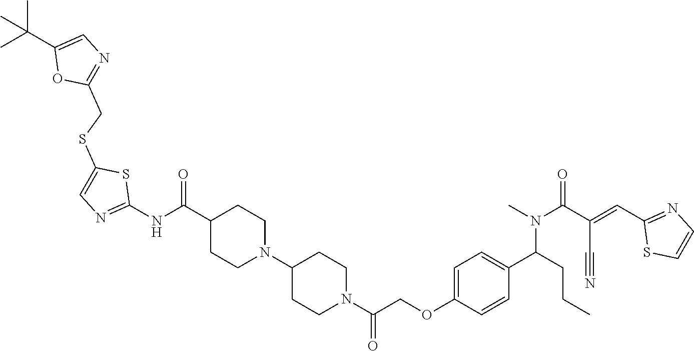 [SMILES: CCCC(c1ccc(OCC(=O)N2CCC(N3CCC(C(=O)Nc4ncc(SCc5ncc(C(C)(C)C)o5)s4)CC3)CC2)cc1)N(C)C(=O)/C(C#N)=C/c1nccs1] | Prepared as described for example A38 | 845.3 |
| A11 | 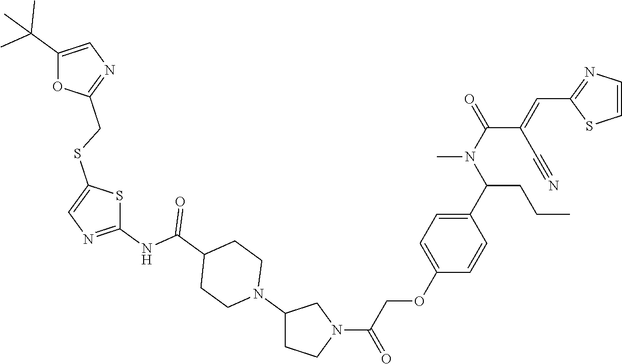 [SMILES: CCCC(c1ccc(OCC(=O)N2CCC(N3CCC(C(=O)Nc4ncc(SCc5ncc(C(C)(C)C)o5)s4)CC3)C2)cc1)N(C)C(=O)/C(C#N)=C/c1nccs1] | Prepared as described for example A38 | 831.3 |
| A10 | 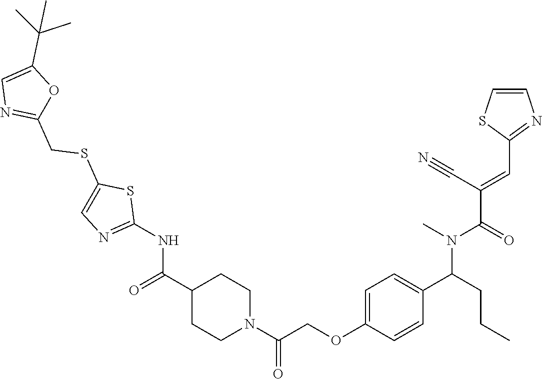 [SMILES: CCCC(c1ccc(OCC(=O)N2CCC(C(=O)Nc3ncc(SCc4ncc(C(C)(C)C)o4)s3)CC2)cc1)N(C)C(=O)/C(C#N)=C/c1nccs1] | Prepared as described for example A38 | 762.3 |

### Example A18: N-(5-(((5-(tert-butyl)oxazol-2-yl)methyl)thio)thiazol-2-yl)-1′-(2-(4-(1-(2-cyano-N-methyl-3-(thiazol-2-yl)propanamido)butyl)phenoxy)acetyl)-[1,4′-bipiperidine]-4-carboxamide

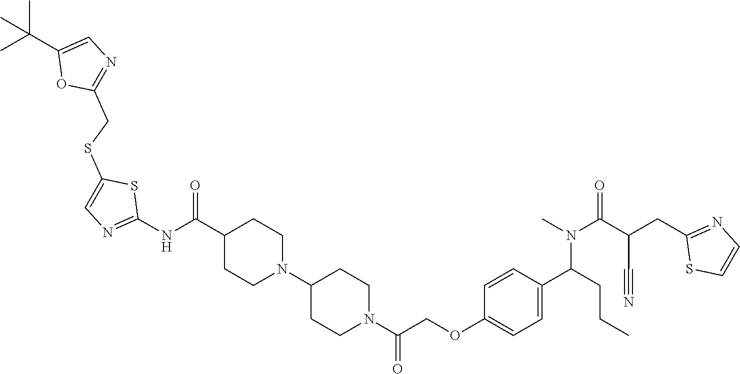 [SMILES: CCCC(c1ccc(OCC(=O)N2CCC(N3CCC(C(=O)Nc4ncc(SCc5ncc(C(C)(C)C)o5)s4)CC3)CC2)cc1)N(C)C(=O)C(C#N)Cc1nccs1]

Preparative compound A12 (14 mg, 1.0 equiv., 17 μmol) was dissolved in THF (0.2 M). Sodium triacetoxyborohydride (11 mg, 3.0 equiv., 50 μmol) was added and the reaction was stirred at rt for 5. A further portion of sodium triacetoxyborohydride (11 mg, 3.0 equiv., 50 μmol) was added and the reaction was stirred at rt for 16 h. The reaction was diluted with water and extracted three times with CH₂Cl₂. Combined organic extracts were reduced in vacuo and purified by preparative HPLC (using a gradient from 5% to 95% of acetonitrile in water containing 0.1% of formic acid over 10 minutes) to furnish the desired product in 43% yield, m/z=848.2 (M+H)⁺.

### Additional Example

### Example A30: N-(5-(((5-(tert-butyl)oxazol-2-yl)methyl)thio)thiazol-2-yl)-1′-(3-(4-(1-(2-cyano-N-methyl-3-(thiazol-2-yl)propanamido)butyl)phenyl)propanoyl)-[1,4′-bipiperidine]-4-carboxamide

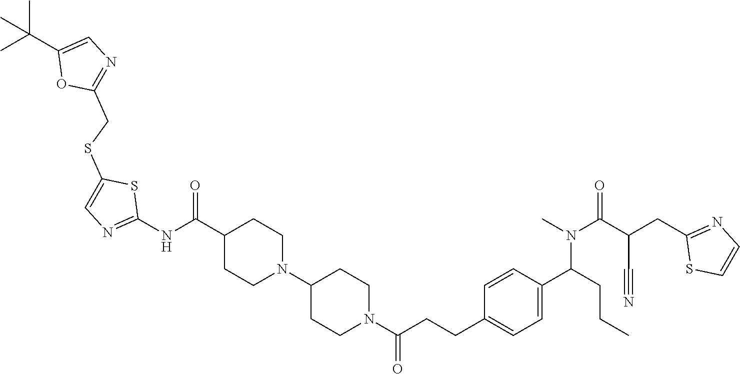 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)C(C#N)Cc1nccs1]

Prepared as described for compound A18 from preparative compound A28 in 74% yield,

m/z=845.4 (M+H)⁺.

### Example 72: tert-butyl 6-(2-oxoethoxy)-1-propyl-3,4-dihydroisoquinoline-2(1H)-carboxylate

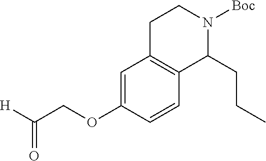 [SMILES: CCCC1c2ccc(OCC=O)cc2CCN1C(=O)OC(C)(C)C]

Prepared following general procedure 29. Obtained 97.6 mg, 65.0% yield. m/z=334.2 (M+H)⁺.

### Additional Examples

| Example | | | m/z |
|---|---|---|---|
| number | Structure | Preparation | (M + H)⁺ |
| 73 | 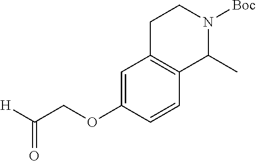 [SMILES: CC1c2ccc(OCC=O)cc2CCN1C(=O)OC(C)(C)C] | Prepared following general procedure 29 | 306.2 |
| 74 | 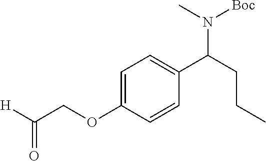 [SMILES: CCCC(c1ccc(OCC=O)cc1)N(C)C(=O)OC(C)(C)C] | Prepared following general procedure 29 | 322.2 |
| 75 | 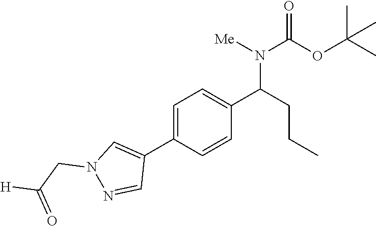 | Prepared following general procedure 29 | 372.2 |
| 76 | 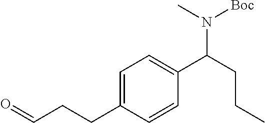 [SMILES: CCCC(c1ccc(CCC=O)cc1)N(C)C(=O)OC(C)(C)C] | Prepared following general procedure 29 | 342 [M + Na] |

### Example 77: tert-butyl 6-(2-(4-((5-(((5-(tert-butyl)oxazol-2-yl)methyl) thio)thiazol-2-yl)carbamoyl)piperidin-1-yl)ethoxy)-1-propyl-3,4-dihydroisoquinoline-2(1H)-carboxylate

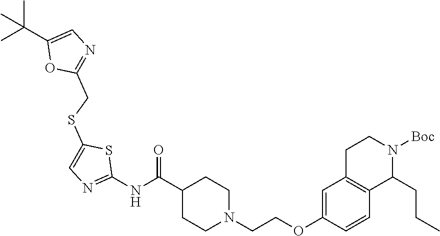 [SMILES: CCCC1c2ccc(OCCN3CCC(C(=O)Nc4ncc(SCc5ncc(C(C)(C)C)o5)s4)CC3)cc2CCN1C(=O)OC(C)(C)C]

SNS032 (55.9 mg, 1.0 equiv., 147 μmol) and 72 (49.0 mg, 1.0 equiv., 147 μmol) were suspended in THF (1.5 mL) and cooled to 0° C. sodium triacetoxyborohydride (62.3 mg, 2.0 equiv., 294 μmol) was added and all suspended solids went into solution. The reaction was warmed to room temperature and left to stir overnight. The reaction was quenched by addition of NaHCO₃ (sat. aq.) and extracted with CH₂Cl₂. The combined organic layers were washed with water and brine, dried over MgSO₄ and concentrated under reduced pressure. Purification by flash chromatography yielded the corresponding amine. Obtained 76.7 mg, 75% yield. m/z=698.3 (M+H)⁺.

This reaction protocol has been exemplified in relation to a THIQ analogue (such as those outlined in section 2.4 but is also applicable to the synthesis of N-alkylated analogues (such as those shown in 2.3).

### Additional Examples

| Example | | | m/z |
|---|---|---|---|
| number | Structure | Preparation | (M + H)⁺ |
| 78 | 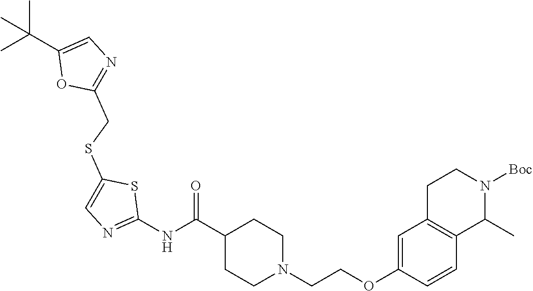 [SMILES: CC1c2ccc(OCCN3CCC(C(=O)Nc4ncc(SCc5ncc(C(C)(C)C)o5)s4)CC3)cc2CCN1C(=O)OC(C)(C)C] | Prepared as described for example 77 | 670.3 |
| 79 | 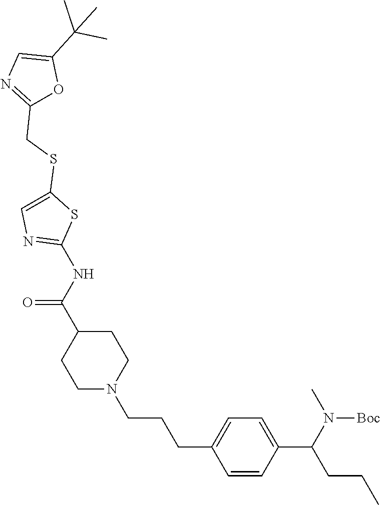 [SMILES: CCCC(c1ccc(CCCN2CCC(C(=O)Nc3ncc(SCc4ncc(C(C)(C)C)o4)s3)CC2)cc1)N(C)C(=O)OC(C)(C)C] | Prepared as described for example 77 | 684.4 |
| 80 | 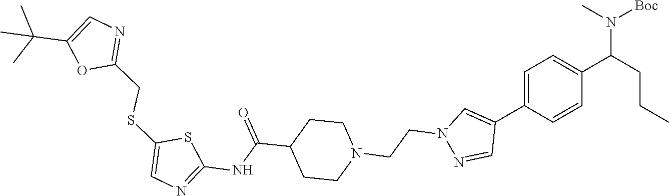 [SMILES: CCCC(c1ccc(-c2cnn(CCN3CCC(C(=O)Nc4ncc(SCc5ncc(C(C)(C)C)o5)s4)CC3)c2)cc1)N(C)C(=O)OC(C)(C)C] | Prepared as described for example 77 | 736.4 |

### Example 81: tert-butyl (1-(4-(2-(4-((5-(tert-butyl)oxazol-2-yl)methyl)thio)thiazol-2-yl)carbamoyl)-[1,4′-bipiperidin]-yl)-2-oxoethoxy)phenyl)butyl) (methyl)carbamate

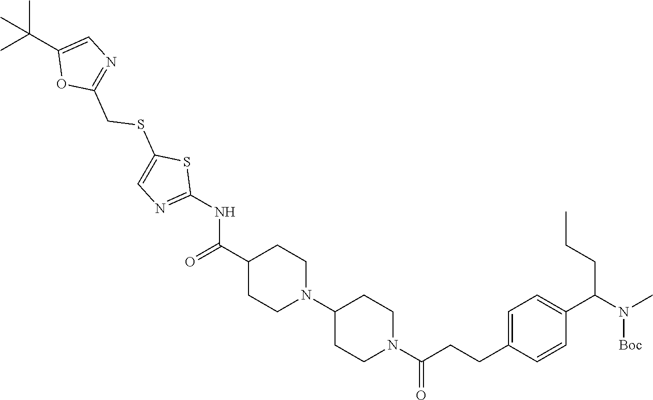 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)OC(C)(C)C]

Compound 66 (49.0 mg, 1.0 equiv., 91 μmol) and compound 41 (46.0 mg, 1.5 equiv., 140 μmol) were coupled following general procedure 31. The product was purified by flash chromatography yielding 81. Obtained 71.0 mg, 95% yield. m/z=781.4 (M+H)⁺.

| Example | | | m/z |
|---|---|---|---|
| number | Structure | Preparation | (M + H)⁺ |
| 82 | 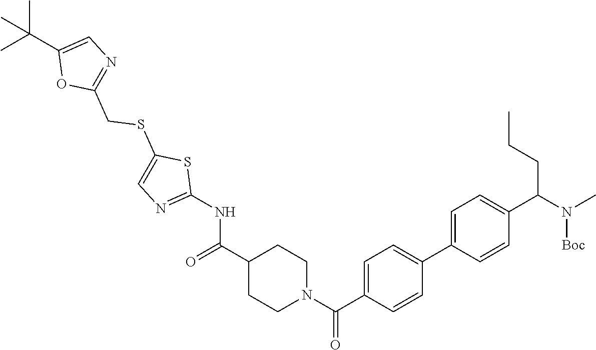 [SMILES: CCCC(c1ccc(-c2ccc(C(=O)N3CCC(C(=O)Nc4ncc(SCc5ncc(C(C)(C)C)o5)s4)CC3)cc2)cc1)N(C)C(=O)OC(C)(C)C] | Prepared as described for example 81 | 746.3 |
| 83 | 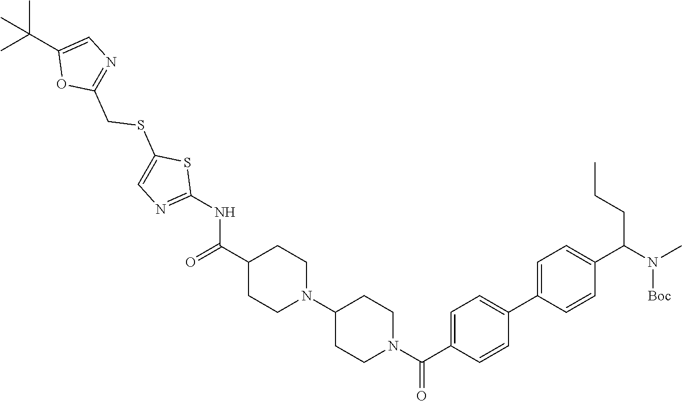 [SMILES: CCCC(c1ccc(-c2ccc(C(=O)N3CCC(N4CCC(C(=O)Nc5ncc(SCc6ncc(C(C)(C)C)o6)s5)CC4)CC3)cc2)cc1)N(C)C(=O)OC(C)(C)C] | Prepared as described for example 81 | 829.4 |
| 84 | 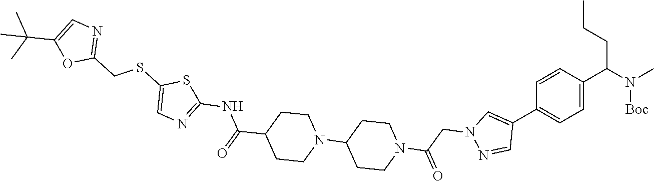 [SMILES: CCCC(c1ccc(-c2cnn(CC(=O)N3CCC(N4CCC(C(=O)Nc5ncc(SCc6ncc(C(C)(C)C)o6)s5)CC4)CC3)c2)cc1)N(C)C(=O)OC(C)(C)C] | Prepared as described for example 81 | 833.2 |
| 85 | 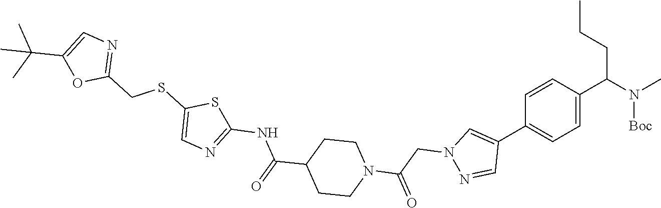 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)OC(C)(C)C] | Prepared as described for example 81 | 750.3 |

### Example A32: (E)-N-(5-(((5-(tert-butyl)oxazol-2-yl)methyl)thio)thiazol-2-yl)-1′-(3-(4-(1-(N-methyl-3-(thiazol-2-yl)acrylamido)butyl)phenyl)propanoyl)-[1,4′-bipiperidine]-4-carboxamide

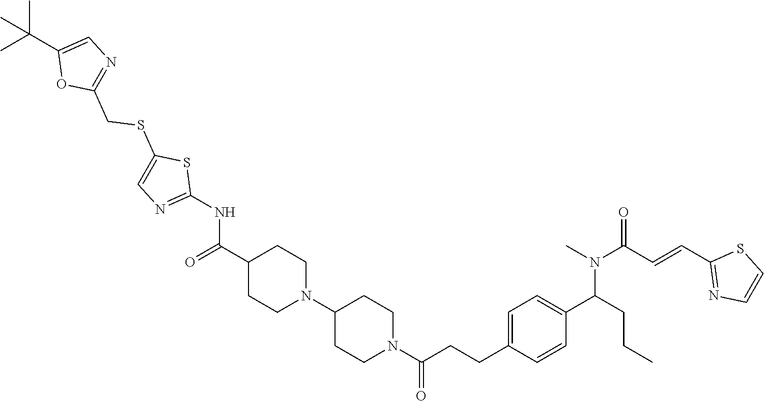 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)/C=C/c1nccs1]

Compound 81 (25.0 mg, 1.0 equiv., 32 μmol) was dissolved in CH₂Cl₂ (1 mL). 4M HCl in dioxane (0.40 mL, 50 equiv., 1.6 mmol) was added and the reaction was stirred at room temperature for 1 hour. The reaction mixture was reduced in vacuo to give the unprotected product. This was then coupled with compound 19 (7.4 mg, 1.5 equiv., 47 μmol) following general procedure 31. The product was purified by preparative HPLC (using a gradient from 5% to 95% of acetonitrile in water containing 0.1% of formic acid over 10 minutes) to furnish A32. Obtained 12.0 mg, 46% yield. m/z=818.3 (M+H)⁺.

### Example 86: N-(5-(((5-(tert-butyl)oxazol-2-yl)methyl)thio)thiazol-2-yl)-1-(2-((2-(2-cyanoacetyl)-1-propyl-1,2,3,4-tetrahydroisoquinolin-6-yl)oxy)ethyl)piperidine-4-carboxamide

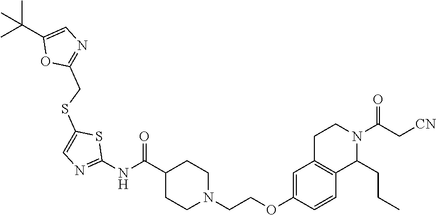 [SMILES: CCCC1c2ccc(OCCN3CCC(C(=O)Nc4ncc(SCc5ncc(C(C)(C)C)o5)s4)CC3)cc2CCN1C(=O)CC#N]

A solution of Compound 77 (76.7 mg, 1.0 equiv., 110 μmol) in CH₂Cl₂ (0.05 M) was treated with HCl (4 M in dioxane, 1.37 mL, 50 equiv., 5.5 mmol)) and the mixture was stirred for 2 h. The volatiles were evaporated under reduced pressure. The residue was dissolved in 1,4-dioxane (0.05 M), treated with Et₃N (80 μL, 4.0 equiv., 440 μmol) and 3-(3,5-dimethyl-1H-pyrazol-1-yl)-3-oxopropanenitrile (19.7 mg, 1.1 equiv., 121 μmol) and the mixture was heated to 90° C. for 2 h. The volatiles were evaporated under reduced pressure. Purification by flash chromatography yielded the desired product (56.8 mg, 77.7% yield). m/z=665.3 (M+H)⁺.

This reaction protocol has been exemplified in relation to a THIQ analogue (such as those outlined in section 2.4 but is also applicable to the synthesis of N-alkylated analogues (such as those shown in 2.3).

### Additional Examples

| Example | | | m/z |
|---|---|---|---|
| number | Structure | Preparation | (M + H)⁺ |
| 87 | 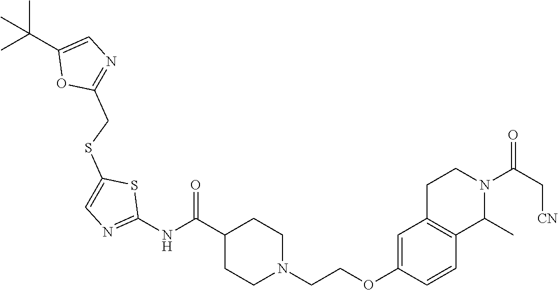 [SMILES: CC1c2ccc(OCCN3CCC(C(=O)Nc4ncc(SCc5ncc(C(C)(C)C)o5)s4)CC3)cc2CCN1C(=O)CC#N] | Prepared as described for example 86 | 637.3 |
| 88 | 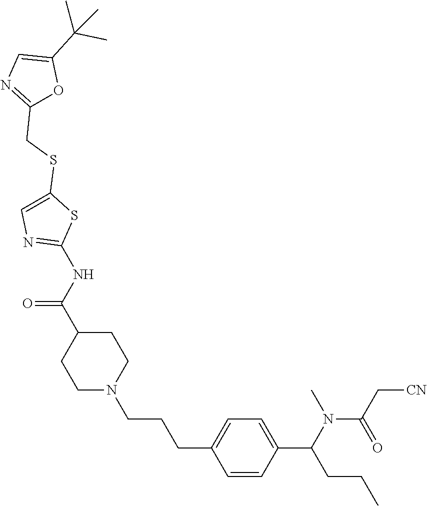 [SMILES: CCCC(c1ccc(CCCN2CCC(C(=O)Nc3ncc(SCc4ncc(C(C)(C)C)o4)s3)CC2)cc1)N(C)C(=O)CC#N] | Prepared as described for example 86 | 651.3 |
| 89 | 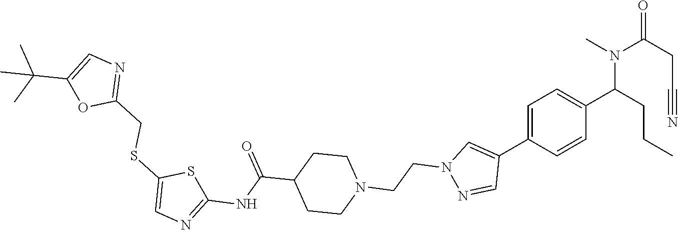 [SMILES: CCCC(c1ccc(-c2cnn(CCN3CCC(C(=O)Nc4ncc(SCc5ncc(C(C)(C)C)o5)s4)CC3)c2)cc1)N(C)C(=O)CC#N] | Prepared as described for example 86 | 703.3 |
| 90 | 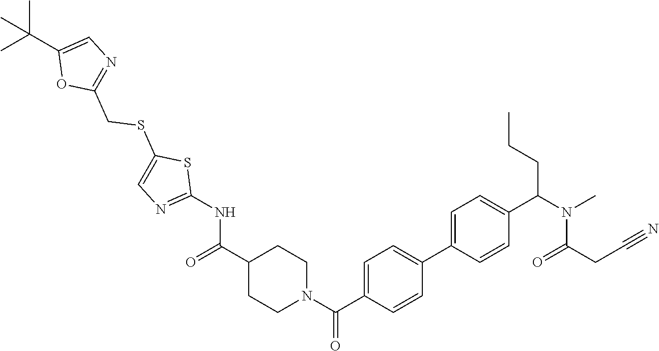 [SMILES: CCCC(c1ccc(-c2ccc(C(=O)N3CCC(C(=O)Nc4ncc(SCc5ncc(C(C)(C)C)o5)s4)CC3)cc2)cc1)N(C)C(=O)CC#N] | Prepared as described for example 86 | 713.2 |
| 91 | 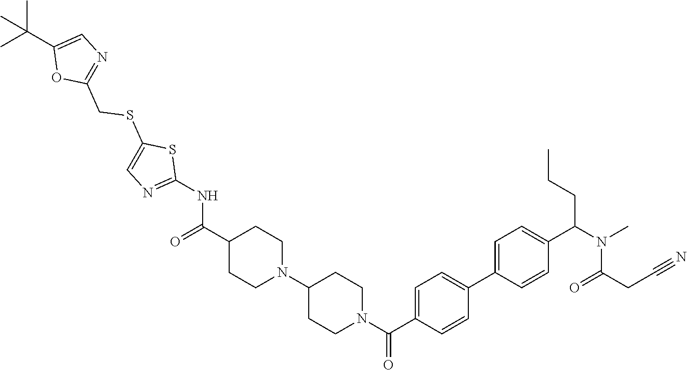 [SMILES: CCCC(c1ccc(-c2ccc(C(=O)N3CCC(N4CCC(C(=O)Nc5ncc(SCc6ncc(C(C)(C)C)o6)s5)CC4)CC3)cc2)cc1)N(C)C(=O)CC#N] | Prepared as described for example 86 | 796.5 |
| 92 | 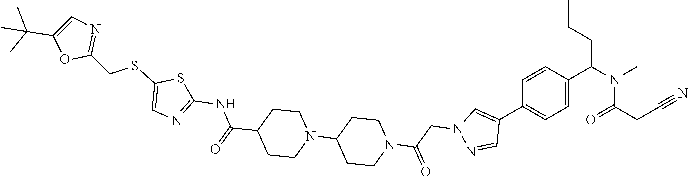 [SMILES: CCCC(c1ccc(-c2cnn(CC(=O)N3CCC(N4CCC(C(=O)Nc5ncc(SCc6ncc(C(C)(C)C)o6)s5)CC4)CC3)c2)cc1)N(C)C(=O)CC#N] | Prepared as described for example 86 | 800.4 |
| 93 | 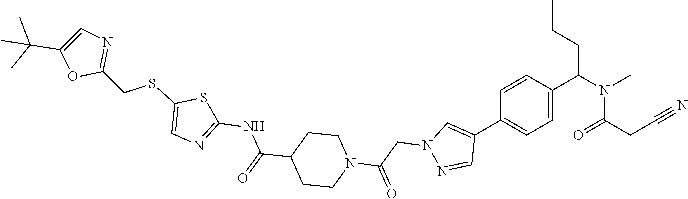 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)CC#N] | Prepared as described for example 86 | 717.3 |

### Example A35: (E)-N-(5-(((5-(tert-butyl)oxazol-2-yl)methyl)thio)thiazol-2-yl)-1-(2-((2-(2-cyano-3-(thiazol-2-yl)acryloyl)-1-propyl-1,2,3,4-tetrahydroisoquinolin-6-yl)oxy)ethyl)piperidine-4-carboxamide

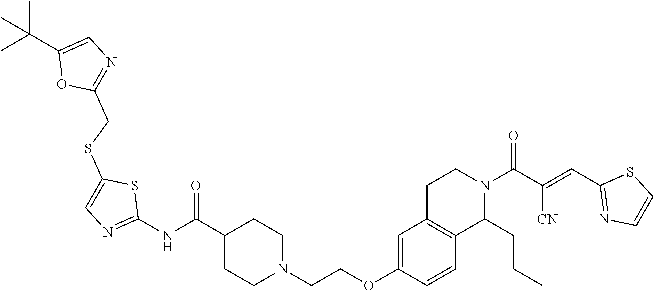 [SMILES: CCCC1c2ccc(OCCN3CCC(C(=O)Nc4ncc(SCc5ncc(C(C)(C)C)o5)s4)CC3)cc2CCN1C(=O)/C(C#N)=C/c1nccs1]

A solution of compound 86 (35 mg, 1.0 equiv., 52.6 μmol) in THF (0.1 M) was treated with thiazole-2-carbaldehyde (28 mg, 5.0 equiv., 263 μmol) and piperidine (105 μL, 2.0 equiv., 105 μmol) and the mixture was heated to 66° C. for 72 h. The volatiles were evaporated under reduced pressure. Purification by flash chromatography yielded A35 (20.9 mg, 52.2% yield). m/z=760.3 (M+H)⁺.

Again, while these general reaction protocols have been exemplified in relation to a THIQ analogue (such as those outlined in section 2.6, it is also applicable to the synthesis of N-alkylated analogues (such as those shown in 2.5).

### Additional Examples

| Example | | | m/z |
|---|---|---|---|
| number | Structure | Preparation | (M + H)⁺ |
| A37 | 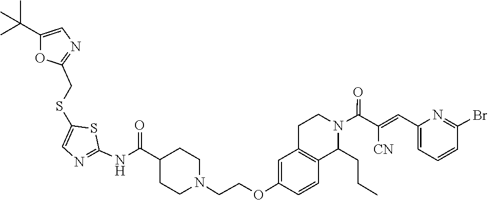 [SMILES: CCCC1c2ccc(OCCN3CCC(C(=O)Nc4ncc(SCc5ncc(C(C)(C)C)o5)s4)CC3)cc2CCN1C(=O)/C(C#N)=C/c1cccc(Br)n1] | Prepared as described for example A35 | 832.2 |
| A36 | 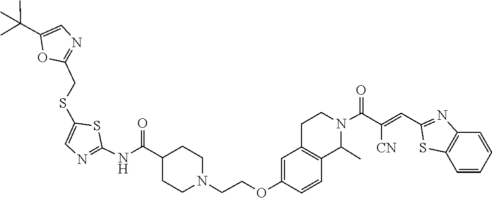 [SMILES: CC1c2ccc(OCCN3CCC(C(=O)Nc4ncc(SCc5ncc(C(C)(C)C)o5)s4)CC3)cc2CCN1C(=O)/C(C#N)=C/c1nc2ccccc2s1] | Prepared as described for example A35 | 782.3 |
| A34 | 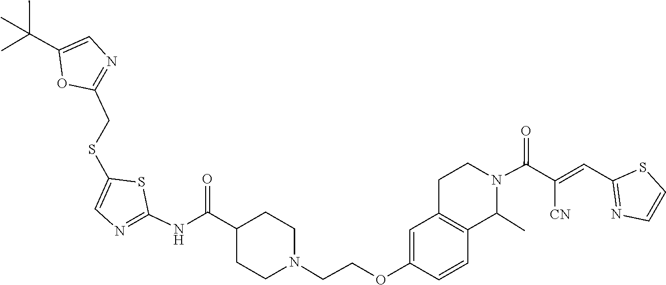 [SMILES: CC1c2ccc(OCCN3CCC(C(=O)Nc4ncc(SCc5ncc(C(C)(C)C)o5)s4)CC3)cc2CCN1C(=O)/C(C#N)=C/c1nccs1] | Prepared as described for example A35 | 732.2 |
| A33 | 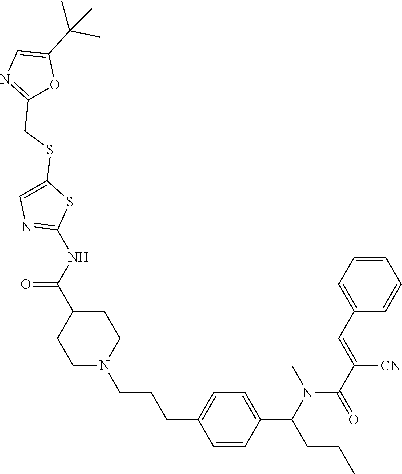 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1ccccc1] | Prepared as described for example A35 | 739.3 |
| A29 | 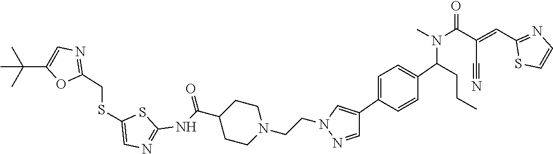 [SMILES: CCCC(c1ccc(-c2cnn(CCN3CCC(C(=O)Nc4ncc(SCc5ncc(C(C)(C)C)o5)s4)CC3)c2)cc1)N(C)C(=O)/C(C#N)=C/c1nccs1] | Prepared as described for example A35 | 798.3 |
| A26 | 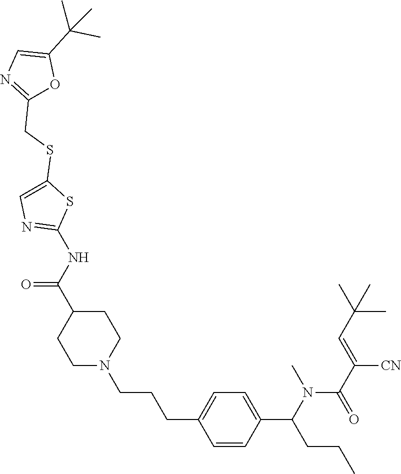 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/C(C)(C)C] | Prepared as described for example A35 | 719.4 |
| A25 | 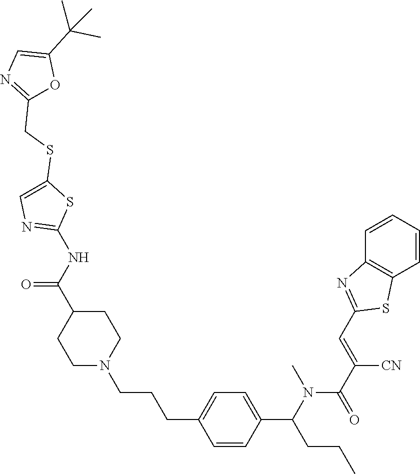 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1nc2ccccc2s1] | Prepared as described for example A35 | 796.3 |
| A24 | 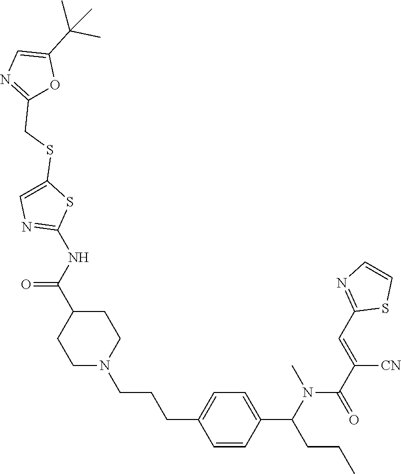 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1nccs1] | Prepared as described for example A35 | 746.3 |
| A23 | 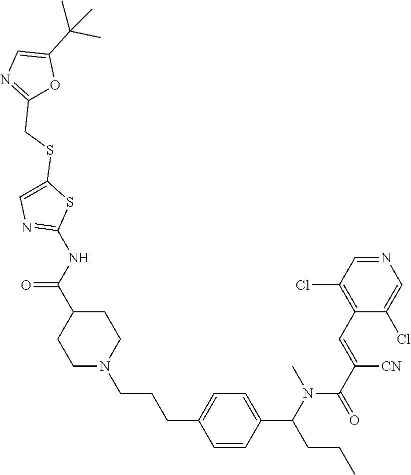 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1c(Cl)cncc1Cl] | Prepared as described for example A35 | 808.3 |
| A22 | 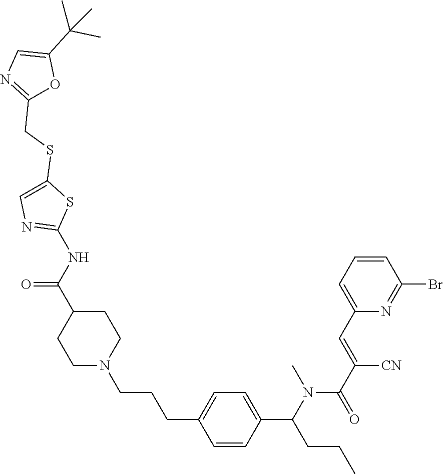 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1cccc(Br)n1] | Prepared as described for example A35 | 818.3 |
| A21 | 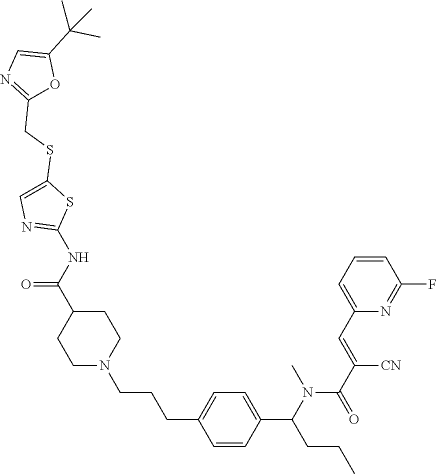 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1cccc(F)n1] | Prepared as described for example A35 | 758.3 |
| A44 | 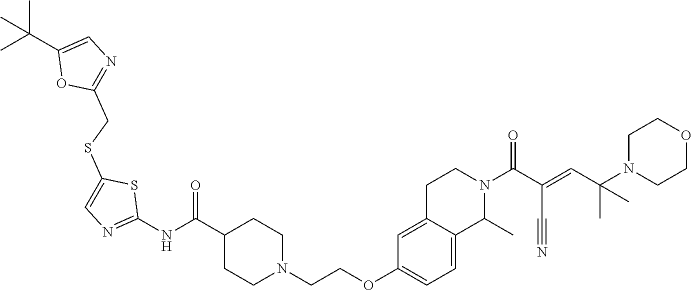 [SMILES: CC1c2ccc(OCCN3CCC(C(=O)Nc4ncc(SCc5ncc(C(C)(C)C)o5)s4)CC3)cc2CCN1C(=O)/C(C#N)=C/C(C)(C)N1CCOCC1] | Prepared as described for example A35 | 777.4 |
| A45 | 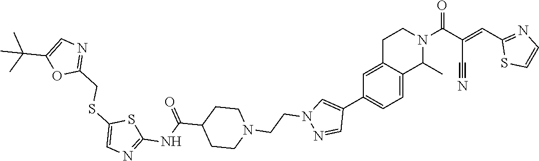 [SMILES: CC1c2ccc(-c3cnn(CCN4CCC(C(=O)Nc5ncc(SCc6ncc(C(C)(C)C)o6)s5)CC4)c3)cc2CCN1C(=O)/C(C#N)=C/c1nccs1] | Prepared as described for example A35 | 783.3 |
| A15 | 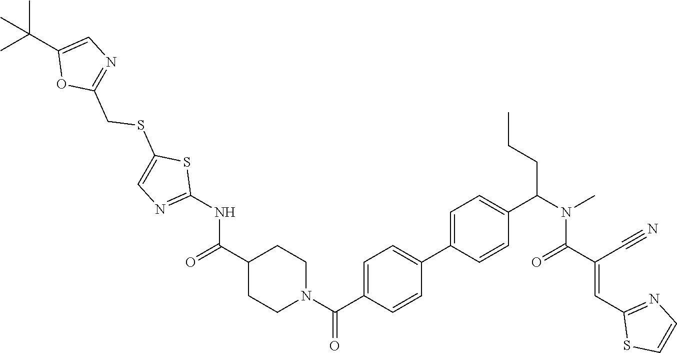 [SMILES: CCCC(c1ccc(-c2ccc(C(=O)N3CCC(C(=O)Nc4ncc(SCc5ncc(C(C)(C)C)o5)s4)CC3)cc2)cc1)N(C)C(=O)/C(C#N)=C/c1nccs1] | Prepared as described for example A35 | 808.4 |
| A17 | 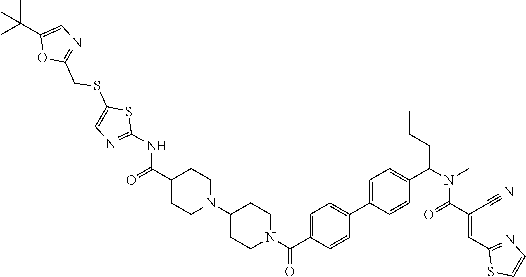 [SMILES: CCCC(c1ccc(-c2ccc(C(=O)N3CCC(N4CCC(C(=O)Nc5ncc(SCc6ncc(C(C)(C)C)o6)s5)CC4)CC3)cc2)cc1)N(C)C(=O)/C(C#N)=C/c1nccs1] | Prepared as described for example A35 | 891.3 |
| A16 | 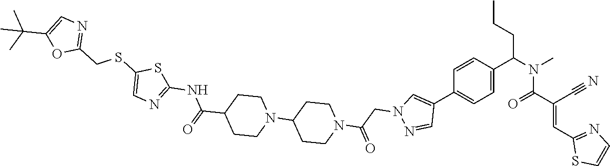 [SMILES: CCCC(c1ccc(-c2cnn(CC(=O)N3CCC(N4CCC(C(=O)Nc5ncc(SCc6ncc(C(C)(C)C)o6)s5)CC4)CC3)c2)cc1)N(C)C(=O)/C(C#N)=C/c1nccs1] | Prepared as described for example A35 | 895.4 |
| A14 | 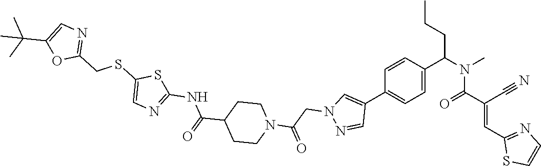 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1nccs1] | Prepared as described for example A35 | 812.3 |

### Example A46: N-[5-[(5-tert-butyloxazol-2-yl)methylsulfanyl]thiazol-2-yl]-1-[2-[4-[4-[1-[[(E)-2-cyano-3-(1-methylimidazol-2-yl)prop-2-enoyl]-methyl-amino]butyl]phenyl]pyrazol-1-yl]acetyl]piperidine-4-carboxamide

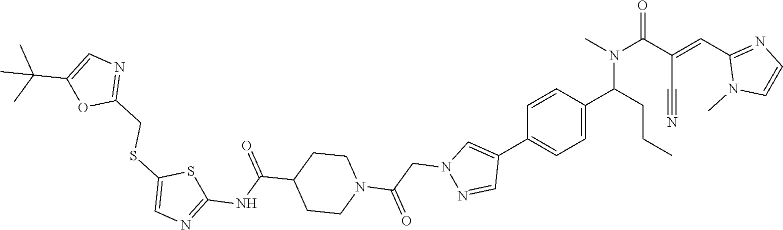

A solution of example 93 (12 mg, 1.0 equiv., 17 μmol) in EtOH (0.01M) was treated with 1-methyl-2-imidazolecarboxaldehyde (3.7 mg, 2.0 equiv., 34 μmol) and piperidine (10 μL, 2.0 equiv., 34 μmol) and the mixture was stirred at room temperature for 18 h. The volatiles were evaporated under reduced pressure. Purification by preparative HPLC (using a gradient from 5% to 95% of acetonitrile in water containing 0.1% of formic acid over 10 minutes) yielded the corresponding product (5.4 mg, 13.8% yield). m/z=810.3 (M+H)⁺

| Example | | | \|m/z |
|---|---|---|---|
| number | Structure | Preparation | (M + H)⁺ |
| A47 | 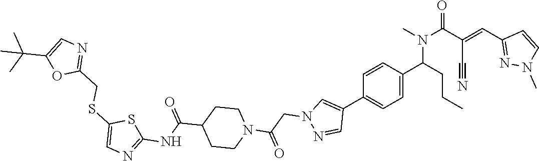 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ccn(C)n1] | Prepared as described for example A46 | 810.3 |
| A48 | 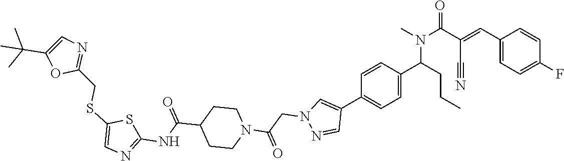 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ccc(F)cc1] | Prepared as described for example A46 | 824.3 |
| A49 | 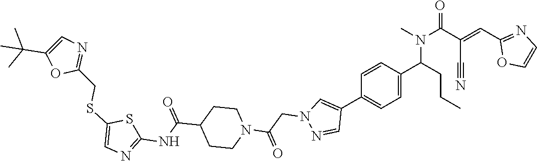 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ncco1] | Prepared as described for example A46 | 796.4 |
| A50 | 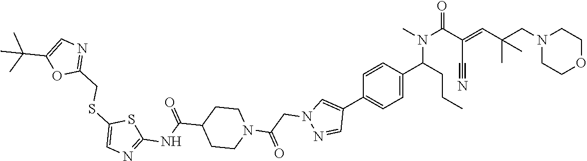 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/C(C)(C)CN1CCOCC1] | Prepared as described for example A46 | 871.4 |
| A51 | 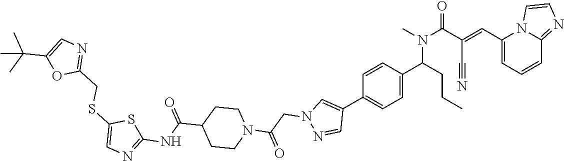 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1cccc2nccn12] | Prepared as described for example A46 | 846.2 |
| A52 | 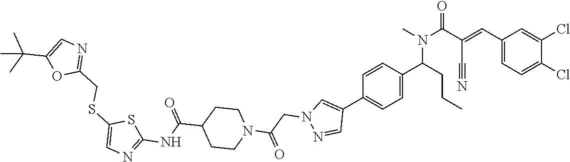 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ccc(Cl)c(Cl)c1] | Prepared as described for example A46 | 873.2 |

### Example 94—5-(((5-(tert-butyl)oxazol-2-yl)methyl)thio)thiazol-2-amine

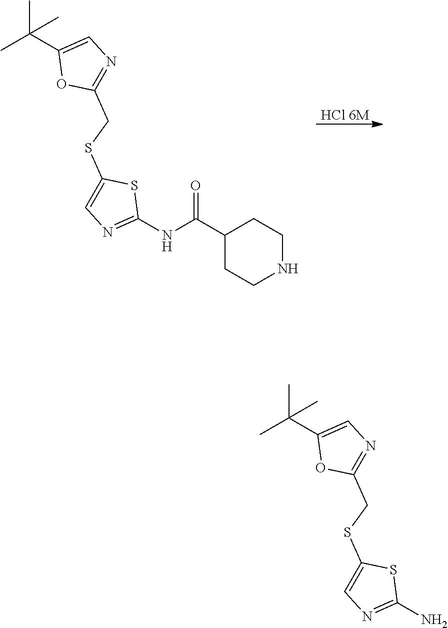 [SMILES: CC(C)(C)c1cnc(CSc2cnc(NC(=O)C3CCNCC3)s2)o1 ; CC(C)(C)c1cnc(CSc2cnc(N)s2)o1]

In a screw-top vial—SNS032 (120 mg, 1 equiv., 315 μmol) was dissolved in HCl (2.63 mL, 6 molar) and heated at 90° C. for 3 hours. LCMS indicates formation of desired product (retention time=1.74 min, M+H=270). This was neutralised with sat. NaHCO₃ and extracted three times with DCM. Combined organic were dried over MgSO₄ and reduced in vacuo to give the desired product as a off-white solid in quantitative yield. m/z=270.1 [m+H⁺].

### Example 95—tert-butyl 4-(((5-(((5-(tert-butyl)oxazol-2-yl)methyl)thio)thiazol-2-yl)amino)methyl)piperidine-1-carboxylate

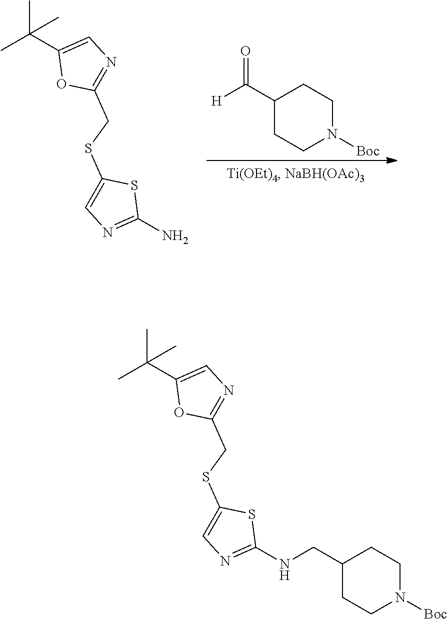 [SMILES: CC(C)(C)c1cnc(CSc2cnc(N)s2)o1 ; CC(C)(C)OC(=O)N1CCC(C=O)CC1 ; CC(C)(C)OC(=O)N1CCC(CNc2ncc(SCc3ncc(C(C)(C)C)o3)s2)CC1]

Compound 94 (52 mg, 1 Eq, 0.19 mmol) and tert-butyl 4-formylpiperidine-1-carboxylate (62 mg, 1.5 Eq, 0.29 mmol) were suspended in THF (2.5 mL) and cooled to 0° C. Sodium triacetoxyborohydride (82 mg, 2 Eq, 0.39 mmol) and titanium (IV) ethoxide (0.14 g, 0.12 mL, 65% Wt, 2 Eq, 0.39 mmol) were added and the reaction left to stir for 4 hours. A further portion of sodium triacetoxyborohydride (82 mg, 2 Eq, 0.39 mmol) was added and left to stir for 1 hour. LCMS indicates complete formation of desired product (retention time=3.25 min, M+H=467). The reaction was quenched by addition of sat. NaHCO₃ solution and left to stir for 15 mins. The reaction was filtered through a pad of Celite, washing with DCM. The filtrate was reduced in vacuo and purified by flash chromatography (4 g column, 0 to 20% MeOH in DCM) to give the desired product (90 mg, 0.19 mmol, 100%) as a yellow oil. m/z=467.2 [m+H⁺].

### Example 96—5-(((5-(tert-butyl)oxazol-2-yl)methyl)thio)-N-(piperidin-4-ylmethyl)thiazol-2-amine (hydrochloride salt)

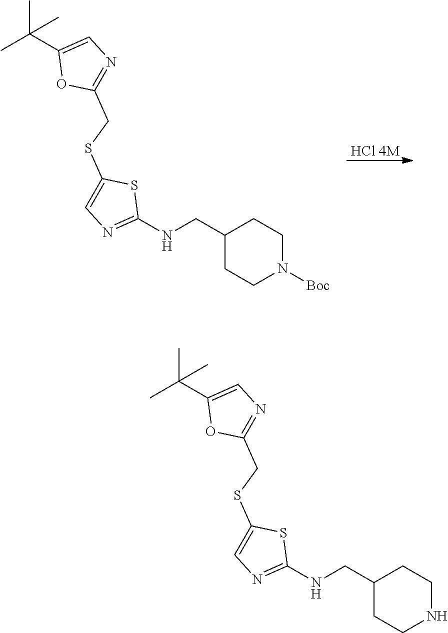 [SMILES: CC(C)(C)OC(=O)N1CCC(CNc2ncc(SCc3ncc(C(C)(C)C)o3)s2)CC1 ; CC(C)(C)c1cnc(CSc2cnc(NCC3CCNCC3)s2)o1]

Compound 95 (90 mg, 1 Eq, 0.19 mmol) was dissolved in DCM (2.5 mL), HCl in dioxane (0.35 g, 2.4 mL, 4 molar, 50 Eq, 9.6 mmol) was added and the reaction was stirred at room temperature for 1 hour. LCMS indicated consumption of the starting material and formation of the desired product (retention time=0.31 min, M+H=367). The reaction was reduced in vacuo and dried under vacuum overnight to give the desired product (85 mg, 0.19 mmol, 100%) as a yellow solid. m/z=367.2 [m+H⁺].

### Example 97—tert-butyl (1-(4-(3-(4-(((5-(((5-(tert-butyl)oxazol-2-yl)methyl)thio)thiazol-2-yl)amino)methyl)piperidin-1-yl)propyl)phenyl)butyl)(methyl)carbamate

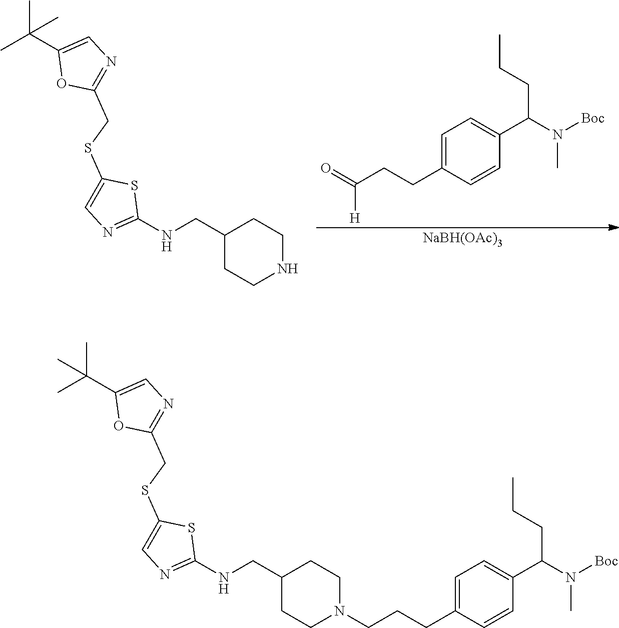

Compound 96 (85 mg, 1 Eq, 0.19 mmol) and compound 76 (0.11 g, 1.8 Eq, 0.35 mmol) were dissolved in THF (3 mL). Sodium triacetoxyborohydride (82 mg, 2 Eq, 0.39 mmol) was added and the reaction was left to stir for 4 hours. LCMS showed formation of desired product (retention time=3.08 min, M+H=670). The reaction was diluted with water and extracted three times with DCM. Combined organic extracts were reduced in vacuo and dry loaded onto silica. The reaction was purified by flash chromatography (12 g column, 0 to 20% MeOH in DCM) to give the desired product (33 mg, 49 μmol, 25%) as a yellow oil. m/z=670.3 [m+H⁺].

### Example 98—5-(((5-(tert-butyl)oxazol-2-yl)methyl)thio)-N-((1-(3-(4-(1-(methylamino)butyl)phenyl)propyl)piperidin-4-yl)methyl)thiazol-2-amine (hydrochloride salt)

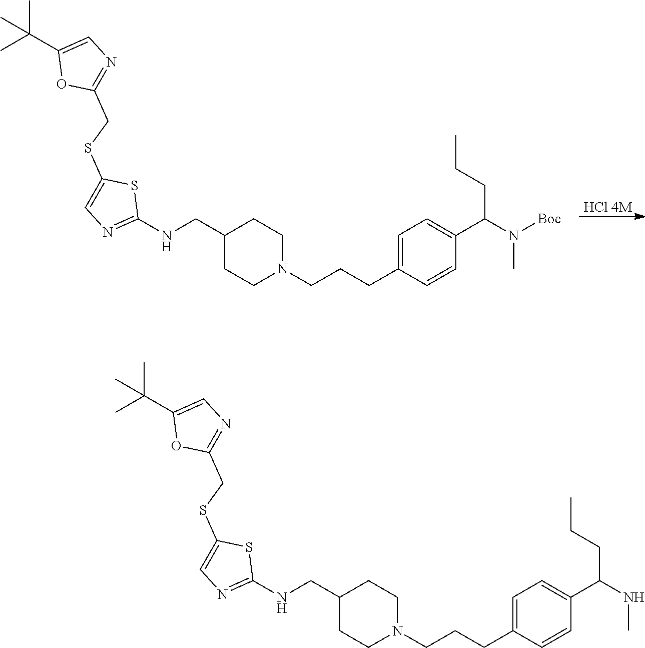 [SMILES: CCCC(c1ccc(CCCN2CCC(CNc3ncc(SCc4ncc(C(C)(C)C)o4)s3)CC2)cc1)N(C)C(=O)OC(C)(C)C ; CCCC(NC)c1ccc(CCCN2CCC(CNc3ncc(SCc4ncc(C(C)(C)C)o4)s3)CC2)cc1]

Compound 97 (33 mg, 1 Eq, 49 μmol) was dissolved in CH₂Cl₂ (2.5 mL). HCl in dioxane (90 mg, 0.62 mL, 4 molar, 50 Eq, 2.5 mmol) was added and the reaction was stirred at room temperature for 1 hour. LCMS indicated consumption of starting material and formation of desired product (retention time=0.28 min, M+H=570). The reaction was reduced in vacuo and dried under vacuum overnight to give the desired product (32 mg, 50 μmol, 100%) as a white solid. m/z=570.3 [m+H⁺].

### Example A39—3-(benzo[d]thiazol-2-yl)-N-(1-(4-(3-(4-(((5-(((5-(tert-butyl)oxazol-2-yl)methyl)thio)thiazol-2-yl)amino)methyl)piperidin-1-yl)propyl)phenyl)butyl)-2-cyano-N-methylacrylamide

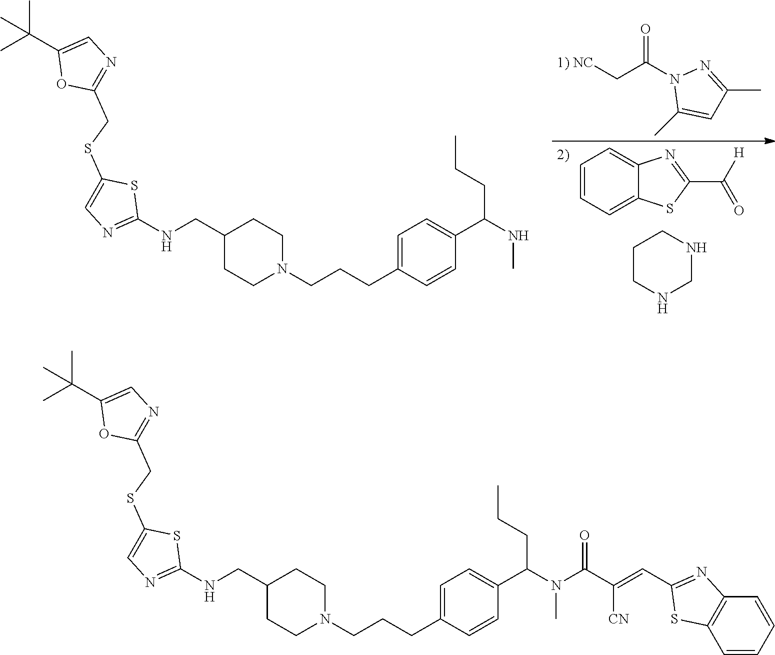

Compound 98 (32 mg, 1 Eq, 50 μmol) was suspended in 1,4-Dioxane (3 mL). 3-(3,5-dimethyl-1H-pyrazol-1-yl)-3-oxopropanenitrile (9.7 mg, 1.2 Eq, 60 μmol) and DIPEA (19 mg, 26 μL, 3 Eq, 0.15 mmol) were added and the reaction was heated at 90° C. for 4 hours. LCMS indicated formation of acylated product (retention time=2.50 min, M+H=637). The reaction was reduced in vacuo and purified by flash chromatography (4 g column, 0 to 20% MeOH in DCM) to give A39 (30 mg, 47 μmol, 95%). This was dissolved in THF (2.5 mL) and transferred to a screw-top vial. Benzo[d]thiazole-2-carbaldehyde (20 mg, 2.5 Eq, 0.12 mmol) and piperidine (2.1 mg, 2.5 μL, 0.5 Eq, 25 μmol) were added, the vial was capped and heated at 70° C. overnight. LCMS indicates formation of desired product (retention time=2.95 min, M+H=782—two peaks present in a 1:1 ratio (E/Z isomers)). A further benzo[d]thiazole-2-carbaldehyde (20 mg, 2.5 Eq, 0.12 mmol) and piperidine (2.1 mg, 2.5 μL, 0.5 Eq, 25 μmol) were added and the reaction heated at 70° C. overnight. LCMS indicated complete consumption of starting material. The reaction mixture was reduced in vacuo and dissolved in 2 mL of MeOH. The product was purified by preparative HPLC (eluting from 20% to 95% of MeCN in H₂O+0.1% formic acid) to give the desired product (14 mg, 18 μmol, 35%, 98% purity). m/z=782.3 [m+H⁺].

### Example 99: N-(1-(3-bromophenyl)propan-2-yl)acetamide

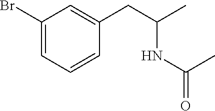

Prepared following general procedure 34. Obtained 809 mg, 49.9% yield, m/z=256.2 (M+H)⁺.

### Example 100: 6-bromo-1,3-dimethyl-3,4-dihydroisoquinoline

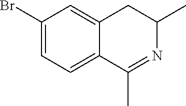

Prepared following general procedure 35. Obtained 526 mg, 69.9% yield, m/z=238.1 (M+H)⁺.

### Example 101: 6-bromo-1,3-dimethyl-1,2,3,4-tetrahydroisoquinoline

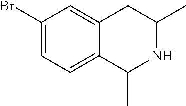

Prepared following general procedure 3. Obtained 460 mg, 86.7% yield, m/z=240.1 (M+H)⁺.

### Example 102: tert-butyl 6-bromo-1,3-dimethyl-3,4-dihydroisoquinoline-2(1H)-carboxylate

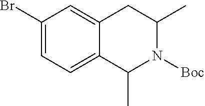 [SMILES: CC1Cc2cc(Br)ccc2C(C)N1C(=O)OC(C)(C)C]

Prepared following general procedure 5. Obtained 486 mg, 74.6% yield, m/z=284.2 ((M-tBu)+H)⁺.

### Example 103: tert-butyl (E)-6-(3-ethoxy-3-oxoprop-1-en-1-yl)-1,3-dimethyl-3,4-dihydroisoquinoline-2(1H)-carboxylate

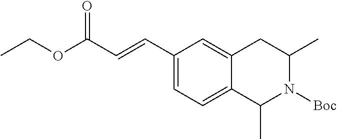 [SMILES: CCOC(=O)/C=C/c1ccc2c(c1)CC(C)N(C(=O)OC(C)(C)C)C2C]

Prepared following general procedure 27. Obtained 313 mg, 60.9% yield, m/z=304.3 ((M-tBu)+H)⁺.

### Example 104: tert-butyl 6-(3-ethoxy-3-oxopropyl)-1,3-dimethyl-3,4-dihydroisoquinoline-2(1H)-carboxylate

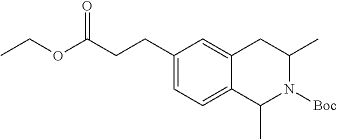 [SMILES: CCOC(=O)CCc1ccc2c(c1)CC(C)N(C(=O)OC(C)(C)C)C2C]

Prepared following general procedure 28. Obtained 311 mg, 98.5% yield, m/z=362.4 ((M)+H)⁺.

### Example 105: 3-(2-(tert-butoxycarbonyl)-1,3-dimethyl-1,2,3,4-tetrahydroisoquinolin-6-yl)propanoic acid

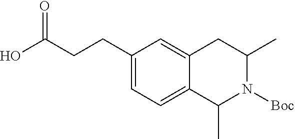 [SMILES: CC1Cc2cc(CCC(=O)O)ccc2C(C)N1C(=O)OC(C)(C)C]

Prepared following general procedure 7. Obtained 120 mg, 86.7% yield, m/z=362.4 ((M)+H)⁺.

### Example 106: 3-(1,3-dimethyl-1,2,3,4-tetrahydroisoquinolin-6-yl)propanoic acid

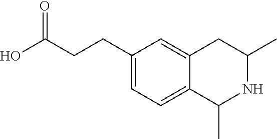 [SMILES: CC1Cc2cc(CCC(=O)O)ccc2C(C)N1]

Prepared following general procedure 8. Obtained 70.1 mg, 72.1% yield, m/z=234.2 ((M)+H)⁺.

### Example 107: 3-(2-(2-cyanoacetyl)-1,3-dimethyl-1,2,3,4-tetrahydroisoquinolin-6-yl)propanoic acid

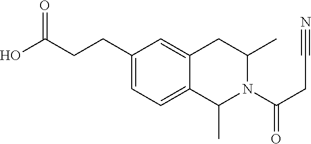 [SMILES: CC1Cc2cc(CCC(=O)O)ccc2C(C)N1C(=O)CC#N]

Prepared following general procedure 9. Obtained 70.1 mg, 72.1% yield, m/z=301.3 ((M)+H)⁺.

### Example 108: (E)-3-(2-(2-cyano-3-(thiazol-2-yl)acryloyl)-1,3-dimethyl-1,2,3,4-tetrahydroisoquinolin-6-yl)propanoic acid

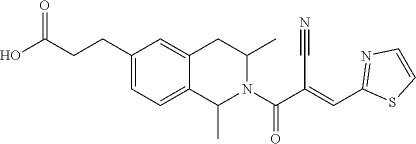 [SMILES: CC1Cc2cc(CCC(=O)O)ccc2C(C)N1C(=O)/C(C#N)=C/c1nccs1]

Prepared following general procedure 10. Obtained 35.7 mg, 43.2% yield, m/z=396.3 ((M)+H)⁺.

### Example 109—N-(2-bromophenethyl)acetamide

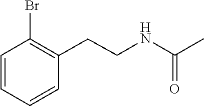

Prepared following general procedure 1. Obtained 60 g, 99% yield, m/z=242.1 [M+H]⁺ 244.1 [M+H]⁺, 1H NMR (400 MHz, DMSO-d₆): 7.96 (s, 1H), 7.59 (d, J=7.6 Hz, 1H), 7.36-7.32 (m, 2H), 7.19-7.16 (m, 1H), 3.30-3.24 (m, 2H), 2.83 (t, J=6.8 Hz, 2H), 1.80 (s, 3H).

### Example 110—5-bromo-1-methyl-3,4-dihydroisoquinoline

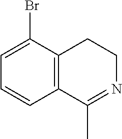

Prepared following general procedure 35. Obtained 25 g, 45% yield, m/z=224.1 [M+H]⁺ 226.0 [M+H]⁺, ¹H NMR (400 MHz, DMSO-d₆): 7.69 (d, J=0.8 Hz, 1H), 7.59 (d, J=7.6 Hz, 1H), 7.30 (t, J=8.0 Hz, 1H), 3.59-3.55 (m, 2H), 2.69 (t, J=7.2 Hz, 2H), 2.31 (s, 3H).

### Example 111—5-bromo-1-methyl-1,2,3,4-tetrahydroisoquinoline

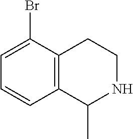

Prepared following general procedure 3. Obtained 14 g, 93% yield, m/z=226.1 [M+H]⁺ 228.2 [M+H]⁺, ¹H NMR (400 MHz, DMSO-d₆): 7.60 (d, J=6.8 Hz, 1H), 7.21-7.17 (m, 1H), 7.12 (t, J=8.0 Hz, 1H), 3.96-3.91 (m, 1H), 3.13-3.11 (m, 1H), 3.11-3.09 (m, 1H), 2.86-2.81 (m, 1H), 2.68-2.62 (m, 2H), 1.33 (d, J=6.8 Hz, 3H).

### Example 112—tert-butyl 5-bromo-1-methyl-3,4-dihydroisoquinoline-2(1H)-carboxylate

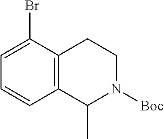 [SMILES: CC1c2cccc(Br)c2CCN1C(=O)OC(C)(C)C]

Prepared following general procedure 5. Obtained 15 g, 72% yield, m/z=226.1 [M−100+H]⁺228.0 [M−100+H]⁺, ¹H NMR (400 MHz, DMSO-d₆): 7.49 (d, J=7.2 Hz, 1H), 7.29 (d, J=7.6 Hz, 1H), 7.16 (t, J=7.6 Hz, 1H), 5.12 (s, 1H), 4.04 (s, 1H), 3.21-3.16 (m, 1H), 2.77 (m, 1H), 2.63-2.59 (m, 1H), 1.47 (m, 12H).

### Example 113—N-(3-bromophenethyl)acetamide

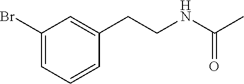

Prepared following general procedure 1. Obtained 60 g, 95% yield, ¹H NMR (400 MHz, DMSO-d₆): 7.91 (s, 1H), 7.42-7.39 (m, 1H), 7.28-7.21 (m, 1H), 3.27 (q, J=7.2 Hz, 2H), 2.70 (t, J=7.2 Hz, 2H), 1.78 (s, 3H).

### Example 114—6-bromo-1-methyl-3,4-dihydroisoquinoline

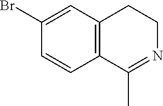

Prepared following general procedure 35. Obtained 37 g, 64% yield, m/z=224.0 [M+H]⁺ 225.9 [M+H]⁺, ¹H NMR (400 MHz, DMSO-d₆): 7.55-7.47 (m, 3H), 3.52 (t, J=2.8 Hz, 2H), 2.65 (t, J=7.2 Hz, 2H), 2.29 (s, 3H).

### Example 115—6-bromo-1-methyl-1,2,3,4-tetrahydroisoquinoline

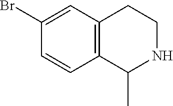

Prepared following general procedure 3. Obtained 25 g, 75% yield, m/z=226.1 [M+H]⁺ 228.2 [M+H]⁺, ¹H NMR (400 MHz, DMSO-d₆): 7.30-7.26 (m, 2H), 7.11 (d, J=8.4 Hz, 1H), 3.88 (q, J=6.4 Hz, 1H), 3.33-3.04 (m, 1H), 2.79-2.71 (m, 3H), 2.59-2.51 (m, 1H), 1.31 (d, J=6.80 Hz, 3H).

### Example 116—tert-butyl 6-bromo-1-methyl-3,4-dihydroisoquinoline-2(1H)-carboxylate

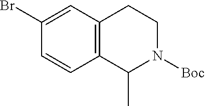 [SMILES: CC1c2ccc(Br)cc2CCN1C(=O)OC(C)(C)C]

Prepared following general procedure 5. Obtained 12 g, 92% yield, m/z=226.1 [M−100+H]⁺

### Example 117—N-(4-bromophenethyl)acetamide

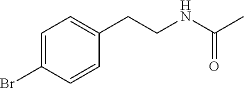

Prepared following general procedure 1. Obtained 60 g, 96% yield, m/z=242.1 [M+H]⁺, 244.1 [M+H]⁺, ¹H NMR (400 MHz, DMSO-d₆): 7.89 (s, 1H), 7.47 (d, J=2.4 Hz, 2H), 7.17 (d, J=2.4 Hz, 2H), 3.24 (q, J=5.6 Hz, 2H), 2.67 (t, J=7.2 Hz, 2H), 1.77 (s, 3H).

### Example 118—7-bromo-1-methyl-3,4-dihydroisoquinoline

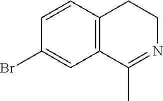

Prepared following general procedure 35. Obtained 45 g, 81% yield, m/z=224.1 [M+H]⁺ 226.0 [M+H]⁺, ¹H NMR (400 MHz, DMSO-d₆): 7.70 (s, 1H), 7.59 (d, J=6.0 Hz, 1H), 7.22 (d, J=8.0 Hz, 1H), 3.54-3.51 (m, 2H), 2.60 (t, J=7.2 Hz, 2H), 2.30 (s, 3H).

### Example 119—7-bromo-1-methyl-1,2,3,4-tetrahydroisoquinoline

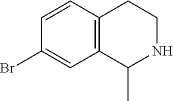

Prepared following general procedure 3. ¹H NMR (400 MHz, DMSO-d₆): 7.32 (s, 1H), 7.26 (d, J=0.4 Hz, 1H), 7.01 (d, J=8.4 Hz, 1H), 3.94-3.89 (m, 1H), 3.09-3.04 (m, 1H), 2.81-2.75 (m, 1H), 2.72-2.66 (m, 1H), 2.64-2.59 (m, 1H), 2.52-2.50 (m, 1H), 1.32 (d, J=6.8 Hz, 3H).

### Example 120—tert-butyl 7-bromo-1-methyl-3,4-dihydroisoquinoline-2(1H)-carboxylate

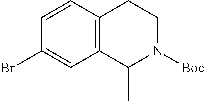 [SMILES: CC1c2cc(Br)ccc2CCN1C(=O)OC(C)(C)C]

Prepared following general procedure 5. Obtained 42 g, 92% yield, m/z=226.1 [M−100+H]⁺228.1 [M−100+H]⁺, ¹H NMR (400 MHz, DMSO-d₆): 7.48 (s, 1H), 7.34 (d, J=2.0 Hz, 1H), 7.11 (d, J=8.4 Hz, 1H), 3.97-3.92 (m, 1H), 3.16-3.09 (m, 1H), 2.73-2.68 (m, 2H), 1.47-1.36 (m, 12H).

### Example 121—tert-butyl 6-(3-((tert-butyldimethylsilyl)oxy)prop-1-yn-1-yl)-1-methyl-3,4-dihydro isoquinoline-2(1H)-carboxylate

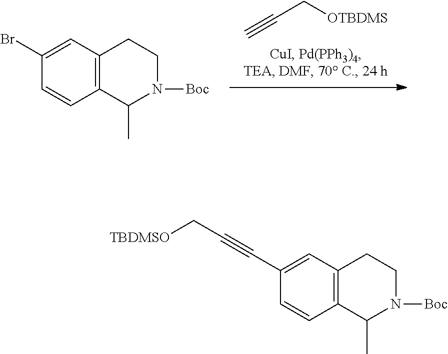 [SMILES: C#CCO[Si](C)(C)C(C)(C)C ; CC1c2ccc(Br)cc2CCN1C(=O)OC(C)(C)C ; CC1c2ccc(C#CCO[Si](C)(C)C(C)(C)C)cc2CCN1C(=O)OC(C)(C)C]

To a stirred solution of compound 116 (5 g, 1.0 eq, 15.33 mmol) and tert-butyldimethyl(prop-2-yn-1-yloxy)silane (3.92 g, 1.5 eq, 22.99 mmol) in DMF (50 ml) was added copper(I) iodide (0.876 g, 0.3 eq, 4.60 mmol), TEA (6.65 ml, 3.0 eq, 46.0 mmol) and the reaction mixture was degassed for 15 min with nitrogen. Pd(PPh₃)₄ (1.77 g, 0.1 eq, 1.53 mmol) added to the reaction mixture which was stirred at 70° C. for 16 h. The reaction mixture was cooled to RT, quenched with ice-water, diluted with EtOAc and layers were separated. The aqueous layer was extracted with EtOAc and the combined organic layers were washed with brine, dried (Na₂SO₄) and filtered. The organic layers were concentrated in vacuo to give the crude product which was purified by flash chromatography (5 to 10% EtOAc in n-hexane) to give the desired product 121 (5 g, 12.03 mmol, 78% yield) as brown liquid.

¹H NMR (400 MHz, CDCl₃) 7.31-7.29 (m, 1H), 7.25-7.28 (m, 1H), 7.08-7.00 (m, 1H), 5.24-5.05 (m, 1H), 4.55 (s, 1H), 4.20-4.03 (m, 2H), 2.88-2.07 (m, 2H), 1.43-1.26 (m, 12H), 0.93 (s, 9H), 0.17 (s, 6H),

### Example 122—tert-butyl 6-(3-ethoxy-3-oxopropyl)-1-methyl-3,4-dihydroisoquinoline-2(1H)-carboxylate

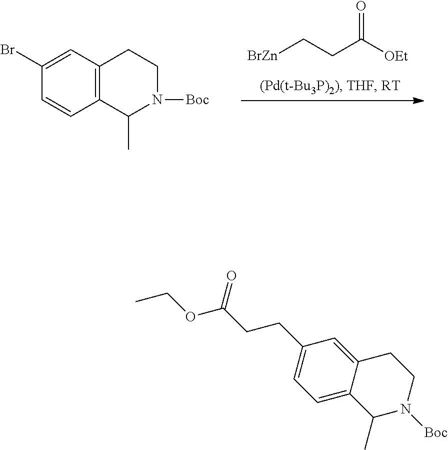

A solution of compound 116 (2.0 g, 1.0 equiv., 6.13 mmol) in THF (30 ml) was degassed with nitrogen for 10 min and Pd(tBu₃P)₂ (0.31 g, 0.1 equiv., 0.61 mmol) was added and again degassed for 10 min. (3-ethoxy-3-oxopropyl)zinc(II) bromide (30.7 ml, 2.5 equiv., 15.33 mmol) was added to the reaction mixture and the reaction was stirred at RT for 16 h. The reaction was diluted with water and extracted with EtOAc. The combined organic layer was dried (Na₂SO₄), filtered and concentrated in vacuo. The crude compound was purified by flash chromatography (10-15% EtOAc in n-hexane) to isolate 122 (1.7 g, 4.89 mmol, 80% yield).

### Example 123—3-(2-(tert-butoxycarbonyl)-1-methyl-1,2,3,4-tetrahydroisoquinolin-6-yl)propanoic acid

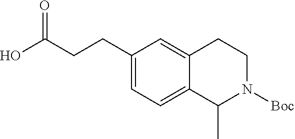 [SMILES: CC1c2ccc(CCC(=O)O)cc2CCN1C(=O)OC(C)(C)C]

Prepared following general procedure 7. Obtained 2.3 g, m/z=220.1 [M+H-100]⁺

### Example 124—tert-butyl 6-(3-(methoxy(methyl)amino)-3-oxopropyl)-1-methyl-3,4-dihydroisoquinoline-2(1H)-carboxylate

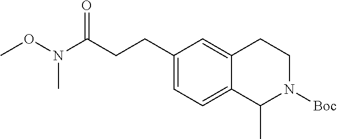 [SMILES: CON(C)C(=O)CCc1ccc2c(c1)CCN(C(=O)OC(C)(C)C)C2C]

Prepared following general procedure 31. Crude material obtained 2.4 g, 77% yield, m/z 263.2 [M+H-100]⁺

### Example 125—tert-butyl 1-methyl-6-(3-oxopropyl)-3,4-dihydroisoquinoline-2(1H)-carboxylate

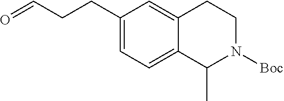 [SMILES: CC1c2ccc(CCC=O)cc2CCN1C(=O)OC(C)(C)C]

Prepared following general procedure 32. Crude material obtained 0.53 g, 64% yield, m/z 204.2 [M+H-100]⁺

### Example 126—tert-butyl (E)-7-(3-ethoxy-3-oxoprop-1-en-1-yl)-1-methyl-3,4-dihydroisoquinoline-2(1H)-carboxylate

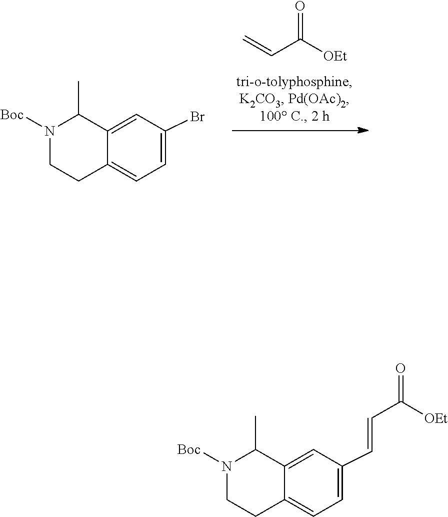

To a degassed solution of compound 120 (7 g, 1.0 equiv., 21.5 mmol) in DMF (50 ml) was added ethyl acrylate (4.57 ml, 2.0 equiv. 42.9 mmol) and tri-o-tolylphosphine (1.31 g, 0.2 equiv., 4.29 mmol) and K₂CO₃ (8.9 g, 3.0 equiv., 64.4 mmol) followed by Pd(OAc)₂ (0.48 g, 0.1 equiv., 2.14 mmol) at RT. The reaction mixture was stirred at 100° C. for 16 h then was filtered through celite. The resulting filtrate was diluted with water and extracted with EtOAc. The organic layers were combined and washed with ice cold water, brine, then dried (Na₂SO₄) and concentrated in vacuo. The resulting residue was purified by flash chromatography (8-10% EtOAc in hexane) to afford 126 (5.7 g, 16.5 mmol, 77% yield).

LCMS m/z=246.3 [M+H-100]⁺

¹H NMR (400 MHz, DMSO-d₆): δ 7.63-7.51 (m, 2H), 7.50 (d, J=1.6 Hz, 1H), 7.19 (d, J=8.0 Hz, 1H), 6.64 (m, 1H), 5.10 (br s, 1H), 4.20 (q, J=7.2 Hz, 2H), 4.05-4.03 (m, 1H), 3.13-3.12 (m, 1H), 2.78 (t, J=2.4 Hz, 2H), 1.44-1.28 (m, 12H), 1.25 (t, J=4.8 Hz, 3H).

### Example 127—tert-butyl 7-(3-ethoxy-3-oxopropyl)-1-methyl-3,4-dihydroisoquinoline-2(1H)-carboxylate

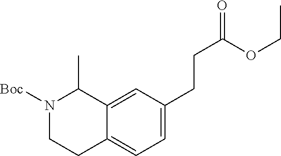 [SMILES: CCOC(=O)CCc1ccc2c(c1)C(C)N(C(=O)OC(C)(C)C)CC2]

To a stirred solution of compound 126 (5.7 g, 1.0 equiv., 16.5 mmol) in EtOH (50 ml) at 0° C. was added NiCl₂·6H₂O (1.96 g, 0.5 equiv., 8.25 mmol) and NaBH₄ (1.87 g, 3.0 equiv., 49.5 mmol). The reaction mixture was stirred at RT for 2 hr then concentrated in vacuo to remove the EtOH. The resulting residue was quenched with ice cold water and extracted with EtOAc. The organic layers were washed with brine, dried (Na₂SO₄) and concentrated in vacuo to afford 127 (5 g, 14.39 mmol, 87% yield)).

LCMS m/z: 248.3 (M+H-100)

¹H NMR (400 MHz, DMSO-d₆): 7.06-6.99 (m, 3H), 5.01 (br s, 1H), 4.06 (q, J=3.2 Hz, 2H), 4.03-3.97 (m, 1H), 3.13-3.12 (m, 1H), 2.78 (t, J=5.6 Hz, 2H), 2.73 (t, J=5.6 Hz, 2H), 2.68 (t, J=2.0 Hz, 2H), 1.43-1.24 (m, 12H), 1.18 (t, J=7.2 Hz, 3H),

### Example 128—3-(2-(tert-butoxycarbonyl)-1-methyl-1,2,3,4-tetrahydroisoquinolin-7-yl)propanoic acid

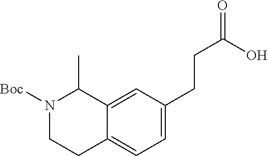 [SMILES: CC1c2cc(CCC(=O)O)ccc2CCN1C(=O)OC(C)(C)C]

Prepared following general procedure 7. Obtained 3.5 g, m/z=318.2 [M−H]⁻; ¹H NMR (400 MHz, DMSO-d₆): 12.10 (br s, 1H), 7.06-6.99 (m, 3H), 5.05 (br s, 1H), 4.04 (t, J=7.2 Hz, 1H), 3.13-3.12 (m, 1H), 2.78 (t, J=5.6 Hz, 2H), 2.73 (t, J=5.6 Hz, 2H), 2.68 (t, J=2.0 Hz, 2H), 1.43-1.24 (m, 12H),

### Example 129—tert-butyl 7-(3-(methoxy(methyl)amino)-3-oxopropyl)-1-methyl-3,4-dihydroisoquinoline-2(1H)-carboxylate

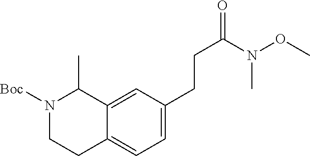 [SMILES: CON(C)C(=O)CCc1ccc2c(c1)C(C)N(C(=O)OC(C)(C)C)CC2]

Prepared following general procedure 31. Flash chromatography (0-50% EtOAc in n-hexane) obtained 3.4 g, 86% yield, m/z 263.2 [M+H-100]⁺; ¹H NMR (400 MHz, DMSO-ds) 7.06-7.02 (m, 3H), 5.03 (br s, 1H), 4.04 (t, J=7.2 Hz, 1H), 3.61 (s, 3H), 3.13-3.12 (m, 1H), 3.08 (s, 3H), 2.79-2.70 (m, 4H), 2.68 (t, J=2.0 Hz, 2H), 1.43 (s, 9H), 1.36 (d, J=4.8 Hz, 3H).

### Example 130—1-methyl-7-(3-oxopropyl)-3,4-dihydroisoquinoline-2 (1H)-carboxylate

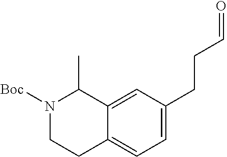 [SMILES: CC1c2cc(CCC=O)ccc2CCN1C(=O)OC(C)(C)C]

Prepared following general procedure 32. Crude material obtained 0.78 g, 99% yield, m/z 204.2 [M+H-100]⁺

### Example 131—tert-butyl (E)-5-(3-ethoxy-3-oxoprop-1-en-1-yl)-1-methyl-3,4-dihydroisoquinoline-2(1H)-carboxylate

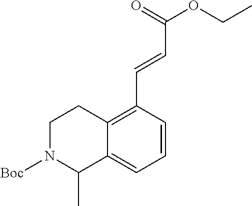 [SMILES: CCOC(=O)/C=C/c1cccc2c1CCN(C(=O)OC(C)(C)C)C2C]

To a nitrogen degassed stirred solution of compound 112 (5.0 g, 1.0 equiv., 15.33 mmol) in DMF (50 ml) was added ethyl acrylate (3.07 g, 2.0 equiv., 30.7 mmol) and tri-o-tolylphosphine (0.933 g, 0.2 equiv., 3.07 mmol) and K₂CO₃ (6.35 g, 3.0 equiv., 46.0 mmol) followed by Pd(OAc)₂ (0.344 g, 0.1 equiv., 1.533 mmol). The reaction mixture was stirred at 100° C. for 16 hr then was filtered through celite. The filtrate was diluted with water and extracted with EtOAc. The combined organic layers were washed with ice cold water, brine, dried (Na₂SO₄), concentrated in vacuo. The resulting residue was purified by flash chromatography (20-30% in EtOAc in n-hexane) to get 131 (3.85 g, 11.15 mmol, 72.7% yield).

¹H NMR (400 MHz, DMSO-d₆) 7.85 (d, J=15.8 Hz, 1H), 7.61 (d, J=6.8 Hz, 1H), 7.18-7.37 (m, 2H), 6.49 (d, J=15.8 Hz, 1H), 4.97-5.21 (m, 1H), 4.20 (q, J=7.1 Hz, 2H), 3.93-4.11 (m, 1H), 3.06-3.27 (m, 1H), 2.75-2.94 (m, 2H), 1.34-1.51 (m, 12H), 1.27 (t, J=7.1 Hz, 3H).

### Example 132—tert-butyl 5-(3-ethoxy-3-oxopropyl)-1-methyl-3,4-dihydroisoquinoline-2(1H)-carboxylate

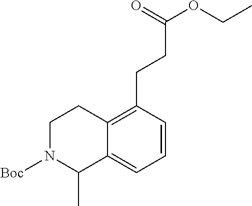 [SMILES: CCOC(=O)CCc1cccc2c1CCN(C(=O)OC(C)(C)C)C2C]

To a stirred solution of 131 (2 g, 1.0 equiv., 5.79 mmol) in EtOH (25 ml) at 0° C. was added NiCl₂·6H₂O (0.69 g, 0.5 equiv., 2.89 mmol) and NaBH₄ (0.66 g, 3.0 equiv., 17.37 mmol). The reaction mixture was stirred at RT for 2 hr then concentrated in vacuo to remove the EtOH. The resulting residue was quenched with ice cold water and extracted with EtOAc. The organic layers were washed with brine, dried (Na₂SO₄) and concentrated in vacuo to afford 132 (1.7 g, 4.89 mmol, 85% yield).

LCMS m/z 248.3 (M+H-100)

¹H NMR (400 MHz, DMSO-d₆) 6.96-7.18 (m, 3H), 4.89-5.20 (m, 1H), 3.87-4.15 (m, 3H), 3.06-3.28 (m, 1H), 2.65-2.89 (m, 4H), 2.56 (br d, J=3.3 Hz, 2H), 1.31-1.50 (m, 12H), 1.17 (t, J=7.1 Hz, 3H).

### Example 133—3-(2-(tert-butoxycarbonyl)-1-methyl-1,2,3,4-tetrahydroisoquinolin-5-yl)propanoic acid

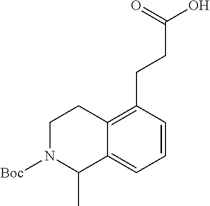 [SMILES: CC1c2cccc(CCC(=O)O)c2CCN1C(=O)OC(C)(C)C]

Prepared following general procedure 7. Obtained 1.0 g, 68% yield, m/z=318.2 [M−H]⁻;

¹H NMR (400 MHz, DMSO-d₆) 11.91-12.32 (m, 1H), 6.98-7.20 (m, 3H), 5.05 (br d, J=5.0 Hz, 1H), 3.88-4.17 (m, 1H), 3.23 (br s, 1H), 2.65-2.84 (m, 6H), 1.37-1.49 (m, 12H).

### Example 134—tert-butyl 5-(3-(methoxy(methyl)amino)-3-oxopropyl)-1-methyl-3,4-dihydroisoquinoline-2(1H)-carboxylate

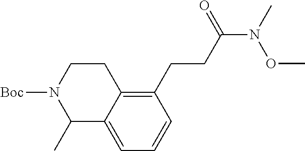 [SMILES: CON(C)C(=O)CCc1cccc2c1CCN(C(=O)OC(C)(C)C)C2C]

Prepared following general procedure 31. Flash chromatography (0-10% MeOH in DCM) obtained 1.0 g, 88% yield, m/z 263.2 [M+H-100]⁺

### Example 135—tert-butyl 1-methyl-5-(3-oxopropyl)-3,4-dihydroisoquinoline-2(1H)-carboxylate

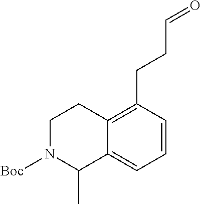 [SMILES: CC1c2cccc(CCC=O)c2CCN1C(=O)OC(C)(C)C]

Prepared following general procedure 32. Crude material obtained 0.34 g, 94% yield, m/z 204.2 [M+H-100]⁺; ¹H NMR (400 MHz, DMSO-d₆) 9.73 (t, J=1.3 Hz, 1H), 6.98-7.22 (m, 3H), 4.92-5.16 (m, 1H), 3.85-4.00 (m, 1H), 2.64-2.87 (m, 4H), 2.30-2.44 (m, 1H), 1.63-1.79 (m, 1H), 1.37-1.46 (m, 12H), 1.36 (br s, 1H).

### Example 136—tert-butyl 5-(3-(4-(5-fluoro-4-(5-fluoro-2-methoxyphenyl)-1H-pyrrolo[2,3-b]pyridin-2-yl)piperidin-1-yl)propyl)-1-methyl-3,4-dihydroisoquinoline-2(1H)-carboxylate

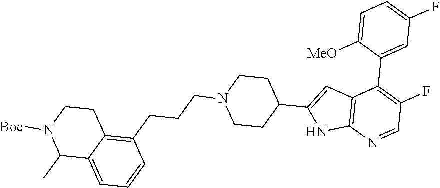 [SMILES: COc1ccc(F)cc1-c1c(F)cnc2[nH]c(C3CCN(CCCc4cccc5c4CCN(C(=O)OC(C)(C)C)C5C)CC3)cc12]

Prepared following general procedure 26. Obtained 370 mg, 76% yield, LCMS m/z=631 [M+H-100].

### Example 137—5-(3-(4-(5-fluoro-4-(5-fluoro-2-methoxyphenyl)-1H-pyrrolo[2,3-b]pyridin-2-yl)piperidin-1-yl)propyl)-1-methyl-1,2,3,4-tetrahydroisoquinoline

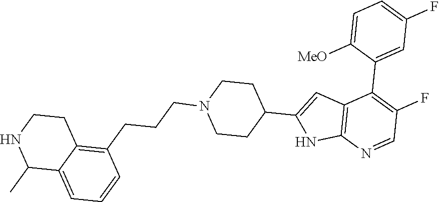 [SMILES: COc1ccc(F)cc1-c1c(F)cnc2[nH]c(C3CCN(CCCc4cccc5c4CCNC5C)CC3)cc12]

Prepared following general procedure 8. Obtained 310 mg, 86% yield, LCMS m/z=531.3 [M+H]⁺.

### Example 138—3-(5-(3-(4-(5-fluoro-4-(5-fluoro-2-methoxyphenyl)-1H-pyrrolo[2,3-b]pyridin-2-yl)piperidin-1-yl)propyl)-1-methyl-3,4-dihydroisoquinolin-2(1H)-yl)-3-oxopropanenitrile

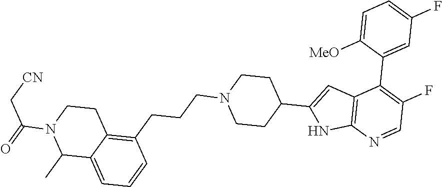 [SMILES: COc1ccc(F)cc1-c1c(F)cnc2[nH]c(C3CCN(CCCc4cccc5c4CCN(C(=O)CC#N)C5C)CC3)cc12]

Prepared following general procedure 9. Obtained 330 mg, 65% yield, LCMS m/z=598.2 [M+H]⁺.

### Example A53—(Z/E)-2-(5-(3-(4-(5-fluoro-4-(5-fluoro-2-methoxyphenyl)-1H-pyrrolo[2,3-b]pyridin-2-yl)piperidin-1-yl)propyl)-1-methyl-1,2,3,4-tetrahydroisoquinoline-2-carbonyl)-3-(thiazol-2-yl)acrylonitrile

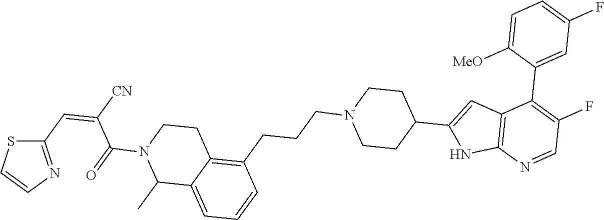 [SMILES: COc1ccc(F)cc1-c1c(F)cnc2[nH]c(C3CCN(CCCc4cccc5c4CCN(C(=O)/C(C#N)=C\c4nccs4)C5C)CC3)cc12]

Prepared following general procedure 10. Obtained 40 mg, 13.8% yield, LCMS m/z=691.2 [M+H]⁺.

### Example 139—tert-butyl 6-(3-(4-((5-(((5-(tert-butyl)oxazol-2-yl)methyl)thio)thiazol-2-yl)carbamoyl)piperidin-1-yl)propyl)-1-methyl-3,4-dihydroisoquinoline-2(1H)-carboxylate

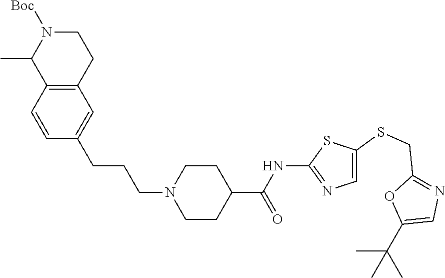 [SMILES: CC1c2ccc(CCCN3CCC(C(=O)Nc4ncc(SCc5ncc(C(C)(C)C)o5)s4)CC3)cc2CCN1C(=O)OC(C)(C)C]

Prepared following general procedure 26. Obtained 0.80 g, 32% yield, LCMS m/z=668 [M+H]).

### Example 140—N-(5-(((5-(tert-butyl)oxazol-2-yl)methyl)thio)thiazol-2-yl)-1-(3-(1-methyl-1,2,3,4-tetrahydroisoquinolin-6-yl)propyl)piperidine-4-carboxamide

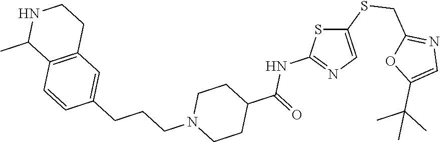 [SMILES: CC1NCCc2cc(CCCN3CCC(C(=O)Nc4ncc(SCc5ncc(C(C)(C)C)o5)s4)CC3)ccc21]

Prepared following general procedure 8. Obtained 0.70 g, 95% yield, LCMS m/z=568 [M+H]⁺.

### Example 141—N-(5-(((5-(tert-butyl)oxazol-2-yl)methyl)thio)thiazol-2-yl)-1-(3-(2-(2-cyanoacetyl)-1-methyl-1,2,3,4-tetrahydroisoquinolin-6-yl)propyl)piperidine-4-carboxamide

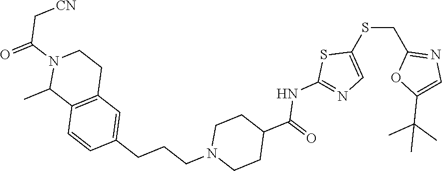 [SMILES: CC1c2ccc(CCCN3CCC(C(=O)Nc4ncc(SCc5ncc(C(C)(C)C)o5)s4)CC3)cc2CCN1C(=O)CC#N]

Prepared following general procedure 9. Obtained 0.70 g, 90% yield, LCMS m/z=635.6 [M+H]⁺.

### Example A54—(E/Z)—N-(5-(((5-(N-butyl)oxazol-2-yl)methyl)thio)thiazol-2-yl)-1-(3-(2-(2-cyano-4-methyl-4-morpholinopent-2-enoyl)-1-methyl-1,2,3,4-tetrahydroisoquinolin-6-yl)propyl)piperidine-4-carboxamide

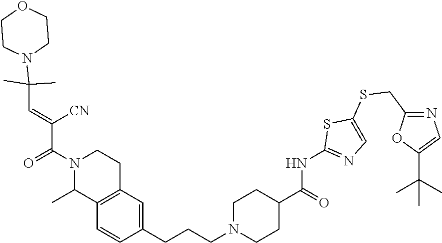 [SMILES: CC1c2ccc(CCCN3CCC(C(=O)Nc4ncc(SCc5ncc(C(C)(C)C)o5)s4)CC3)cc2CCN1C(=O)/C(C#N)=C/C(C)(C)N1CCOCC1]

Prepared following general procedure 10. Obtained 118 mg, 27.5% yield, LCMS m/z=775 [M+H]^{+ 1}H NMR (400 MHz, DMSO-d₆) 12.08-12.36 (m, 1H), 7.38 (s, 1H), 7.11-7.23 (m, 1H), 6.98-7.10 (m, 2H), 6.77-6.88 (m, 1H), 6.72 (s, 1H), 5.25-5.39 (m, 1H), 4.05 (s, 2H), 3.73-3.90 (m, 1H), 3.64 (br d, J=4.0 Hz, 4H), 3.44-3.61 (m, 2H), 2.72-3.01 (m, 4H), 2.27 (br t, J=7.0 Hz, 4H), 1.53-1.93 (m, 12H), 1.42 (br d, J=6.5 Hz, 3H), 1.09-1.33 (m, 15H).

### Example A55—(E/Z)—N-(5-(((5-(tert-butyl)oxazol-2-yl)methyl)thio)thiazol-2-yl)-1-(3-(2-(2-cyano-4,4-dimethyl-5-morpholinopent-2-enoyl)-1-methyl-1,2,3,4-tetrahydroisoquinolin-6-yl)propyl)piperidine-4-carboxamide

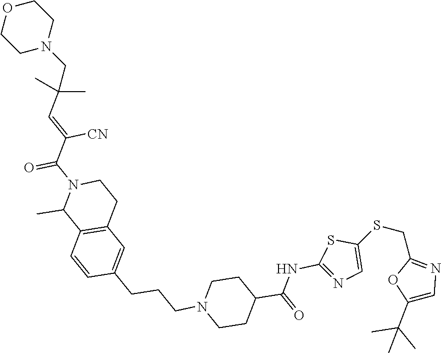 [SMILES: CC1c2ccc(CCCN3CCC(C(=O)Nc4ncc(SCc5ncc(C(C)(C)C)o5)s4)CC3)cc2CCN1C(=O)/C(C#N)=C/C(C)(C)CN1CCOCC1]

Prepared following general procedure 10. Obtained 18 mg, 4.7% yield, LCMS m/z=788 [M+H]⁺

### Example 142—tert-butyl 6-(3-hydroxyprop-1-yn-1-yl)-1-methyl-3,4-dihydroisoquinoline-2(1H)-carboxylate

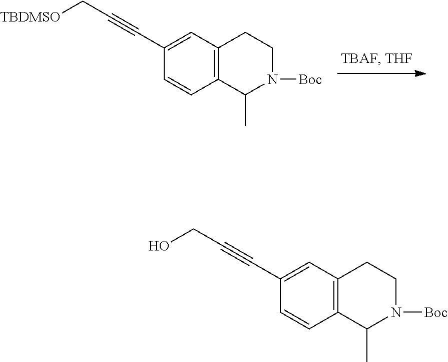 [SMILES: CC1c2ccc(C#CCO[Si](C)(C)C(C)(C)C)cc2CCN1C(=O)OC(C)(C)C ; CC1c2ccc(C#CCO)cc2CCN1C(=O)OC(C)(C)C]

To a mixture of compound 121 (2.2 g, 1.0 eq, 5.29 mmol) in THF (10 ml) was added TBAF (26.5 ml, 5.0 eq, 26.5 mmol) and the reaction mixture was stirred at RT for 3 h. The reaction mixture was diluted with water, and extracted with EtOAc. The combined organic layers were dried (Na₂SO₄) and concentrated in vacuo. The crude product was purified by flash chromatography (15% to 25% EtOAc in n-hexane) to give the desired product 142 (0.98 g, 3.25 mmol, 61.4% yield) as a clear oil.

¹H NMR (400 MHz, DMSO-d₆) 7.23-7.21 (m, 3H), 5.30 (t, J=6.00 Hz, 1H), 5.08 (m, 1H), 4.29 (d, J=6.00 Hz, 2H), 4.03-3.92 (m, 1H), 3.20-3.08 (m, 2H), 2.76-2.73 (m, 2H), 1.43-1.36 (m, 12H).

### Example 143—tert-butyl 6-(3-bromoprop-1-yn-1-yl)-1-methyl-3,4-dihydroisoquinoline-2(1H)-carboxylate

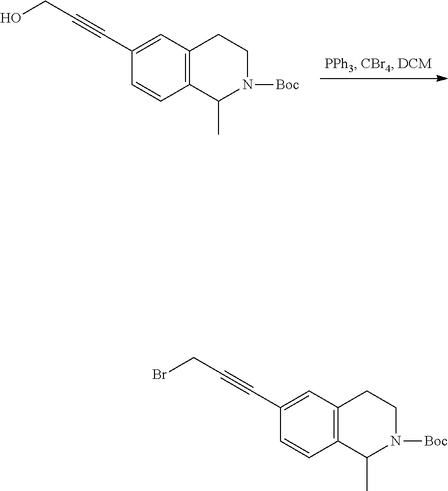 [SMILES: CC1c2ccc(C#CCO)cc2CCN1C(=O)OC(C)(C)C ; CC1c2ccc(C#CCBr)cc2CCN1C(=O)OC(C)(C)C]

To a stirred solution of compound 142 (0.2 g, 1 eq, 0.664 mmol) in DCM (10 ml) at RT under nitrogen was added triphenylphosphine (0.226 g, 1.3 eq, 0.863 mmol) and CBr₄ (0.286 g, 1.3 eq, 0.863 mmol). The reaction mixture was stirred for 3 h at RT then was purified by flash column purification (7% to 10% EtOAc in n-hexane) to afford 143 (0.24 g, 0.659 mmol, 99% yield) as clear oil.

¹H NMR (400 MHz, DMSO-d₆) 7.26 (s, 3H), 5.08 (m, 1H), 4.51 (s, 2H), 4.06-3.92 (m, 1H), 3.21-3.12 (m, 1H), 2.77-2.74 (m, 2H), 1.43-1.36 (m, 12H),

### Example 144: tert-butyl 6-(3-(4-((5-(((5-(tert-butyl)oxazol-2-yl)methyl)thio)thiazol-2-yl)carbamoyl)piperidin-1-yl)prop-1-yn-1-yl)-1-methyl-3,4-dihydroisoquinoline-2(1H)-carboxylate

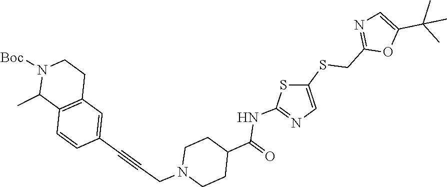 [SMILES: CC1c2ccc(C#CCN3CCC(C(=O)Nc4ncc(SCc5ncc(C(C)(C)C)o5)s4)CC3)cc2CCN1C(=O)OC(C)(C)C]

Prepared following general procedure 30. Obtained 190 mg, 59.7% yield, m/z=664.8 [M+H]⁺

### Example 145: N-(5-(((5-(tert-butyl)oxazol-2-yl)methyl)thio)thiazol-2-yl)-1-(3-(1-methyl-1,2,3,4-tetrahydroisoquinolin-6-yl)prop-2-yn-1-yl)piperidine-4-carboxamide

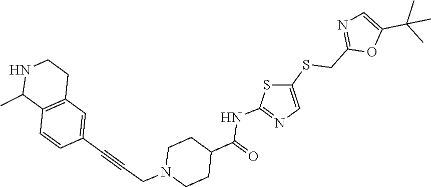 [SMILES: CC1NCCc2cc(C#CCN3CCC(C(=O)Nc4ncc(SCc5ncc(C(C)(C)C)o5)s4)CC3)ccc21]

Prepared following general procedure 8. Obtained 150 mg, 98% yield, m/z=564.8 [M+H]⁺

### Example 146: N-(5-(((5-(tert-butyl)oxazol-2-yl)methyl)thio)thiazol-2-yl)-1-(3-(2-(2-cyanoacetyl)-1-methyl-1,2,3,4-tetrahydroisoquinolin-6-yl)prop-2-yn-1-yl)piperidine-4-carboxamide

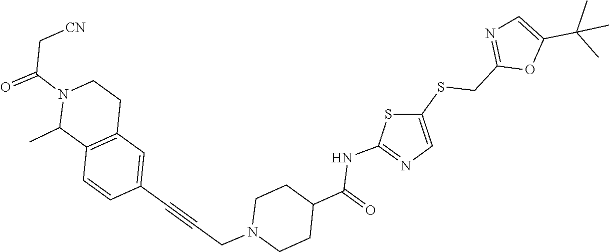 [SMILES: CC1c2ccc(C#CCN3CCC(C(=O)Nc4ncc(SCc5ncc(C(C)(C)C)o5)s4)CC3)cc2CCN1C(=O)CC#N]

Prepared following general procedure 9. Obtained 155 mg, 84% yield, m/z=631.4 [M+H]⁺

### Example A56: (E)-N-(5-(((5-(tert-butyl)oxazol-2-yl)methyl)thio)thiazol-2-yl)-1-(3-(2-(2-cyano-3-(4,5,6,7-tetrahydrobenzo[d]thiazol-2-yl)acryloyl)-1-methyl-1,2,3,4-tetrahydroisoquinolin-6-yl)prop-2-yn-1-yl)piperidine-4-carboxamide

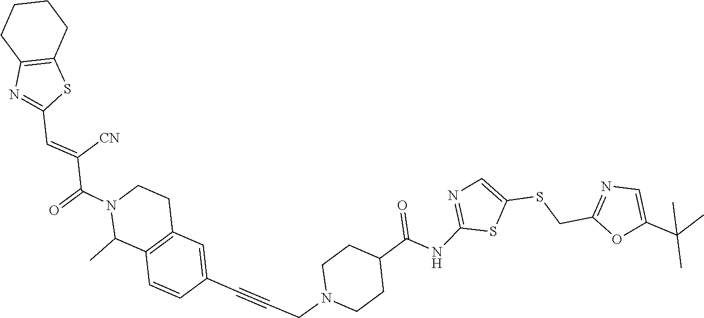 [SMILES: CC1c2ccc(C#CCN3CCC(C(=O)Nc4ncc(SCc5ncc(C(C)(C)C)o5)s4)CC3)cc2CCN1C(=O)/C(C#N)=C/c1nc2c(s1)CCCC2]

Prepared following general procedure 10 in EtOH. Obtained via reverse phase preparative HPLC 6 mg, 9% yield, LCMS m/z=780.4 [M+H]⁺

### Example 147—tert-butyl ((4-bromophenyl)(cyclopropyl)methyl)(methyl)carbamate

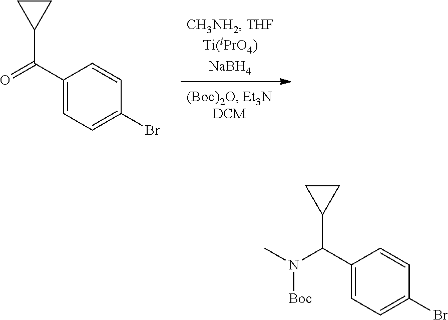 [SMILES: O=C(c1ccc(Br)cc1)C1CC1 ; CN(C(=O)OC(C)(C)C)C(c1ccc(Br)cc1)C1CC1]

To a stirred solution of (4-bromophenyl)(cyclopropyl)methanone (15.0 g, 1 equiv., 66.6 mmol) and methylamine (2M in THF) (100 ml, 3 equiv., 200 mmol) was added titanium(IV) isopropoxide (26.3 ml, 1.3 equiv., 87 mmol) at RT. The reaction mixture was stirred at RT for 20 h, before the addition of NaBH₄ (3.78 g, 1.5 equiv., 100 mmol) at 0° C. The reaction mixture was warmed to RT and stirred for 16 h before being quenched with aqueous NaHCO₃ solution at 0° C. The reaction mixture was diluted with EtOAc and filtered through celite. The filtrate was washed with brine, dried (Na₂SO₄) and concentrated in vacuo to give 1-(4-bromophenyl)-1-cyclopropyl-N-methylmethanamine (15 g, 62.5 mmol) as a colourless oil. The crude oil was dissolved in DCM (150 ml) and triethylamine (26.1 ml, 3.0 equiv., 187 mmol) was added at 0° C. To this mixture Boc-anhydride (21.5 ml, 1.5 equiv., 94 mmol) was added slowly and the resulting reaction mixture was stirred for 24 h at RT under N₂ atmosphere. The reaction mixture was diluted with DCM and extracted with water. The organic layer was dried (Na₂SO₄) and concentrated in vacuo. The crude product was purified by flash chromatography (0-100% EtOAc in n-hexane) to give desired product 147 (16.5 g, 48.5 mmol, 78% yield).

LCMS: Mass found; [M+H]⁺ 340 and [M+H+2]⁺ 342.

### Example 148—tert-butyl (cyclopropyl(4-vinylphenyl)methyl)(methyl)carbamate

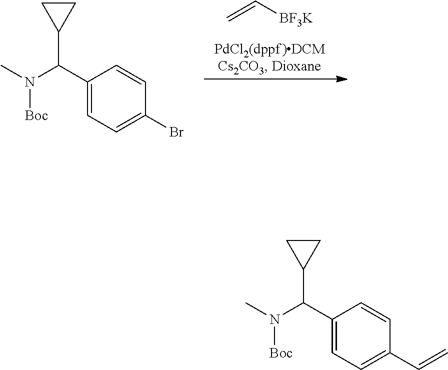

To a stirred solution of compound 147 (16.0 g, 1 equiv., 47 mmol), potassium vinyltrifluoroborate (18.9 g, 3.0 equiv., 141 mmol) and aqueous Cs₂CO₃ (2 M in water) (23.51 ml, 1 equiv., 47 mmol) in 1,4-dioxane (180 ml) was purged with nitrogen for 20 min followed by the addition of PdCl₂(dpp)-DCM adduct (3.84 g, 0.1 equiv., 4.70 mmol). The reaction mixture was stirred at 85° C. for 18 h then was quenched with water and extracted with EtOAc. The combined organic layers were washed with brine, dried (Na₂SO₄) and concentrated in vacuo to give 148 (11.0 g, 38.3 mmol, 81% yield).

¹H NMR (400 MHz, DMSO-d₆) 7.70-7.46 (m, 2H), 7.36-7.32 (m, 2H), 6.73 (dd, J=17.8, 10.9 Hz, 1H), 5.82 (dd, J=17.8, 0.8 Hz, 1H), 5.25 (dd, J=10.9, 0.8 Hz, 1H), 4.23-4.12 (m, 1H), 3.28-3.18 (m, 1H), 2.68 (s, 3H), 1.39 (br s, 9H), 0.79-0.75 (m, 2H), 0.41-0.31 (m, 2H).

### Example 149—tert-butyl (cyclopropyl(4-formylphenyl)methyl)(methyl)carbamate

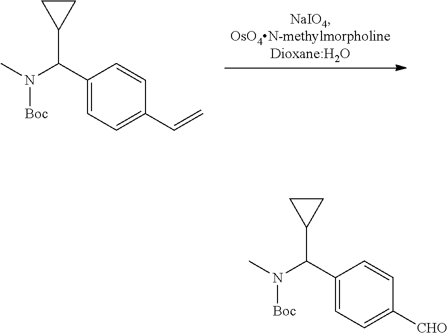 [SMILES: C=Cc1ccc(C(C2CC2)N(C)C(=O)OC(C)(C)C)cc1 ; CN(C(=O)OC(C)(C)C)C(c1ccc(C=O)cc1)C1CC1]

To a solution of compound 148 (11.0 g, 1.0 equiv., 38.3 mmol) in 1,4-dioxane (300 ml) and water (30 ml) was added sodium periodate (16.4 g, 2.0 equiv., 77 mmol) and N-methylmorpholine (1.94 g, 0.5 equiv., 19.14 mmol) at 0° C. followed by slow addition of osmium tetroxide (30.0 ml, 0.1 equiv., 3.83 mmol). The reaction mixture was and stirred at RT for 16 h then concentrated in vacuo. The crude residue was diluted with water:EtOAc (1:1), filtered and extracted with EtOAc. The combined organic layers were dried (Na₂SO₄), concentrated in vacuo then purified by flash columnatography (0-25% EtOAc in n-hexane). The appropriate fractions were combined and concentrated in vacuo to give 149 (8.0 g, 27.6 mmol, 72.2% yield).

### Example 150—tert-butyl (cyclopropyl(4-((4-hydroxypiperidin-1-yl)methyl)phenyl)methyl)(methyl) carbamate

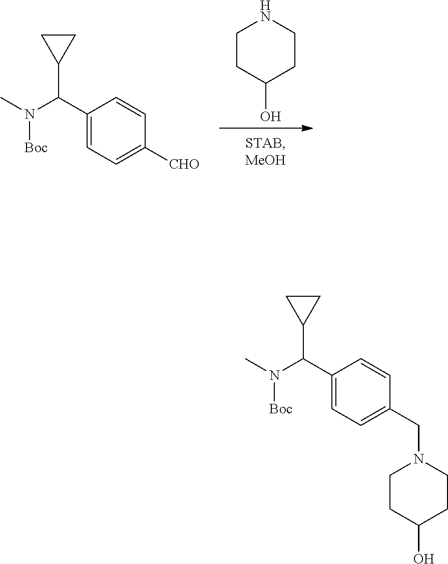 [SMILES: CN(C(=O)OC(C)(C)C)C(c1ccc(C=O)cc1)C1CC1 ; OC1CCNCC1 ; CN(C(=O)OC(C)(C)C)C(c1ccc(CN2CCC(O)CC2)cc1)C1CC1]

Prepared following general procedure 26. Obtained 3.0 g, 55% yield, m/z=375.4 [M+H]⁺

### Example 151—tert-butyl (cyclopropyl(4-((4-oxopiperidin-1-yl)methyl)phenyl)methyl)(methyl) carbamate

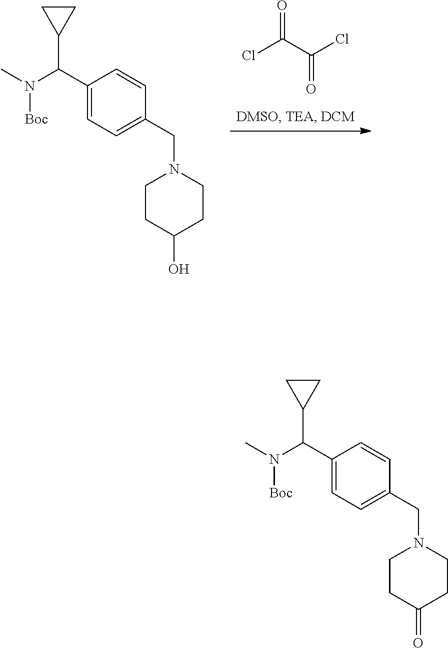 [SMILES: CN(C(=O)OC(C)(C)C)C(c1ccc(CN2CCC(O)CC2)cc1)C1CC1 ; O=C(Cl)C(=O)Cl ; CN(C(=O)OC(C)(C)C)C(c1ccc(CN2CCC(=O)CC2)cc1)C1CC1]

To a stirred solution of oxalyl chloride (0.11 ml, 1.2 equiv., 1.28 mmol) in DCM (10 ml) at −78° C. was added DMSO (0.19 ml, 2.1 equiv., 2.67 mmol) and the reaction was stirred for 10 min. To this mixture was added compound 150 (0.40 g, 1.0 equiv., 1.07 mmol) in DCM (10 ml) and the mixture was stirred for 30 min at −78° C. The reaction mixture was quenched with Et₃N (0.744 ml, 5.0 equiv., 5.34 mmol) and allowed to warm to RT. Saturated aqueous ammonium chloride solution was added then the organic phase was washed with water, brine, dried (Na₂SO₄), and concentrated in vacuo. The crude residue was purified by flash chromatography (0-10% MeOH in DCM). The desired fractions were combined and concentrated in vacuo to give 151 (0.2 g, 0.53 mmol, 49.8% yield).

LCMS m/z=[M+H]⁺ 373.2

### Example 152—tert-butyl ((4-((4-(((5-(((5-(tert-butyl)oxazol-2-yl)methyl)thio)thiazol-2-yl)amino)methyl)-[1,4′-bipiperidin]-1′-yl)methyl)phenyl)(cyclopropyl)methyl)(methyl) carbamate

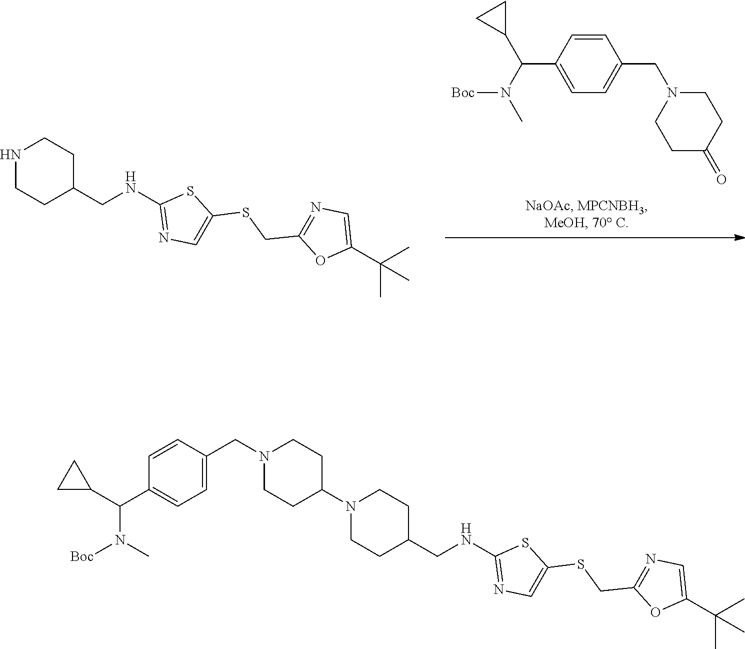 [SMILES: CC(C)(C)c1cnc(CSc2cnc(NCC3CCNCC3)s2)o1 ; CN(C(=O)OC(C)(C)C)C(c1ccc(CN2CCC(=O)CC2)cc1)C1CC1 ; CN(C(=O)OC(C)(C)C)C(c1ccc(CN2CCC(N3CCC(CNc4ncc(SCc5ncc(C(C)(C)C)o5)s4)CC3)CC2)cc1)C1CC1]

Prepared following general procedure 26. Obtained 180 mg, 91% yield, m/z=723.6 [M+H]⁺, ¹H NMR (400 MHz, DMSO-d₆) 9.89 (br s, 1H), 8.05 (s, 1H), 7.31 (m, 4H), 6.91 (s, 1H), 6.73 (s, 1H), 4.13-4.04 (m, 1H), 3.51 (m, 2H), 3.33 (m, 2H), 3.18-2.92 (m, 6H), 2.68-2.51 (m, 4H), 1.92-1.82 (m, 10H), 1.39 (s, 9H), 1.23-1.16 (m, 12H), 0.90-0.71 (m, 4H).

### Example 153—5-(((5-(tert-butyl)oxazol-2-yl)methyl)thio)-N-((1′-(4-(cyclopropyl(methylamino)methyl) benzyl)-[1,4′-bipiperidin]-4-yl) methyl)thiazol-2-amine

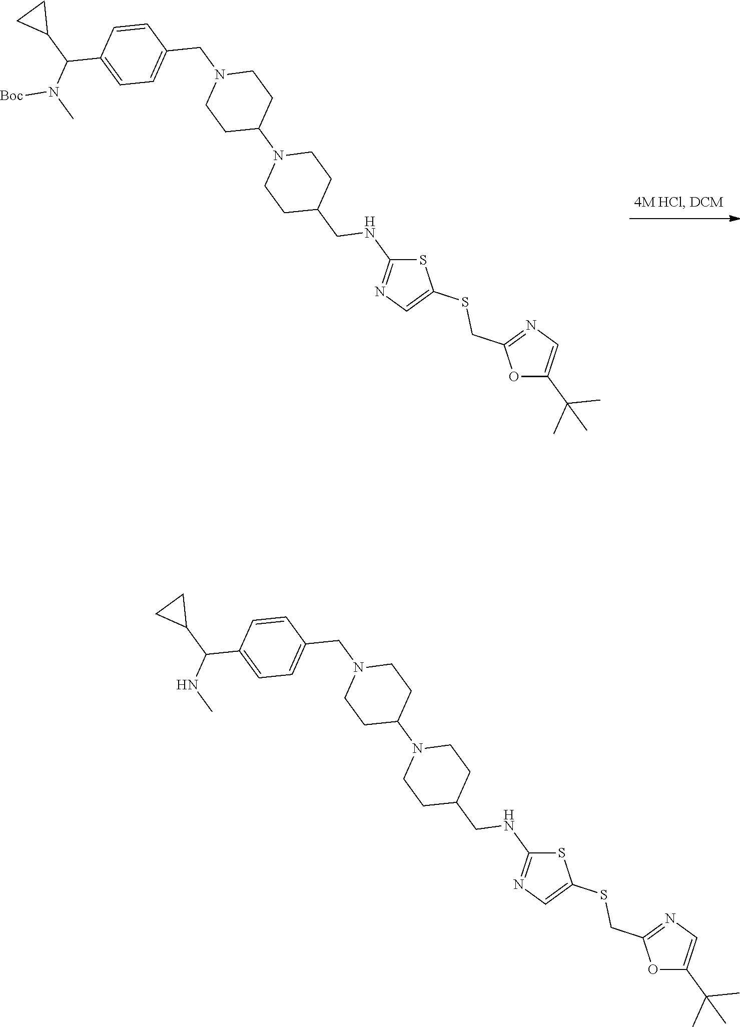 [SMILES: CN(C(=O)OC(C)(C)C)C(c1ccc(CN2CCC(N3CCC(CNc4ncc(SCc5ncc(C(C)(C)C)o5)s4)CC3)CC2)cc1)C1CC1 ; CNC(c1ccc(CN2CCC(N3CCC(CNc4ncc(SCc5ncc(C(C)(C)C)o5)s4)CC3)CC2)cc1)C1CC1]

Prepared following general procedure 8. Obtained 130 mg, 89% yield, m/z=623.6 [M+H]⁺

### Example 154—N-((4-((4-(((5-(((5-(tert-butyl)oxazol-2-yl)methyl)thio)thiazol-2-yl)amino)methyl)-[1,4′-bipiperidin]-1′-yl)methyl)phenyl)(cyclopropyl)methyl)-2-cyano-N-methylacetamide

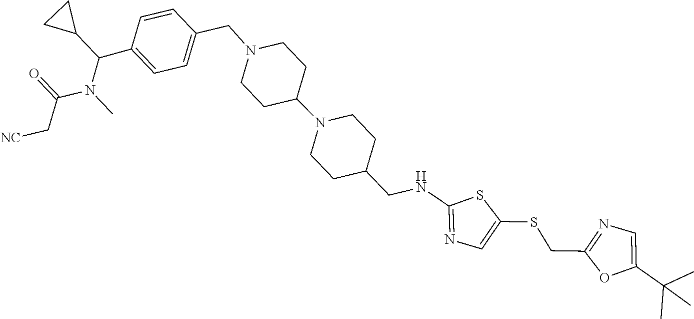 [SMILES: CN(C(=O)CC#N)C(c1ccc(CN2CCC(N3CCC(CNc4ncc(SCc5ncc(C(C)(C)C)o5)s4)CC3)CC2)cc1)C1CC1]

Prepared following general procedure 9. Obtained 120 mg, 79% yield, m/z=690.4 [M+H]⁺, ¹H NMR (400 MHz, DMSO-d₆) 10.12 (br s, 1H), 7.19-7.50 (m, 4H), 6.91 (s, 1H), 6.73 (s, 1H), 4.74 (br d, J=10.1 Hz, 1H), 4.04-4.27 (m, 3H), 3.94 (s, 2H), 3.51 (br s, 2H), 3.02-3.22 (m, 3H), 2.64-2.99 (m, 6H), 1.61-2.09 (m, 7H), 1.33-1.57 (m, 4H), 1.14-1.33 (m, 9H), 0.72-0.96 (m, 4H), 0.22-0.65 (m, 3H).

### Example A57—(E/Z)—N-((4-((4-(((5-(((5-(tert-butyl)oxazol-2-yl)methyl)thio)thiazol-2-yl)amino)methyl)-[1,4′-bipiperidin]-1′-yl)methyl)phenyl)(cyclopropyl)methyl)-2-cyano-N-methyl-3-phenylacrylamide

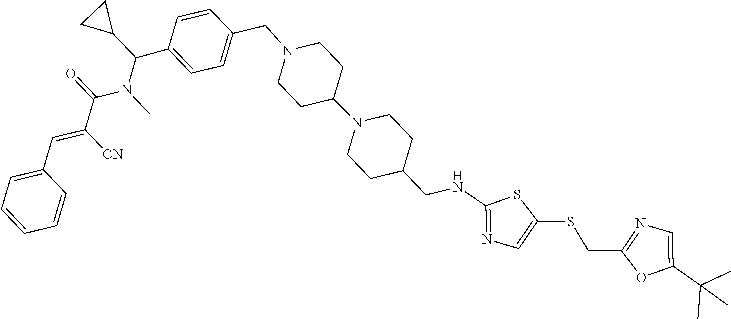 [SMILES: CN(C(=O)/C(C#N)=C/c1ccccc1)C(c1ccc(CN2CCC(N3CCC(CNc4ncc(SCc5ncc(C(C)(C)C)o5)s4)CC3)CC2)cc1)C1CC1]

Prepared following general procedure 10 in THF. Obtained via reverse phase preparative HPLC, 11 mg, 16% yield, LCMS m/z=778.5 [M+H]; ¹H NMR (400 MHz, DMSO-d₆) 8.21 (s, 1H), 7.93 (br d, J=5.3 Hz, 2H), 7.56 (br s, 2H), 7.47 (br d, J=7.0 Hz, 1H), 7.40 (br s, 2H), 7.27-7.37 (m, 3H), 6.89 (s, 1H), 6.72 (s, 1H), 3.93 (s, 2H), 3.43 (br s, 4H), 2.93-3.08 (m, 4H), 2.85 (br s, 4H), 2.09 (br t, J=10.9 Hz, 2H), 1.90 (br t, J=10.9 Hz, 2H), 1.60-1.71 (m, 4H), 1.38-1.56 (m, 5H), 1.22 (s, 9H), 1.06-1.17 (m, 2H), 0.38-0.90 (m, 4H).

### Example 155—tert-butyl ((4-((4-((5-(((5-(tert-butyl)oxazol-2-yl)methyl)thio)thiazol-2-yl) carbamoyl)-[1,4′-bipiperidin]-1′-yl)methyl)phenyl)(cyclopropyl)methyl) (methyl)carbamate

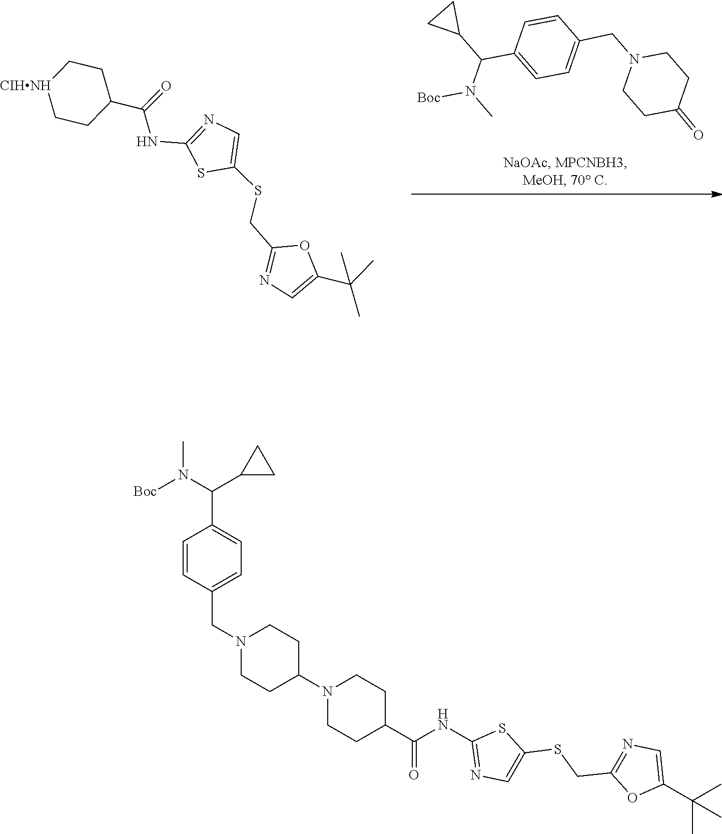

Prepared following general procedure 26. Obtained 180 mg, 91% yield, m/z=737.6 [M+H]⁺

### Example 156—N-(5-(((5-(tert-butyl)oxazol-2-yl)methyl)thio)thiazol-2-yl)-1′-(4-(cyclopropyl(methylamino)methyl)benzyl)-[1,4′-bipiperidine]-4-carboxamide hydrochloride

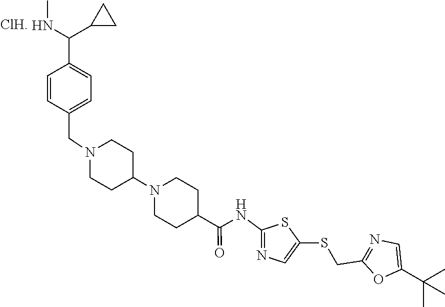 [SMILES: CNC(c1ccc(CN2CCC(N3CCC(C(=O)Nc4ncc(SCc5ncc(C(C)(C)C)o5)s4)CC3)CC2)cc1)C1CC1]

Prepared following general procedure 8. Obtained 140 mg, 79% yield, m/z=637.6 [M+H]⁺

### Example 157—N-(5-(((5-(tert-butyl)oxazol-2-yl)methyl)thio)thiazol-2-yl)-1′-(4-((2-cyano-N-methylacetamido)(cyclopropyl)methyl)benzyl)-[1,4′-bipiperidine]-4-carboxamide

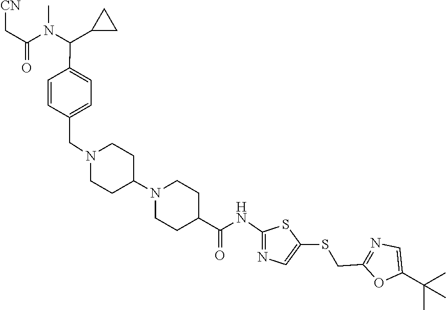 [SMILES: CN(C(=O)CC#N)C(c1ccc(CN2CCC(N3CCC(C(=O)Nc4ncc(SCc5ncc(C(C)(C)C)o5)s4)CC3)CC2)cc1)C1CC1]

Prepared following general procedure 9. Obtained 120 mg, 62% yield, m/z=704.2 [M+H]⁺

### Example A58: (E/Z)—N-(5-(((5-(tert-butyl)oxazol-2-yl)methyl)thio)thiazol-2-yl)-1′-(4-((2-cyano-N-methyl-3-phenylacrylamido)(cyclopropyl)methyl)benzyl)-[1,4′-bipiperidine]-4-carboxamide

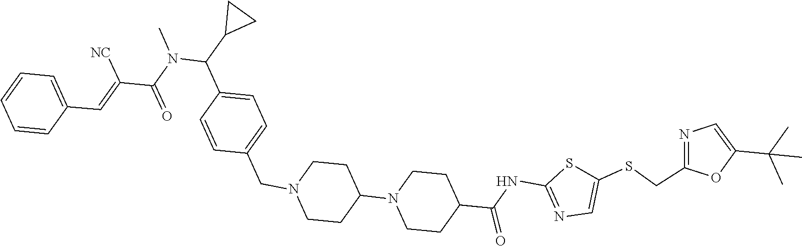 [SMILES: CN(C(=O)/C(C#N)=C/c1ccccc1)C(c1ccc(CN2CCC(N3CCC(C(=O)Nc4ncc(SCc5ncc(C(C)(C)C)o5)s4)CC3)CC2)cc1)C1CC1]

Prepared following general procedure 10 in EtOH. Obtained via reverse phase preparative HPLC 9.6 mg, 14% yield, LCMS m/z=792.5 [M+H]; ¹H NMR (400 MHz, DMSO-d₆) 12.13-12.31 (m, 1H), 7.88-7.98 (m, 2H), 7.78-7.86 (m, 1H), 7.52-7.60 (m, 3H), 7.39-7.45 (m, 2H), 7.38 (s, 1H), 7.30-7.36 (m, 2H), 6.72 (s, 1H), 4.05 (s, 2H), 3.44 (s, 4H), 2.94-3.03 (m, 2H), 2.87 (br dd, J=18.9, 11.1 Hz, 4H), 2.06-2.28 (m, 4H), 1.91 (br t, J=10.5 Hz, 2H), 1.65-1.80 (m, 4H), 1.50-1.62 (m, 3H), 1.39-1.49 (m, 2H), 1.18 (s, 9H), 0.80-0.90 (m, 1H), 0.36-0.71 (m, 3H).

### Example 158—tert-butyl 7-(3-(4-(5-fluoro-4-(5-fluoro-2-methoxyphenyl)-1H-pyrrolo[2,3-b]pyridin-2-yl)piperidin-1-yl)propyl)-1-methyl-3,4-dihydroisoquinoline-2(1H)-carboxylate

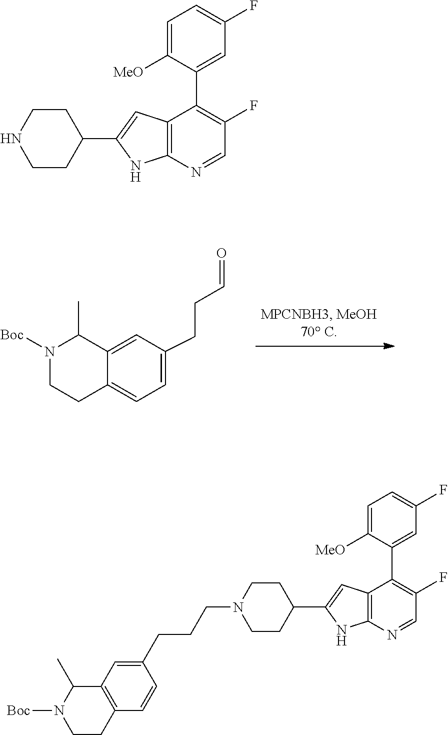 [SMILES: COc1ccc(F)cc1-c1c(F)cnc2[nH]c(C3CCNCC3)cc12 ; CC1c2cc(CCC=O)ccc2CCN1C(=O)OC(C)(C)C ; COc1ccc(F)cc1-c1c(F)cnc2[nH]c(C3CCN(CCCc4ccc5c(c4)C(C)N(C(=O)OC(C)(C)C)CC5)CC3)cc12]

Prepared following general procedure 26 (using amine as prepared in WO2014139328, the entire contents of which are incorporated herein by reference). Obtained 500 mg, 63.5% yield, m/z=631.3 [M+H-100]⁺

### Example 159—7-(3-(4-(5-fluoro-4-(5-fluoro-2-methoxyphenyl)-1H-pyrrolo[2,3-b]pyridin-2-yl)piperidin-1-yl) propyl)-1-methyl-1,2,3,4-tetrahydroisoquinoline

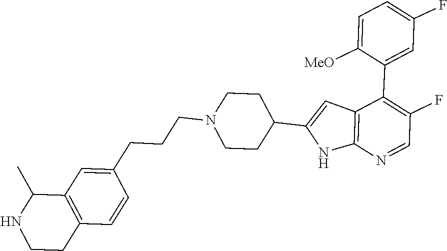 [SMILES: COc1ccc(F)cc1-c1c(F)cnc2[nH]c(C3CCN(CCCc4ccc5c(c4)C(C)NCC5)CC3)cc12]

Prepared following general procedure 8. Obtained 500 mg, 81% yield, m/z=531.3 [M+H]⁺

### Example 160—3-(7-(3-(4-(5-fluoro-4-(5-fluoro-2-methoxyphenyl)-1H-pyrrolo[2,3-b]pyridin-2-yl)piperidin-1-yl)propyl)-1-methyl-3,4-dihydroisoquinolin-2(1H)-yl)-3-oxopropanenitrile

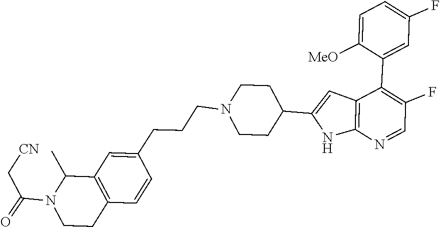 [SMILES: COc1ccc(F)cc1-c1c(F)cnc2[nH]c(C3CCN(CCCc4ccc5c(c4)C(C)N(C(=O)CC#N)CC5)CC3)cc12]

Prepared following general procedure 10. Obtained 370 mg, 52% yield, m/z=598.2 [M+H]⁺

### Example A59—(E/Z)-2-(7-(3-(4-(5-fluoro-4-(5-fluoro-2-methoxyphenyl)-1H-pyrrolo[2,3-b]pyridin-2-yl)piperidin-1-yl)propyl)-1-methyl-1,2,3,4-tetrahydroisoquinoline-2-carbonyl)-3-(thiazol-2-yl)acrylonitrile

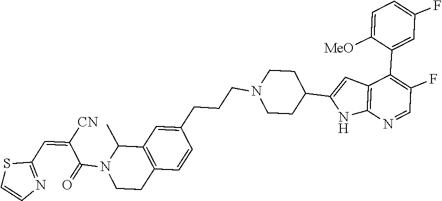 [SMILES: COc1ccc(F)cc1-c1c(F)cnc2[nH]c(C3CCN(CCCc4ccc5c(c4)C(C)N(C(=O)/C(C#N)=C\c4nccs4)CC5)CC3)cc12]

Prepared following general procedure 10 in EtOH. Obtained via reverse phase preparative HPLC 47 mg, 11% yield, LCMS m/z=693.2 [M+H].

### Example 161—tert-butyl (1-(4-(3-(4-(5-fluoro-4-(5-fluoro-2-methoxyphenyl)-1H-pyrrolo[2,3-b]pyridin-2-yl) piperidin-1-yl)propyl)phenyl)butyl)(methyl)carbamate

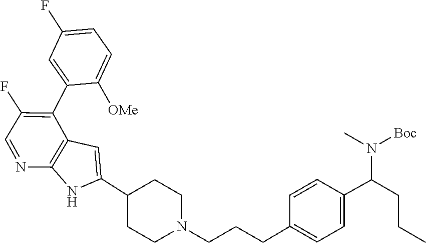 [SMILES: CCCC(c1ccc(CCCN2CCC(c3cc4c(-c5cc(F)ccc5OC)c(F)cnc4[nH]3)CC2)cc1)N(C)C(=O)OC(C)(C)C]

Prepared following general procedure 26 (using amine as prepared in WO2014139328, the entire contents of which are incorporated herein by reference). Obtained 1.4 g, 53.4% yield, m/z=647.4 [M+H]⁺

### Example 162—1-(4-(3-(4-(5-fluoro-4-(5-fluoro-2-methoxyphenyl)-1H-pyrrolo[2,3-b]pyridin-2-yl)piperidin-1-yl)propyl)phenyl)-N-methylbutan-1-amine

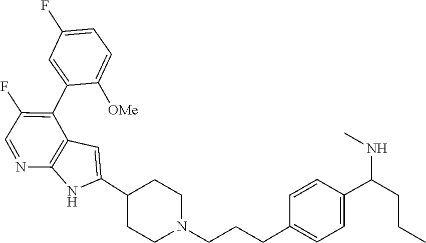 [SMILES: CCCC(NC)c1ccc(CCCN2CCC(c3cc4c(-c5cc(F)ccc5OC)c(F)cnc4[nH]3)CC2)cc1]

Prepared following general procedure 8. Obtained 0.91 g, 98% yield, m/z=547.4[M+H]⁺

### Example 163—2-cyano-N-(1-(4-(3-(4-(5-fluoro-4-(5-fluoro-2-methoxyphenyl)-1H-pyrrolo[2,3-b]pyridin-2-yl) piperidin-1-yl)propyl)phenyl)butyl)-N-methylacetamide

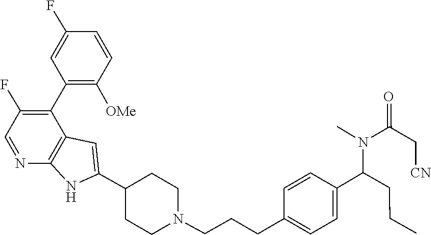 [SMILES: CCCC(c1ccc(CCCN2CCC(c3cc4c(-c5cc(F)ccc5OC)c(F)cnc4[nH]3)CC2)cc1)N(C)C(=O)CC#N]

Prepared following general procedure 10. Obtained 850 mg, 35.5% yield, m/z=614.4 [M+H]⁺

### Example A60—(E/Z)-2-cyano-N-(1-(4-(3-(4-(5-fluoro-4-(5-fluoro-2-methoxyphenyl)-1H-pyrrolo[2,3-b]pyridin-2-yl)piperidin-1-yl)propyl)phenyl)butyl)-N-methyl-3-(thiophen-2-yl)acrylamide

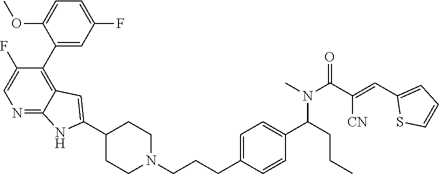 [SMILES: CCCC(c1ccc(CCCN2CCC(c3cc4c(-c5cc(F)ccc5OC)c(F)cnc4[nH]3)CC2)cc1)N(C)C(=O)/C(C#N)=C/c1cccs1]

Prepared following general procedure 10 in EtOH. Obtained via reverse phase preparative HPLC, 45 mg, 13% yield, LCMS m/z=708.2 [M+H].

### Example 164: [5-[4-[2-fluoro-5-[(4-oxo-3H-phthalazin-1-yl)methyl]benzoyl]piperazin-1-yl]-5-oxo-pentyl]ammonium;chloride

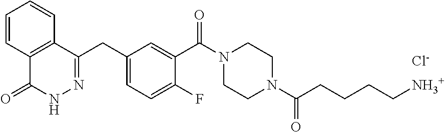 [SMILES: [NH3+]CCCCC(=O)N1CCN(C(=O)c2cc(Cc3n[nH]c(=O)c4ccccc34)ccc2F)CC1 ; [Cl-]]

4-(4-fluoro-3-(piperazine-1-carbonyl)benzyl)phthalazin-1(2H)-one hydrochloride salt (1.00 eq, 61 mg, 0.153 mmol) and 5-(Boc-amino)valeric acid (1.00 eq, 33 mg, 0.153 mmol) were suspended n DMF (0.6 mL). HATU (1.00 eq, 58 mg, 0.153 mmol) was added, followed by N,N-Diisopropylethylamine (5.00 eq, 0.13 mL, 0.765 mmol). The reaction mixture was left to react for 1 hour, diluted to 2 mL with ACN/H₂O and purified by preparative HPLC using a gradient from 10% to 95% of ACN in Water with 0.1% of formic acid over 10 minutes. Fractions contained the desired product were evaporated to dryness. The residue was dissolved in DCM (2 mL) and treated with HCl in Dioxane (52.3 eq, 2.0 mL, 8.00 mmol) 4 M for 1 hour. Volatiles were removed to obtain the title compound (m/z=466.22, (M+H⁺), 77 mg, 56.5% yield.

### Additional Examples

| Example | | m/z |
|---|---|---|
| number | Structure/Preparation | (M + H)⁺ |
| 165 | 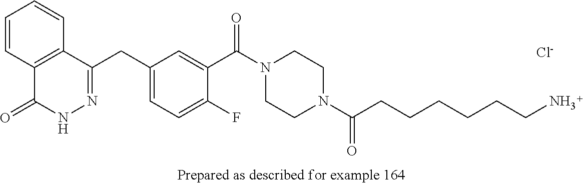 [SMILES: NCCCCCCC(=O)N1CCN(C(=O)c2cc(Cc3n[nH]c(=O)c4ccccc34)ccc2F)CC1 ; [Cl-]] | 494.3 |
| 166 | 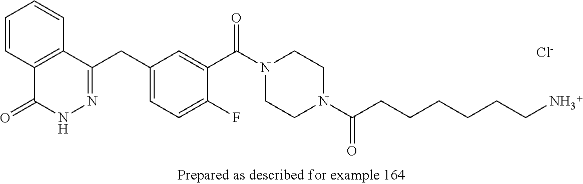 [SMILES: NCCCCCCC(=O)N1CCN(C(=O)c2cc(Cc3n[nH]c(=O)c4ccccc34)ccc2F)CC1 ; [Cl-]] | 522.2 |

### Example A61 (E)-2-cyano-N-(1-(4-(3-((5-(4-(2-fluoro-5-((4-oxo-3,4-dihydrophthalazin-1-yl)methyl)benzoyl)piperazin-1-yl)-5-oxopentyl)amino)-3-oxopropyl)phenyl)butyl)-N-methyl-3-(thiazol-2-yl)acrylamide

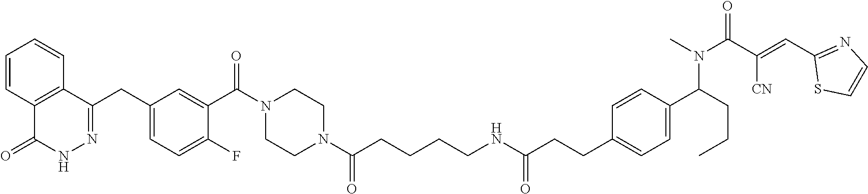 [SMILES: CCCC(c1ccc(CCC(=O)NCCCCC(=O)N2CCN(C(=O)c3cc(Cc4n[nH]c(=O)c5ccccc45)ccc3F)CC2)cc1)N(C)C(=O)/C(C#N)=C/c1nccs1]

To a solution of compound 164 (0.05 M in DMF, 0.50 mL, 0.0252 mmol), compound 44 (1.00 eq, 10 mg, 0.0252 mmol) and N,N-Diisopropylethylamine (5.00 eq, 0.022 mL, 0.126 mmol) in DMF (0.5 mL) were added, followed by HATU (1.00 eq, 9.6 mg, 0.0252 mmol). The reaction was left to stir at r.t. for 2 hours, then diluted with MeOH and purified by preparative HPLC using a gradient from 5% to 95% of ACN in Water containing 0.1% of formic acid to obtain the desired product (m/z=845.35, (M+H⁺), as white solid, 14.28 mg, 65% yield.

### Additional Examples

| Example | | m/z |
|---|---|---|
| number | Structure/Preparation | (M + H)⁺ |
| A62 | 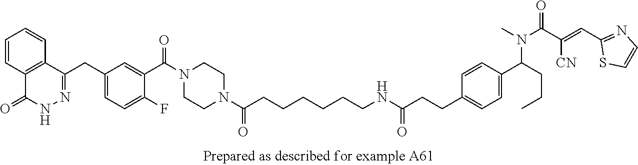 [SMILES: CCCC(c1ccc(CCC(=O)NCCCCCCC(=O)N2CCN(C(=O)c3cc(Cc4n[nH]c(=O)c5ccccc45)ccc3F)CC2)cc1)N(C)C(=O)/C(C#N)=C/c1nccs1] | 873.4 |
| A63 | 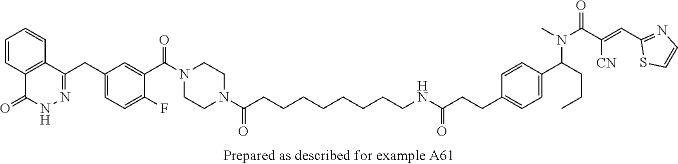 [SMILES: CCCC(c1ccc(CCC(=O)NCCCCCCCCC(=O)N2CCN(C(=O)c3cc(Cc4n[nH]c(=O)c5ccccc45)ccc3F)CC2)cc1)N(C)C(=O)/C(C#N)=C/c1nccs1] | 901.4 |

### Example 167: tert-butyl (S)-4-(7-(8-chloronaphthalen-1-yl)-2-(((S)-1-((2-(trimethylsilyl)ethoxy)carbonyl)pyrrolidin-2-yl)methoxy)-5,6,7,8-tetrahydropyrido[3,4-d]pyrimidin-4-yl)-2-(cyanomethyl)piperazine-1-carboxylate

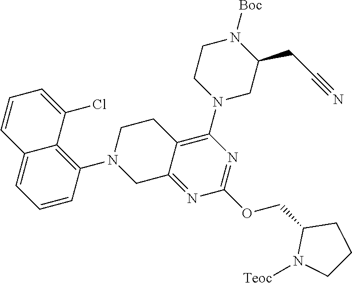

To a solution of 2-trimethylsilylethyl (2S)-2-(hydroxymethyl)pyrrolidine-1-carboxylate (1.69 g, 2 equiv., 6.88 mmol) in THF (10 mL) was added sodium t-butoxide (661 mg, 2 equiv., 6.88 mmol) followed by a portion-wise addition of tert-butyl (2S)-4-[7-(8-chloro-1-naphthyl)-2-methylsulfinyl-6,8-dihydro-5H-pyrido[3,4-d]pyrimidin-4-yl]-2-(cyanomethyl)piperazine-1-carboxylate (2 g, 1 equiv., 3.44 mmol, prepared as described in WO2019/99524, the entire contents of which are incorporated herein by reference). The reaction mixture was stirred at rt for 16 h then was quenched with water and extracted with EtOAc. The combined organics were washed with brine, dried over MgSO₄, filtered and concentrated in vacuo. Purification by flash chromatography (DCM/MeOH (0-20%)) yielded the desired product as a pale brown solid (2.11 g, 2.77 mmol, 80.4% yield).

m/z=762.8 (M+H)⁺

### Example 168: tert-butyl (S)-4-(7-(8-chloronaphthalen-1-yl)-2-(((S)-pyrrolidin-2-yl)methoxy)-5,6,7,8-tetrahydropyrido[3,4-d]pyrimidin-4-yl)-2-(cyanomethyl)piperazine-1-carboxylate

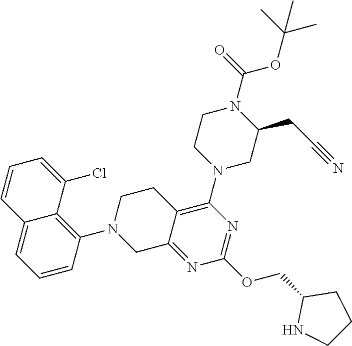 [SMILES: CC(C)(C)OC(=O)N1CCN(c2nc(OC[C@@H]3CCCN3)nc3c2CCN(c2cccc4cccc(Cl)c24)C3)C[C@@H]1CC#N]

To a solution of compound 167 (2.11 g, 1 equiv., 2.77 mmol) in THF (10 mL) was added TBAF (1M in THF) (3.6 mL, 1.3 equiv., 3.60 mmol) and the reaction was stirred at rt for 16 h. Additional TBAF (6.0 mL, 2.2 equiv., 6.0 mmol) added and reaction was stirred at rt for 4 h. The reaction was diluted in DCM and was washed with sat. NH₄Cl. The organics were dried (MgSO₄), filtered and concentrated in vacuo. Purification by flash chromatography (DCM/MeOH (0-20%)) yielded the crude product, which was dissolved in EtOAc and washed with brine, dried (MgSO₄) and concentrated in vacuo to give the desired product (0.90 g, 1.02 mmol, 36.8% yield) as a brown solid.

A sample of the crude was purified by preparative HPLC using a gradient from 5% to 95% of Acetonitrile in water (containing 0.1% of formic acid) for analytical purposes.

NMR: ¹H NMR (400 MHz, CDCl3) δ 7.77-7.74 (m, 1H), 7.64-7.58 (m, 1H), 7.53-7.49 (m, 1H), 7.47-7.37 (m, 1H), 7.35-7.31 (m, 1H), 7.25-7.16 (m, 1H), 4.64-4.52 (m, 2H), 4.50-4.33 (m, 1H), 4.32-4.18 (m, 1H), 4.14-3.96 (m, 2H), 3.94-3.70 (m, 2H), 3.60-3.50 (m, 1H), 3.44-3.32 (m, 2H), 3.29-3.17 (m, 2H), 3.16-3.01 (m, 4H), 2.96-2.86 (m, 1H), 2.75-2.65 (m, 2H), 2.62-2.50 (m, 1H), 2.20-1.85 (m, 4H), 1.52-1.48 (br, 9H).

m/z=618.7 (M+H)⁺

### Example 169: 2-(trimethylsilyl)ethyl (4-hydroxybutyl)carbamate

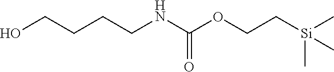

To a solution of 4-amino-1-butanol (1.0 mL, 1 equiv., 11.2 mmol) and triethylamine (2.0 mL, 1.3 equiv., 14.6 mmol) in DCM (40 mL) was added (2,5-dioxopyrrolidin-1-yl) 3-trimethylsilylpropanoate (3.0 g, 1.1 equiv., 12.3 mmol) portion-wise. The reaction was quenched with sat. NH₄Cl and diluted with DCM. The layers were separated, and the organics dried and concentrated in vacuo. Purification by flash chromatography (Hex/EtOAc (0-100%)) yielded the desired product as a colourless oil (2.5 g, 10.8 mmol, 96.3% yield).

¹H NMR (400 MHz, CDCl3) δ 4.20-4.09 (m, 2H), 3.72-3.61 (m, 2H), 3.24-3.17 (m, 2H), 1.66-1.52 (m, 4H), 1.03-0.93 (m, 2H), 0.03 (s, 9H).

### Example 170: 2-(trimethylsilyl)ethyl (4-oxobutyl)carbamate

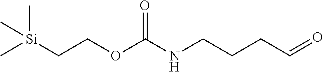

To a solution of dimethyl sulfoxide (0.11 ml, 2.5 eq, 1.5 mmol) in DCM (2.5 ml) was added oxalyl chloride (0.6 ml, 2 eq, 1.2 mmol) dropwise at −78° C. The mixture was stirred for 20 min and a solution of compound 169 (140 mg, 1 eq, 0.6 mmol) in DCM (3.0 ml) was added dropwise at −78° C. The mixture was stirred for an additional 45 min. DIPEA (0.82 ml, 8 eq, 4.8 mmol) was added dropwise and the mixture was warmed up at 0° C. and was stirred for an additional 2 h. The mixture was quenched with water (10 ml) and the aqueous phase was extracted with DCM. The solvent was removed under reduced pressure to give the product in quantitative yield.

NMR: ¹H NMR (400 MHz, CDCl₃) δ 11.28 (s, 1H), 4.27-4.17 (m, 2H), 3.61-3.49 (m, 1H), 3.42-3.26 (m, 1H), 2.13-1.78 (m, 4H), 1.06-0.98 (m, 2H), 0.06-0.02 (m, 9H).

m/z=254.2 (M+Na)⁺

### Example 171: tert-butyl (S)-4-(7-(8-chloronaphthalen-1-yl)-2-(((S)-1-(4-(((2-(trimethylsilyl)ethoxy)carbonyl)amino)butyl)pyrrolidin-2-yl)methoxy)-5,6,7,8-tetrahydropyrido[3,4-d]pyrimidin-4-yl)-2-(cyanomethyl)piperazine-1-carboxylate

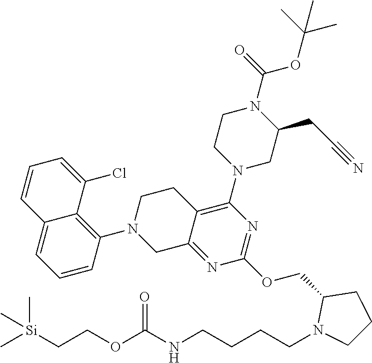 [SMILES: CC(C)(C)OC(=O)N1CCN(c2nc(OC[C@@H]3CCCN3CCCCNC(=O)OCC[Si](C)(C)C)nc3c2CCN(c2cccc4cccc(Cl)c24)C3)C[C@@H]1CC#N]

To a solution of compound 170 (40 mg, 1 eq, 0.065 mol) in DCM (0.5 ml) was added a solution of 2-trimethylsilylethyl N-(4-oxobutyl)carbamate (30 mg, 2.00 eq, 0.129 mmol) at room temperature. The mixture was stirred for 20 min and sodium triacetoxyborohydride (34 mg, 2.5 eq, 0.162 mmol) was added and the mixture which was stirred for an additional 2 h at room temperature.

The reaction was quenched with water and dissolved in DCM. The organic phase was washed with water and the organic phase was dried and evaporated under reduced pressure. The crude product was purified by HPLC (using a gradient from 5% to 95% of acetonitrile in water containing 0.1% of formic acid over 10 minutes) to give pure product as a yellowish solid (42 mg, 0.050 mmol, 78% yield)

NMR: ¹H NMR (400 MHz, CDCl₃) δ 7.76-7.73 (m, 1H), 7.63-7.58 (m, 1H), 7.53-7.48 (m, 1H), 7.47-7.41 (m, 1H), 7.35-7.30 (m, 1H), 7.25-7.19 (m, 1H), 4.90-4.80 (m, 1H), 4.65-4.55 (m, 1H), 4.54-4.48 (m, 1H), 4.45-4.34 (m, 1H), 4.15-4.02 (m, 3H), 4.01-3.72 (m, 3H), 3.70-3.61 (m, 1H), 3.60-3.52 (m, 1H), 3.50-3.23 (m, 3H), 3.23-3.10 (m, 4H), 3.09-2.89 (m, 4H), 2.81-2.65 (m, 2H), 2.62-2.53 (m, 1H), 2.31-2.15 (m, 2H), 2.13-1.75 (m, 6H), 1.66-1.55 (m, 2H), 1.55-1.46 (m, 9H), 0.02-−0.02 (br, 9H).

m/z=833.9 (M+H)⁺

### Example 172: tert-butyl (2S)-4-(7-(8-chloronaphthalen-1-yl)-2-(((2S)-1-(4-(3-(4-(1-((E)-2-cyano-N-methyl-3-(thiazol-2-yl)acrylamido)butyl)phenyl)propanamido)butyl)pyrrolidin-2-yl)methoxy)-5,6,7,8-tetrahydropyrido[3,4-d]pyrimidin-4-yl)-2-(cyanomethyl)piperazine-1-carboxylate

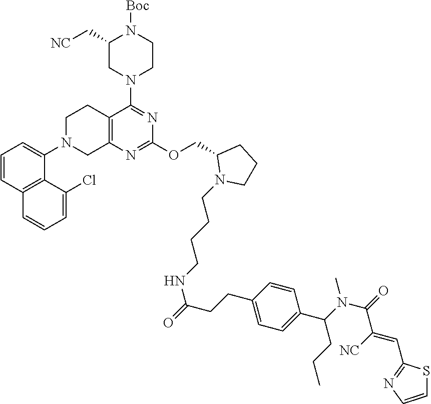 [SMILES: CCCC(c1ccc(CCC(=O)NCCCCN2CCC[C@H]2COc2nc3c(c(N4CCN(C(=O)OC(C)(C)C)[C@@H](CC#N)C4)n2)CCN(c2cccc4cccc(Cl)c24)C3)cc1)N(C)C(=O)/C(C#N)=C/c1nccs1]

To a solution of compound 171 (50 mg, 1 equiv., 0.06 mmol) in DMF (1 mL) was added TBAF (1M in THF) (0.12 mL, 2 equiv., 0.12 mmol) and the reaction was stirred at rt for 2 h. HPLC analysis showed complete deprotection. The reaction was diluted in DCM and was washed with sat. NaHCO₃. The organics were dried (MgSO₄), filtered and concentrated in vacuo. The crude was dissolved in DMF (0.5 mL), compound 44 (24 mg, 0.06 mmol) was added, followed by HATU (26 mg, 1.5 equiv., 0.07 mmol) and DIPEA (31 μL, 0.18 mmol). The reaction mixture was stirred for 30 min then was purified on preparative HPLC (using a gradient from 5% to 95% of acetonitrile in water containing 0.1% of formic acid over 10 minutes) to give the purified product 172 (55 mg, 0.05 mmol) as a pale brown solid.

m/z=1069.2 (M+H

### Example 173: (E)-N-(1-(4-(3-((4-((S)-2-(((7-(8-chloronaphthalen-1-yl)-4-((S)-3-(cyanomethyl)piperazin-1-yl)-5,6,7,8-tetrahydropyrido[3,4-d]pyrimidin-2-yl)oxy)methyl)pyrrolidin-1-yl)butyl)amino)-3-oxopropyl)phenyl)butyl)-2-cyano-N-methyl-3-(thiazol-2-yl)acrylamide (hydrochloride salt)

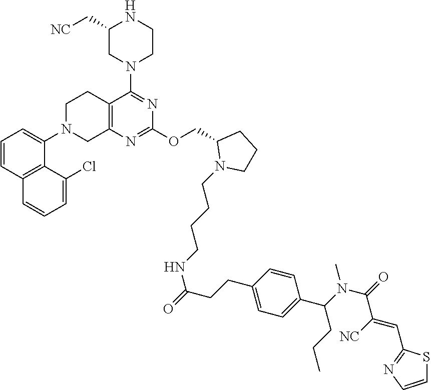 [SMILES: CCCC(c1ccc(CCC(=O)NCCCCN2CCC[C@H]2COc2nc3c(c(N4CCN[C@@H](CC#N)C4)n2)CCN(c2cccc4cccc(Cl)c24)C3)cc1)N(C)C(=O)/C(C#N)=C/c1nccs1]

To a solution of 172 (55 mg, 1 equiv., 0.05 mmol) in DCM (0.2 mL) was added HCl (4M in dioxane) (0.2 mL, 15.5 equiv., 0.8 mmol). The reaction was stirred at rt for 1 hr then the volatiles were removed in vacuo to give the desired product 173 (25 mg, 0.02 mmol, 46.6% yield) as a tan solid.

m/z=969.1 (M+H)⁺

### Example A64: (E)-N-[1-[4-[3-[4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]butylamino]-3-oxo-propyl]phenyl]butyl]-2-cyano-N-methyl-3-thiazol-2-yl-prop-2-enamide

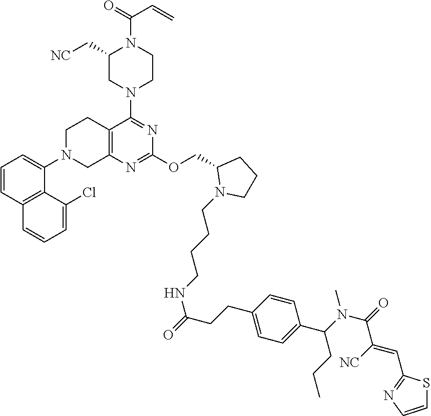 [SMILES: C=CC(=O)N1CCN(c2nc(OC[C@@H]3CCCN3CCCCNC(=O)CCc3ccc(C(CCC)N(C)C(=O)/C(C#N)=C/c4nccs4)cc3)nc3c2CCN(c2cccc4cccc(Cl)c24)C3)C[C@@H]1CC#N]

To a solution of compound 173 (12 mg, 1 equiv., 0.01 mmol) and triethylamine (8.3 uL, 5 equiv., 0.06 mmol) in DCM (0.5 mL) was added prop-2-enoyl chloride (2.7 uL, 3 equiv., 0.04 mmol). The reaction mixture was stirred for 30 min at rt then concentrated in vacuo. Purification by preparative HPLC (using a gradient from 5% to 95% of acetonitrile in water containing 0.1% of formic acid over 10 minutes) gave the purified product A64 (1.7 mg, 0.002 mmol, 13.3% yield) as a white solid

m/z=1023.2 (M+H)⁺

### Example 174—tert-butyl (1-(4-(3-(4-(2,6-dimethoxy-4-(2-methyl-1-oxo-1,2-dihydro-2,7-naphthyridin-4-yl)benzyl)piperazin-1-yl)propyl)phenyl)butyl)(methyl)carbamate

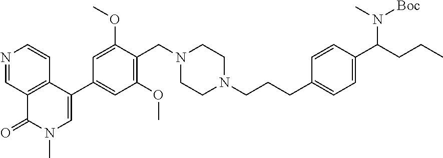 [SMILES: CCCC(c1ccc(CCCN2CCN(Cc3c(OC)cc(-c4cn(C)c(=O)c5cnccc45)cc3OC)CC2)cc1)N(C)C(=O)OC(C)(C)C]

Prepared following general procedure 26 using amine synthesised according to J. Med. Chem. 2019, 62, 2, 699-726. Obtained 72 mg, 66% yield, LCMS m/z=698.9 [M+H]⁺.

### Example 175—4-(3,5-dimethoxy-4-((4-(3-(4-(1-(methylamino)butyl)phenyl)propyl)piperazin-1-yl)methyl)phenyl)-2-methyl-2,7-naphthyridin-1(2H)-one

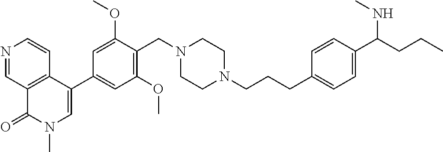 [SMILES: CCCC(NC)c1ccc(CCCN2CCN(Cc3c(OC)cc(-c4cn(C)c(=O)c5cnccc45)cc3OC)CC2)cc1]

Prepared following general procedure 8. Used without further purification. LCMS m/z=598.7 [M+H]⁺.

### Example 176: 2-cyano-N-(1-(4-(3-(4-(2,6-dimethoxy-4-(2-methyl-1-oxo-1,2-dihydro-2,7-naphthyridin-4-yl)benzyl)piperazin-1-yl)propyl)phenyl)butyl)-N-methylacetamide

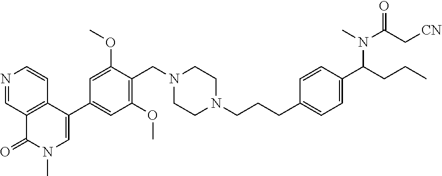 [SMILES: CCCC(c1ccc(CCCN2CCN(Cc3c(OC)cc(-c4cn(C)c(=O)c5cnccc45)cc3OC)CC2)cc1)N(C)C(=O)CC#N]

Prepared following general procedure 9. Obtained 12 mg, 17% yield (over 2 steps).

LCMS m/z=665.8 (M+H)⁺.

### Example A65: (E)-2-cyano-N-(1-(4-(3-(4-(2,6-dimethoxy-4-(2-methyl-1-oxo-1,2-dihydro-2,7-naphthyridin-4-yl)benzyl)piperazin-1-yl)propyl)phenyl)butyl)-N-methyl-3-(thiazol-2-yl)acrylamide

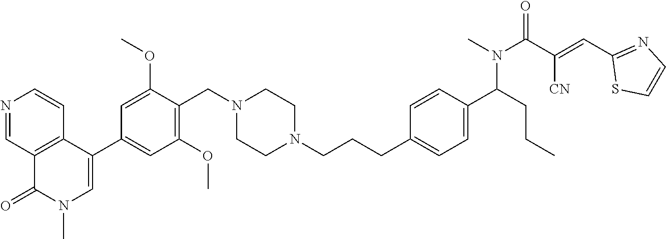 [SMILES: CCCC(c1ccc(CCCN2CCN(Cc3c(OC)cc(-c4cn(C)c(=O)c5cnccc45)cc3OC)CC2)cc1)N(C)C(=O)/C(C#N)=C/c1nccs1]

Prepared following general procedure 10. Obtained 7 mg, 50% yield. LCMS m/z=760.9 [M+H]⁺.

### Example A66—(E)-2-cyano-N-(1-(4-(3-(4-(2,6-dimethoxy-4-(2-methyl-1-oxo-1,2-dihydro-2,7-naphthyridin-4-yl)benzyl)piperazin-1-yl)-3-oxopropyl)phenyl)butyl)-N-methyl-3-(thiazol-2-yl)acrylamide

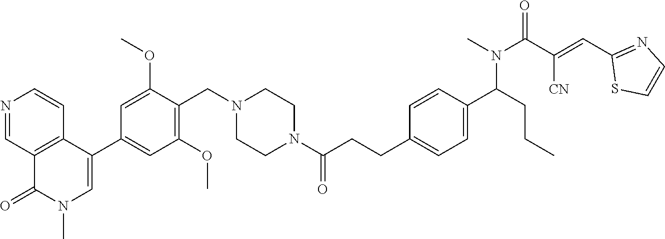 [SMILES: CCCC(c1ccc(CCC(=O)N2CCN(Cc3c(OC)cc(-c4cn(C)c(=O)c5cnccc45)cc3OC)CC2)cc1)N(C)C(=O)/C(C#N)=C/c1nccs1]

Prepared following general procedure 31 using amine synthesised according to J. Med. Chem. 2019, 62, 2, 699-726. Obtained 12 mg, 45% yield, LCMS m/z 774.8 [M+H]⁺.

### Example 177—1-isopropyl-6-((2-(4-methoxypiperidin-1-yl)pyrimidin-4-yl)amino)-1H-pyrazolo[4,3-c]pyridine-3-carboxylate

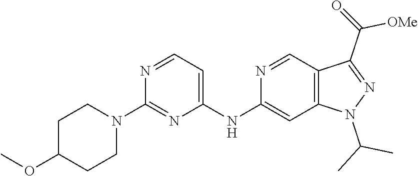 [SMILES: COC(=O)c1nn(C(C)C)c2cc(Nc3ccnc(N4CCC(OC)CC4)n3)ncc12]

To a stirred solution of methyl 6-chloro-1-isopropyl-1H-pyrazolo[4,3-c]pyridine-3-carboxylate (prepared according to WO2014210354, the entire contents of which are incorporated herein by reference) (3 g, 1.0 equiv, 11.83 mmol) and 2-(4-methoxypiperidin-1-yl)pyrimidin-4-amine (prepared according to WO2014210354, the entire contents of which are incorporated herein by reference) (2.96 g, 1.2 equiv., 14.19 mmol) in 1,4-dioxane (30 ml), was added Cs₂CO₃ (7.71 g, 2.0 equiv., 23.65 mmol). The reaction mixture was degassed with N₂ for 15 min followed by the addition of XPhos (0.56 g, 0.1 equiv., 1.18 mmol) and Pd₂(dba)₃ (1.62 g, 0.15 equiv., 1.77 mmol) at RT. The reaction mixture was heated to 100° C. for 16 h then was filtered through celite and the solid was washed with EtOAc. The filtrate was concentrated in vacuo and the resulting residue was purified by flash chromatography (EtOAc in n-hexane (30% to 40%)) as an eluent. The product containing fractions were concentrated in vacuo obtain compound 177 (3.1 g, 7.01 mmol, 59.3% yield).

LCMS m/z=426.0 [M+H]⁺.

### Example 178—1-isopropyl-6-((2-(4-methoxypiperidin-1-yl)pyrimidin-4-yl)amino)-1H-pyrazolo[4,3-c]pyridine-3-carboxylic acid

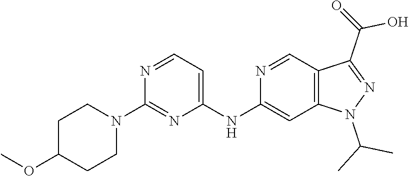 [SMILES: COC1CCN(c2nccc(Nc3cc4c(cn3)c(C(=O)O)nn4C(C)C)n2)CC1]

Prepared following general procedure 7 in THF, MeOH and H₂O. Obtained 2.1 g, 71.6% yield, LCMS m/z=412.3 [M+H].

### Example 179—(3-(4-(3-aminopropyl)piperazin-1-yl)propyl)carbamate

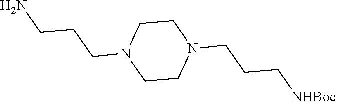 [SMILES: CC(C)(C)OC(=O)NCCCN1CCN(CCCN)CC1]

Prepared following general procedure 5 in acetone. Obtained via reverse phase preparative HPLC, 800 mg, 24% yield, LCMS m/z=301.2 [M+H]⁺.

### Example 180—tert-butyl (3-(4-(3-(1-isopropyl-6-((2-(4-methoxypiperidin-1-yl)pyrimidin-4-yl)amino)-1H-pyrazolo[4,3-c]pyridine-3-carboxamido) propyl) piperazin-1-yl)propyl)carbamate

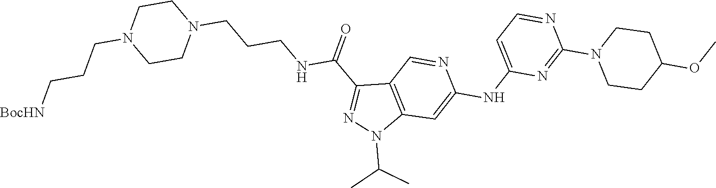 [SMILES: COC1CCN(c2nccc(Nc3cc4c(cn3)c(C(=O)NCCCN3CCN(CCCNC(=O)OC(C)(C)C)CC3)nn4C(C)C)n2)CC1]

Prepared following general procedure 31. Obtained 120 mg, 62.6% yield, LCMS m/z=694.7 [M+H]⁺.

### Example 181—N-(3-(4-(3-aminopropyl)piperazin-1-yl)propyl)-1-isopropyl-6-((2-(4-methoxypiperidin-1-yl)pyrimidin-4-yl)amino)-1H-pyrazolo[4,3-c]pyridine-3-carboxamide hydrochloride

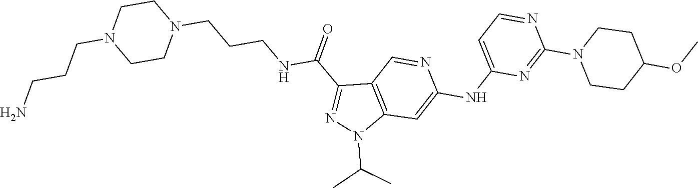 [SMILES: COC1CCN(c2nccc(Nc3cc4c(cn3)c(C(=O)NCCCN3CCN(CCCN)CC3)nn4C(C)C)n2)CC1]

Prepared following general procedure 8. Obtained 100 mg, 85% yield, LCMS m/z=594.7 [M+H]⁺.

### Example A67—(E)-N-(3-(4-(3-(3-(4-(1-(2-cyano-N-methyl-3-(thiazol-2-yl)acrylamido) butyl)phenyl)propanamido)propyl)piperazin-1-yl)propyl)-1-isopropyl-6-((2-(4-methoxypiperidin-1-yl)pyrimidin-4-yl)amino)-1H-pyrazolo[4,3-c]pyridine-3-carboxamide

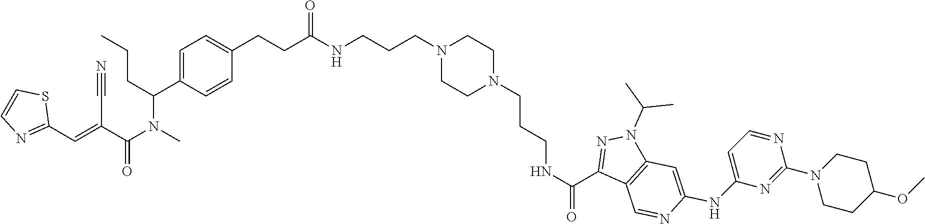 [SMILES: CCCC(c1ccc(CCC(=O)NCCCN2CCN(CCCNC(=O)c3nn(C(C)C)c4cc(Nc5ccnc(N6CCC(OC)CC6)n5)ncc34)CC2)cc1)N(C)C(=O)/C(C#N)=C/c1nccs1]

Prepared following general procedure 31. Purified by preparative HPLC. Obtained 45 mg, 28.1% yield, LCMS m/z=973.7 [M+H]⁺.

### PART B—BIOLOGICAL DATA

The bifunctional compounds were assayed to investigate their ability to degrade target proteins in accordance with the following general procedures.

1.1 Assay 1—Degradation of HiBit-BRD4 in HEK293

HEK293 containing a HiBit insertion for BRD4 were plated in 384-well tissue culture plates at a density of 8×10⁴ per well in a volume of 36 μL and incubated overnight at 37° C. and 5% CO₂. Wells were treated with test compounds for 6 h prior to addition of the NanoLuc substrate and reading on a ClarioSLARIOstar Plus. Degradation data was plotted and analysed using Prism 86 (Graphpad).

1.2 Assay 2—CDK9 Degradation in MV4;11

MV4;11 (0.8×10⁶ cells/mL) were seeded in 6-well plates (3 mL IMDM supplemented with 10% FBS and L-glutamine) overnight before treatment with compounds at the desired concentration and with a final DMSO concentration of 0.1% v/v. After 8 h incubation time, cells were washed with DPBS (Gibco) and lysed using 85 μL RIPA buffer (Sigma-Aldrich) supplemented with cOmplete Mini EDTA-free protease inhibitor cocktail (Roche) and benzonase. Lysates were clarified by centrifugation (20000 g, 10 min, 4° C.) and the total protein content of the supernatant was quantified using a BCA assay. Samples were prepared using equal amounts of total protein and LDS sample buffer (Invitrogen). For immunoblot analysis, the following antibodies were used: anti-CDK9 (CST-2316S, 1:1,000 dilution), and anti-GAPDH hFAB-rhodamine (BioRad, 12004168, 1:5000 dilution). Band intensities were normalized to the GAPDH loading control and reported as % of the average 0.1% DMSO vehicle intensity.

1.3 Assay 3—MV4;11 Cell Viability Using CellTiter-Glo Assay

The anti-proliferative effects of representative compounds were measured using the CellTiter-Glo assay (Promega). MV4;11 cells were seeded into sterile, white, clear-bottomed 384-well cell-culture microplates (Greiner Bio-one), at 2× concentration in IMDM media and a volume of 25 μL. Test compounds were serially diluted (11-pt dose-response from 1 μM) in IMDM media to 2× concentration, then added to cells to make a final volume of 50 μL. Final DMSO concentration was 0.05%. After 48 h incubation, 25 μL of CellTiter-Glo reagent was added to each well. Following a 15 minutes incubation the luminescence signal was read on a CLARIOStar Plus. Data was processed and dose-response curves were generated using Prism 8 (Graphpad).

1.4 Assay 4—Endpoint Degradation Using HiBit-CDK9.

HiBit-CDK9 HEK293 cells were diluted in OptiMEM media with 4% FBS to 2.2×10⁵ cells/mL, and dispensed into a sterile, white, clear-bottomed 384-well cell-culture microplate (Greiner Bio-one) at a volume of 36 μL. Plates were incubated for 24 h at 37° C. Test compounds were serially diluted in OptiMEM to 10× their desired final concentration and added to the assay plate at a volume of 4 μL. After 6 h incubation, Nanoluc substrate was diluted to 1× in OptiMEM media and added to each well at a volume of 10 μL. The plate was read immediately on a Clariostar-Plus (BMG). Dose-response curves were generated in Prism 8 (Graphpad).

1.5 Assay 5—Degradation of BRD9 by Immunoblot.

HEK293 cells (0.4×10⁶) were seeded in a 12-well plate (1 mL medium) overnight prior to 24 h treatment with test compounds at the desired concentration. After 24 h incubation, cells were washed with PBS and lysed with RIPA buffer (Sigma) supplemented with 1× protease inhibitor cocktail (Roche) and 1 U/mL Benzonase (Merck). Lysates were clarified by centrifugation (17,000×g, 20 min, 4° C.) and the total protein content of the supernatant was quantified using a BCA assay. Samples were prepared using equal amounts of total protein and LDS sample buffer (Invitrogen). Samples were resolved by SDS-PAGE using NuPAGE 4-12% Bis-Tris midi gels (Invitrogen, followed by transfer to Amersham Protran 0.45 NC nitrocellulose membrane (GE Healthcare) using wet transfer. Precision Plus Protein All Blue (Bio-Rad) protein ladder was used as a standard. The membrane was blocked with 5% powdered skimmed milk (Marvel) in Tris-buffered saline with 0.1% Tween-20 (TBST). Blots were probed (overnight at 4° C.) using the following primary antibodies (diluted in 5% BSA in TBST) as appropriate: anti-BRD9 (Bethyl A303-781A, 1:1000) and anti-BRD7 (Cell Signalling #15125, rabbit, 1:2000). The next day, blots were washed with TBST and incubated (1 h at RT) with anti-Tubulin hFAB-rhodamine (BioRad, 12004166. 1:30000) and and anti-GAPDH hFAB-rhodamine (#12004168, 1:5000) primary antibodies, plus either anti-rabbit IRDye 800CW (Licor 1:10,000 dilution) or anti-mouse IRDye 800CW (Licor 1:10,000 dilution) secondary antibody. Blots were visualised using a Bio-Rad ChemiDoc MP Imaging System, and band quantification was performed using Image Studio software (LiCor). Band intensities were normalized to the loading controls and reported as % of the average 0.1% DMSO vehicle intensity. Degradation data was plotted and analysed using Prism (Graphpad, version 8).

1.6 Assay 6—Degradation of KRas-G12C by Immunoblot.

MIA-PaCa-2 cells were seeded in 6- or 12-well plates overnight prior to 24 h treatment with test compounds at the desired concentration (DMSO final concentration 0.1%). After 24 h incubation, cells were washed with PBS and lysed with RIPA buffer (Sigma) supplemented with 1 mM MgCl₂, 1 U/mL Benzonase (Sigma), and 1× cOmplete Mini EDTA-free Protease Inhibitor Cocktail (Roche). Lysates were clarified by centrifugation (15,000×g, 20 min, 4° C.) and the total protein content of the supernatant was quantified using a BCA assay. Samples were prepared using equal amounts of total protein and LDS sample buffer (Invitrogen). Samples were resolved by SDS-PAGE using NuPAGE 4-12% Bis-Tris midi gels (Invitrogen, followed by transfer to Amersham Protran 0.45 NC nitrocellulose membrane (GE Healthcare) using wet transfer. Precision Plus Protein All Blue (Bio-Rad) protein ladder was used as a standard. Membranes were blocked with 5% powdered skimmed milk (Marvel) in Tris-buffered saline with 0.1% Tween-20 (TBST), or 5% PhosphoBlocker (Cell BioLabs). Blots were probed (overnight at 4° C.) using the following primary antibodies (diluted in 5% BSA in TBST) as appropriate: panKras (Sigma, SAB1404011, 1:2,000), panKras (Abcam, ab275876, 1:1,000), p44/42 Erk1/2 (AF647 conjugate, Cell Signaling Technologies 5376, 1:2,000), phosphor-p44/42 Erk1/2 (Thr202/Tyr204) (AF488 conjugate, Cell Signaling Technologies 13214, 1:1,000). The next day, blots were washed with TBST and incubated (1 h at RT) with anti-Tubulin hFAB-rhodamine (BioRad, 12004166. 1:10,000) and and anti-GAPDH hFAB-rhodamine (BioRad, #12004168, 1:10,000) primary antibodies, plus either anti-rabbit IRDye 800CW (Licor 1:10,000), anti-Rabbit StarBright Blue 700 (BioRad, 1:10,000), Goat Anti-Mouse StarBright Blue 700 (BioRad, 1:10,000) or GoatAnti-Mouse StarBright Blue 520 (BioRad, 1:10,000) secondary antibody as appropriate. Blots were visualised using a Bio-Rad ChemiDoc MP Imaging System, and band quantification was performed using Image Studio software (LiCor). Band intensities were normalized to the loading controls and reported as % of the average 0.1% DMSO vehicle intensity. Degradation data was plotted and analysed using Prism (Graphpad, version 8).

1.7 Assay 7—Degradation of PARP1 by Capillary Electrophoresis.

HCC1937 cells were seeded (0.5 million cells/well) in 24-well plates overnight prior to 24 h treatment with test compounds at the desired concentration (DMSO final concentration 0.2%). After 24 h incubation, cells were washed with PBS and lysed with RIPA buffer (Sigma) containing 1× cOmplete Mini EDTA-free Protease Inhibitor Cocktail (Roche). Lysates were clarified by centrifugation (10,000 rpm, 10 min, 4° C.) and the total protein content of the supernatant was quantified using a BCA assay. Capillary-based immunoassays were performed using a standard WES (Simple Western) protocol (ProteinSimple). Lysates were loaded onto WES plates at 1.5 μg/well total protein. The following antibodies and antibody concentrations were used: Anti-PARP(CST #9532, 1:250 dilution), Anti-Tubulin (CST #2125, 1:250 dilution), secondary Anti-Rabbit(CST #7074S, 1:500 dilution). Data was produced by the WES Compass software as chemiluminescent counts and displayed as an electropherogram, and the chemiluminescent peak area value was used for all calculations. The amount of target protein was normalized to the loading control and reported as % of the average 0.1% DMSO vehicle intensity. Degradation data was plotted and analysed using Prism (Graphpad, version 8).

1.8 Assay 8—Degradation of Mutant EGFR by Immunoblot.

NCI-H1975 (1.5×10⁵) cells were seeded in 12-well plates (1 mL medium) overnight prior to 24 h treatment with test compounds at the desired concentration (DMSO final concentration 0.1%). After 24 h incubation, cells were washed with PBS and lysed with RIPA buffer (Sigma) supplemented with 1× protease inhibitor cocktail (Roche) and 1 U/mL Benzonase (Merck). Lysates were clarified by centrifugation (17,000×g, 20 min, 4° C.) and the total protein content of the supernatant was quantified using a BCA assay. Samples were prepared using equal amounts of total protein and LDS sample buffer (Invitrogen). Samples were resolved by SDS-PAGE using NuPAGE 4-12% Bis-Tris midi gels (Invitrogen, followed by transfer to Amersham Protran 0.45 NC nitrocellulose membrane (GE Healthcare) using wet transfer. Precision Plus Protein All Blue (Bio-Rad) protein ladder was used as a standard. The membrane was blocked with 5% powdered skimmed milk (Marvel) in Tris-buffered saline with 0.1% Tween-20 (TBST). Blots were probed (overnight at 4° C.) using the following primary antibodies (diluted in 5% BSA in TBST) as appropriate: total EGFR (Cell Signaling Technology, CST #4267, 1:2000), L858R EGFR (Cell Signaling Technology, CST #3197, 1:1000) and phospho-EGFR (Cell Signaling Technology, CST #3777,1:2000). The next day, blots were washed with TBST and incubated (1 h at RT) with anti-Tubulin hFAB-rhodamine (BioRad, 12004166. 1:20000) and and anti-GAPDH hFAB-rhodamine (#12004168, 1:20000) primary antibodies, plus either anti-rabbit IRDye 800CW (Licor 1:20,000 dilution) or anti-mouse IRDye 800CW (Licor 1:20,000 dilution) secondary antibody. Blots were visualised using a Bio-Rad ChemiDoc MP Imaging System, and band quantification was performed using Image Studio software (LiCor). Band intensities were normalized to loading controls and reported as % of the average 0.1% DMSO vehicle intensity. Degradation data was plotted and analysed using Prism (Graphpad, version 8).

### Example 1

The impact on cell viability of MV4;11 cells was evaluated according to the procedure outlined in assay 3 for compounds A1, A2, A8 and A9. The IC₅₀-values are shown in table 1 below.

| Compound No. | IC₅₀ (nM) |
|---|---|
| A1 | 48 |
| A2 | 2 |
| A8 | 1 |
| A9 | 3 |

Table 1 shows IC₅₀ parameters for compounds A1, A2, A8 and A9.

It was surprisingly observed that modifications to the amido group on the warhead (as present in A2, A8 and A9) gave significant improvements in terms of the anti-proliferative activity (cell potency) of the bifunctional molecule as evidenced by their significantly lower IC₅₀ values.

The enhanced cell potency of these compounds has been shown to correlate well with significantly improved BET degradation, particularly lower DC₅₀ and higher Dₘₐₓ parameters. For example, the correlation between the IC₅₀ and DC₅₀ values for a number of bifunctional molecules is shown in FIG. 1.

### Example 2—Efficacy of A2 in a Cancer Cell Panel

Cancer cell panel screening was provided as a service from OncoLead GmbH & Co. KG. After a lag phase of 48 h, each cell line was treated with six different concentrations (10⁻¹⁰, 10⁻⁹, 10⁻⁸, 10⁻⁷, 10⁻⁶, 10⁻⁵ M) of either A2 or I-BET726 for 72 h. Concentrations to give half-maximal growth inhibition (GI₅₀) were determined using the Sulforhodamine B method. Log(GI₅₀) values were plotted as single points (I-BET726, cross; A2, dot) superimposed on the bar graph and plotted along the right y axis. Log ratio of the GI₅₀ determined for I-BET726 versus the GI₅₀ determined for A2 were plotted for each cell line tested (bars, left y axis).

Values>0 indicate cell lines where BET-degradation by A2 shows greater efficacy than the inhibitor I-BET726 due to catalytic activity, whereas values<0 indicate cell lines where BET degrader A2 is less efficacious than BET-inhibition with I-BET726. Where no specific GI₅₀ value could be determined, a GI₅₀ of at least 10⁻⁵ M was assumed to calculate the inhibitor vs degrader ratio.

The results are illustrated on FIG. 2 and show that A2 shows a broad efficacy against a wide range of tumour cell lines.

Similar studies have previously been carried out on CRBN PROTAC (dBET6) and VHL PROTAC (MZ1) (see, for example, Ottis et al, ACS Chem. Biol. 2019, 14, 2215-2223 under identical assay conditions). A comparison of these results is shown in table 2 below.

GI₅₀ comparison (BET degrader: up to 94 tumor
cell lines) CRBN (dBET6) vs VHL (MZ1) vs A2

| | # tumor cell lines | | |
|---|---|---|---|
| | Highly | Low sensitivity/ | |
| Degrader Treatment | sensitive | Resistant | GI₅₀, <1 μM |
| CRBN PROTAC (dBET6) | 29% | 52% | 58% |
| VHL PROTAC (MZ1) | 47% | 26% | 83% |
| A2 | 66% | 15% | 100% |

Table 2 shows GI₅₀ comparison (BET degrader: up to 94 tumor cell lines) for CRBN (dBET6), VHL (MZ1) and A2. Highly sensitive defined as degrader GI₅₀>3 fold more potent than inhibitor. Low sensitivity/resistant defined as degrader GI₅₀<inhibitor. Other cell lines showed intermediate activity.

The data demonstrate that A2 shows a broader range of efficacy across the tumour cell lines tested in comparison to the CRBN PROTAC degrader (dBET6) and VHL PROTAC degrader (MZ1).

Further N-alkylated warheads were then investigated to determine their ability to promote selective protein degradation in two test systems (one in which the target protein was BRD4 and the other in which the target protein was the kinase CDK9).

### Example 3a—BRD4 Degradation

The degradation of target protein BRD4 was detected according to the procedure outlined in assay 1 for the following compounds.

| No. | Structure |
|---|---|
| A2 | 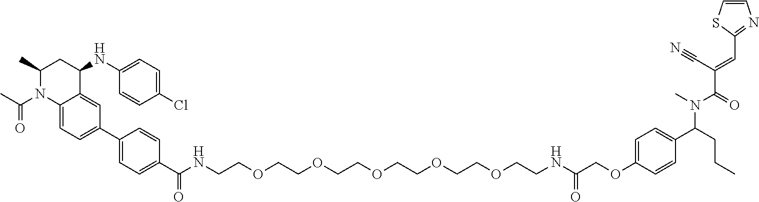 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)/C(C#N)=C/c1nccs1] |
| A3 | 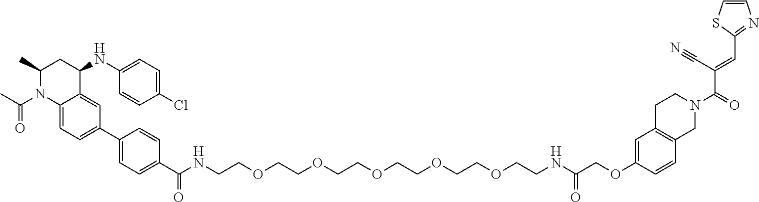 [SMILES: CC(=O)N1c2ccc(-c3ccc(C(=O)NCCOCCOCCOCCOCCOCCNC(=O)COc4ccc5c(c4)CCN(C(=O)/C(C#N)=C/c4nccs4)C5)cc3)cc2[C@H](Nc2ccc(Cl)cc2)C[C@@H]1C] |
| A4 | 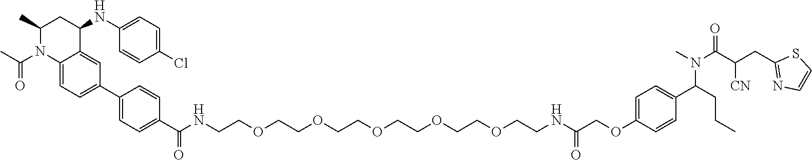 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)C(C#N)Cc1nccs1] |
| A5 | 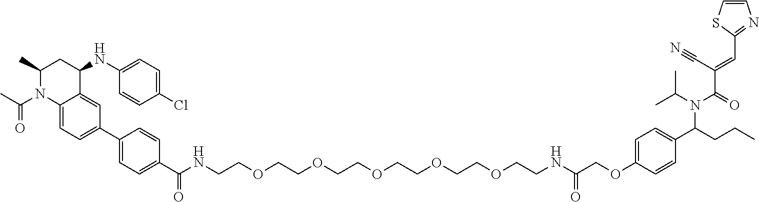 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C(=O)/C(C#N)=C/c1nccs1)C(C)C] |
| A6 | 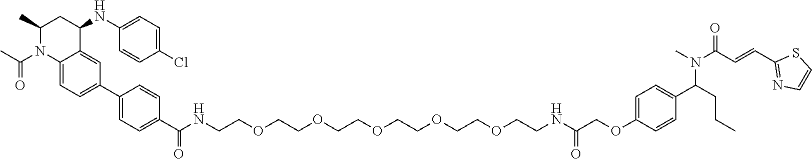 [SMILES: CCCC(c1ccc(OCC(=O)NCCOCCOCCOCCOCCOCCNC(=O)c2ccc(-c3ccc4c(c3)[C@H](Nc3ccc(Cl)cc3)C[C@H](C)N4C(C)=O)cc2)cc1)N(C)C(=O)/C=C/c1nccs1] |
| A7 | 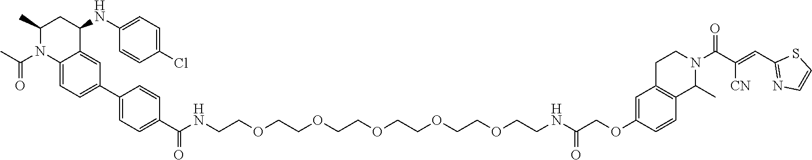 [SMILES: CC(=O)N1c2ccc(-c3ccc(C(=O)NCCOCCOCCOCCOCCOCCNC(=O)COc4ccc5c(c4)CCN(C(=O)/C(C#N)=C/c4nccs4)C5C)cc3)cc2[C@H](Nc2ccc(Cl)cc2)C[C@@H]1C] |
| A8 | 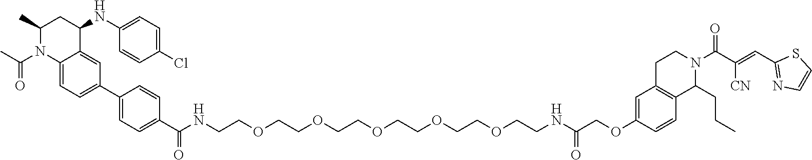 [SMILES: CCCC1c2ccc(OCC(=O)NCCOCCOCCOCCOCCOCCNC(=O)c3ccc(-c4ccc5c(c4)[C@H](Nc4ccc(Cl)cc4)C[C@H](C)N5C(C)=O)cc3)cc2CCN1C(=O)/C(C#N)=C/c1nccs1] |
| A9 | 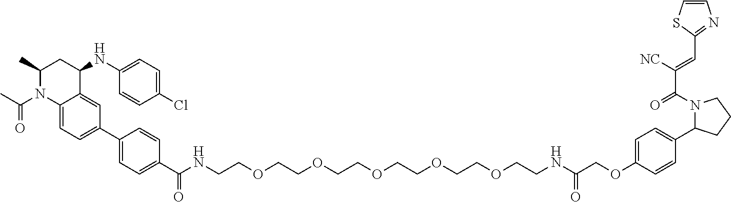 [SMILES: CC(=O)N1c2ccc(-c3ccc(C(=O)NCCOCCOCCOCCOCCOCCNC(=O)COc4ccc(C5CCCN5C(=O)/C(C#N)=C/c5nccs5)cc4)cc3)cc2[C@H](Nc2ccc(Cl)cc2)C[C@@H]1C] |

Table 3 shows the bifunctional molecules that were analysed in accordance with the procedure outlined in assay 1.

The DC₅₀ values for compounds A2 to A5 and A7 to A9 were found to be less than 1000 nM. The DC₅₀ for compound A6 was found to be less than 10000 nM. These molecules are all considered to be effective degraders.

### Example 4

The degradation of target protein CDK9 was detected according to the procedure outlined in assay 2 for the following compounds.

| Compound | |
|---|---|
| no. | Structure |
| A10 | 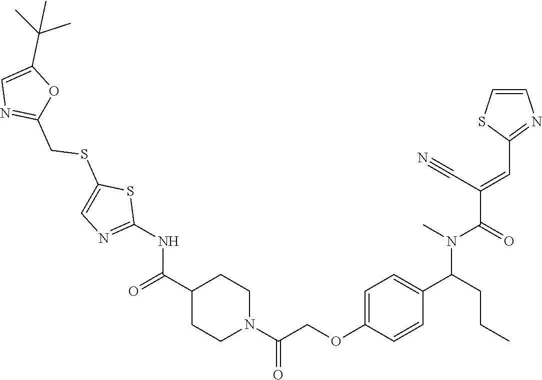 [SMILES: CCCC(c1ccc(OCC(=O)N2CCC(C(=O)Nc3ncc(SCc4ncc(C(C)(C)C)o4)s3)CC2)cc1)N(C)C(=O)/C(C#N)=C/c1nccs1] |
| A11 | 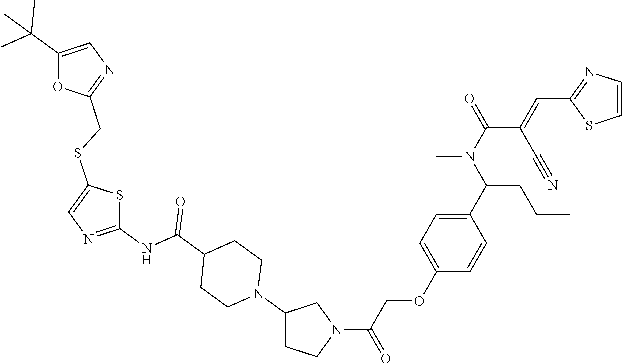 [SMILES: CCCC(c1ccc(OCC(=O)N2CCC(N3CCC(C(=O)Nc4ncc(SCc5ncc(C(C)(C)C)o5)s4)CC3)C2)cc1)N(C)C(=O)/C(C#N)=C/c1nccs1] |
| A12 | 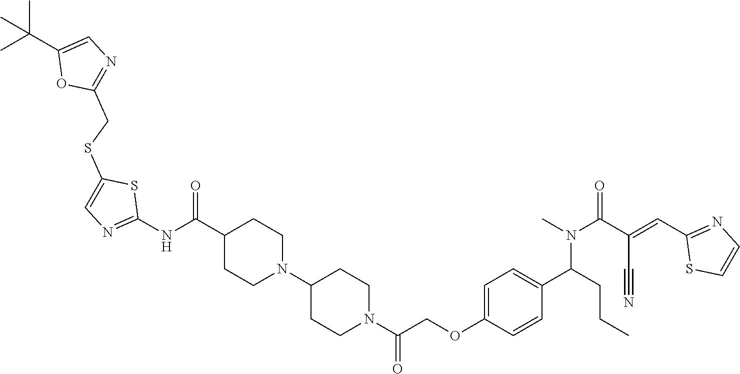 [SMILES: CCCC(c1ccc(OCC(=O)N2CCC(N3CCC(C(=O)Nc4ncc(SCc5ncc(C(C)(C)C)o5)s4)CC3)CC2)cc1)N(C)C(=O)/C(C#N)=C/c1nccs1] |
| A13 | 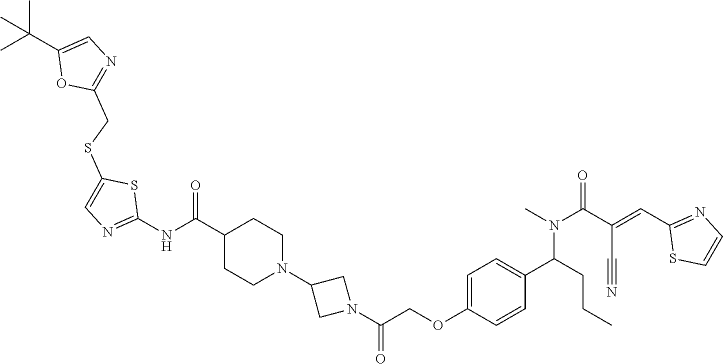 [SMILES: CCCC(c1ccc(OCC(=O)N2CC(N3CCC(C(=O)Nc4ncc(SCc5ncc(C(C)(C)C)o5)s4)CC3)C2)cc1)N(C)C(=O)/C(C#N)=C/c1nccs1] |
| A14 | 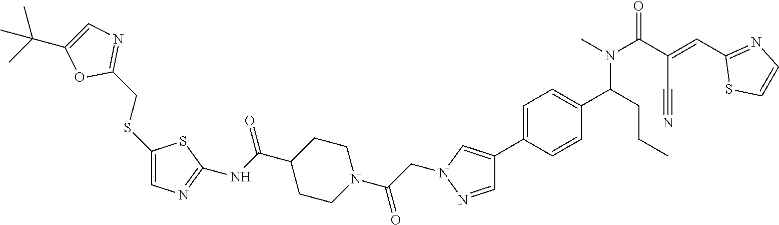 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1nccs1] |
| A15 | 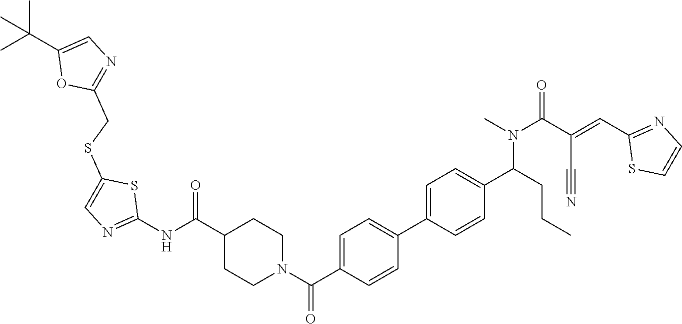 [SMILES: CCCC(c1ccc(-c2ccc(C(=O)N3CCC(C(=O)Nc4ncc(SCc5ncc(C(C)(C)C)o5)s4)CC3)cc2)cc1)N(C)C(=O)/C(C#N)=C/c1nccs1] |
| A16 | 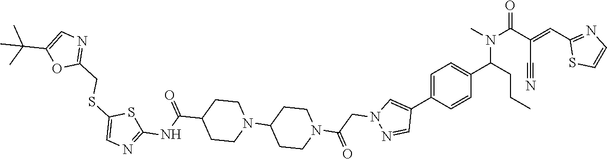 [SMILES: CCCC(c1ccc(-c2cnn(CC(=O)N3CCC(N4CCC(C(=O)Nc5ncc(SCc6ncc(C(C)(C)C)o6)s5)CC4)CC3)c2)cc1)N(C)C(=O)/C(C#N)=C/c1nccs1] |
| A17 | 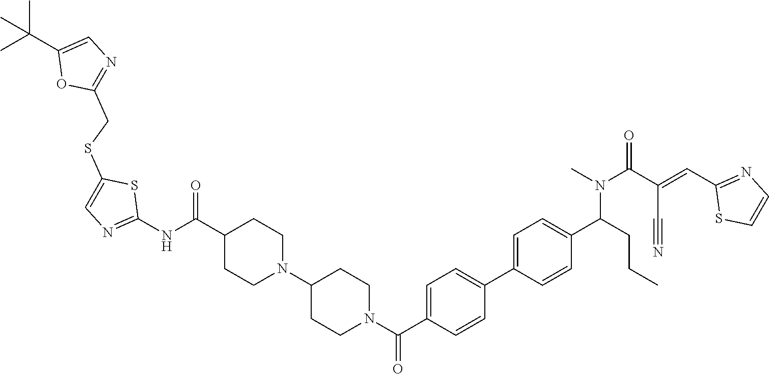 [SMILES: CCCC(c1ccc(-c2ccc(C(=O)N3CCC(N4CCC(C(=O)Nc5ncc(SCc6ncc(C(C)(C)C)o6)s5)CC4)CC3)cc2)cc1)N(C)C(=O)/C(C#N)=C/c1nccs1] |
| A18 | 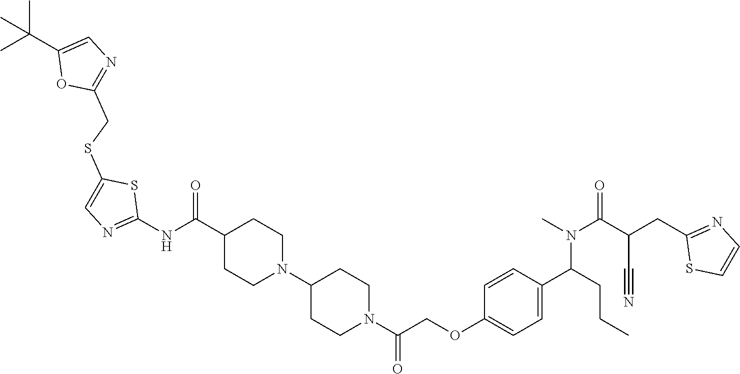 [SMILES: CCCC(c1ccc(OCC(=O)N2CCC(N3CCC(C(=O)Nc4ncc(SCc5ncc(C(C)(C)C)o5)s4)CC3)CC2)cc1)N(C)C(=O)C(C#N)Cc1nccs1] |
| A19 | 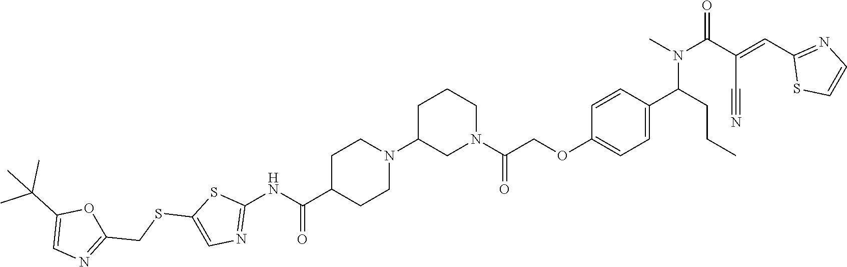 [SMILES: CCCC(c1ccc(OCC(=O)N2CCCC(N3CCC(C(=O)Nc4ncc(SCc5ncc(C(C)(C)C)o5)s4)CC3)C2)cc1)N(C)C(=O)/C(C#N)=C/c1nccs1] |
| A20 | 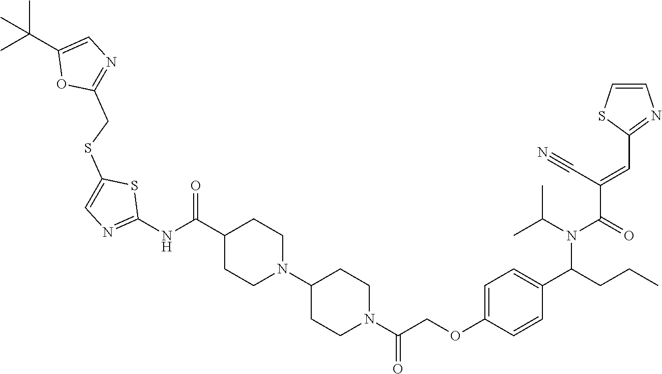 [SMILES: CCCC(c1ccc(OCC(=O)N2CCC(N3CCC(C(=O)Nc4ncc(SCc5ncc(C(C)(C)C)o5)s4)CC3)CC2)cc1)N(C(=O)/C(C#N)=C/c1nccs1)C(C)C] |
| A21 | 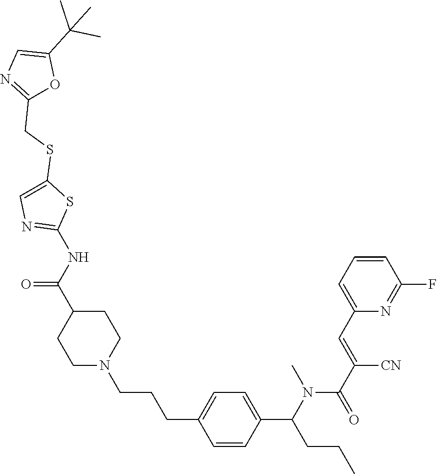 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1cccc(F)n1] |
| A22 | 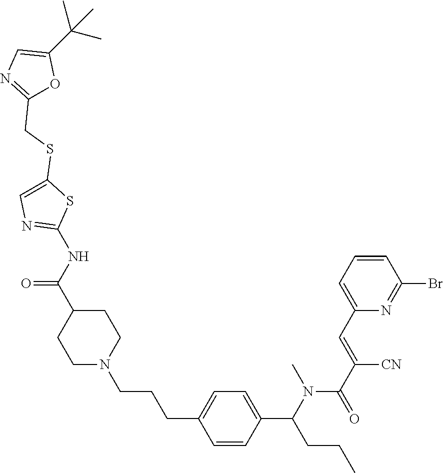 [SMILES: CCCC(c1ccc(CCCN2CCC(C(=O)Nc3ncc(SCc4ncc(C(C)(C)C)o4)s3)CC2)cc1)N(C)C(=O)/C(C#N)=C/c1cccc(Br)n1] |
| A23 | 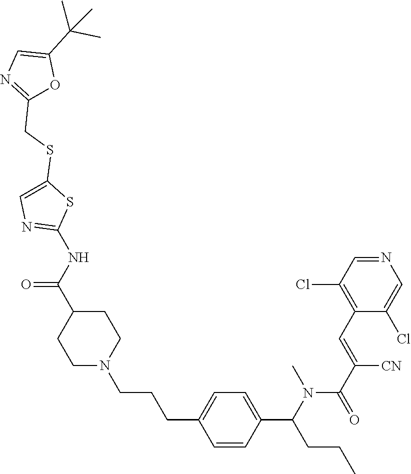 |
| A24 | 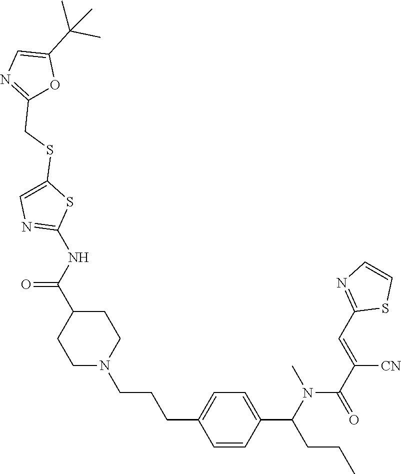 |
| A25 | 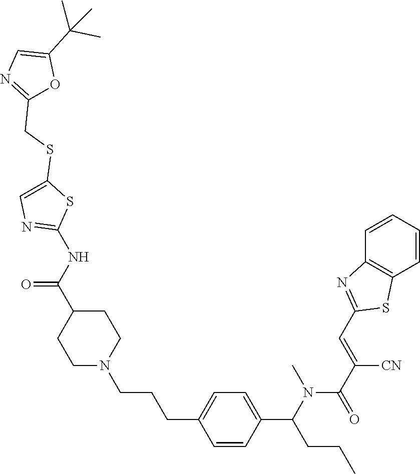 |
| A26 | 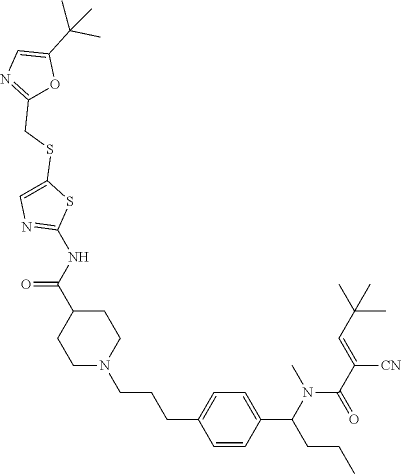 |
| A27 | 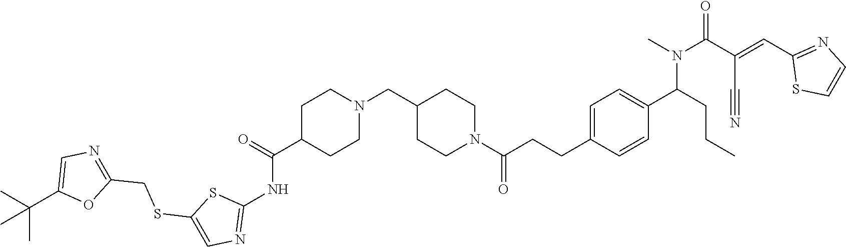 [SMILES: CCCC(c1ccc(CCC(=O)N2CCC(CN3CCC(C(=O)Nc4ncc(SCc5ncc(C(C)(C)C)o5)s4)CC3)CC2)cc1)N(C)C(=O)/C(C#N)=C/c1nccs1] |
| A28 | 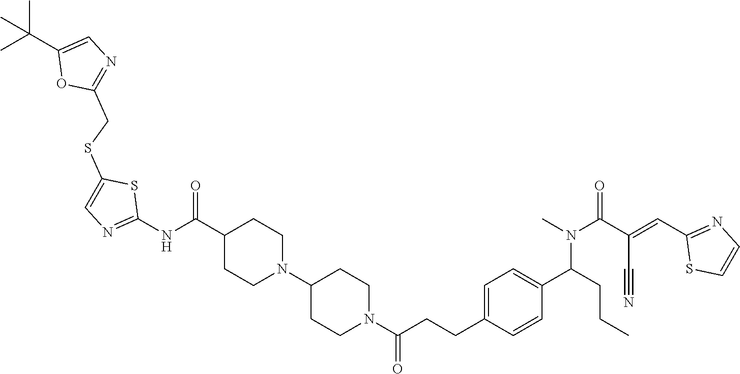 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)/C(C#N)=C/c1nccs1] |
| A29 | 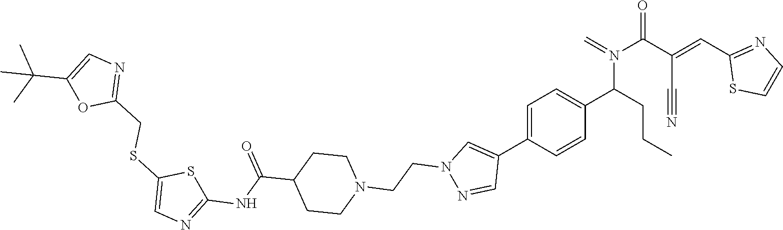 |
| A30 | 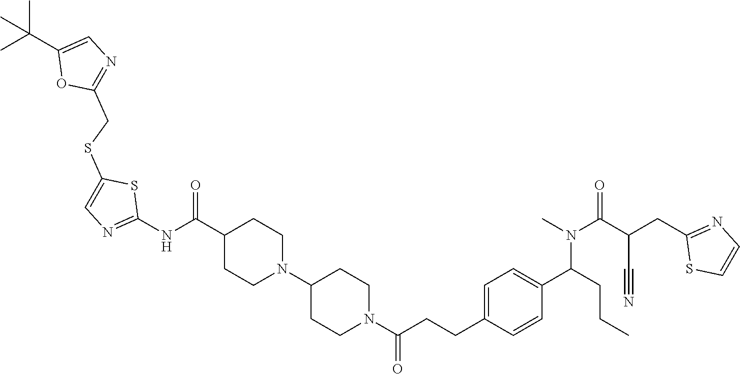 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)C(C#N)Cc1nccs1] |
| A31 | 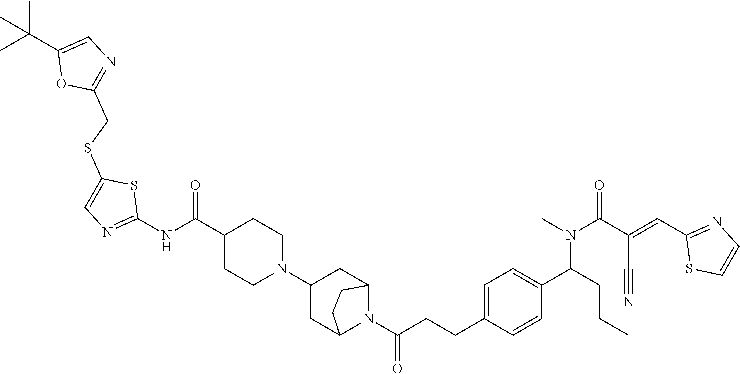 [SMILES: CCCC(c1ccc(CCC(=O)N2C3CCC2CC(N2CCC(C(=O)Nc4ncc(SCc5ncc(C(C)(C)C)o5)s4)CC2)C3)cc1)N(C)C(=O)/C(C#N)=C/c1nccs1] |
| A32 | 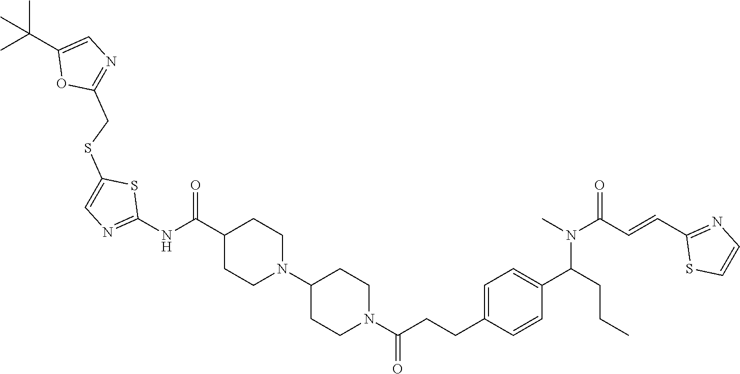 [SMILES: CCCC(c1ccc(CCC(=O)N2CCC(N3CCC(C(=O)Nc4ncc(SCc5ncc(C(C)(C)C)o5)s4)CC3)CC2)cc1)N(C)C(=O)/C=C/c1nccs1] |
| A33 | 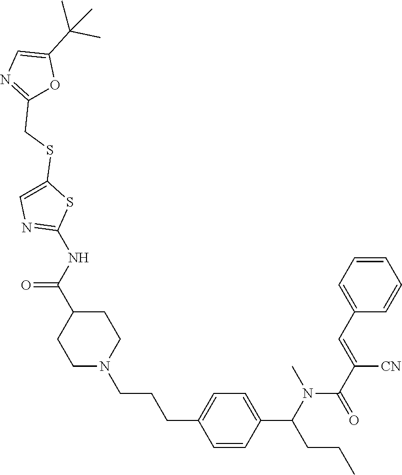 |
| A34 | 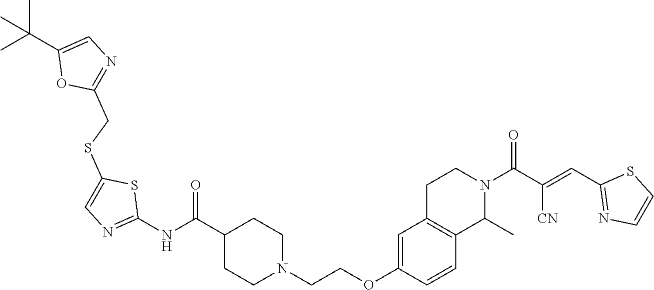 [SMILES: CC1c2ccc(OCCN3CCC(C(=O)Nc4ncc(SCc5ncc(C(C)(C)C)o5)s4)CC3)cc2CCN1C(=O)/C(C#N)=C/c1nccs1] |
| A35 | 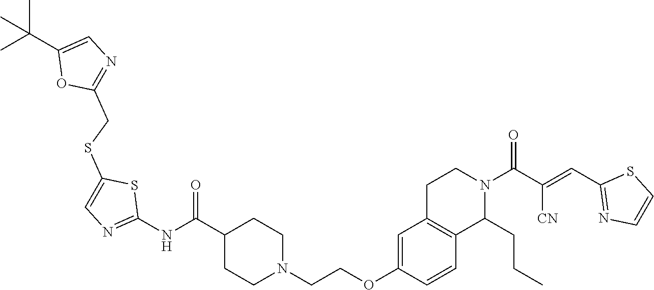 [SMILES: CCCC1c2ccc(OCCN3CCC(C(=O)Nc4ncc(SCc5ncc(C(C)(C)C)o5)s4)CC3)cc2CCN1C(=O)/C(C#N)=C/c1nccs1] |
| A36 | 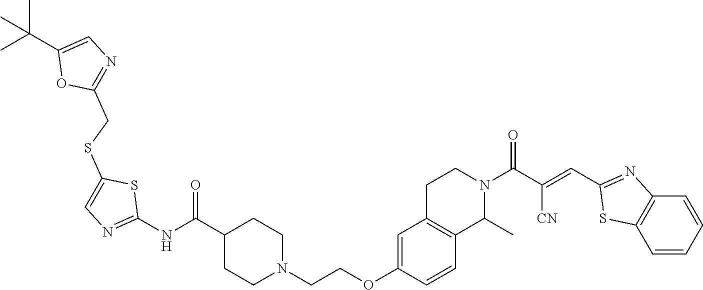 [SMILES: CC1c2ccc(OCCN3CCC(C(=O)Nc4ncc(SCc5ncc(C(C)(C)C)o5)s4)CC3)cc2CCN1C(=O)/C(C#N)=C/c1nc2ccccc2s1] |
| A37 | 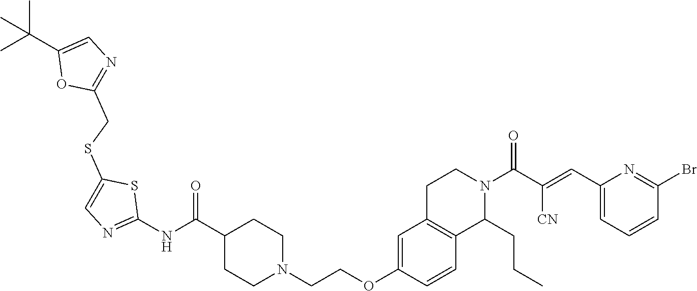 [SMILES: CCCC1c2ccc(OCCN3CCC(C(=O)Nc4ncc(SCc5ncc(C(C)(C)C)o5)s4)CC3)cc2CCN1C(=O)/C(C#N)=C/c1cccc(Br)n1] |
| A38 | 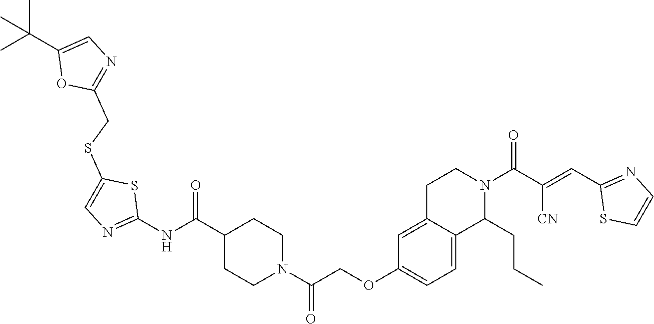 [SMILES: CCCC1c2ccc(OCC(=O)N3CCC(C(=O)Nc4ncc(SCc5ncc(C(C)(C)C)o5)s4)CC3)cc2CCN1C(=O)/C(C#N)=C/c1nccs1] |
| A39 | 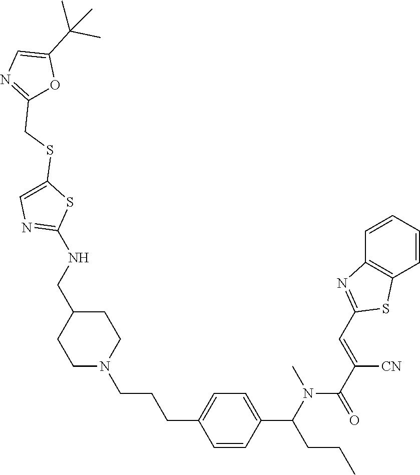 |

Table 4 shows the bifunctional molecules that were analysed in accordance with the procedure outlined in assay 2, in particular to determine the residual CDK9 abundance after 8 h of treatment with 100 nM of the bifunctional molecule.

In all cases, the residual CDK9 abundance after 8 h treatment with 100 nM of the bifunctional molecule was found to be less than 70%. Thus, all the above bifunctional molecules are considered to be effective degraders.

### Example 5

The degradation of target protein CDK9 was detected according to the procedure outlined in assay 4 for the following compounds.

| | |
|---|---|
| A44 | 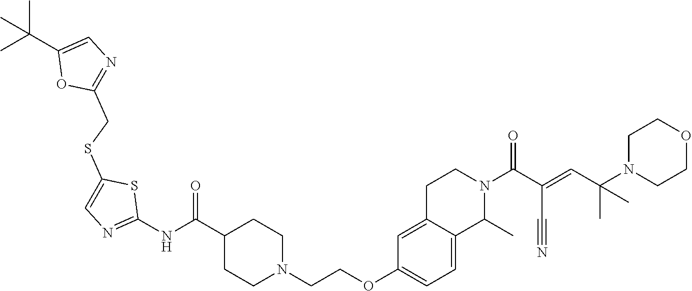 [SMILES: CC1c2ccc(OCCN3CCC(C(=O)Nc4ncc(SCc5ncc(C(C)(C)C)o5)s4)CC3)cc2CCN1C(=O)/C(C#N)=C/C(C)(C)N1CCOCC1] |
| A57 | 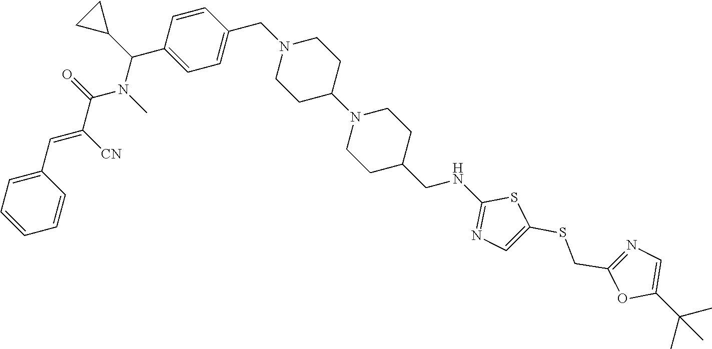 [SMILES: CN(C(=O)/C(C#N)=C/c1ccccc1)C(c1ccc(CN2CCC(N3CCC(CNc4ncc(SCc5ncc(C(C)(C)C)o5)s4)CC3)CC2)cc1)C1CC1] |
| A58 | 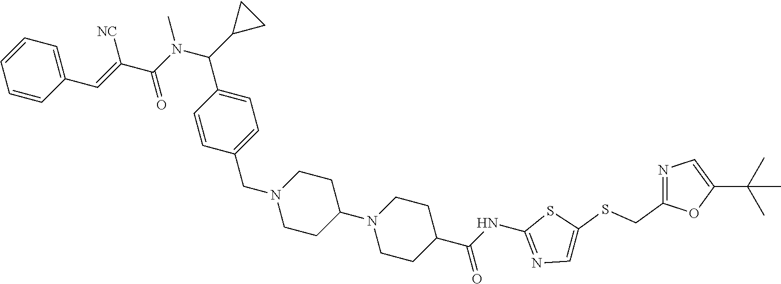 [SMILES: CN(C(=O)/C(C#N)=C/c1ccccc1)C(c1ccc(CN2CCC(N3CCC(C(=O)Nc4ncc(SCc5ncc(C(C)(C)C)o5)s4)CC3)CC2)cc1)C1CC1] |
| A45 | 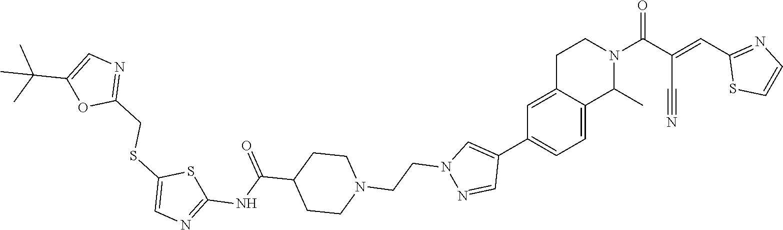 [SMILES: CC1c2ccc(-c3cnn(CCN4CCC(C(=O)Nc5ncc(SCc6ncc(C(C)(C)C)o6)s5)CC4)c3)cc2CCN1C(=O)/C(C#N)=C/c1nccs1] |
| A46 | 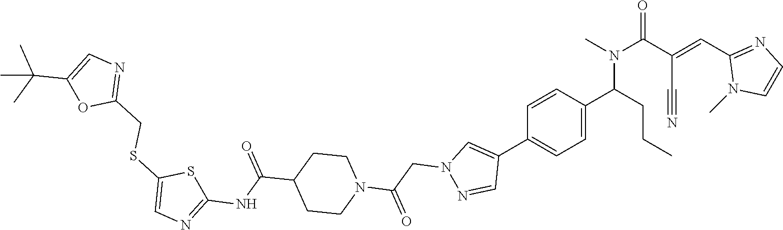 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1nccn1C] |
| A47 | 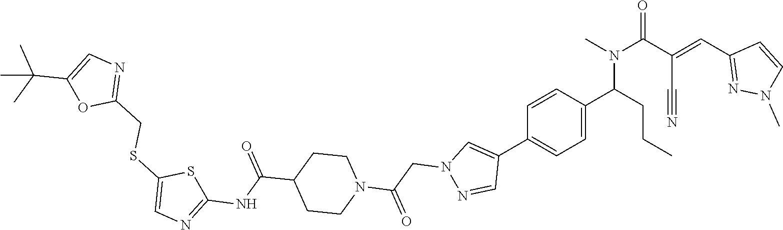 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ccn(C)n1] |
| A48 | 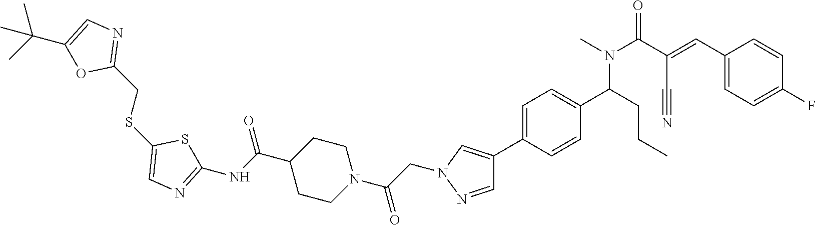 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ccc(F)cc1] |
| A49 | 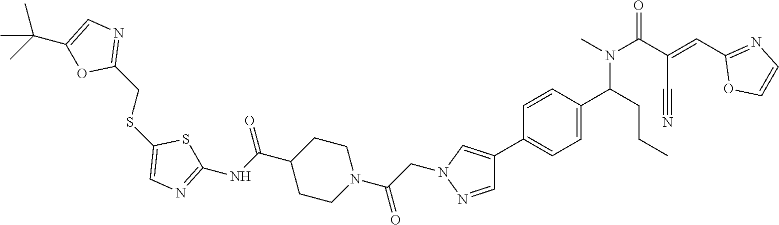 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1ncco1] |
| A50 | 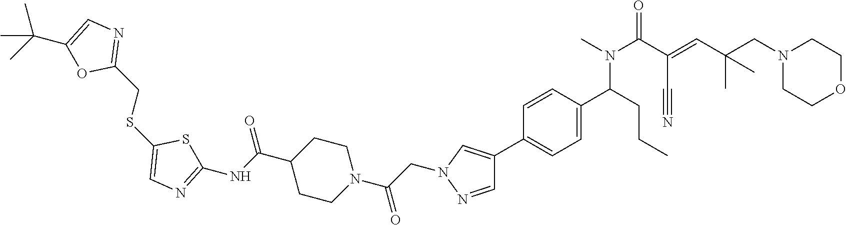 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/C(C)(C)CN1CCOCC1] |
| A51 | 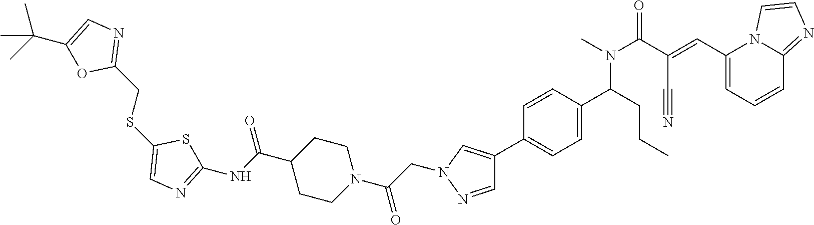 [SMILES: CCCC(c1ccc(-c2cnn(CC(=O)N3CCC(C(=O)Nc4ncc(SCc5ncc(C(C)(C)C)o5)s4)CC3)c2)cc1)N(C)C(=O)/C(C#N)=C/c1cccc2nccn12] |
| A56 | 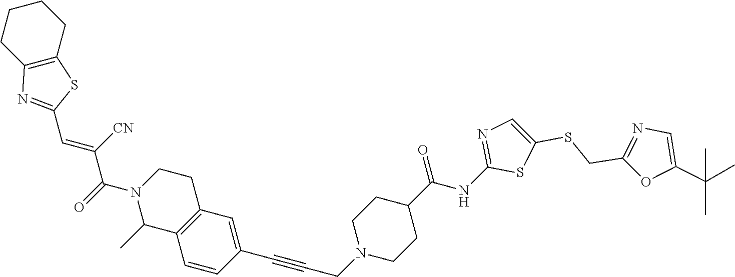 [SMILES: CC1c2ccc(C#CCN3CCC(C(=O)Nc4ncc(SCc5ncc(C(C)(C)C)o5)s4)CC3)cc2CCN1C(=O)/C(C#N)=C/c1nc2c(s1)CCCC2] |
| A54 | 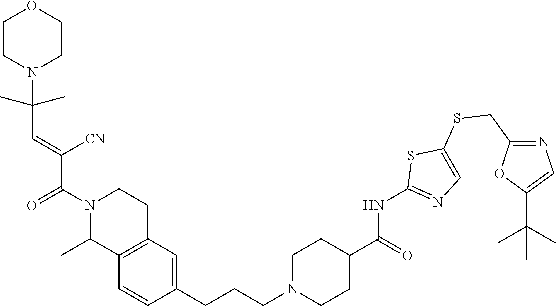 [SMILES: CC1c2ccc(CCCN3CCC(C(=O)Nc4ncc(SCc5ncc(C(C)(C)C)o5)s4)CC3)cc2CCN1C(=O)/C(C#N)=C/C(C)(C)N1CCOCC1] |
| A59 | 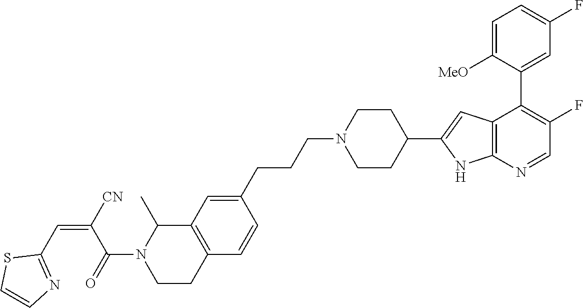 [SMILES: COc1ccc(F)cc1-c1c(F)cnc2[nH]c(C3CCN(CCCc4ccc5c(c4)C(C)N(C(=O)/C(C#N)=C\c4nccs4)CC5)CC3)cc12] |
| A53 | 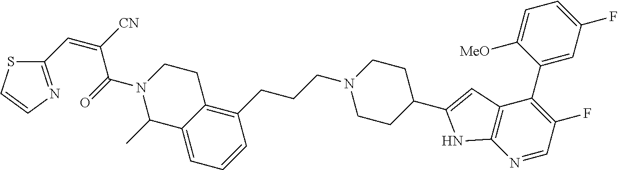 [SMILES: COc1ccc(F)cc1-c1c(F)cnc2[nH]c(C3CCN(CCCc4cccc5c4CCN(C(=O)/C(C#N)=C\c4nccs4)C5C)CC3)cc12] |
| A55 | 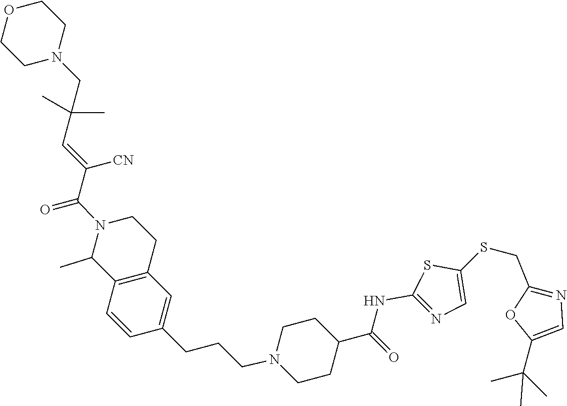 [SMILES: CC1c2ccc(CCCN3CCC(C(=O)Nc4ncc(SCc5ncc(C(C)(C)C)o5)s4)CC3)cc2CCN1C(=O)/C(C#N)=C/C(C)(C)CN1CCOCC1] |
| A60 | 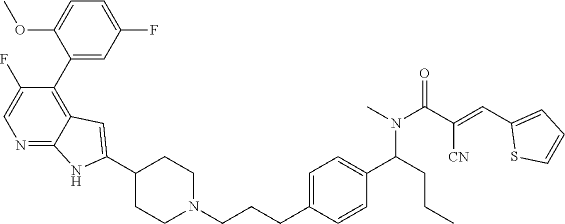 [SMILES: CCCC(c1ccc(CCCN2CCC(c3cc4c(-c5cc(F)ccc5OC)c(F)cnc4[nH]3)CC2)cc1)N(C)C(=O)/C(C#N)=C/c1cccs1] |

The DC₅₀ values for compounds in table 5 were found to be less than 1000 nM. These molecules are all considered to be effective degraders.

### Example 6

The ability of compounds to demonstrate in vivo systemic drug exposure after oral dosing in rodents was assessed using the following protocol:

Test compound was administered to C57BL/6 mice, (6-8 weeks, 18-20 g, female, N=6, purchased from JH Laboratory Animal Co. with free access to food and water) at the indicated dose level via oral gavage (p.o., 10 mL/kg, 5% DMSO+95%(15% HP-β-CD in Water) vehicle, oral dosing leg) or tail vein (i.v., 5 mL/kg, 5% DMSO+95%(15% HP-β-CD in Water) vehicle, intravenous leg). The animal was restrained manually at designated time points up to 24 h, approximately 110 μL of blood sample was collected via facial vein into K2EDTA tubes. Blood sample was put on ice and centrifuged at 2000 g for 5 min to obtain plasma sample within 15 minutes which was analysed on an LC-MS/MS-19 (Triple Quad 5500) in positive ion ESI mode following sample prep and HPLC elution:

Mobile Phase:
Mobile Phase A: H₂O—0.025% FA—1 mM NH₄OAC
Mobile Phase B: MeOH—0.025% FA—1 mM NH₄OAc

| Time (min) | Mobile Phase B (%) |
|---|---|
| 0.20 | 5 |
| 0.50 | 95 |
| 1.30 | 95 |
| 1.31 | 5 |
| 1.80 | stop |

Column: ACQUITY UPLC BEH C18 2.1*50 mm 1.7 um
Flow rate: 0.60 mL/min
Column temperature: 60° C.

Example A37 showed AUC_{(0-INF)} 841 hr*ng/mL following an intravenous 1 mg/kg dose and AUC_{(0-INF)} 605 hr*ng/mL following an oral 10 mg/kg dose to give an oral bioavailability of 7.2%

### Example 7

The ability of compounds to demonstrate in vivo drug exposure in the brain after systemic dosing in rodents was assessed using the following protocol:

Test compound was administered to C57BL/6 mice, (6-8 weeks, 18-20 g, female, N=6, purchased from JH Laboratory Animal Co. with free access to food and water) at the indicated dose level via tail vein (i.v., 5 mL/kg, 5% DMSO+95%(15% HP-β-CD in Water) vehicle).

Blood collection: The animal was restrained manually at designated time points up to 24 h, approximately 110 μL of blood sample was collected via facial vein into K2EDTA tubes. Blood sample was put on ice and centrifuged at 2000 g for 5 min (4° C.) to obtain plasma sample within 15 minutes.

Brain collection: The animal was euthanized under CO₂. A mid-line incision was made in the animal's scalp and skin retracted. The skull overlying the brain was removed. The whole brain was collected, rinsed with cold saline, dried on filtrate paper, weighted, snap frozen by placing into dry-ice. The sample was homogenized with 3 volumes (v/w) of PBS prior to analysis. Samples were analysed on an LC-MS/MS-19 (Triple Quad 5500) in positive ion ESI mode following sample prep and HPLC elution:

Mobile Phase:
Mobile Phase A: H₂O—0.025% FA—1 mM NH₄OAC
Mobile Phase B: MeOH—0.025% FA—1 mM NH₄OAc

| Time (min) | Mobile Phase B (%) |
|---|---|
| 0.20 | 5 |
| 0.60 | 95 |
| 1.20 | 95 |
| 1.21 | 5 |
| 1.80 | stop |

Column: waters BEH C18 (2.1×50 mm, 1.7 μm)
Flow rate: 0.60 mL/min
Column temperature: 60° C.

Example A39 showed a plasma AUC_{(0-INF)} 1336 hr*ng/mL and brain AUC_{(0-INF)} 845 hr*ng/mL following an intravenous 5 mg/kg dose showing a high level of brain exposure (brain:plasma 0.6).

This high level of brain exposure is in contrast to many other types of bifunctional degraders, such as CRBN and VHL PROTACs, which do not routinely allow for CNS (central nervous system) penetration.

### Example 8

The degradation of target protein PARP1 was detected according to the procedure outlined in assay 7 for the following compounds: A61, A62, A63.

In all cases, the residual PARP1 abundance after 24 h treatment with 100 nM of the bifunctional molecule was found to be less than 50%. Thus, all the above bifunctional molecules are considered to be effective degraders.

### Example 9

The degradation of target protein mutant KRas (G12C) was detected according to the procedure outlined in assay 8 for the compound A64.

The residual KRas (G12C) abundance after 24 h treatment with 1000 nM of the bifunctional molecule was found to be less than 50%. Thus, the bifunctional molecule is considered to be an effective degrader.

### Example 10

The degradation of target protein BRD9 was detected according to the procedure outlined in assay 5 for the compounds A65, A66.

The residual BRD9 abundance after 24 h treatment with 100 nM of the bifunctional molecule was found to be less than 50%. Thus, the above bifunctional molecules are considered to be effective degraders.

### Example 11

The degradation of target protein mutant EGFR (L858R) was detected according to the procedure outlined in assay 8 for the compound A67.

The residual EGFR (L858R) abundance after 24 h treatment with 1000 nM of the bifunctional molecule was found to be less than 50%. Thus, the bifunctional molecule is considered to be an effective degrader.

1. A bifunctional molecule comprising the general formula:
TBL-L-Z
wherein TBL is a target protein binding ligand;
L is a linker; and
Z comprises a structure according to formula (I): 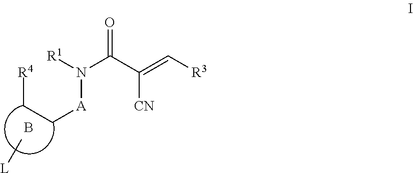
wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR²R^{2′};
B is selected from aryl, heteroaryl, substituted aryl and substituted heteroaryl;
R² and R^{2′} are each independently selected from H and C₁ to C₆ alkyl, optionally wherein the C₁ to C₆ alkyl is substituted with one or more heteroatoms selected from N, O or S, or wherein R² and R^{2′} together form a 3-, 4-, 5- or 6-membered carbocyclic or heterocyclic ring;
R³ is selected from C₁ to C₆ alkyl, aryl, heteroaryl, substituted aryl, substituted heteroaryl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
R⁴ is H, C₁ to C₆ alkyl, optionally wherein the C₁ to C₆ alkyl is substituted with one or more heteroatoms selected from N, O or S;
or wherein R¹ and R⁴ together form a 5-, 6-, or 7-membered heterocyclic ring;
or wherein when A is CR²R^{2′}:
R¹ and R² together form a 5-, 6-, or 7-membered heterocyclic ring; or
R² and R⁴ together form a 5-, 6-, or 7-membered heterocyclic or carbocyclic ring; and
L shows the point of attachment of the linker.

2. A bifunctional molecule according to claim 1, wherein:
(i) when R¹ and R⁴ together form a 5-, 6-, or 7-membered heterocyclic ring, Z is represented by formula (Ia): 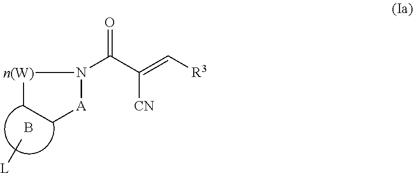
wherein A, B, R³ and L are as defined for formula (I); and
n is 1, 2 or 3;
W is selected from CR^{W1}R^{W2}, O, NR^{W3} and S;
R^{W1}, R^{W2} and R^{W3} are each independently selected from H and C₁ to C₆ alkyl; and wherein when n is 2 or 3, each W is independently selected from CR^{W1}R^{W2}, O, NR^{W3}, and S;
(ii) when R¹ and R² together form a 5-, 6-, or 7-membered heterocyclic ring, Z is represented as formula (Ib): 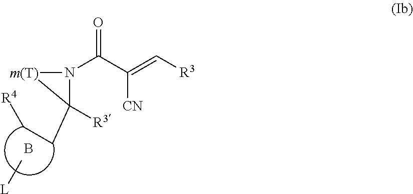
Wherein B, R^{2′}, R³, R⁴ and L are as defined for formula (I);
m is 3, 4 or 5;
each T is independently selected from CR^{T1}R^{T2}, O, NR^{T3} and S; and
R^{T1}, R^{T2} and R^{T3} are each independently selected from H and C₁ to C₆ alkyl; or
(iii) when R² and R⁴ together form a 5-, 6-, or 7-membered heterocyclic or carbocyclic ring, Z is represented as formula (Ic): 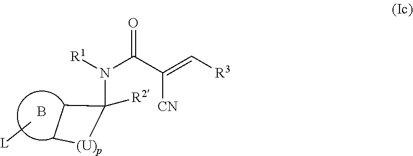
Wherein B, R¹, R^{2′}, R³ and L are as defined for formula (I);
p is 2, 3 or 4; and
each U is independently selected from CR^{U1}R^{U2}, O, NR^{U3} and S; and
R^{U1}, R^{U2} and R^{U3} are each independently selected from H and C₁ to C₆ alkyl.

3. The bifunctional molecule according to claim 1, wherein R³ is a heteroaryl, substituted heteroaryl, aryl, substituted aryl, or a C₁-C₆ alkyl substituted with a heterocyclic group,
optionally wherein R³ is selected from: 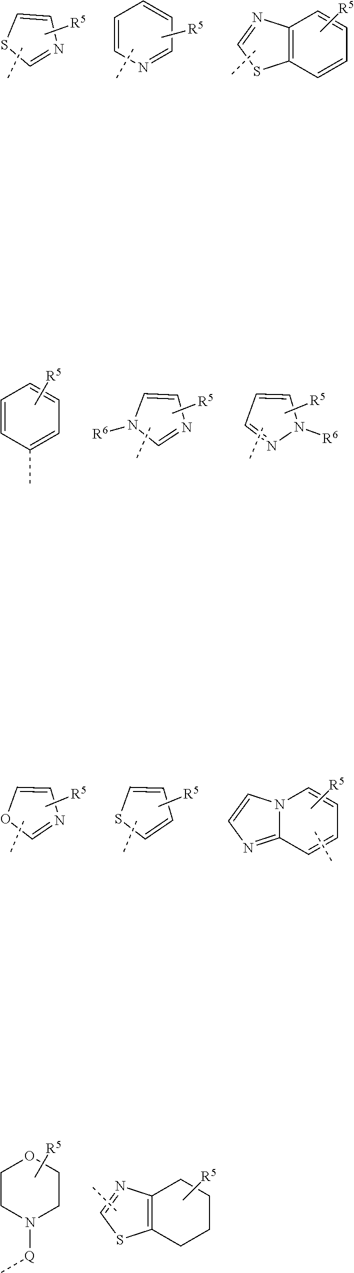
wherein the dotted line indicates the position at which each of the respective R³ groups is joined to the structure shown in formula (I) to (Ic), or wherein when the dotted line is not appended to an atom, the dotted line indicates that each of the respective R³ groups is joined to the structure via any position on the aromatic or heteroaromatic ring;
R⁵ is absent or is selected from halo, CF₃, —CH₂F, —CHF₂, C₁ to C₆ alkyl, —CN, —OH, —OMe, —SMe, —SOMe, —SO₂Me, —NH₂, —NHMe, —NMe₂, CO₂Me, —NO₂, CHO and COMe;
R⁶ is C₁ to C₆ alkyl; and
Q is C₁ to C₆ alkylene.

4. The bifunctional molecule according to claim 1, wherein A is CR²R^{2′}, optionally wherein one of R² and R^{2′} is a hydrogen and the other is C₁ to C₆ alkyl.

5. The bifunctional molecule according to claim 1, wherein B is a phenyl group.

6. The bifunctional molecule according to claim 1, wherein Z is represented as formula (IIaa): 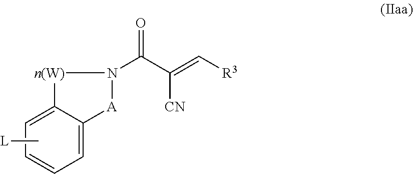
wherein A, R³, and L are as defined for formula (I);
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

7. The bifunctional molecule according to claim 1, wherein Z is represented as formula (IIa) 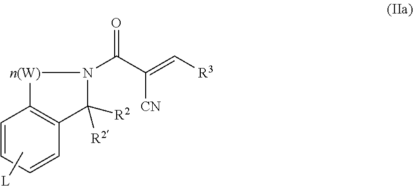
wherein R², R^{2′}, R³ and L are as defined in claim 2,
n is 1, 2 or 3; and
W is selected from CR^{W1}R^{W2}, Q, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, Q, NR^{W3}, and S.

8. The bifunctional molecule according to claim 1, wherein Z is represented as formula (IIb) 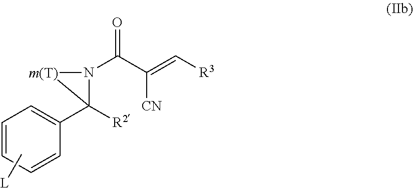
wherein R^{2′}, R³ and L are as defined in claim 1;
m is 3, 4 or 5; and
each T is independently selected from CR^{T1}R^{T2}, O, NR^{T3} and S; and
R^{T1}, R^{T2} and R^{T3} are each independently selected from H and C₁ to C₆ alkyl.

9. The bifunctional molecule according to any one of the preceding claim 1, wherein the structure of the linker (L) is:
(Lₓ)_{q}
wherein each Lx represents a subunit of L that is independently selected from CR^{L1}R^{L2}, O, C═O, S, SO, SO₂, NR^{L3}, SONR^{L4}, SONR^{L5}C═O, CONR^{L6}, NR^{L7}CO, C(R^{L8})═C(R^{L9}), C≡C, aryl, substituted aryl, heteroaryl, substituted heteroaryl, carbocyclyl, substituted carbocyclyl, heterocyclyl and substituted heterocyclyl groups;
wherein R^{L1}, R^{L2}, R^{L3}, R^{L4}, R^{L5}, R^{L6}, R^{L7}, R^{L8} and R^{L9} are each independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —SO₂(C₁ to C₆ alkyl), —CO₂(C₁ to C₆ alkyl), and —CO(C₁ to C₆ alkyl); and
q is an integer between 1 and 30.

10. The bifunctional molecule according to claim 1, wherein the target protein binding ligand (TBL) is selected from the group consisting of (i) binders to kinases, (ii) compounds binding to bromodomain-containing proteins, (iii) epigenetic modulator compounds, (iv) binders to transcription factors, (v) binders to GTPases, (vi) binders of phosphatases, (vii) binders of ubiquitin E3 ligases, (viii) immunosuppressive and immunomodulatory compounds, (ix) modulators of nuclear receptors, (x) binders to aggregation-prone proteins, (xi) binders to apoptotic & anti-apoptotic factors, and (xii) binders to polymerases.

11. A pharmaceutical composition comprising the bifunctional molecule according to claim 10, together with a pharmaceutically acceptable carrier, optionally wherein the bifunctional molecule is present in the composition as a pharmaceutically acceptable salt, solvate or derivative.

12. The bifunctional molecule according to claim 10, for use in medicine.

13. The bifunctional molecule for use of claim 12, wherein the use comprises the treatment and/or prevention of any disease or condition which is associated with and/or is caused by an abnormal level of protein activity.

14. The bifunctional molecule of claim 12, for use in the treatment and/or prevention of cancer.

15. A method of selectively degrading and/or increasing proteolysis of a target protein in a cell, the method comprising contacting and/or treating the cell with a bifunctional molecule as defined in claim 1.

16. A method of selectively degrading and/or increasing proteolysis of a target protein in a subject in need thereof, the method comprising administering to the subject a therapeutically effective amount of a bifunctional molecule as defined in claim 1.

17. Use of a moiety Z as defined in any one of formula (I) to (IIa) in a method of targeted protein degradation.

18. Use of a moiety Z as defined in any one of formula (I) to (IIa) in the manufacture of a bifunctional molecule suitable for targeted protein degradation.

19. A compound comprising the Z moiety according to formula (IV): 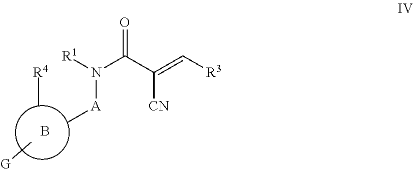
wherein A, B, R¹, R³ and R⁴ are as defined in claim 1; and
G is configured to enable attachment of the Z moiety to another chemical structure via formation of a new covalent bond.

20. A compound comprising the structure:
L-Z
wherein Z is as defined in claim 8; and
L is a linker.

21. A method of making a bifunctional molecule as defined in claim 1.

22. A method of obtaining bifunctional molecules according to claim 1, comprising:
a. providing a bifunctional molecule comprising:
(i) a first ligand comprising a structure according to Z as defined in any one of claims 1 to 8;
(ii) a second ligand that binds to a target protein; and
(iii) a linker that covalently attaches the first and second ligands;
b. contacting a cell with the bifunctional molecule;
c. detecting degradation of the target protein in the cell;
d. detecting degradation of the target protein in the cell in the absence of the bifunctional molecule; and
e. comparing the level of degradation of the target protein in the cell contacted with the bifunctional molecule to the level of degradation of the target protein in the absence of the bifunctional molecule;
wherein an increased level of degradation of the target protein in the cell contacted with the bifunctional molecule indicates that the bifunctional molecule has facilitated and/or promoted the degradation of the target protein,
optionally wherein detecting degradation of the target protein comprises detecting changes in the levels of the target protein in the cell.

23. A compound library comprising a plurality of bifunctional molecules according to claim 1.
